SEQUENCE LISTING (3 sequences)

SEQ ID NO: 1 - DNA, 57 bp, synthetic construct
  misc_feature 1..57: note = WNT8B targeting shRNA
  source 1..57: mol_type = other DNA
gatccccatgaaacgcacgtgtaattcaagagattacacgtgcgtttcatggttttt

SEQ ID NO: 2 - DNA, 57 bp, synthetic construct
  misc_feature 1..57: note = WNT8B targeting shRNA
  source 1..57: mol_type = other DNA
gatcccctagcctatcaacccttattcaagagataagggttgataggctaggttttt

SEQ ID NO: 3 - DNA, 57 bp, synthetic construct
  misc_feature 1..57: note = WNT8B targeting shRNA
  source 1..57: mol_type = other DNA
gatccctctggagtgctaacttgattcaagagatcaagttagcactccagagttttt

(19) United States
(12) Patent Application Publication
(10) Pub. No.: US 2026/0297514 A1
(43) Pub. Date: Oct. 1, 2026

(54) EFFICIENT, SCALABLE PRODUCTION OF HUMAN RETINAL PROGENITORS IN VITRO

(71) Applicant: Wisconsin Alumni Research Foundation, Madison, WI (US)

(72) Inventors: Su-Chun Zhang, Waunakee, WI (US); Yunlong Tao, Madison, WI (US)

(21) Appl. No.: 19/374,772

(22) Filed: Oct. 30, 2025

Related U.S. Application Data
(62) Division of application No. 16/876,353, filed on May 18, 2020.
(60) Provisional application No. 62/849,294, filed on May 17, 2019.

(51) Int. Cl. C12N 5/0793; C12N 15/85; G01N 33/50

(52) U.S. Cl. CPC C12N 5/062; C12N 15/85; G01N 33/5058; C12N 2500/32; C12N 2503/00; C12N 2506/02; C12N 2506/45

(57) ABSTRACT

Provided herein are efficient, scalable methods for in vitro production of human retinal progenitor cells. In addition, provided herein are methods for isolating pure populations of in vitro produced human retinal progenitor cells as well as kits and compositions comprising such pure populations of human retinal progenitor cells.

### CROSS-REFERENCE TO RELATED APPLICATIONS

This application is a divisional application of U.S. patent application Ser. No. 16/876,353, filed May 18, 2020, which claims priority to U.S. Provisional Application No. 62/849,294, filed May 17, 2019, which is incorporated herein by reference in its entirety.

### STATEMENT REGARDING FEDERALLY FUNDED RESEARCH OR DEVELOPMENT

This invention was made with government support under NS086604 awarded by the National Institutes of Health. The government has certain rights in the invention.

### SEQUENCE LISTING

This application is being filed electronically via EFS-Web and includes an electronically submitted Sequence Listing in xml format. The .txt file contains a sequence listing entitled “20-1403-US-DIV_Sequence Listing.xml” created on Jul. 23, 2025 and is 5 kilobytes in size. The Sequence Listing contained in this .xml file is part of the specification and is hereby incorporated by reference herein in its entirety.

### BACKGROUND

Retinal diseases, especially those caused by degeneration of photoreceptor cells, affect a large population worldwide. Studies on the disease process are limited by the available animal models which often do not reflect the nature of the disease in human patients. Consequently, current treatments focus primarily on alleviating symptoms through pharmacological and surgical interventions.

Human pluripotent stem cells, including induced pluripotent stem cells (iPSCs) from patients with retinal diseases and those genetically modified to carry retinal diseases, offer a cellular model for investigating the retinal disease process and a source for regenerative therapy. The key step is to generate highly enriched, bona fide retinal cells. However, current methods produce a mixture of cells that contain a small population of retinal cells (<30%) and a much larger population of neural cells and often require manual selection of retinal progenitors based on morphological criteria. This hinders large-scale, standardized production of retinal cells from human stem cells for disease modeling, drug development, and cell therapies. Accordingly, there remains a need in the art for large-scale, standardized production of retinal cells from hPSCs and neural stem cells (NSCs, also known as neuroepithelial cells) for disease modeling, drug development, and cell therapy-based treatment options that regenerate the lost photoreceptors.

### SUMMARY OF THE DISCLOSURE

In a first aspect, provided herein is a method of producing a substantially pure population of human retinal progenitor cells. The method can comprise or, in some cases, consist essentially of the steps of: (a) culturing human pluripotent stem cells (hPSCs) in suspension culture for about 6 days in a neural induction medium whereby embryoid bodies are formed, wherein the neural induction medium is supplemented with N2 supplement and Non-Essential Amino Acid (NEAA) cell culture supplement beginning on culturing day 3; (b) dissociating the embryoid bodies formed in step (a) into a single cell suspension; (c) culturing the single cell suspension as an adherent monolayer for about 15 to about 22 days in a retinal differentiation medium, whereby a substantially pure population comprising human retinal progenitor cells is obtained. The method can further comprise sorting the cell population of (c) to isolate Pax6D-expressing human retinal progenitor cells from non-Pax6D-expressing cells. The Pax6D-expressing human retinal progenitor cells can be selected and sorted based on expression of a Pax6D-reporter construct. The neural induction medium can be a chemically defined medium comprising DMEM/F-12. The neural induction medium can be E8 medium. The retinal differentiation medium can be a chemically defined medium comprising DMEM/F-12, B27 supplement, and NEAA cell culture supplement. The method can further comprise introducing into the hPSCs an agent that reduces expression of WNT8B and increases expression of retinal progenitor-specific genes. The agent can be a WNT8B short hairpin interfering RNA (shRNA).

In another aspect, provided herein is a substantially pure population of human retinal progenitor cells comprising an exogenous nucleotide sequence encoding a detectable reporter operably linked to a nucleotide sequence encoding human pax6D.

In another aspect, provided herein is a method of testing a compound. The method can comprise or, in some cases, consist essentially of contacting a test compound to the human retinal progenitor cells obtained according to methods of this disclosure and examining the effect of the compound on the cells.

In another aspect, provided herein is a substantially pure population of human retinal progenitor cells obtained according to a method of this disclosure.

In a further aspect, provided herein is a kit for differentiating human pluripotent stem cells into human retinal progenitor cells. The kit can comprise or, in some cases, consist essentially of one or more of (i) a neural induction medium; (ii) a retinal differentiation medium; (iii) a PAX6D reporter construct; (iv) reagents for genetic modification of cells to achieve inducible expression of Pax6D; (v) an agent that reduces expression of WNT8B; and (vi) instructions describing a method for generating substantially pure populations of human retinal progenitor cells, the method employing one or more of the culture medium, the PAX6D reporter construct, the genetic modification reagents, and the agent. The agent can be a WNT8B short hairpin interfering RNA (shRNA). The retinal differentiation medium can be chemically defined medium comprising DMEM/F-12, B27 supplement, and NEAA cell culture supplement. The neural induction medium can be E8 medium.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The present invention will be better understood and features, aspects and advantages other than those set forth above will become apparent when consideration is given to the following detailed description thereof. Such detailed description makes reference to the following drawings, wherein:

FIG. 1 is a schematic illustration of a protocol for directed differentiation of retinal progenitor cells.

FIGS. 2A-2C demonstrate expression of PAX6 isoforms in forebrain and retinal development. Panel (A) provides a schematic diagram of the PAX6 isoforms in human. Panel (B) demonstrates Western blot detection of PAX6 isoforms in human fetal forebrain and retina. The antibody used targets the C terminus of PAX6 and can detect all the 3 isoforms at different molecular weight. The green arrow indicates the PAX6A&B isoforms. The red arrow indicates the PAX6D isoform. Panel (C) demonstrates Western blot detection of PAX6 isoforms in cells during forebrain and retina differentiation from hESCs. D10 indicates day 10 of differentiation.

FIGS. 3A-3E demonstrate effects of PAX6 knockout on retinal differentiation. Panel (A) provides a schematic strategy for generating PAX6 knockout in hESCs. The exon 8 is shared by all the isoforms and was replaced by the PGK-Puromycin element. The primers indicated in the pane are used to detect the gene knockout. Panel (B) demonstrates qPCR quantification of PAX6 expression in Wildtype and PAX6 KO during retinal differentiation. All the data are presented as mean±SD in figures. “*” indicates the p value is less than 0.05. “**” indicates the p value is less than 0.01. “***” indicates the p value is less than 0.001. Panel (C) demonstrates Western blot detection of PAX6 in Wildtype and PAX6 KO cells at day 20 of retinal differentiation. (D) Immunostaining of PAX6 in Wildtype and PAX6 KO cells reveals that C′-PAX6 antibody detects PAX6A, B and D, while N′-PAX6 antibody detects PAX6 A and B but not D. Scale bars indicate 50 μm. Panel (E) demonstrates qPCR quantification of retinal related genes in Wildtype and PAX6 KO cells. Samples were collected at day 6, 10, 14 and 19 after the start of differentiation.

FIGS. 4A-4J demonstrate generation of PAX6 isoform specific knockouts and their effects on retinal differentiation. (A) Schematic diagram for generating PAX6D knockout (PAX6D KO). The exon α was replaced by PGK-Puromycin. Primers indicated in this panel are used to detect the PAX6D KO. (B) qPCR verification of PAX6D KO efficiency. PAX6A&B and PAX6D isoforms were also evaluated by qPCR with isoform specific primers. (C) Western blot detection of PAX6 isoforms in wildtype and PAX6D KO cells. (D) qPCR quantification of retinal gene VSX2 in PAX6D KO cells. (E) qPCR quantification of retinal gene SIX6 in PAX6D KO cells. (F) Immunostaining of VSX2 in wildtype and PAX6D KO cells. Scale bar indicates 100 μm. (G) GO analysis of differentially expressed genes in PAX6 KO cells. Up-regulated genes and down-regulated genes are analyzed separately. The biological processes are ranked by p-value. Only top 15 terms were shown. (H) GO analysis of differentially expressed genes in PAX6D KO cells. Up-regulated genes and down-regulated genes are analyzed separately. The biological processes are ranked by p-value. Only the top 15 terms were shown. (I) Venn diagram shows the overlap of differentially regulated genes in PAX6 KO and PAX6D KO cells. The numbers indicate the number of genes in each area. (J) GO analysis of biological process for overlap genes in panel I. The numbers in the cycle are the ratio of genes. The GO terms are ranked by p-value in the clockwise direction.

FIGS. 5A-5E demonstrate that PAX6A&B and PAX6D play different roles in retina differentiation. (A) Immunostaining of SOX1 and SOX2 in human fetal forebrain and retinal tissues. Scale bars indicate 50 μm. (B) Flow cytometry analysis of day 7 cell cultures under retinal differentiation from wildtype, PAX6 KO, and PAX6D KO cell lines. (C) Flow cytometry analysis of day 20 cell cultures under retinal differentiation from wildtype, PAX6 KO, and PAX6D KO cell lines. (D) Quantification of SOX1/SOX2+ ratio in panel C. (E) SOX1 mRNA level in wildtype, PAX6 KO, and PAX6D KO cell at day 20 of retinal differentiation.

FIGS. 6A-6F demonstrate effects of PAX6A&B and PAX6D on RPC differentiation. (A) Schematic diagram for generating PAX6A (PAX6A TetOn in PAX6KO) and PAX6D (PAX6D TetOn in PAX6 KO) inducible cell lines in PAX6 KO hESCs. With this design, PAX6A or PAX6D is induced separately with the doxycycline (DOX) treatment. (B) qPCR quantification of PAX6A and PAX6D gene expression under the treatment of DOX. (C) Western blot analysis of PAX6A and PAX6D protein expression under the treatment of DOX. (D) qPCR quantification of SOX1 gene expression in the PAX6A TetOn and PAX6D TetOn cells. (E) Expression of retinal marker expression in PAX6D TetOn cells without DOX treatment. (F) Cell population composition analysis with SOX1 and SOX2 staining from flow cytometry. SOX1/SOX2 double positive cells represent the neuroepithelial fate while the SOX1 negative and SOX2 positive cells represent the neuroretinal fate.

FIGS. 7A-7D demonstrate identification of PAX6D targets by ChIP-seq. (A) Experimental design for the ChIP samples. PAX6A TetOn and PAX6D TetOn cells were differentiated towards the retinal lineage. At day 6 the cells were treated with DOX to turn on PAX6A or PAX6D expression. After 3 days of treatment, the cells were collected at day 10 and used for ChIP experiment. (B) The fragment enrichment evaluated by SES plot. (C) GO analysis of the PAX6A targets and PAX6D targets identified by ChIP-Seq. PAX6A only, PAX6A and PAX6D shared, and PAX6D only targets are analyzed separately. (D) Verification of PAX6D target genes using PAX6D TetOn cells. Two sets of genes (neural and retinal) are directly regulated by PAX6D in opposite directions.

FIGS. 8A-8E demonstrate retinal specification regulated by PAX6D is WNT dependent. (A) qPCR quantification of WNT8B in PAX6D KO cells at multiple time points during retinal differentiation. (B) qPCR quantification of retinal gene expression under the effect of WNT agonist CHIR99021 and WNT antagonist IWR1 on retinal differentiation. (C) qPCR quantification of retinal gene expression in PAX6D KO cells treated with WNT antagonist IWR1 to rescue the retinal differentiation. (D) qPCR quantification of retinal gene expression in PAX6D TetOn cells when PAX6D was induced by DOX in the presence of WNT agonist CHIR99021. (E) A model describing the role of a unique function for PAX6D isoform in regulating retinal specification. Data demonstrate that the choice between retinal versus forebrain fate is controlled by PAX6D via regulation of a set of neural and retinal genes.

FIGS. 9A-9F present schematic diagrams of (A) primer design for PAX6 isoforms, (B) a human forebrain differentiation protocol from hPSC, and (C) a human retinal differentiation protocol from hPSC. Also demonstrated are (D) overall PAX6 gene expression during human forebrain and retinal differentiation, (E) PAX6A&B isoform expression during human forebrain and retinal differentiation, an (F) PAX6D isoform expression during human forebrain and retinal differentiation. All the data are presented as mean±SD.

FIGS. 10A-10C demonstrate (A) PCR genotyping of PAX6 KO candidate colonies after puromycin screening, (B) flow cytometry analysis of PAX6 expression in day 6 retinal differentiation cells from wildtype and PAX6 KO cells, and (C) immunostaining of VSX2 and PAX6 in differentiating retinal cells from wildtype and PAX6 KO.

FIGS. 11A-11B demonstrate (A) PCR genotyping of PAX6D KO candidate colonies after puromycin screening, and (B) a Venn diagram showing the overlap of differentially regulated genes in PAX6 KO and PAX6D KO cells. 290 genes showed only in PAX6 KO/wildtype group were further analyzed by gene ontology enrichment. The GO terms were ranked by p-value in the clockwise direction.

FIG. 12 demonstrates immunostaining of SOX1 and PAX6 in human fetal retinal and forebrain tissue. Scale bars indicate 50 μm.

FIG. 13A demonstrates immunostaining of SOX1 and PAX6 in retinal differentiating cells from PAX6A TetOn and PAX6D TetOn cell lines without DOX. Scale bars indicate 100 μm.

FIG. 13B demonstrates (B) Immunostaining of SOX2 and PAX6 in retinal differentiating cells from PAX6A TetOn and PAX6D TetOn cell lines without DOX. Scale bars indicate 100 μm.

FIG. 14 is a schematic illustrating construction of a PAX6D reporter line. PAX6D transcription is controlled by promoter alpha which contains the intron after exon4, exon α and exon 5. PAX6D translation starts at exon 7. PAX6D coding sequence is replaced by tdTomato coding sequence, and the resulting construct is inserted in the AAVSI site of the genome. tdTomato will be expressed in cells in which Pax6D is expressed.

FIGS. 15A-15B demonstrate the effects on generation of retinal cells of repressing WNT8B expression in human pluripotent stem cells.

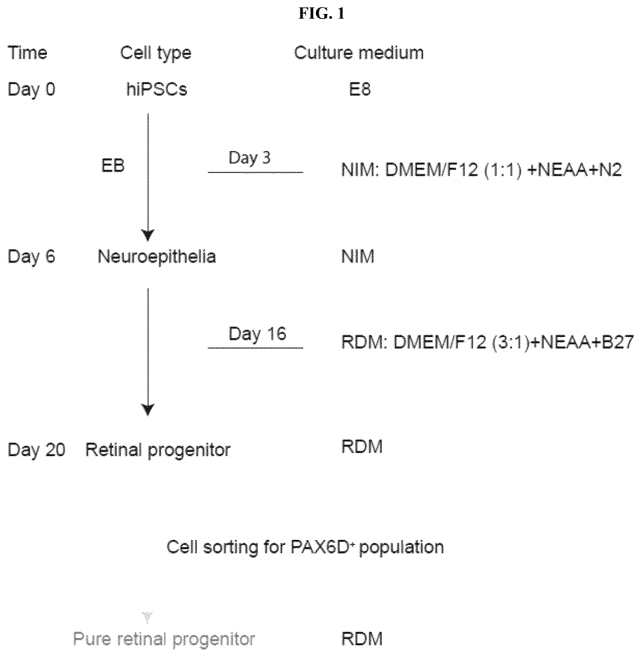

### DETAILED DESCRIPTION

The methods and compositions provided herein are based at least in part on the inventors' development of efficient, scalable methods for generating highly enriched retinal progenitor cells (RPC) from human pluripotent stem cells (hPSCs). In particular, the inventors identified a PAX6 isoform, PAX6D, as being necessary and sufficient to guide differentiation of neural stem cells to produce RPCs. The inventors further uncovered the molecular pathways that are targeted by PAX6D. Existing methods for generating retinal progenitor cells (RPCs) is to induce human stem cells to neural stem cells (NSCs or neuroepithelia) first and then guide NSCs to RPCs. Because of the lack of knowledge and tool to turn NSCs to RPCs, the methods produce a mixture of cells that contain a small population of retinal cells and a much larger population of neural cells. It often requires manual selection of RPCs based on morphological criteria such as “optic cup-like” appearance. Even so, cell populations obtained by these conventional differentiation methods comprise less than 30% RPCs. In comparison, the methods and compositions of this disclosure are advantageous over conventional RPC differentiation methods. In particular, the methods and compositions described herein enable large-scale, standardized production of human retinal cells from hPSCs and neural stem cells for disease modeling, drug development, and cell therapies.

### I. Definitions

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

In describing the embodiments and claiming the invention, the following terminology will be used in accordance with the definitions set out below.

As used herein, the term “pluripotent stem cell” (hPSC) means a cell capable of continued self-renewal and of capable, under appropriate conditions, of differentiating into cells of all three germ layers. hPSCs exhibit a gene expression profile that includes SOX2⁺ and OCT4⁺. Examples of human PSCs (hPSCs) include human embryonic stem cells (hESCs) and human induced pluripotent stem cells (hiPSCs). As used herein, “iPS cells” or “iPSCs” refer to cells that are substantially genetically identical to their respective differentiated somatic cell of origin and display characteristics similar to higher potency cells, such as ES cells, as described herein. The cells can be obtained by reprogramming non-pluripotent (e.g., multipotent or somatic) cells.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “chemically defined culture medium” or “chemically defined medium,” as used herein, means that the molecular identity, chemical structure, and quantity of each medium ingredient is definitively known. The term “ingredient,” as used herein, refers to a component the molecular identity and quantity of which is known. In some cases, a chemically defined medium is made xeno-free, and incorporate human proteins, which can be produced using recombinant technology or derived from placenta or other human tissues in lieu of animal-derived proteins. In some embodiments, all proteins added to the medium are recombinant proteins.

As used herein, “a medium consisting essentially of” means a medium that contains the specified ingredients and those that do not materially affect its basic characteristics.

“Supplemented,” as used herein, refers to a composition, e.g., a medium comprising a supplemented component (e.g., B27, N2). For example a medium “further supplemented” with B27 or N2 supplement, refers to the medium comprising B27 or N2 supplement, and not to the act of introducing the B27 or N2 supplement to the medium.

The terms “purified” or “enriched” cell populations are used interchangeably herein, and refer to cell populations, in vitro or ex vivo, that contain a higher proportion of a specified cell type or cells having a specified characteristic than are found in vivo (e.g., in a tissue).

As used herein, “serum-free” means that a medium does not contain serum or serum replacement, or that it contains essentially no serum or serum replacement. For example, an essentially serum-free medium can contain less than about 1%, 0.9%, 0.8%, 0.7%, 0.6%, 0.5%, 0.4%, 0.3%, 0.2% or 0.1% serum, wherein the culturing capacity of the medium is still observed.

### II. Methods

Accordingly, in a first aspect, this disclosure provides in vitro methods for efficiently and robustly producing retinal progenitor cells (RPCs), preferably RPCs suitable for use in drug screening applications and for regenerative cell therapies. The methods enable scalable, industrial production of enriched or purified human RPCs.

In certain embodiments, an in vitro method of producing human RPCs comprises or consists essentially of the following steps: (a) culturing human pluripotent stem cells (hPSCs) for about 6 days in a neural differentiation medium whereby embryoid bodies are formed, wherein the neural differentiation medium is supplemented with N-2 supplement and Non-Essential Amino Acid (NEAA) cell culture supplement beginning on culturing day 3; (b) dissociating the embryoid bodies formed in step (a) into a single cell suspension; and (c) culturing the single cell suspension in an adherent monolayer for between 15 days and 22 days in a retinal differentiation medium, whereby a cell population comprising human retinal progenitor cells is obtained.

As used herein the term “embryoid bodies” (EBs) refers to three-dimensional multicellular aggregates of differentiated and undifferentiated cells derivatives of three embryonic germ layers. EBs can be obtained by any suitable method. In some cases, human pluripotent cells are cultured under conditions that promote the formation of stem cell aggregates and spontaneous formation of EBs, which contain a mixture of undifferentiated and differentiated cell types of the three primary germ layers. In some cases, hPSCs are cultured in low adhesion tissue culture dishes or in liquid suspension culture to promote aggregate formation. As used herein, the term “suspension culture” refers the low adhesion or liquid suspension culture conditions under which pluripotent stem cells are cultured to promote aggregation of the pluripotent stem cells and spontaneous formation of EBs from the aggregates.

Upon formation, EBs are gradually transitioned from a chemically defined basal culture medium (e.g., E8 medium) to a neural induction medium (NIM) that comprises or consists essentially of DMEM/F12 (1:1), 1% N2 supplement, and 1× Non-Essential Amino Acid (NEAA) supplement, by replacing the chemically defined basal culture medium with a 3:1 ratio of E8 medium to NIM on retinal differentiation day 1, a ratio of 1:1 on day 2, and 100% NIM on day 3. EBs are cultured in NIM for another 4 days. On retinal differentiation day 7, EBs were seeded onto plates with NIM containing 5% fetal bovine serum (FBS). The medium was changed to serum-free NIM one day later, and the EBs were fed with fresh NIM every other day. On differentiation day 16, NIM was removed and replaced with retinal differentiation medium (RDM) that comprises or consists essentially of DMEM/F12 (3:1) supplemented with 2% B27 supplement (without vitamin A) and 1×NEAA, with daily medium changes. On day 20 (after about 4 days of culture in RDM), cells were treated with trypsinLE to detach the cells and to obtain a single cell suspension.

In some cases, the neural induction medium is a chemically defined medium. “Neural induction medium,” as used herein, refers to a medium capable of promoting and supporting differentiation of human pluripotent stem cells towards a neural lineage, e.g., towards neuroectoderm and neuroepithelium. A neural induction base medium can include, but is not limited to E6 medium, as described herein and in U.S. Patent Publication No. 2014/0134732. Preferably, the chemically defined medium comprises DMEM/F-12. In some cases, the neural induction medium is E8 medium. As used herein, the terms “E8 culture medium” and “E8” are used interchangeably and refer to a chemically defined culture medium comprising or consisting essentially of DF3S supplemented by the addition of insulin (20 μg/mL), transferrin (10.67 ng/ml), human FGF2 (100 ng/mL), and human TGFβ1 (Transforming Growth Factor Beta 1) (1.75 ng/mL). The medium can be prepared based on the formula in previous publication (Chen et al., (2011) Nature Methods. 8 (4), 424-429). As an alternative, the medium is also available from Thermal Fisher/Life Technologies Inc. as Essential 8, or from Stem Cell Technologies as TeSR-E8.

As used herein, the term “N2 Supplement” (also known as N-2 Supplement) refers to a chemically-defined, serum-free nutritional supplement. In some cases, N2 supplement is added to a basal culture medium such as DMEM. As used herein, the term “B27 Supplement” refers to a serum-free nutritional supplement that promotes long term survival of in vitro cultured neurons. N2 supplement and B27 supplement are available from various commercial vendors such as ThermoFisher.

As used herein, “pluripotent stem cells” appropriate for use according to a method of the invention are cells having the capacity to differentiate into cells of all three germ layers. Suitable pluripotent cells for use herein include human embryonic stem cells (hESCs) and human induced pluripotent stem (iPS) cells. As used herein, “embryonic stem cells” or “ESCs” mean a pluripotent cell or population of pluripotent cells derived from an inner cell mass of a blastocyst. See Thomson et al., Science 282:1145-1147 (1998). These cells express Oct-4, SSEA-3, SSEA-4, TRA-1-60 and TRA-1-81, and appear as compact colonies having a high nucleus to cytoplasm ratio and prominent nucleolus. ESCs are commercially available from sources such as WiCell Research Institute (Madison, Wis.). As used herein, “induced pluripotent stem cells” or “iPS cells” mean a pluripotent cell or population of pluripotent cells that may vary with respect to their differentiated somatic cell of origin, that may vary with respect to a specific set of potency-determining factors and that may vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318:1917-1920 (2007).

Induced pluripotent stem cells exhibit morphological properties (e.g., round shape, large nucleoli and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60 or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natal individual.

Subject-specific somatic cells for reprogramming into induced pluripotent stem cells can be obtained or isolated from a target tissue of interest by biopsy or other tissue sampling methods. In some cases, subject-specific cells are manipulated in vitro prior to use in a three-dimensional tissue construct of the invention. For example, subject-specific cells can be expanded, differentiated, genetically modified, contacted to polypeptides, nucleic acids, or other factors, cryo-preserved, or otherwise modified prior to differentiation into retinal progenitor cells according to the methods of this disclosure.

Preferably, human pluripotent stem cells (e.g., human ESCs or iPS cells) are cultured in the absence of a feeder layer (e.g., a fibroblast layer), a conditioned medium, or a culture medium comprising poorly defined or undefined components. As used herein, the terms “chemically defined medium” and “chemically defined cultured medium” also refer to a culture medium containing formulations of fully disclosed or identifiable ingredients, the precise quantities of which are known or identifiable and can be controlled individually. As such, a culture medium is not chemically defined if (1) the chemical and structural identity of all medium ingredients is not known, (2) the medium contains unknown quantities of any ingredients, or (3) both. Standardizing culture conditions by using a chemically defined culture medium minimizes the potential for lot-to-lot or batch-to-batch variations in materials to which the cells are exposed during cell culture. Accordingly, the effects of various differentiation factors are more predictable when added to cells and tissues cultured under chemically defined conditions. As used herein, the term “serum-free” refers to cell culture materials that are free of or substantially free of serum obtained from animal (e.g., fetal bovine) blood.

In some embodiments, any of the above-referenced cells are cultured in a xeno-free cell culture medium. Of central importance for clinical therapies is the absence of xenogeneic materials in the derived cell populations, i.e., no non-human cells, cell fragments, sera, proteins, and the like. Culturing cells or tissues in the absence of animal-derived materials (i.e., under conditions free of xenogeneic material) reduces or eliminates the potential for cross-species viral or prion transmission.

Prior to culturing hPSCs (e.g., hESCs or hiPSCs) under suspension conditions that promote embryoid body formation, hPSCs can be cultured in the absence of a feeder layer (e.g., a fibroblast layer) on a substrate suitable for proliferation of hPSCs, e.g., Matrigel®, vitronectin, a vitronectin fragment, or a vitronectin peptide, or Synthemax®. In some cases, the hPSCs are passaged at least 1 time to at least about 5 times in the absence of a feeder layer. Suitable culture media for passaging and maintenance of hPSCs include, but are not limited to, mTeSR® and E8™ media. In some embodiments, the hPSCs are maintained and passaged under xeno-free conditions, where the cell culture medium is a chemically defined medium such as E8 or mTeSR, but the cells are maintained on a completely defined, xeno-free substrate such as human recombinant vitronectin protein or Synthemax® (or another type-of self-coating substrate). In one embodiment, the hPSCs are maintained and passaged in E8 medium on human recombinant vitronectin or a fragment thereof, a human recombinant vitronectin peptide, or a chemically defined self-coating substrate such as Synthemax®.

Any appropriate method can be used to detect expression of biological markers characteristic of cell types described herein. For example, the presence or absence of one or more biological markers can be detected using, for example, RNA sequencing, immunohistochemistry, polymerase chain reaction, qRT-PCR, or other technique that detects or measures gene expression. Suitable methods for evaluating the above-markers are well known in the art and include, e.g., qRT-PCR, RNA-sequencing, and the like for evaluating gene expression at the RNA level. Differentiated cell identity is also associated with downregulation of pluripotency markers such as NANOG and OCT4 (relative to human ES cells or induced pluripotent stem cells). Quantitative methods for evaluating expression of markers at the protein level in cell populations are also known in the art. For example, flow cytometry is typically used to determine the fraction of cells in a given cell population that express (or do not express) a protein marker of interest (e.g., PAX6). In some cases, cell populations obtained by the RPC differentiation methods of this disclosure comprise at least 80%, 85%, 90%, 95% and preferably at least 98% RPCs.

As used herein, “gene expression” refers to the relative levels of expression and/or pattern of expression of a gene in a biological sample, such as retinal progenitor cells, or population of cells comprising retinal progenitor cells. The expression of a gene, such as a biological marker (“biomarker”) of retinal differentiation (e.g., VSX2, HES5, LHX2, NR2E1, RAX, and VAX2), may be measured at the level of cDNA, RNA, mRNA, or combinations thereof. In some cases, altered expression a gene, such as a retinal progenitor biomarker such as VSX2, HES5, LHX2, NR2E1, RAX, and VAX2, is measured at the protein level. In some cases, the level of gene expression or protein expression of a biomarker of interest is multiple fold (e.g., 2, 3, 4, 5, 10, 20, 50, 100) higher or lower than in untreated cells or in cells treated with a control agent.

In some cases, a Pax6D reporter construct is used to sort human RPCs from other cell types. For example, a reporter construct (such as a construct encoding a detectable protein or protein fragment) is operably linked to the human pax6D promoter, whereby the reporter construct is expressed under control of the pax6D promoter. Since Pax6D is uniquely expressed in retinal progenitor cells, expression of the reporter construct is indicative of a Pax6D-positive cell and, thus, a retinal progenitor cell. If the differentiation methods provided herein are performed using human pluripotent stem cells or neuroepithelial cells comprising the Pax6D reporter construct, the resulting cell population comprising retinal progenitor cells (RPCs) can be sorted on the basis of reporter expression. For example, the pax6d promoter is fused with tdTomato and mix cell populations can be detected and sorted on the basis of tdTomato expression in order to enrich for human retinal progenitors.

As used herein, the term “operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

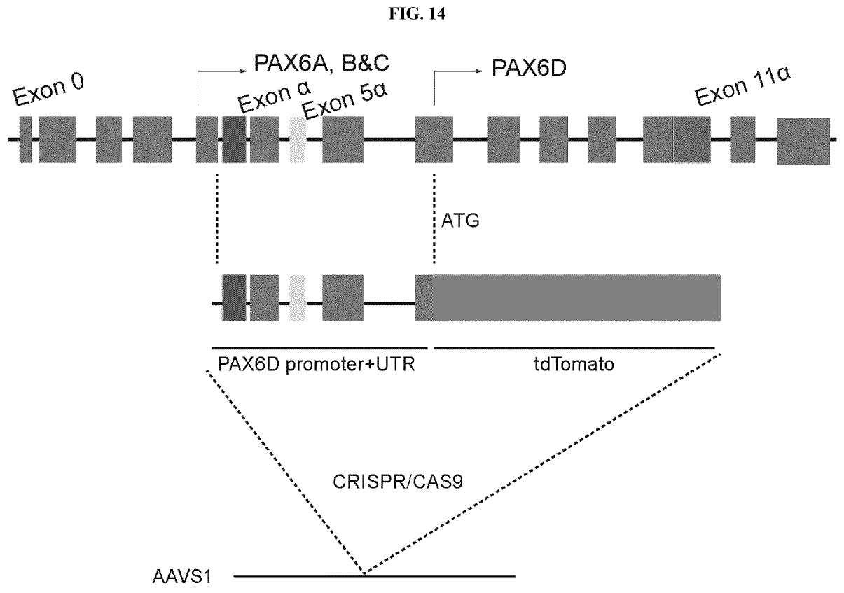

In another aspect, provided herein are methods for promoting neuroepithelial cells to differentiate along the human retinal cell lineage, thereby producing a high proportion of retinal progenitor cells from precursors. The methods of this disclosure include genetic and non-genetic ways of manipulating Pax6D expression at the neuroepithelial stage to promote differentiation of RPCs. In some cases, neuroepithelial cells are genetically modified for inducible expression of Pax6D. In some cases, the RPCs express a detectable label or reporter for cell sorting. Retinal progenitor cells (the Pax6D-positive cells) are sorted from Pax6D-negative cells from a cell population produced according to the differentiation protocols provided herein in order to produce a pure or substantially pure population of RPCs. In this method, progenitor cells (e.g., human pluripotent stem cells, neuroepithelial cells) are genetically manipulated to express a reporter construct, for example a transgene that encodes a detectable marker such a Green Fluorescent Protein (GFP) or a Red Fluorescent Protein (RFP) (td-Tomato). Referring to FIG. 14, a PAX6D reporter line can be constructed by inserting a reporter construct such as tdTomato coding sequence upstream of the PAX6D translation start site in exon 7 of the human Pax6D gene. The resulting construct is inserted in the AAVSI site of the genome.

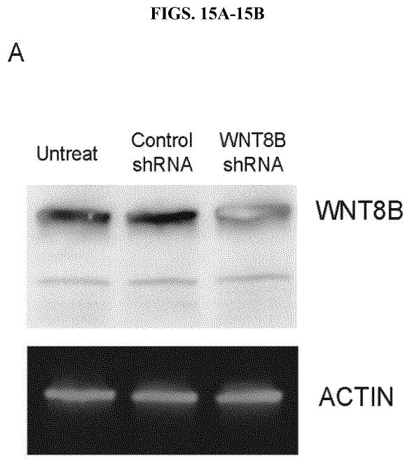

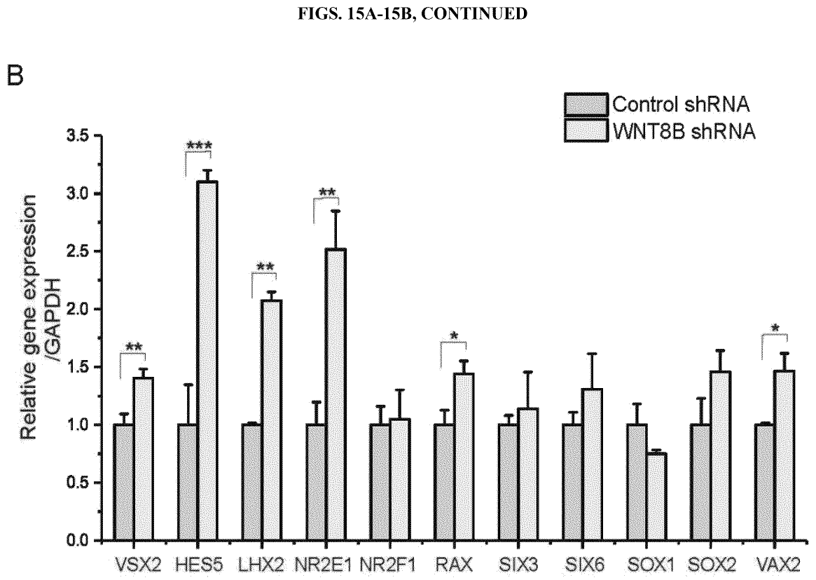

Referring to FIGS. 15A-15B, inhibition of expression of a downstream mediator of PAX6D signal transduction, and a resulting increase in expression of retinal progenitor biomarkers, can be achieved using RNA interference. In some cases, RNA interference is used to reduce expression of WNT8B. For example, one or more short hairpin interfering RNAs (shRNAs) configured to target WNT8B can be introduced into a cell (e.g., a human pluripotent stem cell) to reduce WNT8B expression. Such WNT8B shRNAs may be introduced into cells as synthetic shRNAs by a number of standard methods known in the art. Alternatively, shRNA sequences may be expressed from an expression vector, e.g., from a plasmid expression vector, a recombinant retrovirus, or a recombinant lentivirus. The expression of a biological marker (“biomarker”) of retinal differentiation (e.g., VSX2, HES5, LHX2, NR2E1, RAX, and VAX2), may be measured at the level of cDNA, RNA, mRNA, or combinations thereof. In some cases, altered expression a retinal progenitor biomarker such as VSX2, HES5, LHX2, NR2E1, RAX, and VAX2, is measured at the protein level. In some cases, the level of gene expression or protein expression of VSX2, HES5, LHX2, NR2E1, RAX, and VAX2 is multiple fold (e.g., 2, 3, 4, 5, 10, 20, 50, 100) higher than in cells not treated with an agent that reduces expression of WNT8B as described herein.

The term “detect” or “detection” as used herein indicates the determination of the existence, presence or fact of a target or signal in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through visually detectable signals: fluorescence, spectra, or images from a target of interest or a label attached to the target.

Establishment of stepwise and chemically defined culture systems for directed differentiation of human pluripotent stem cells to RPCs offers an unprecedented system for screening toxic and therapeutic agents. This system is preferable to conventional use of animals, animal cell cultures, or genetically abnormal human cell lines, particularly because human pluripotent stem cells and their differentiation to RPCs represent a normal process of human retinal development. Hence, the cell populations described herein will be amenable to screen agents that affect normal human retinal development or those that potentially result in abnormal retinal development, as well as those that may stimulate regeneration of RPCs in diseased conditions. In addition, the described system can be readily modified to mimic pathological processes that lead to death of RPCs, which may be effectively used to screen therapeutic agents that are designed to treat these diseases.

Accordingly, in another aspect, provided herein are methods of screening in vitro generated human RPCs. For example, human RPCs obtained according to the methods of this disclosure can be exposed to a test compound and examined for changes in gene expression, developmental characteristics, or other characteristics relative to a control cell population that has not been exposed to the test compound. One could understand whether a particular test compound affected the cell population by examining characteristics of the culture and comparing them to known developmental characteristics contained within the present application.

In some cases, using the genetic tagging and isolating methods described herein, pure populations of reporter-expressing RPCs can be screening to identify agents (e.g., small molecules, drugs, protein factors) that modulate Pax6D expression. Agents identified in the screen that increases Pax6D expression relative to controls can be used to promote Pax6D expression and, thus, RPC differentiation during retinal differentiation. For example, one may derive a small molecule cocktail to specifically activate PAX6D at particular stage of retinal cell differentiation.

In some cases, it may be advantageous to genetically modify RPCs obtained according to the methods provided herein. For example, it can be advantageous in some instances to obtain recombinant and genetically-modified RPCs that produce recombinant cell products, growth factors, hormones, peptides or proteins for a continuous amount of time or as needed when biologically, chemically, or thermally signaled due to the conditions present in culture. In some cases, genetic modifications are produced using a form of gene editing. The term “gene editing” and its grammatical equivalents as used herein can refer to genetic engineering in which one or more nucleotides are inserted, replaced, or removed from a genome. For example, gene editing can be performed using a nuclease (e.g., a natural-existing nuclease or an artificially engineered nuclease). In some cases, gene editing is performed using a CRISPR/cas system (e.g., a type II CRISPR/cas system). In some cases, the protein expression of one or more endogenous genes is reduced using a CRISPR/cas system. In other cases, a CRISPR/Cas system can be used to perform site specific insertion. For example, a nick on an insertion site in the genome can be made by CRISPR/cas to facilitate the insertion of a transgene at the insertion site. Other methods of making genetic modifications suitable for use according to the methods provided herein include but are not limited to somatic cell nuclear transfer (SCNT) and introduction of a transgene. As used herein, the term “transgene” refers to a gene or genetic material that can be transferred into an organism or a cell thereof. Procedures for obtaining recombinant or genetically modified cells are generally known in the art, and are described in Sambrook et al, Molecular Cloning, A Laboratory Manual, Cold Spring Harbor Press, Cold Spring Harbor, N.Y. (1989), incorporated herein by reference.

In another aspect, provided herein is a use of RPCs comprising a PAX6D reporter as described herein for drug screening, drug discovery, or drug response. For example, one could expose such cell populations provided herein to a test compound and compare the results of such exposure to a control cell population that has not been exposed to the test compound. One could understand whether a particular test compound affected the cell population by examining characteristics of the culture and comparing them to known developmental characteristics of retinal lineage cells. In some cases, screening comprises detecting a positive or negative change in a particular biological property or activity of a RPC. In some cases, detecting and/or measuring a positive or negative change in a level of expression of at least one gene following exposure (e.g., contacting) of a RPC to a test compound comprises whole transcriptome analysis using, for example, RNA sequencing. In such cases, gene expression is calculated using, for example, data processing software programs such as Light Cycle, RSEM (RNA-seq by Expectation-Maximization), Excel, and Prism. See Stewart et al., PLoS Comput. Biol. 9: e1002936 (2013). In some cases, detecting comprises performing a method such as RNA sequencing, gene expression profiling, transcriptome analysis, metabolome analysis, detecting reporter or sensor, protein expression profiling, Förster resonance energy transfer (FRET), metabolic profiling, and microdialysis.

In another aspect, retinal progenitor cells obtained according to the disclosed methods may be further differentiated to other cell types such as photoreceptors, which could be used clinically to treat or prevent degenerative eye diseases.

### III. Articles of Manufacture

In another aspect, provided herein is a kit for generating substantially pure populations of human retinal progenitor cells. In exemplary embodiments, the kit comprises one or more of (i) a culture medium suitable for differentiating human pluripotent stem cells into retinal progenitor cells; (ii) a PAX6D reporter construct; (iii) reagents for genetic modification of cells to achieve inducible expression of Pax6D; (iv) an agent that reduces expression of WNT8B; and (v) instructions describing a method for generating substantially pure populations of human retinal progenitor cells, the method employing one or more of the culture medium, the PAX6D reporter construct, the genetic modification reagents, and the agent.

In some cases, the kit comprises one or more of (i) a neural induction medium; (ii) a retinal differentiation medium; (iii) a PAX6D reporter construct; (iv) reagents for genetic modification of cells to achieve inducible expression of Pax6D; (v) an agent that reduces expression of WNT8B; and (vi) instructions describing a method for generating substantially pure populations of human retinal progenitor cells, the method employing one or more of the neural induction medium, the retinal differentiation medium, the PAX6D reporter construct, the genetic modification reagents, and the agent.

In some cases, the materials described above as well as other materials can be packaged together in any suitable combination as a kit useful for performing, or aiding in the performance of, a method provided herein. It is useful if the kit components in a given kit are designed and adapted for use together in the disclosed method. For example, disclosed herein are kits comprising genetically modified human RPCs produced by the disclosed methods.

Nucleic acids and/or other constructs of the invention may be isolated. As used herein, “isolated” means to separate from at least some of the components with which it is usually associated whether it is derived from a naturally occurring source or made synthetically, in whole or in part. In some embodiments, kits also can contain a cell culture medium, labels, and/or other reagents for the cell culture and detection of biological markers, polypeptides, or nucleic acids of interest in the genetically modified RPCs.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions (e.g., cell population) described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

In interpreting this disclosure, all terms should be interpreted in the broadest possible manner consistent with the context. Unless defined otherwise, all technical and scientific terms used herein have the same meaning as commonly understood by one of ordinary skill in the art to which the invention belongs. It is understood that certain adaptations of the invention described in this disclosure are a matter of routine optimization for those skilled in the art, and can be implemented without departing from the spirit of the invention, or the scope of the appended claims.

As used in this specification and the appended claims, the singular forms “a,” “an,” and “the” include plural references unless the context clearly dictates otherwise. Any reference to “or” herein is intended to encompass “and/or” unless otherwise stated.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The terms “comprising”, “comprises” and “comprised of as used herein are synonymous with “including”, “includes” or “containing”, “contains”, and are inclusive or open-ended and do not exclude additional, non-recited members, elements, or method steps. The phraseology and terminology used herein is for the purpose of description and should not be regarded as limiting. The use of “including,” “comprising,” “having,” “containing,” “involving,” and variations thereof, is meant to encompass the items listed thereafter and additional items. Embodiments referenced as “comprising” certain elements are also contemplated as “consisting essentially of” and “consisting of” those elements. Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

The terms “about” and “approximately” shall generally mean an acceptable degree of error for the quantity measured given the nature or precision of the measurements. Typical, exemplary degrees of error are within 10%, and preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The invention will be more fully understood upon consideration of the following non-limiting Examples. It is specifically contemplated that the methods disclosed are suited for pluripotent stem cells generally. All papers and patents disclosed herein are hereby incorporated by reference as if set forth in their entirety.

### EXAMPLE

### PAX6 Isoforms are Differentially Expressed in the Forebrain and Retinas

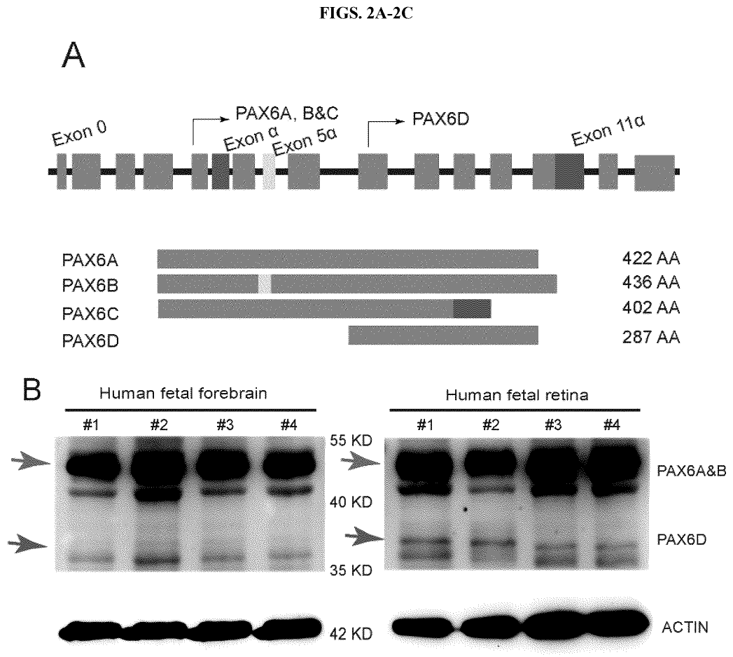

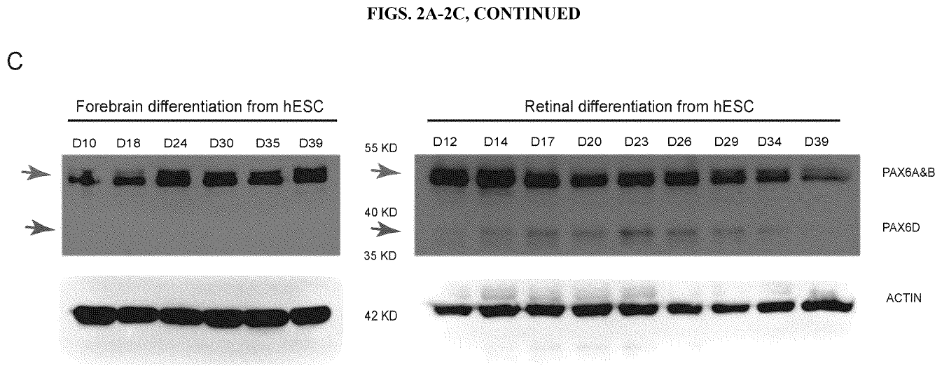

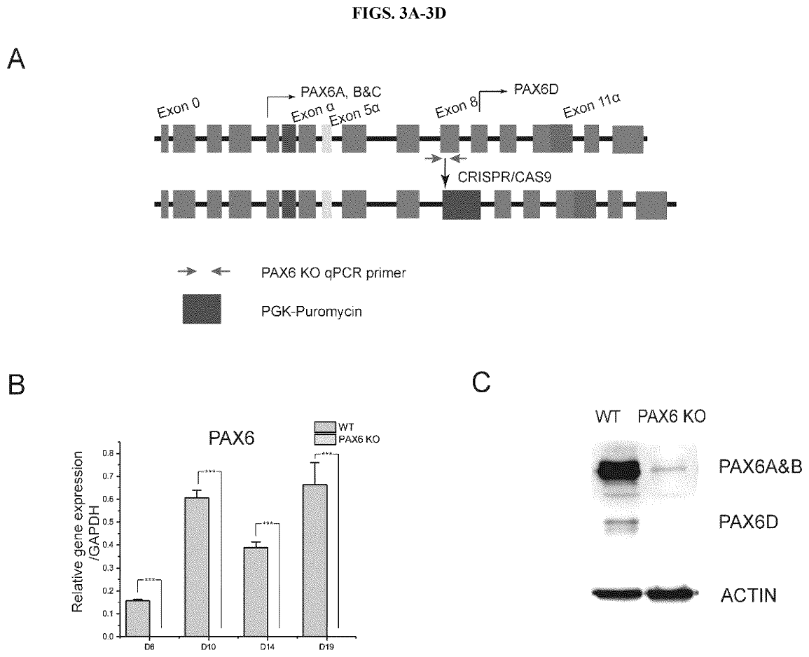

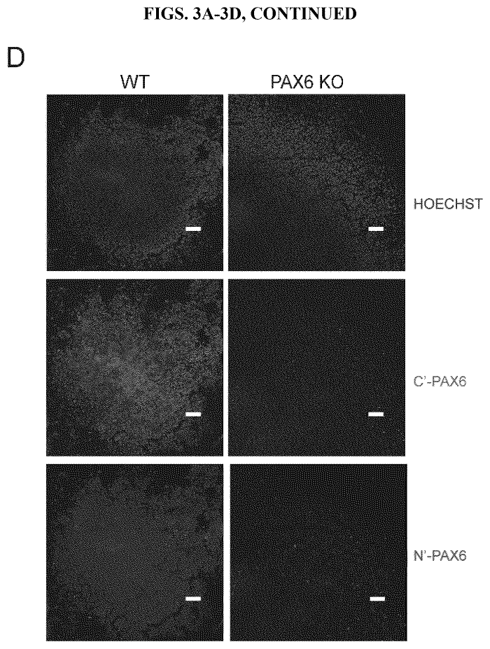

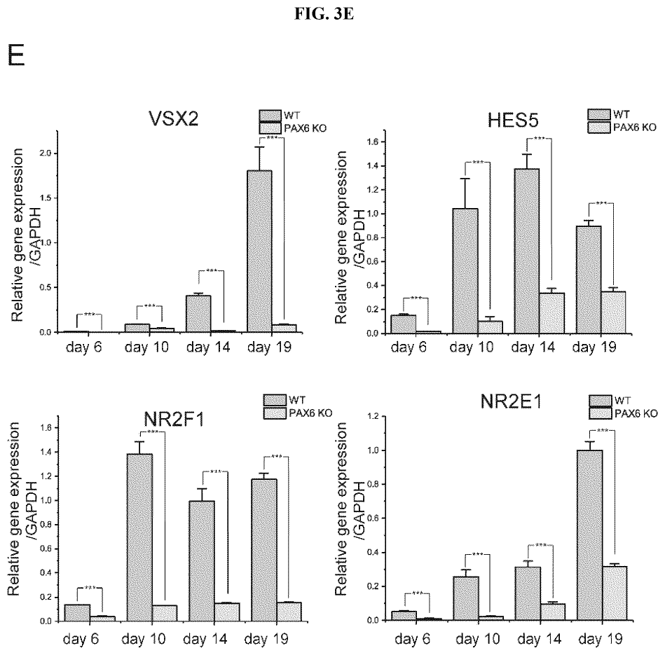

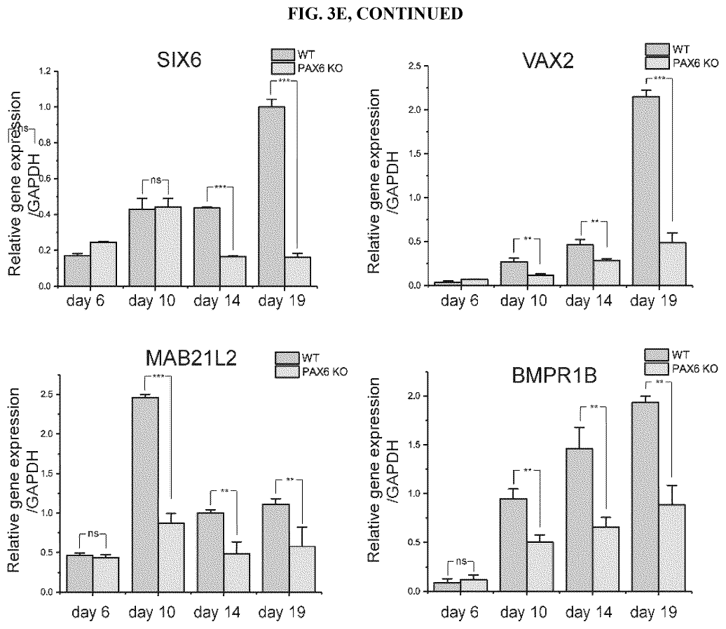

PAX6 has 4 isoforms (PAX6A, PAX6B, PAX6C and PAX6D) in human (FIG. 2A) and 3 isoforms (Pax6, Pax6 (5a) and Pax6ΔPD) in mice. The protein sequences of PAX6A, PAX6B and PAX6D (homolog of Pax6, Pax6 (5a) and Pax6ΔPD, respectively) are highly conserved among species, indicating their important and conserved functions. Western blotting analysis on 12-week-old embryos revealed that PAX6 isoforms exhibited differential expression patterns in human fetal forebrain and retinas. PAX6D was expressed uniquely in human fetal retinal tissues while PAX6A&B isoforms were expressed in both human fetal forebrain and retinal tissues (FIG. 2B), suggesting a unique role of PAX6D in retinal development.

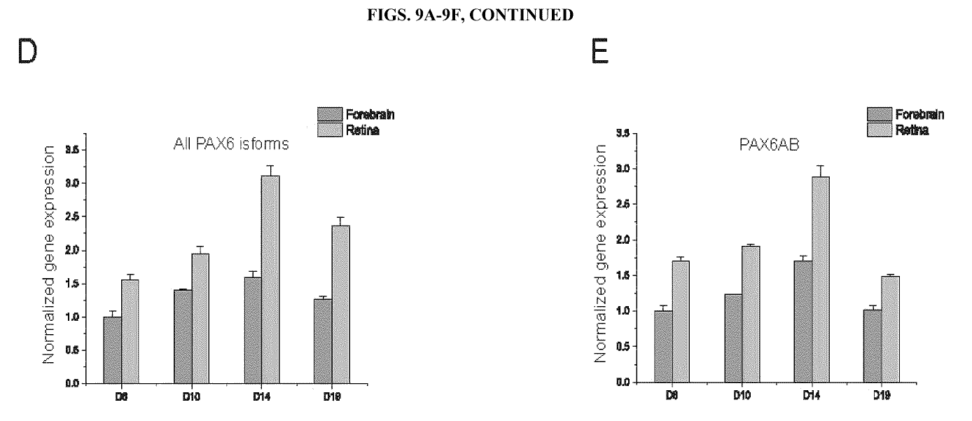

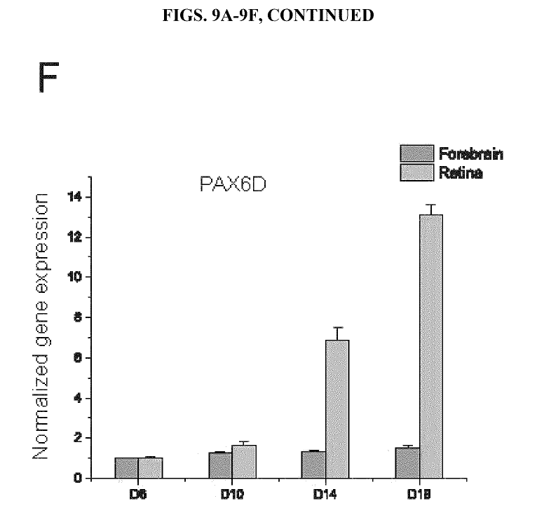

Human pluripotent stem cell differentiation in vitro provides an ideal model to study the early human neural development (Tao and Zhang, 2016). By differentiating H9 hESCs to forebrain and retinal cells (Wang et al., 2015; Zhong et al., 2014) (FIGS. 9B-9C), we found that overall PAX6 mRNA increased during the differentiation to both forebrain and retinal cells (FIG. 9D). Consistent with the previous gene profile study (Huang et al., 2016), we found that PAX6A and PAX6B increased in both forebrain and retinal differentiation (FIG. 9E). Interestingly, PAX6D isoform showed a differential expression pattern between forebrain and retinal differentiation. It was expressed only in retinal differentiation but not in the forebrain neuroepithelial differentiation (FIG. 2C & FIG. 9F). Western blotting using a PAX6 antibody targeting the C terminus, which can detect both PAX6A&B and PAX6D, validated the expression pattern of PAX6A&B and D isoforms. In particular, PAX6D was detected only in retinal differentiation. It began to express at day 12, reaching a peak at day 23, and then decreasing along differentiation (FIG. 2C). Thus, PAX6 isoforms are differentially expressed in forebrain neuroepithelia and retinas during development and hESC differentiation with PAX6D specifically in retinal progenitors.

PAX6 KO Prevents hESCs from Entering the Neural Retinal Fate

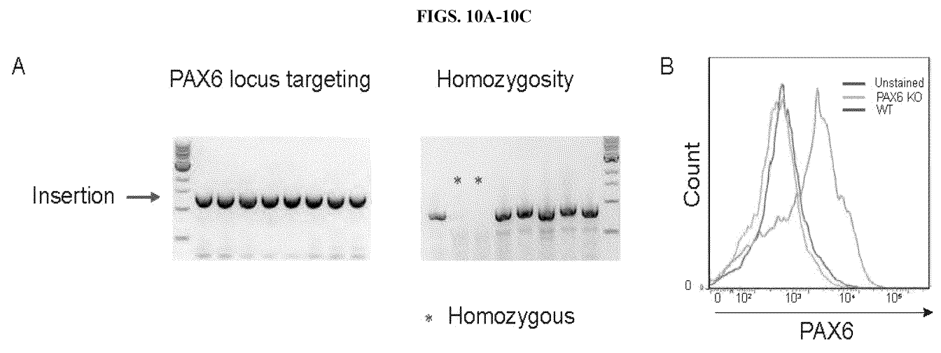

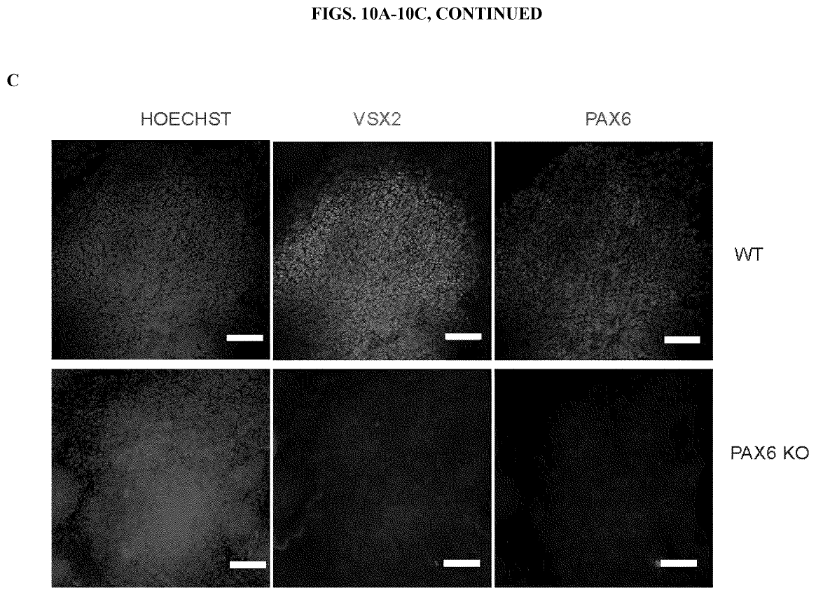

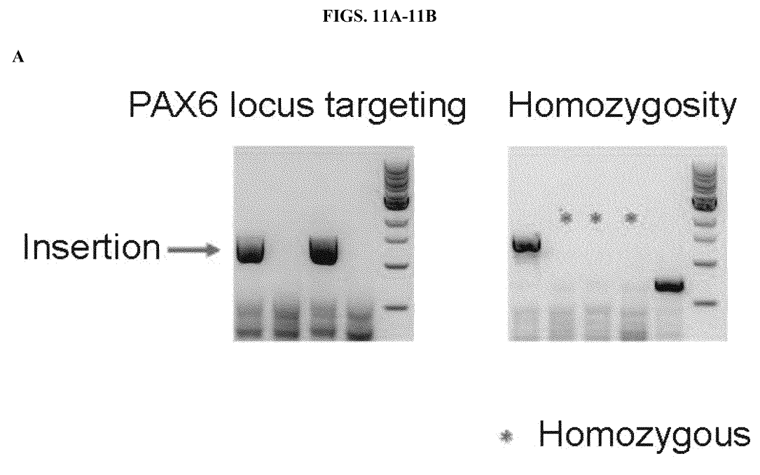

The differential expression pattern of PAX6 isoforms suggests their disparate roles in neural and retinal development. We firstly generated PAX6 KO hESC lines by targeting exon 8 which is shared by all the isoforms in H9 hESC line using CRISPR/CAS9 (FIG. 3A). The PAX6 KO line lose all the 3 isoforms after the exon 8 was replaced with the PGK-puromycin element (FIG. 10A). PAX6 mRNA levels were undetectable at multiple time points along retinal differentiation (FIG. 3B). Western blot showed that both the PAX6A&B and PAX6D isoforms were removed in PAX6 KO cells (FIG. 3C). Antibodies targeting the N-terminus and C-terminus failed to detect any PAX6 protein expression in the differentiating cells from the PAX6 KO line (FIG. 3D). These data demonstrate that our PAX6 KO line is loss of all the PAX6 isoforms.

qRT-PCR analysis revealed that the expression of retinal lineage markers, including VSX2 (formerly Chx10), VAX2, HES5, SIX6, MAB21L2, NR2E1 and NR2F1, were dramatically lower in the PAX6 KO cells (FIG. 3E). Some of them (VSX2, VAX2, SIX6, NR2E1 and NR2F1) were not upregulated during the retinal differentiation process. In particular, VSX2, critical for retinal development, was absent in PAX6 KO cells (FIG. 3E), which was confirmed by immunostaining (FIG. 10C), indicating a failure in retinal specification.

### PAX6D is Required for NR Specification

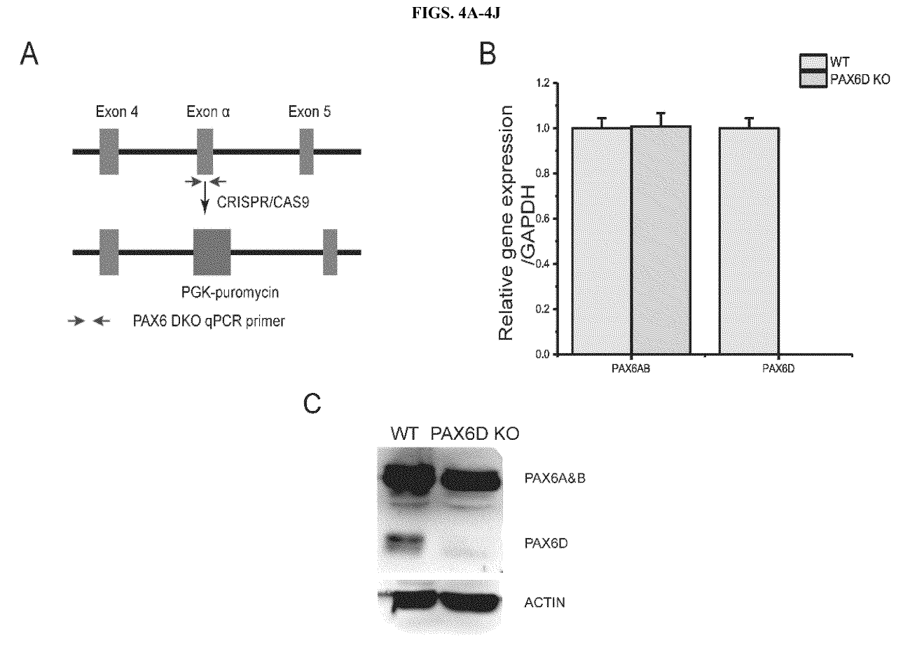

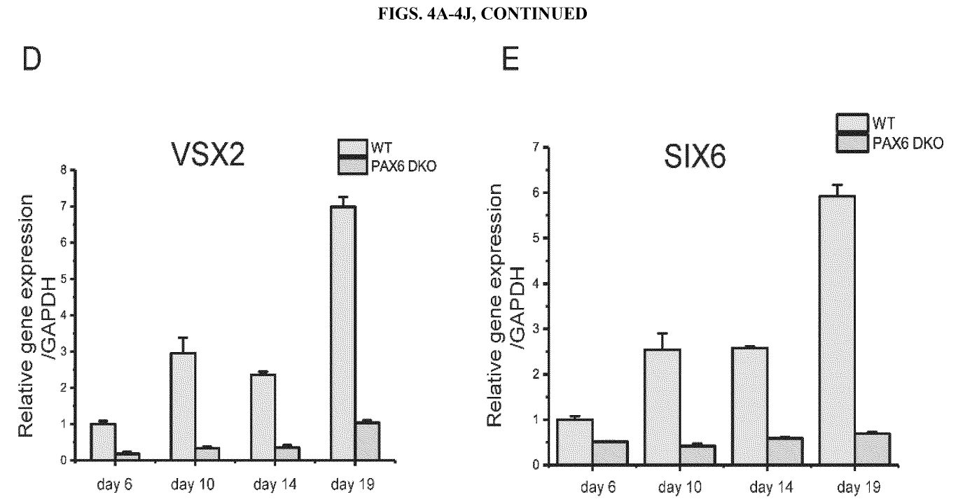

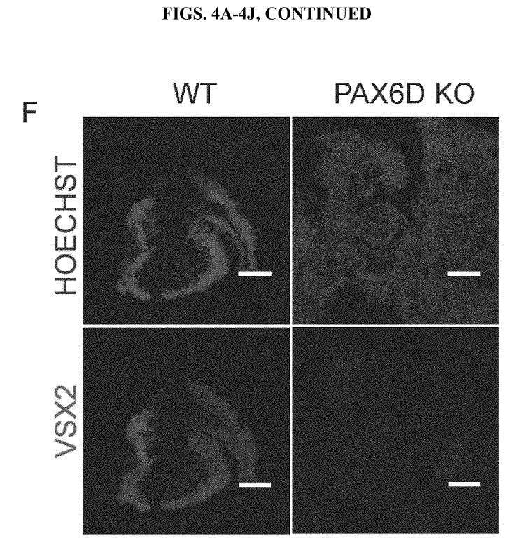

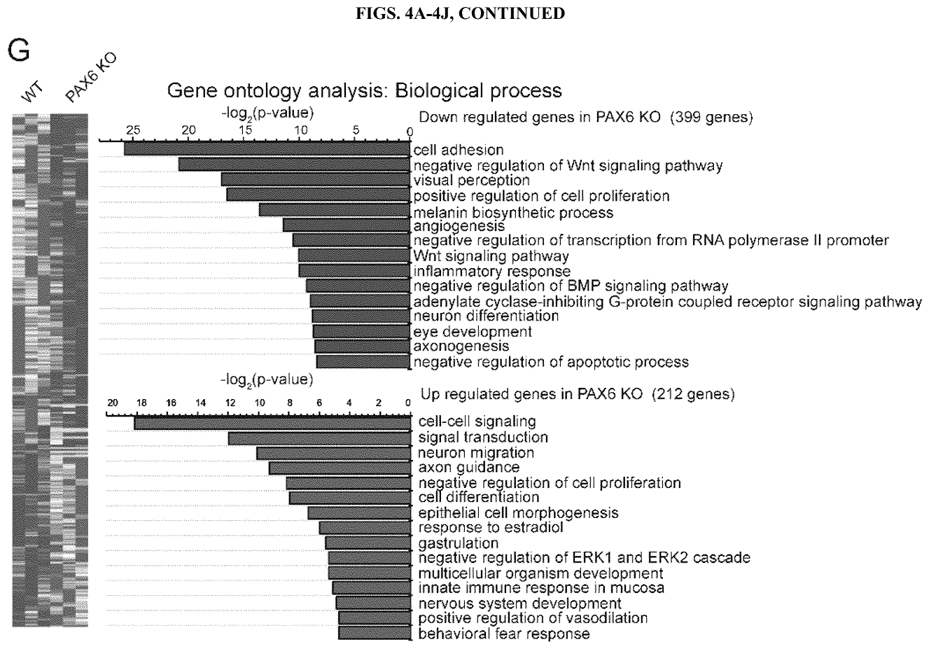

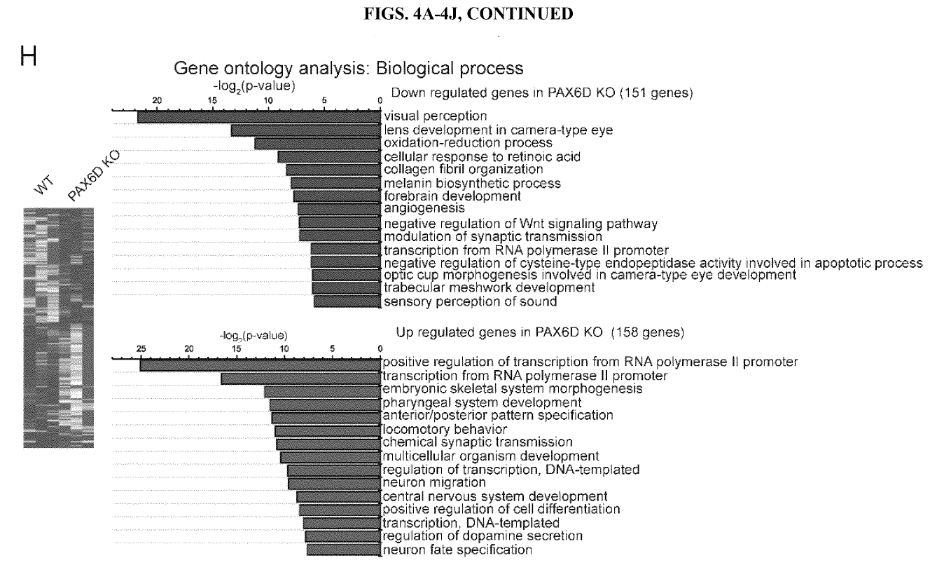

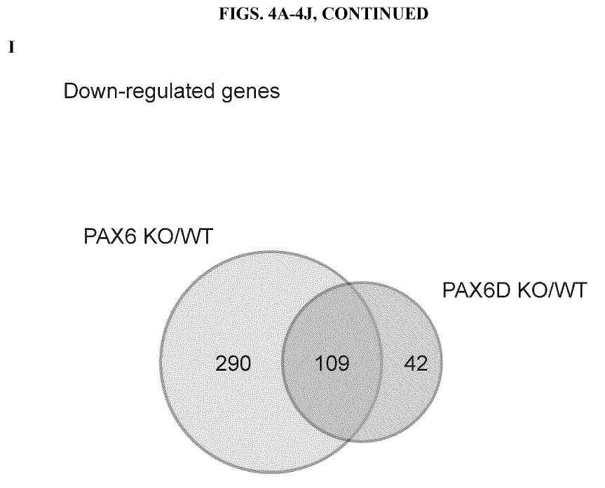

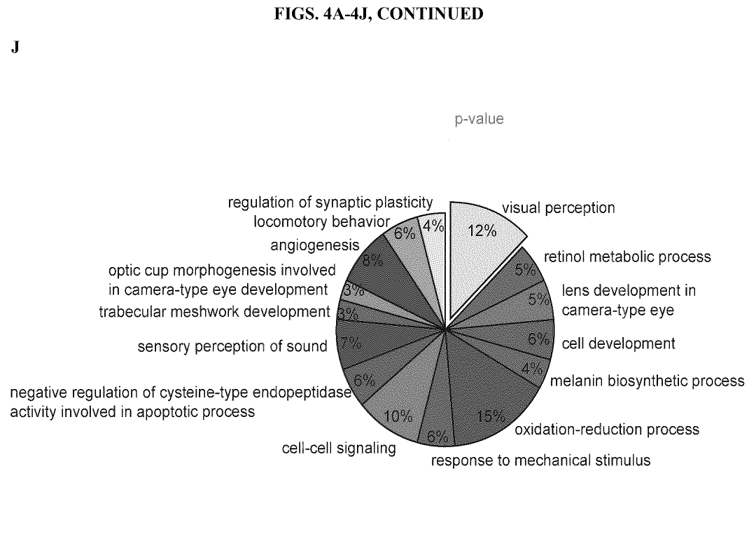

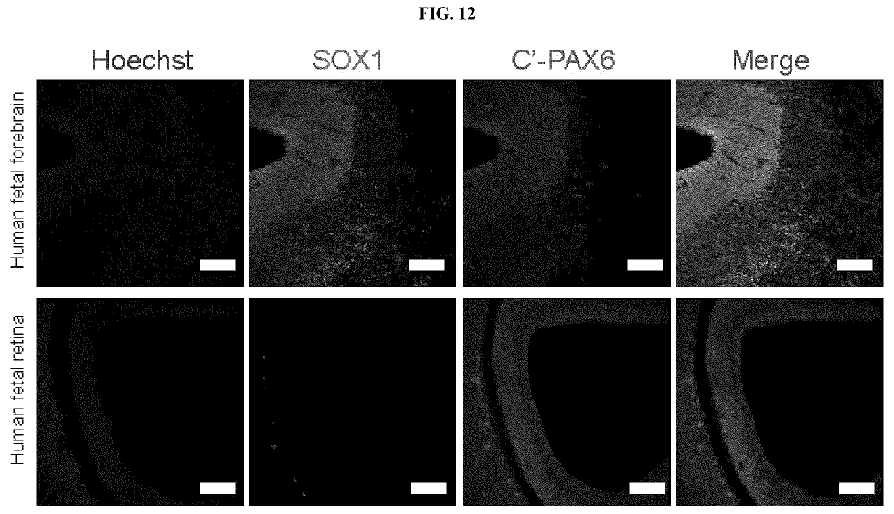

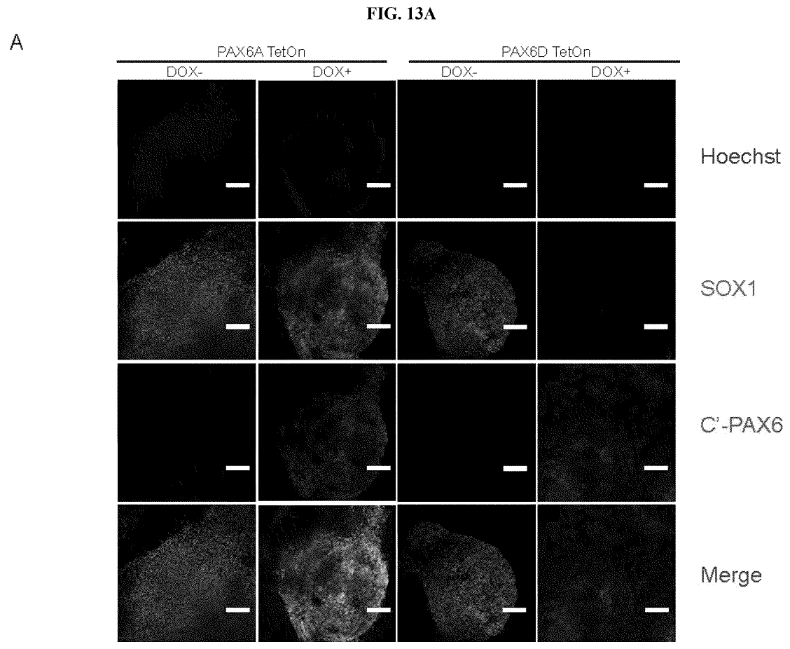

The requirement of PAX6 for human retinal fate specification and the differential expression pattern of PAX6 isoforms led us to hypothesize that PAX6D is required for retinal specification. We then generated a PAX6D isoform-specific knockout hESC line. All the PAX6D coding sequences (CDS) are shared by PAX6A and PAX6B (FIG. 2A). Deletion of any exons within the CDS will affect all the isoforms. However, PAX6 isoform transcription is controlled by separate promoters and PAX6D has an additional exon (exon alpha) in its 5′-UTR (Kammandel et al., 1999; Lakowski et al., 2007; Marquardt et al., 2001). Based on the unique feature of PAX6D, we successfully generated PAX6D specific knockout by partially replacing Exon α with the PGK-puromycin element using CRISPR/CAS9 (FIG. 4A, FIG. 12). We confirmed PAX6D KO by qPCR and western blot (FIGS. 4B-4C). PAX6D mRNA and protein were reduced to undetectable in PAX6D KO cells. Western blot also validated the isoform knockout specificity by using the PAX6 antibodies that recognize the PAX6 C-terminus, which detects both PAX6A&B and PAX6D. It showed that PAX6A&B was expressed at a similar level in the PAX6D KO cells (FIGS. 4B-4C), indicating only PAX6D was knocked out. Knockout of PAX6D in hESCs caused a similar phenotype as PAX6 KO cells. The key EFTFs such as VSX2 and SIX6 were not upregulated in PAX6D KO cells along the retinal differentiation (FIGS. 4D-4E). Immunostaining analysis showed the absence of VSX2 expression in PAX6D KO cells (FIG. 4F).

To systematically analyze the gene profile of cells with PAX6D knockout, we performed RNA-Seq for wildtype, PAX6 KO and PAX6D KO cells (Table 2). Pairwise comparison of PAX6 KO with WT showed that 611 genes were differentially expressed (2 fold; p<0.05). Among them, 399 genes were down-regulated in PAX6 KO cells, which are highly related to retinal development or retinal function, including “visual perception”, “melanin biosynthetic process” and “eye development” (FIG. 4G). In addition, cell proliferation and neural tube patterning signaling pathways, such as WNT and BMP, were also down-regulated in PAX6 KO cells (FIG. 4G). These results are consistent with our findings above that PAX6 KO cells were defective in retinal fate specification and suggest a potential role of PAX6 in regulating retinal differentiation by modulating WNT or BMP pathway. Gene ontology (GO) analysis for the up-regulated 212 genes in PAX6 KO cells showed that “negative regulation of cell proliferation”, “cell differentiation and nervous system development” were among the top enriched biological processes, suggesting a role of PAX6 in regulating cell proliferation and neural fate determination.

Deletion of PAX6D isoform was associated with altered gene expression in retinal development (FIG. 4H). There were 309 genes that were differentially expressed between wildtype and PAX6D KO cells. Among them, 151 were down-regulated in PAX6D KO cells, which were enriched in gene ontologies such as “visual perception”, “lens development in camera-type eye”, “melanin biosynthetic process”, “WNT pathway” and “optical cup morphogenesis in camera-type eye development” (FIG. 4H). This profile suggests a failure of retinal differentiation in PAX6D KO cells. The 158 up-regulated genes in PAX6D KO cells were enriched in biological processes such as “anterior/posterior pattern specification”, “multicellular organism development”, “neuron migration”, “central nervous system development”, “positive regulation of cell differentiation” and “neuron fate specification”. This profile highlights the tendency of the PAX6D KO cells towards neuroepithelia.

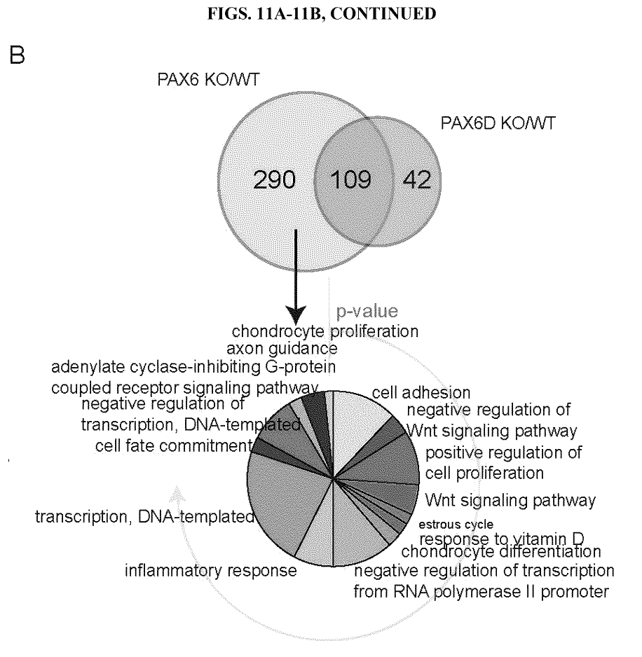

Comparing the down-regulated genes between PAX6 KO and PAX6D KO cells, we found two-thirds (109 out of 151) of the differentially expressed genes in PAX6D KO cells are overlapped with those in PAX6 KO cells (FIG. 4I). GO analysis of the shared 109 genes highlights the role of PAX6D in retinal fate determination since the top GO terms are related to retinal functionality and optic cup morphogenesis (FIG. 4J). Among the shared down-regulated genes, 12% of the genes such as VSX2, VAX2 and PAX2 are in the “visual perception” category (FIG. 4J & Table 3). The 290 genes that are uniquely down-regulated in PAX6 KO cells but not in PAX6D KO cells would signal the function of other PAX6 isoforms. GO analysis of this set of genes showed that “cell adhesion”, “negative regulation of Wnt signaling pathway” and “positive regulation of cell proliferation” are the top affected biological processes (FIG. 11B & Table 3). This is consistent with the function of Pax6 and Pax6(5α) revealed in model animals (Jami et al., 2013; Jones et al., 2002; Klimova and Kozmik, 2014). Together, our RNA-Seq analysis further suggests that the failed retinal differentiation seen in PAX6 KO cells is essentially attributed to the loss of PAX6D function.

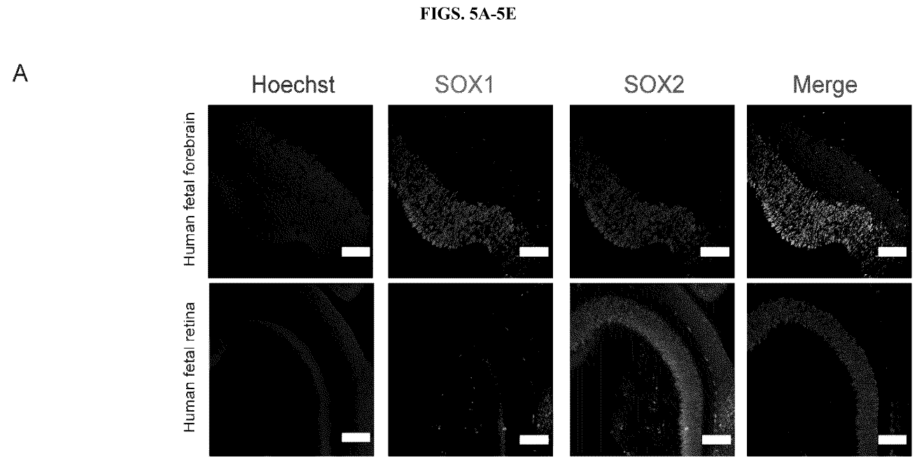

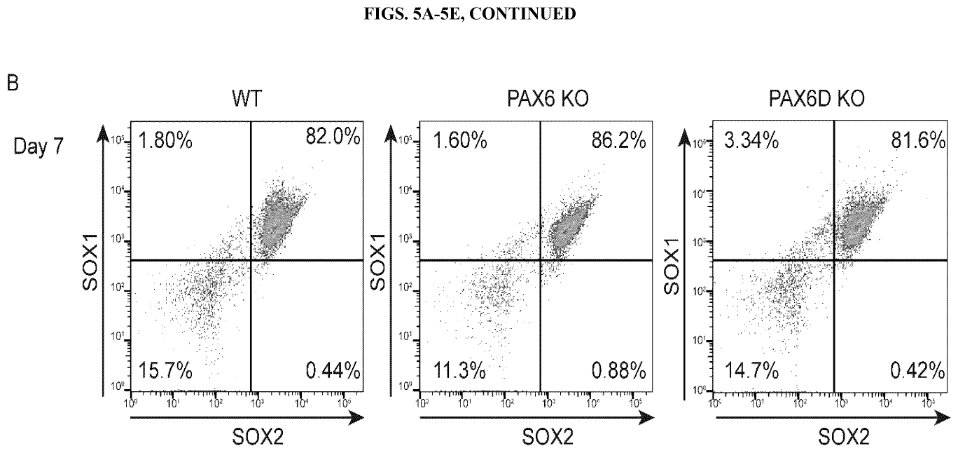

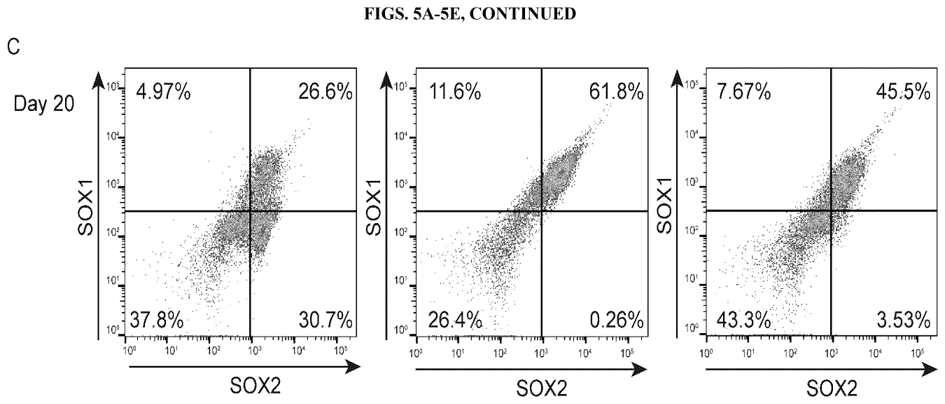

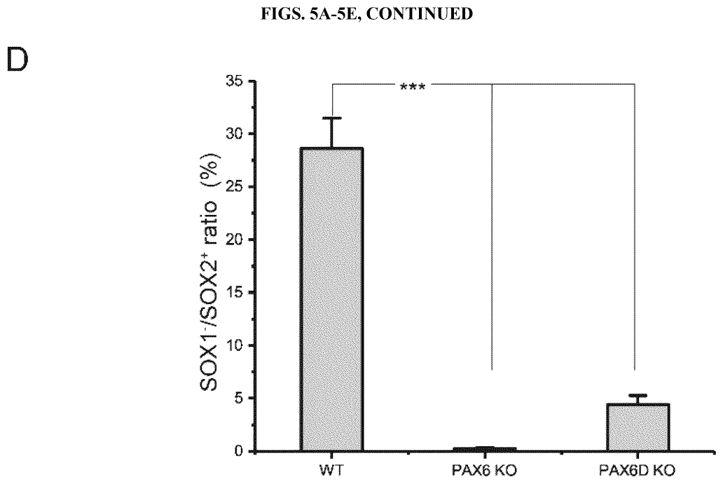

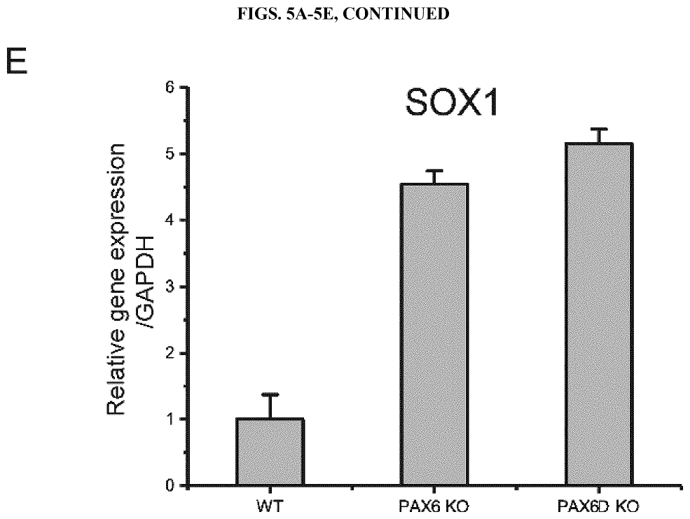

At the cellular level, the retinal differentiation culture contains both neuroepithelia and retinal cells. The EFTFs, including PAX6, used to define the early retinal cells are also expressed in neuroepithelia. SOX1, a neuroepithelial marker, is absent in PAX6⁺ and SOX2⁺ early RPCs (Chen et al., 2017; Kamachi et al., 1998). Indeed, immunostaining of 12-week human fetal tissues indicated the expression of PAX6, SOX1, and SOX2 in the developing human forebrain but with lack of SOX1 in retinas (FIG. 5A, FIG. 12). Thus, we used SOX1 and SOX2 to monitor the retinal fate specification (SOX1⁻/SOX2⁺) from neuroepithelia (SOX1⁺/SOX2⁺). FACS analysis of retinal differentiation cultures at day 7 (before NR differentiation) indicated that more than 80% of the cells were neuroepithelial cells (SOX1⁺/SOX2⁺) among all lines (FIG. 5B). Few SOX1⁻/SOX2⁺ cells were present. At day 20, about 30% of the cells were SOX1⁻/SOX2⁺ in WT but only 0.3% and 3.5% in the PAX6 KO and PAX6D KO groups, respectively (FIGS. 5C-5D). Consistent with the reduced SOX1⁻/SOX2⁺ population in PAX6 KO and PAX6D KO cells, SOX1 mRNA levels support the trend. Its relative expression was much higher in PAX6 KO and PAX6D KO cells than WT (FIG. 5E). The absence of SOX1/SOX2+ population demonstrates the defect in early retinal specification in both PAX6 KO and PAX6D KO cells.

PAX6D is Sufficient for Retinal Specification from Neuroepithelia

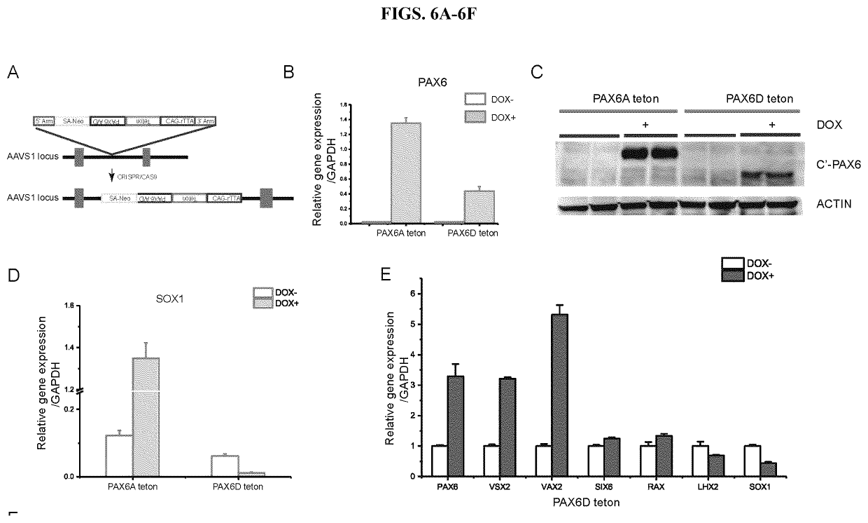

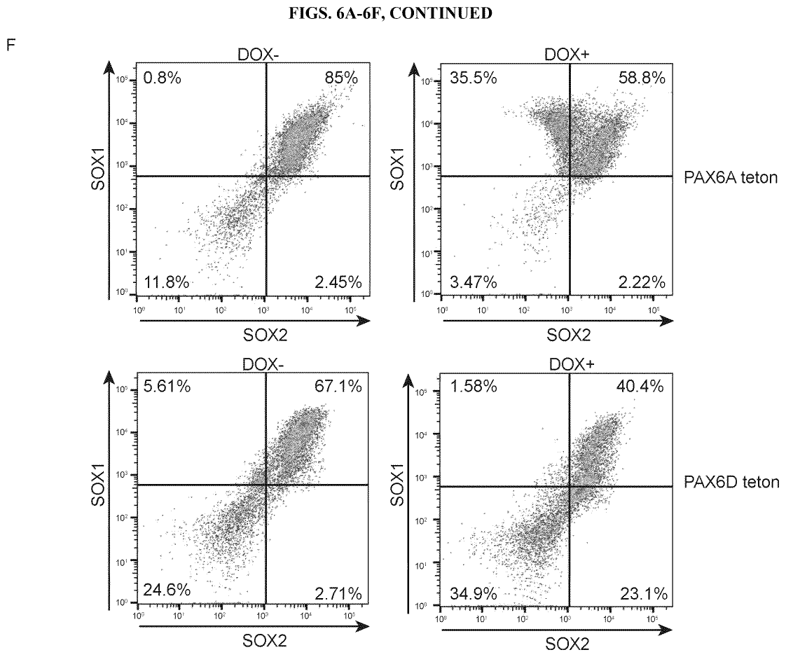

The observation that PAX6D KO results in the loss of retinal differentiation led us to hypothesize that PAX6D may be sufficient for retinal specification from neuroepithelia. To test his hypothesis, we introduced the PAX6D isoform into the PAX6 KO background by establishing the Tet-on inducible cell lines (FIG. 6A). We also made the PAX6A inducible cell lines as a control (FIG. 6A). As indicated by qPCR and Western blot, only PAX6A or PAX6D expression was induced in the respective cell line by doxycycline (DOX, 0.5 μg/ml) (FIGS. 6B-6C).

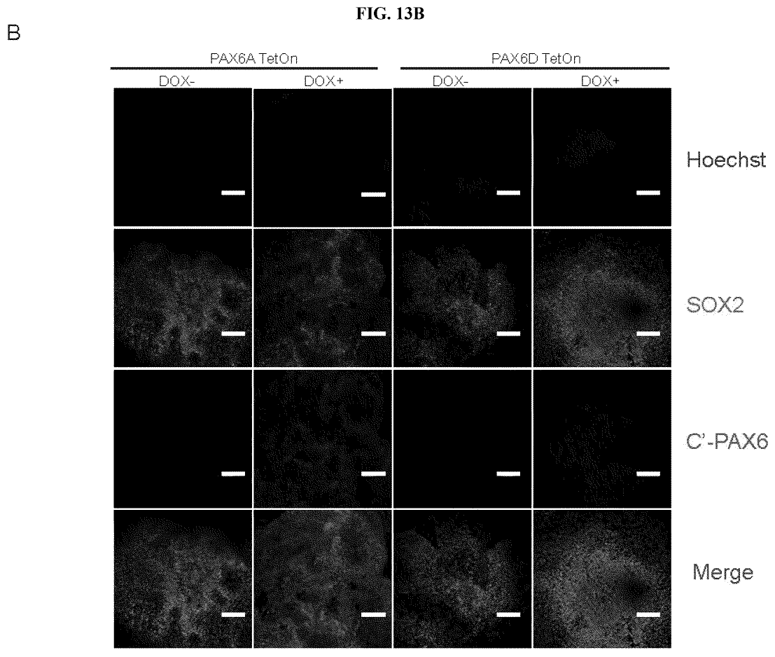

We then examined the retinal progenitor population when PAX6A or PAX6D was induced. We turned on PAX6A and PAX6D separately in PAX6 KO cells after neuroepithelial cells were generated at day 6 and monitored the retinal progenitor generation along retinal differentiation. Induction of PAX6D with DOX for 7 days from day 6 of retinal differentiation, resulted in the elevated expression of retinal lineage markers such as VSX2 (3-fold increase), VAX2 (2-fold increase) and SIX6 (FIG. 6E). The expression of the neuroepithelial gene SOX1 was downregulated at the mRNA (FIGS. 6D-6E) and cellular level (FIG. 13A). In contrast, induction of PAX6A upregulated SOX1 expression level (FIG. 6D). Correspondingly, the SOX1⁻/SOX2⁺ retinal progenitor population, assayed by flow cytometry, increased from 2.71% to 23.1% when PAX6D was induced whereas this population did not obviously change (from 2.45% to 2.22%) when PAX6A was induced (FIG. 6F). Thus, PAX6D but not PAX6A restores the PAX6 function in retinal specification.

PAX6D Represses Neural while Activating Retinal Lineage Genes

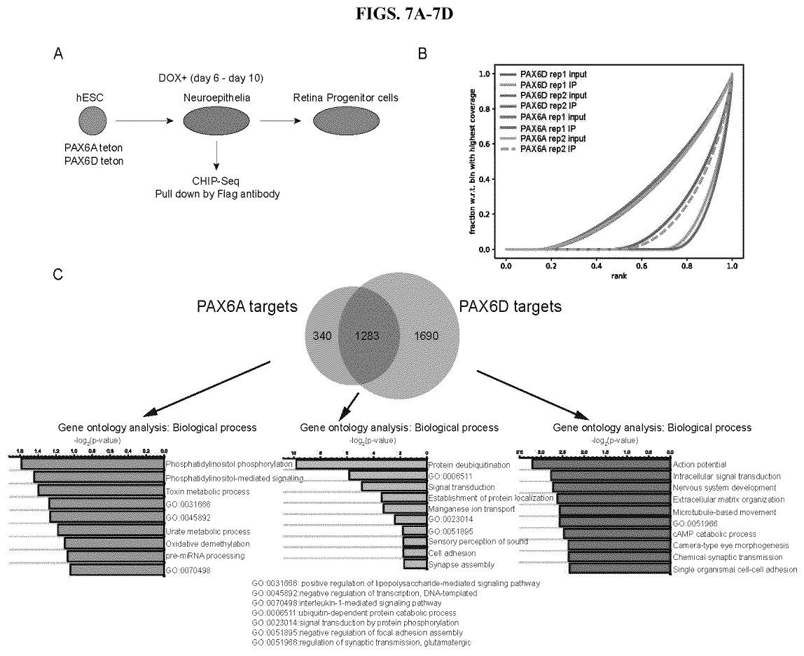

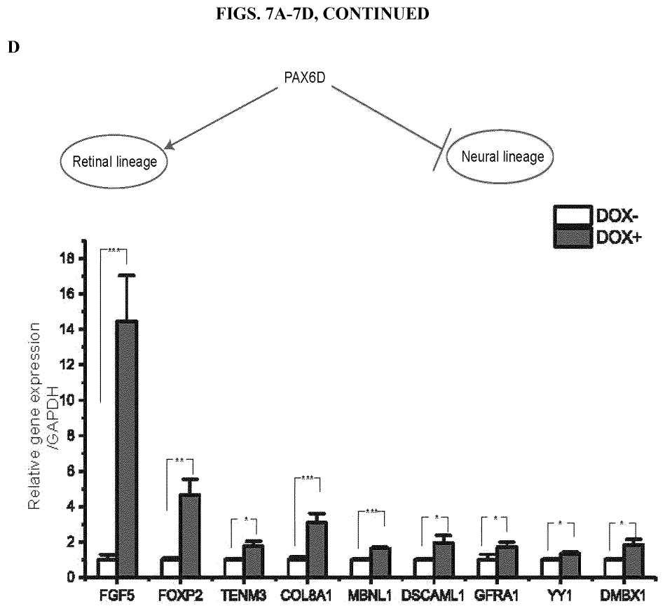

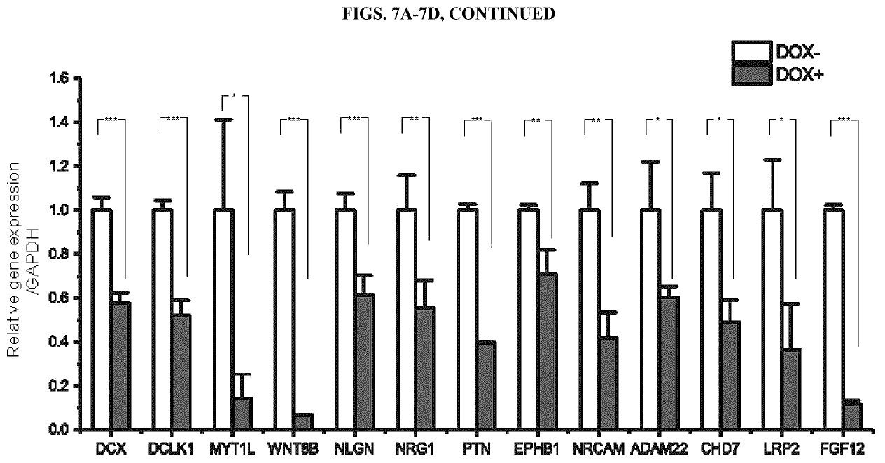

ChIP-Seq was performed to determine how PAX6D regulates retinal differentiation. Technically, there are no antibodies which can only detect PAX6D due to the identical C-terminus among PAX6A, PAX6B and PAX6D (FIG. 2A). To overcome this, we built PAX6A and PAX6D inducible cell lines under the PAX6 KO background with PAX6A and PAX6D tagged by 3× Flag, respectively (FIG. 5A). We used PAX6 KO cells as our target to eliminate or minimize the influence of endogenous PAX6 isoforms, which may compete with the induced isoforms for binding sites. We applied DOX at day 6 of retinal differentiation for 3 days and harvested cells at day 10 for ChIP-Seq (FIG. 7A). We found that the IP strength in separate ChIP experiments for the FLAG antibody pull-down was similar for the replicates and the target DNA fragments were highly enriched (FIG. 7B). Analysis of the pull-down DNA fragments by ChIP-Seq revealed that PAX6A and PAX6D shared many downstream genes. There were 1281 genes predicted to be their common targets, which includes genes related to “nervous system development” such as DSCAM, APP, BDNF, DCX, DLG2 and NLGN1 (Table 3), genes for “neuron migration” such as SPOCK1, ADGRL3, PCM1, PEX7 and SEMA3A, and genes for “cell adhesion” such as ACTN2, CTNNA1, CTNNA3, COL28A1, DST, IBSP, ITGB3BP and OPCML. The result suggests that PAX6A and PAX6D have redundant roles in these biological processes.

Gene ontology analysis of the unique genes targeted by PAX6A or PAX6D indicated that PAX6D was specifically involved in such biological processes as “nervous system development” and “camera-type eye morphogenesis” while PAX6A was associated with the cell functionality such as signal transduction and metabolic process (FIG. 7C & Table 4).

Carefully analyzing PAX6D target genes, we identified a set of retinal lineage genes and a set of neural lineage genes that are regulated by PAX6D based on prediction from our ChIP-Seq data. By using the PAX6D TetOn cells, we confirmed that most of the genes were responsive to PAX6D induction (FIG. 7D). The retinal lineage genes upregulated by PAX6D induction include FGF5, highly expressed in primate retinal cells and rescues the retinal degeneration in animal models (Green et al., 2001; Kitaoka et al., 1994), FOXP2, uniquely expressed in and defines a subset of retinal ganglion cells (Rousso et al., 2016; Sato et al., 2017), TENM3, required for topography in the ipsilateral retino-collicular pathway and specifies morphological and functional connectivity of retinal ganglion cells (Antinucci et al., 2013; Dharmaratne et al., 2012; Glendining et al., 2017), YY1 predominantly expressed in optic vesicle and retinal cells (Bernard and Voisin, 2008; Kwon and Chung, 2003; Vuong et al., 2012) and required for normal eye development (Lorente et al., 2006), DMBX1, necessary for cell cycle exit in developing RPCs (Miles and Tropepe, 2016; Wong et al., 2015; Wong et al., 2010) and MBNL1 which regulates photoreceptor development (Huang et al., 2008). In contrast, the neural lineage genes repressed by PAX6D induction include MYTIL, sufficient to convert fetal and postnatal human fibroblasts into induced neuronal cells (Mall et al., 2017), WNT8B, restricted in dorsal forebrain (Lako et al., 1998) and inhibits retinal differentiation (Liu, 2012), NLGN, essential for presynaptic terminal maturation (Wittenmayer et al., 2009), and DCX, predominantly expressed in immature neurons (Capes-Davis et al., 2005; Couillard-Despres et al., 2005) (FIG. 7D). This result suggests that PAX6D regulates retinal differentiation by targeting retinal genes while repressing other neural genes.

### PAX6D Instructs a Retinal Fate by Regulating WNTs.

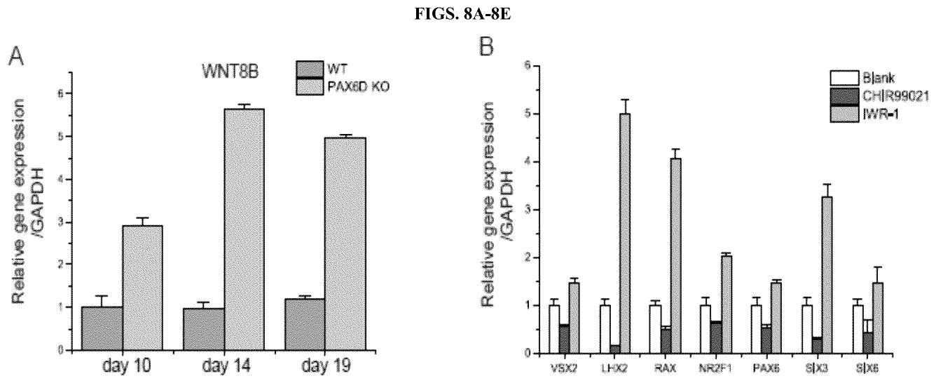

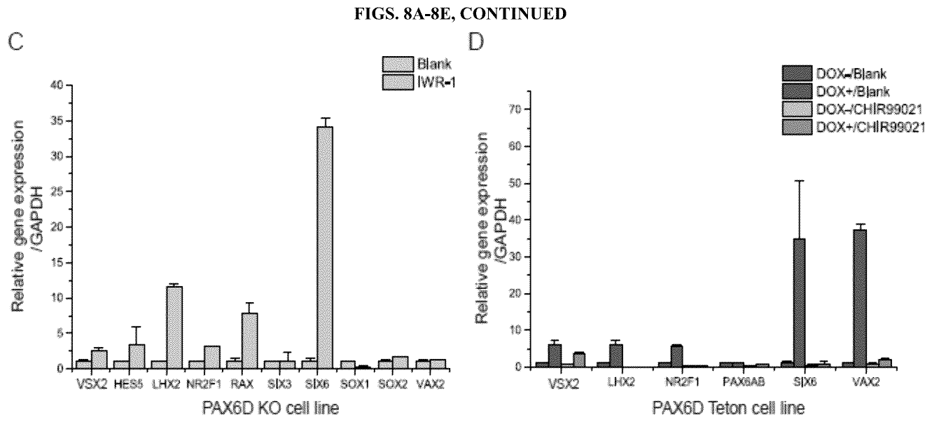

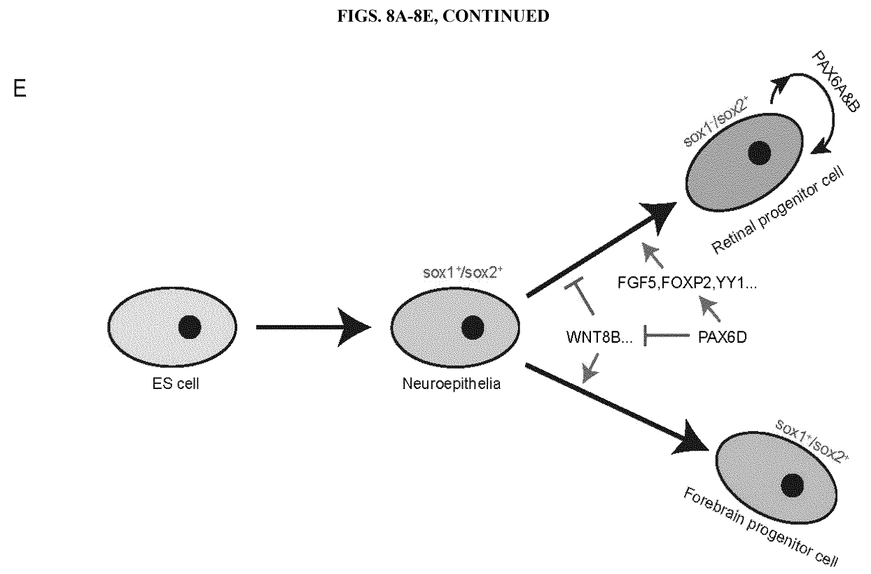

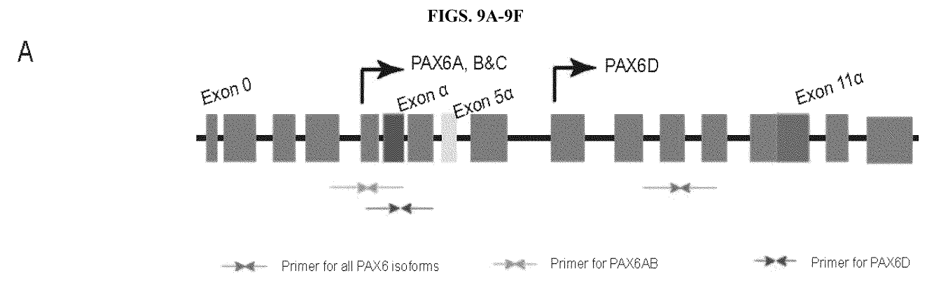

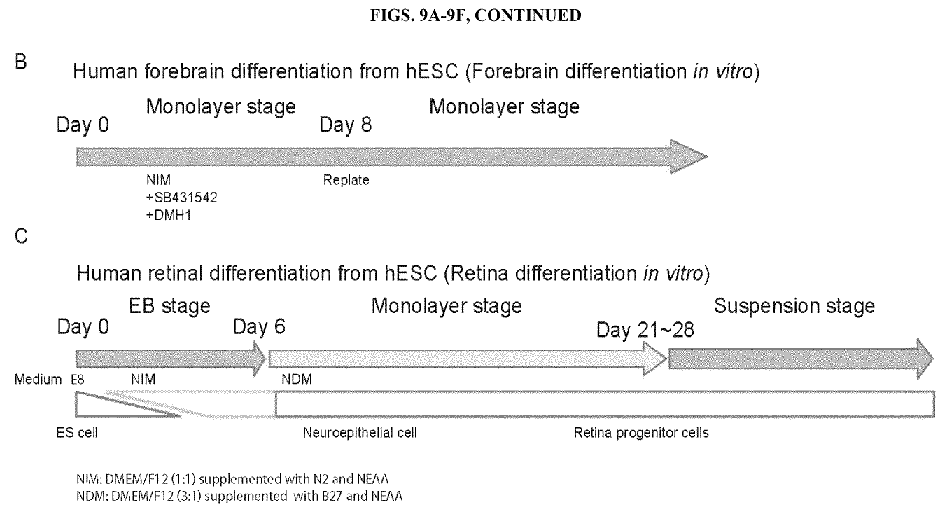

The highly upregulated WNT-related GO terms in PAX6D KO cells revealed by RNA-Seq (FIGS. 4G-4H) and the identification of WNT8B as one of the PAX6D targets from ChIP-seq (FIG. 7D) suggest that PAX6D may regulate retinal specification by modulating WNT pathway. WNT8B has a restricted expression in the developing dorsal forebrain (Lako et al., 1998) and plays a repressive role in retinal fate specification (Liu et al., 2010). We found that WNT8B expression was much higher in PAX6D KO cells than wildtype cells at multiple time points along retinal differentiation (FIG. 8A). Given the direct connection between PAX6D and WNT8B, we hypothesized that PAX6D regulates retinal specification by repressing WNT8B transcription and thus WNT activity. Indeed, treatment with WNT agonist (CHIR99021, 3 μM) in wildtype cells from day 0 to day 10 suppressed the expression EFTFs. In contrast, treatment with a WNT antagonist (IWR1, 2.5 μM) in wildtype cells enhanced the expression of EFTFs (FIG. 8B). These results suggest that WNT signaling represses retinal specification from the neuroepithelia.

We then asked if the failure of retinal specification in the PAX6D KO cells is rescued by regulating WNT signaling. Treatment of the PAX6D KO cells with IWR1 during retinal differentiation from day 0 to day 10 increased the expression of retinal related genes, including VSX2, LHX2, HES5, SIX3 and SIX6 (FIG. 8C). This result suggests that WNT signaling is indeed downstream of PAX6D function in retinal specification. To further corroborate the role of WNTs in mediating the function of PAX6D in retinal specification, we then activated WNT pathway signaling under PAX6D induction along retinal differentiation. As indicated by expression of retinal genes (VSX2, LHX2, SIX6, NR2F1 and VAX2) at day 10 of retinal differentiation, induction of PAX6D by DOX enhanced retinal differentiation. Such PAX6D-induced retinal differentiation was completely blocked by treatment with WNT agonist CHIR99021 from day 0 to day 10 (FIG. 8D). These results indicate that regulation of retinal differentiation by PAX6D is dependent on the canonical WNT pathway.

We found that isoforms of PAX6 are differentially expressed in neuroepithelial and retinal tissues with PAX6D specifically expressed in retinal cells during human eye development and along hESC differentiation to retinal cells. Deletion of PAX6D down-regulates retinal gene profiles and blocks retinal differentiation, similar to that with complete PAX6 KO. Induced expression of PAX6D, but not PAX6A, restores the retinal differentiation capacity of the PAX6-null cells. These results indicate that PAX6D is necessary and sufficient for retinal lineage specification from neuroepithelia (FIG. 8E). By identifying the PAX6D targets and by regulating one of its targets, WNTs, we are able to rescue the retinal differentiation deficit in PAX6D KO cells and block retinal differentiation when PAX6D is induced, revealing how PAX6D regulates retinal specification (FIG. 8E).

Genetic studies in multiple model organisms have firmly established that PAX6 is essential for retinal development (Collinson et al., 2000; Grindley et al., 1995; Hogan et al., 1986; Li et al., 2007; Quinn et al., 1996). PAX6 is also required for neuroectoderm development, especially the dorsal forebrain (Georgala et al., 2011; Jones et al., 2002; Zhang et al., 2010) from which the retinal is specified. This raises a question of how PAX6 instructs neuroepithelial cells to RPCs. Our present observation indicates that PAX6D is uniquely expressed in NR during human development and along hESC retinal differentiation. This is consistent with observations made in model animals that Pax6ΔPD is expressed exclusively in NR but not the developing lens or cornea (Kim and Lauderdale, 2006, 2008) and that the promoter (exon α) controlling Pax6ΔPD transcription also has a restricted distribution in NR (Kim and Lauderdale, 2008; Marquardt et al., 2001). However, the exact role of Pax6ΔPD (or PAX6D in human) remains unknown (Shaham et al., 2012), possibly due to the lack of the Pax6ΔPD loss of function animal model. This is probably because of the difficulty to create such a model with the traditional genetic tools, as Pax6ΔPD shares all the coding sequences with other isoforms. By using CRISPR/CAS9 to target the exon α which only exists in PAX6D 5′ UTR, we generated the PAX6D specific knockout. This model enables us to learn the physiological function of PAX6D in retinal development. Indeed, knockout of PAX6D in hESC prevents the cells from becoming retinal cells. This is confirmed by the lack of SOX1⁻/SOX2⁺ retinal progenitors and the substantial down regulation of retinal lineage genes, revealed by RNA sequencing. This phenotype is very similar to complete PAX6 KO in which the differentiated cells remain as neuroepithelia. In contrast, introduction of PAX6D but not PAX6A into PAX6 KO neuroepithelial cells is sufficient to convert the neuroepithelial cells (SOX1⁺/SOX2⁺) to RPCs (SOX1⁻/SOX2⁺). Therefore, PAX6D is necessary and sufficient for specifying neuroepithelia to retinal cells.

The question arises is how PAX6D converts neuroepithelia to RPCs. Our ChIP-seq revealed genes that are targeted by PAX6D, including a set of retinal genes and neural genes that are predicted based on the enrichment of peaks in genomic genes and validated by our transgenic analysis. The neuroectoderm genes repressed by PAX6D include critical transcription factors like MYTIL that is sufficient for neuronal fate reprograming from fibroblast cells (Mall et al., 2017) and signaling molecules such as WNT8B which inhibits retinal differentiation (Liu, 2012). It is conceivable that repression of the neuroepithelial transcription factors and associated signaling endows the neuroepithelia with the potential to become RPCs. Then it is the retinal genes and related signaling activated by PAX6D that enable the conversion of the neuroepithelia to RPCs. Indeed, our Chip-Seq revealed retinal associated genes that are activated by PAX6D, such as FGF5, FOXP2, TENM3, YY1, DMBX1 and MBNL1. VSX2, a master regulator of RPCs (Burmeister et al., 1996), is also regulated by PAX6D (FIG. 6E). In our present study, we have shown that VSX2 is essentially absent when PAX6D is knocked out. In contrast, PAX6D induces VSX2 expression and retinal differentiation from hESCs even when PAX6 is deleted. Therefore, PAX6D specifies the retinal fate from neuroepithelia by repressing neuroectodermal genes, activating retinal genes and modulating related signaling molecules.

One of the signaling pathways that mediates retinal specification and that is targeted by PAX6D is WNT8B. WNT8B is expressed in the dorsal forebrain (Lako et al., 1998) and antagonizes retinal specification (Cavodeassi et al., 2005). WNT activity is absent in distal optic vesicle (prospective NR) at the early stage of retinal fate specification (Liu et al., 2003; Liu et al., 2006), highlighting the requirement of Wnt8B suppression for retinal specification (Liu et al., 2010). Consistent with the phenomena observed in animals, WNT8B expression is upregulated in PAX6D KO cells, suggesting the role of WNTs in mediating the effect of PAX6D. Indeed, chemical inhibition of WNTs rescues retinal differentiation from neuroepithelia with PAX6D KO. In contrast, chemical activation of WNT signaling blocks retinal differentiation from neuroepithelia even when PAX6D is over expressed. Thus, one of the mechanisms by which PAX6D specifies forebrain neuroepithelia to RPCs is to regulate WNT activity.

To further explore the role of WNT pathway signaling in retinal development, short hairpin RNAs (shRNA) were designed to target and knock down expression of WNT8B. Generally, nucleotide sequence encoding an shRNA is operably linked to an RNA polymerase promoter and are introduced into a cell via an expression vector such as a viral vector. Three shRNAs targeting WNT8B were designed and used in the following assay. The hairpin sequences of the 3 WNT8B shRNAs were:

(SEQ ID NO: 1)
GATCCCCATGAAACGCACGTGTAATTCAAGAGATTACACGTGCGTTTCAT
GGTTTTT;
(SEQ ID NO: 2)
GATCCCCTAGCCTATCAACCCTTATTCAAGAGATAAGGGTTGATAGGCTA
GGTTTTT;
and
(SEQ ID NO: 3)
GATCCCTCTGGAGTGCTAACTTGATTCAAGAGATCAAGTTAGCACTCCAG
AGTTTTT.

All lentivirus expression constructs were produced by Santa Cruz Biotechnology (sc-41118). The control shRNA was obtained from Santa Cruz Biotechnology (sc-108080).

The WNT8B shRNA was introduced by contacting human pluripotent stem cells to WNT8B shRNA viruses (or control viruses) at day 0. Following introduction, retinal differentiation was performed using the published protocol (Zhong et al., 2014). Expression of retinal progenitor markers was detected on day 10 of retinal differentiation. As shown in FIG. 15A, introduction of a WNT8B shRNA reduced WNT8B expression relative to expression in the presence of a control shRNA or in untreated cells. As shown in FIG. 15B, knocking down expression of WNT8B using the WNT8B shRNA increased expression of retinal progenitor markers such as VSX2, HES5, LHX2, NR2E1, RAX, and VAX2. These data further demonstrate that WNTs mediate the effect of PAX6D on retinal cell differentiation by modulating expression of such retinal associated genes.

In summary, this example demonstrates a role for PAX6D in retinal specification. Deletion of PAX6D prevents neuroepithelia from differentiating to retinal cells whereas induction of PAX6D, but not PAX6A, enables retinal differentiation even when PAX6 is knocked out. Therefore, PAX6D is both necessary and sufficient for retinal specification from neuroepithelia. PAX6D specifies the retinal fate from neuroepithelia by repressing neuroectodermal genes, activating retinal genes and modulating signaling molecules. Our findings resolve the mystery how PAX6, expressed in both forebrain neuroepithelia and NR, instructs a retinal fate versus neural fate through the differential use of its isoforms. Our findings also open avenues to guide or convert neuroepithelia to highly enriched RPCs by inducing PAX6D expression or to isolate RPCs by targeting PAX6D.

### Materials and Methods

hPSCs: H9 ESC and genetically modified cell lines derived from H9 (PAX6 KO, PAX6D KO, PAX6A TetOn, PAX6D TetOn) were either maintained on irradiated mouse embryonic fibroblast (MEF) feeder (Du et al., 2015) or feeder-free system with Matrigel® or Vitronectin (Yuan et al., 2015). Briefly, cells maintained on MEF were passaged weekly by dispase (1 mg/ml) and plating on MEF (WiCell) with the hPSC culture medium consisting of DMEM/F12 basal medium, 20% KnockOut serum replacement, 0.1 mM β-mercaptoethanol, 1 mM L-glutamine, nonessential amino acids (NEAA), and 4 ng/ml FGF-2. For cells maintained on Matrigel® or Vitronectin, the cells were passaged every 4-5 days by EDTA. Rho Kinase Inhibitor (0.5 μM) was added in E8 to help the survival of hPSCs.

Human subjects: The human fetal tissue used in this study was derived from patients who required termination of pregnancy. All procedures were approved by the Ethics Committee of Tongji Hospital affiliated to Tongji Medical College of Huazhong University of Science and Technology, with informed consent. And all human body materials are treated with special care in accordance with the requirements and regulations established by the Ethics Committee. Fetal tissues were obtained within 2 hours after abortion, and the developmental stages of these fetal specimens were determined based on clinical diagnosis and related examinations. After the fetal tissue was obtained, the tissue was preserved in 9% physiological saline, and the retina, lens, brain, and spinal cord in the tissue were separated under a stereo microscope (SZX12, Stereo Microscope, OLYMPUS, Japan) as soon as possible.

Generation of PAX6 KO, PAX6D KO, PAX6A TetOn and PAX6D TetOn cell lines: PAX6 KO and PAX6D KO cell lines were designed by inserting the PGK-Puromycin in exon 8 and exon alpha of the PAX6 gene, respectively, which terminates the normal translation of PAX6 genes early and PAX6D isoform specifically. The PAX6 KO cells were then used to generate PAX6A TetOn and PAX6D TetOn by inserting the TetOn-PAX6A-3XFLAG and TetOn-PAX6D-3XFLAG into the AAVSI site of PAX6 KO genome.

Genome editing was performed using CRISPR/CAS9 following published method (Chen et al., 2015). Guide RNAs (gRNAs) were designed according to the protocol described by Feng Zhang's laboratory (available at crispr.mit.edu/ on the World Wide Web). Briefly, Human H9 cells or H9 derived PAX6 KO cells were cultured in the hPSC medium with Rho Kinase Inhibitor (0.5 μM) for 24 hours prior to electroporation. Cells were digested by TrypLE express Enzyme for 3-4 minutes and harvested in hPSC medium with Rho Kinase Inhibitor. Cells were dispersed into single cells, and 1×10⁷ cells were electroporated with appropriate combination of plasmids in 500 μl of DMEM/F12 medium using the Gene Pulser Xcell System (Bio-Rad) at 250 V, 500 μF in 0.4-cm cuvettes. Cas9 plasmid (15 μg), sgRNA plasmid (15 μg) and donor plasmid (30 μg) were used for electroporation. Cells were subsequently plated onto MEF in 6-well plates in MEF-conditioned hPSC medium with ROCK-inhibitor. The medium was changed to MEF-conditioned medium without ROCK-inhibitor 24 hours later. Three days later after electroporation, puromycin (0.5 μg/ml) was added into the MEF-conditioned medium to select the positive clones for PAX6 KO and PAX6D KO for two weeks. G418 (50 μg/ml) was used to select the positive clones for PAX6A TetOn and PAX6D TetOn derived from PAX6 KO cells. After drug selection, cells were treated with Rho Kinase Inhibitor for 24 hours, and then individual colonies were picked up, mechanically disaggregated and replated onto MEF in 24-well plates in MEF-conditioned hPSC medium with Rho Kinase Inhibitor for the first 24 hours. Positive colonies were identified by genomic PCR and replated onto MEF in 6-well plates with MEF-conditioned hPSC medium.

Forebrain and retinal differentiation from hPSCs: Forebrain neuroepithelial differentiation from hPSCs was performed by dual SMAD inhibition (Chambers et al., 2009). Briefly, hESCs were treated with the neural differentiation medium comprised of DMEM/F12: neurobasal (1:1), 1×N2, 1×B27, 1× nonessential amino acids (NEAA), 1% GlutaMAX, 2 μM SB431542 and 2 μM DMH1 for 1 week. The culture medium was changed every other day. One day 7, cells were split and replated to fresh MEF in the same neural differentiation medium with Rho Kinase Inhibitor (0.5 μM). Cells were maintained with forebrain differentiation medium by feeding every other day.

Retinal differentiation from human pluripotent stem cells was performed using the published protocol (Zhong et al., 2014). Briefly, hESCs were passaged to Vitronectin-coated plates and cultured to 60-70% confluency. On day 0 of retinal differentiation, cells were enzymatically detached by dispase (1 mg/ml) and cultured in suspension with E8 medium to form embryoid bodies (EB). EBs were gradually transitioned into neural induction medium (NIM) containing DMEM/F12 (1:1), 1% N2 and 1×NEAA by replacing the medium with a 3:1 ratio of E8/NIM on day 1, 1:1 on day 2, and 100% NIM on day 3. On day 7, EBs were seeded onto plates with NIM containing 5% FBS. The medium was changed to NIM one day later and fed with NIM every other day. On day 16 the medium was changed to RDM (retinal differentiation medium) containing DMEM/F12 (3:1) supplemented with 2% B27 (without vitamin A) and 1×NEAA with daily medium change.

qPCR analysis: Total RNA was isolated with the RNeasy Plus Mini Kit according to the manufacturer's instructions. For quantitative PCR (qPCR), cDNA was synthesized using PrimeScrip RT Reagent Kit. qPCR was performed using iTaq Universal SYBR Green Supermix. GAPDH gene was used as an internal control to equalize cDNA.

Immunocytochemistry, western blotting and flow cytometry: Immunocytochemistry and western blotting were performed as described previously (Huang et al., 2016). In brief, cells on coverslips were fixed in 4% neutral-buffered paraformaldehyde (PFA) for 10 minutes at room temperature. Following rinsing with PBS, they were incubated in 0.2% triton x-100 (in PBS) for 10 min followed by 10% donkey serum (in PBS) at room temperature for 1 hour. They were then incubated with primary antibodies diluted in 5% donkey serum in 0.1% triton x-100 (in PBS) at 4° C. overnight, followed by fluorescently conjugated secondary antibodies at room temperature for 30 minutes. The nuclei were stained with Hoechst. Images were collected with a Nikon A1 laser-scanning confocal microscope. For western blotting, cells were lysed in 1×RIPA and 1× protease inhibitor cocktail. Proteins (15 μg) in the supernatant were boiled in SDS-PAGE sample buffer and separated by 10% SDS-PAGE.

Flow cytometry was performed using Transcription Factor Buffer Set which is designed for transcription factor staining following manufacturer's instruction. Briefly, single cells were prepared using TrypLE Express Enzyme and fixed in the fixation buffer provided by the kit at 2-8° C. for 45 min. After 3 washings with the permeable buffer, the primary antibodies (PE-SOX1, V450-SOX2) were added to cells for 45 min at 2-8° C. in a light-tight box. The cells were washed 3 times before analyzed by flow cytometry (BD LSR or BD LSRII). Data analysis was performed using FlowJo.

RNA-Seq, ChIP-Seq and data analysis: RNAs were prepared as for qPCR. The RNA samples (2 μg each) were processed by HiSeq at 1×100 with 3 samples per lane. RNA-Seq was performed at DNA Sequencing Facility in the University of Wisconsin-Madison Biotechnology Center. RNA-Seq data were processed following quality control, mapping and analysis of transcripts using Galaxy following FastQC, HISAT2, Cufflinks and DESeq2 pipelines.

ChIP-Seq samples were prepared by using Magnetic ChIP Kit following manufacturer's instruction. Briefly, the cultures were digested with TrypLE to become single cells and then were crosslinked using 1% formaldehyde provided by the kit. The cells were incubated at room temperature for 10 mins in a chemical fume hood. After incubation, the cells were treated with glycine solution for 5 mins at room temperature. After washing with cold PBS twice, the cells were lysed with the lysis buffer in the presence of protease inhibitors and MNase (provided by the kit to digest DNA). They were then sonicated (three 20-second pulses at 3 watts power) to yield DNA fragments of about 150 basepairs (bp) to 1000 bp. After taking 10 μl samples as an INPUT the rest samples were incubated with 1 μg FLAG antibody in 100 μl IP buffer provided by the kit overnight at 2-8° C. The pull-downs were harvested by ChIP Grade Protein A/G Magnetic Beads. The DNAs recovered from the INPUTs and IPs were submitted to University of Wisconsin-Madison Biotechnology Center for sequencing using HiSeq at 1×100 bp. The Seq data were processed using Galaxy following FastQC, Trimmomatic, Bowtie2 and MACS2 callpeak pipeline. The regional bed files were annotated by PAVIS.

TABLE 1
Reagents and Resources

| REAGENT or RESOURCE | SOURCE | IDENTIFIER |
|---|---|---|
| Antibodies | | |
| C′-PAX6 | Covance | Cat# PRB-278P; |
| | | RRID: AB_291612 |
| N′-PAX6 | Santa Cruz | Cat# sc-32766 X; |
| | Biotechnology | RRID: AB_628107 |
| Pax6 Alexa Fluor ® 488 | BD Biosciences | Cat# 561664 |
| SOX2-V450 | BD Biosciences | Cat# 561610 |
| SOX1-PE | BD Biosciences | Cat# 561592 |
| VSX2 | exalpha | Cat# X1180P |
| β-Actin | Sigma-Aldrich | A1978; |
| | | RRID: AB_476692 |
| SOX2 | R&D Systems | AF2018; |
| | | RRID: AB_355110 |
| SOX1 | R&D Systems | AF3369; |
| | | RRID: AB_2239879 |
| Monoclonal ANTI-FLAG ® M2 antibody | Sigma-Aldrich | F1804; |
| | | RRID: AB_262044 |
| Alexa Fluor 488 donkey anti-goat IgG (H + L) | Molecular Probes | Cat# A11055; |
| | | RRID: AB_142672 |
| Alexa Fluor 546 Donkey Anti-Mouse IgG | Molecular Probes | Cat# A10036; |
| | | RRID: AB_2534012 |
| Alexa Fluor 488, Donkey anti-Rabbit IgG (H + L) | Life Technologies | Cat# A21206; |
| | | RRID: AB_141708 |
| Alexa Fluor 546 donkey anti-sheep IgG (H + L) | Molecular Probes | Cat# A21098; |
| | | RRID: AB_1500708 |
| Alexa Fluor 546 donkey anti-rabbit IgG (H + L) | Life Technologies | Cat# A10040; |
| | | RRID: AB_2534016 |
| Biological Samples | | |
| Human embryo tissue | Tongji Hospital (see | |
| | english.tjh.com.cn on | |
| | the World Wide Web | |
| | (WWW)) | |
| Chemicals, Peptides, and Recombinant Proteins | | |
| CHIR99021 | Stemgent | Cat# 04-0004-10 |
| SB431542 | Stemgent | Cat# 04-0010-10 |
| DMH1 | Tocris Bioscience | Cat# 4126 |
| IWR1 | Sigma-Aldrich | Cat# I0161 |
| protease inhibitor cocktail | Sigma-Aldrich | Cat# P8340 |
| Rho Kinase Inhibitor | MilliporeSigma | Cat# 555550 |
| Critical Commercial Assays | | |
| Magnetic ChIP Kit | Thermo Scientific | Cat# 26157 |
| Transcription Factor Buffer Set | BD Biosciences | Cat# 562574 |
| RNeasy mini kit | QIAGEN | Cat# 74106 |
| PrimeScrip RT Reagent Kit | Clontech | Cat# RR037A |
| Deposited Data | | |
| Experimental Models: Cell Lines | | |
| H9 human embryonic stem cells | WiCell Research | WA09 |
| | Institute | |
| PAX6 KO cell line derived from H9 hESC | This paper | N/A |
| PAX6D KO cell line derived from H9 hESC | This paper | N/A |
| PAX6A TetOn cell line derived from PAX6 KO cell line | This paper | N/A |
| PAX6D TetOn cell line derived from PAX6 KO cell line | This paper | N/A |
| Oligonucleotides | | |
| Recombinant DNA | | |
| PL552 | (Chen et al., 2015) | Addgene: |
| | | Plasmid #68407 |
| PAX6-Exon8-puromycin donor plasmid | This paper | N/A |
| PAX6A-3XFLAG-TETON donor plasmid | This paper | N/A |
| PAX6D-3XFLAG-TETON donor plasmid | This paper | N/A |
| Software and Algorithms | | |
| FlowJo | Available at | RRID: SCR_008520 |
| | flowjo.com on the | |
| | WWW | |
| Galaxy | Galaxy (available at | RRID: SCR_006281 |
| | usegalaxy.org/ on the | |
| | WWW) | |
| PAVIS | NIH (available at | |
| | manticore.niehs.nih.go | |
| | v/pavis2 on the | |
| | WWW) | |
| Other | | |
| E8 | WiCell Research | |
| | Institute | |
| Matrigel ® | Coming Life Sciences | Cat# 2014-06-03 |
| Vitronectin | StemCell | Cat# 07180 |
| | Technologies | |
| N2 | ThermoFisher | Cat# 17502048 |
| B27 | ThermoFisher | |
| DMEM/F12 1:1 | ThermoFisher | Cat# 11330-057 |
| Neurobasal | ThermoFisher | |
| Dispase | ThermoFisher | Cat# 17105-041 |
| NEAA | ThermoFisher | Cat# 11140050 |
| GlutaMAX | ThermoFisher | Cat# 35050061 |
| FBS | | |
| RIPA | Cell Signaling | Cat# 9806S |
| | Technology | |
| TrypLE | ThermoFisher | Cat# 12604021 |

TABLE 2
PAX6KO VS WT

| tracking_id | gene | WT_1 | WT_0 | WT_2 | KO_0 | KO_1 | KO_2 | sample_1 |
|---|---|---|---|---|---|---|---|---|
| XLOC_002465 | KMO | 5.58462 | 16.448 | 13.9268 | 0 | 0 | 0.007775 | WT |
| XLOC_014867 | VSX2 | 24.0414 | 42.2013 | 44.5609 | 0.01864 | 0.034462 | 0.03074 | WT |
| XLOC_032376 | CBR1 | 4.59249 | 2.70212 | 5.44573 | 0 | 0.02221 | 0 | WT |
| XLOC_051344 | ALDH1A1 | 268.278 | 337.186 | 344.585 | 0.785441 | 0.99738 | 0.361109 | WT |
| XLOC_019330 | HSD17B2 | 108.573 | 79.3757 | 74.2518 | 0.398511 | 0.219897 | 0.130751 | WT |
| XLOC_053625 | XIST, | 10.1235 | 32.7084 | 11.2266 | 0.126763 | 0.011328 | 0.027222 | WT |
| | XIST_intron, | | | | | | | |
| | Xist_exon4 | | | | | | | |
| XLOC_027099 | IAH1 | 6.35612 | 26.3106 | 8.48768 | 0.019664 | 0 | 0.107888 | WT |
| XLOC_000776 | FOXE3 | 14.8087 | 39.8849 | 41.069 | 0.132008 | 0.135586 | 0.064502 | WT |
| XLOC_023004 | TBX2-AS1 | 8.78707 | 13.6519 | 21.7809 | 0 | 0.197674 | 0 | WT |
| XLOC_049879 | TYRP1 | 235.705 | 54.4611 | 38.9429 | 0.425613 | 0.608062 | 0.466756 | WT |
| XLOC_021600 | TBX2 | 6.63337 | 6.93052 | 16.2837 | 0.042118 | 0.107251 | 0 | WT |
| XLOC_017703 | CYP19A1 | 0.203446 | 0.452523 | 0.301805 | 0 | 0.00595 | 0 | WT |
| XLOC_039936 | BHMT | 37.3323 | 111.92 | 120.503 | 1.15546 | 0.834651 | 0.47749 | WT |
| XLOC_014052 | DCT | 116.653 | 24.9895 | 9.93978 | 0.491043 | 0.44155 | 0.740795 | WT |
| XLOC_048042 | CLVS1 | 24.3687 | 22.2627 | 29.6563 | 0.338402 | 0.408967 | 0.12562 | WT |
| XLOC_025713 | ZNF667-AS1 | 4.73676 | 9.32947 | 4.82475 | 0 | 0.01833 | 0.202607 | WT |
| XLOC_036590 | AC020636.2, | 1.64049 | 4.57607 | 3.36258 | 0.066074 | 0.061078 | 0 | WT |
| | CLRN1, | | | | | | | |
| | MINDY4B | | | | | | | |
| XLOC_023764 | CDH20 | 37.685 | 38.8728 | 33.3169 | 0.571475 | 0.836264 | 0.161578 | WT |
| XLOC_026995 | ZNF667 | 1.0593 | 3.4673 | 1.3381 | 0.073367 | 0.015706 | 0 | WT |
| XLOC_002958 | AL391650.1 | 15.5575 | 10.81 | 14.5734 | 0.326529 | 0.196753 | 0.116703 | WT |
| XLOC_006922 | PITX3 | 1.52713 | 5.05783 | 6.0417 | 0.053264 | 0.098475 | 0.058559 | WT |
| XLOC_034614 | SYNPR | 15.8505 | 11.9411 | 10.1719 | 0.291191 | 0.115033 | 0.234572 | WT |
| XLOC_036565 | CP | 6.26707 | 50.9784 | 42.8121 | 0.244886 | 0.895046 | 0.645878 | WT |
| XLOC_018597 | CLEC19A | 23.9987 | 10.9556 | 18.88 | 0.693659 | 0.404247 | 0.026297 | WT |
| XLOC_030767 | IQCA1 | 3.49601 | 1.23853 | 3.80359 | 0.157587 | 0.024472 | 0.019377 | WT |
| XLOC_008243 | TYR | 6.37815 | 2.43474 | 0.474991 | 0.042033 | 0.019445 | 0.159472 | WT |
| XLOC_024400 | FAM69C | 11.6468 | 9.87495 | 8.56703 | 0.138958 | 0.278207 | 0.306086 | WT |
| XLOC_048281 | ERICH5 | 16.2999 | 41.7988 | 46.1951 | 0.503349 | 1.93694 | 0.239438 | WT |
| XLOC_033225 | CRYBB3 | 1.85882 | 7.03654 | 4.079 | 0.079355 | 0.110033 | 0.174487 | WT |
| XLOC_050766 | BARHL1 | 7.75494 | 3.28797 | 51.4482 | 0.367364 | 1.01455 | 0.516136 | WT |
| XLOC_027566 | ATP6V1B1 | 4.78079 | 11.3255 | 5.64947 | 0.156006 | 0.278237 | 0.250328 | WT |
| XLOC_001908 | PAPPA2 | 11.3668 | 17.3899 | 23.9001 | 0.554404 | 1.01577 | 0.198088 | WT |
| XLOC_040715 | ZNF454 | 1.62015 | 1.23638 | 1.42324 | 0.040327 | 0.066599 | 0.040361 | WT |
| XLOC_005323 | AL117339.1, | 5.32891 | 7.22129 | 4.17298 | 0.202319 | 0.050182 | 0.360196 | WT |
| | AL117339.2, | | | | | | | |
| | AL117339.5, | | | | | | | |
| | AL133217.1, | | | | | | | |
| | HSD17B7P2, | | | | | | | |
| | ZNF37A | | | | | | | |
| XLOC_025547 | ZNF528 | 12.445 | 18.014 | 11.5862 | 0.671718 | 0.322339 | 0.603468 | WT |
| XLOC_041138 | AC034238.1, | 1.17155 | 1.49775 | 7.77941 | 0.09496 | 0.290675 | 0.043725 | WT |
| | ESM1 | | | | | | | |
| XLOC_030921 | LAMP5 | 3.28604 | 11.1172 | 8.30284 | 0.282797 | 0.420105 | 0.235206 | WT |
| XLOC_002728 | MIR34A, | 1.4038 | 120.464 | 2.53687 | 2.12085 | 1.40552 | 1.6788 | WT |
| | MIR34AHG | | | | | | | |
| XLOC_025503 | SIGLEC7 | 0.157275 | 0.423744 | 0.404266 | 0 | 0.019089 | 0.022702 | WT |
| XLOC_017273 | AC015712.1, | 61.8026 | 83.4586 | 50.6442 | 2.74631 | 2.67884 | 3.08856 | WT |
| | AC015712.4, | | | | | | | |
| | AC015712.5, | | | | | | | |
| | AC015712.6, | | | | | | | |
| | ALDH1A3 | | | | | | | |
| XLOC_048386 | AC064802.1 | 0.102552 | 4.43215 | 0.158353 | 0.053631 | 0.099148 | 0.058961 | WT |
| XLOC_048495 | AC104257.1 | 0.095814 | 8.87889 | 0.098629 | 0.24289 | 0.138958 | 0.055087 | WT |
| XLOC_005835 | PAX2 | 1.3601 | 1.26266 | 2.89939 | 0.136754 | 0.064097 | 0.08609 | WT |
| XLOC_027327 | QPCT | 2.4727 | 4.20444 | 2.60496 | 0.237099 | 0.217064 | 0.037368 | WT |
| XLOC_041832 | PPP2R2B, | 370.411 | 12.3097 | 10.1457 | 6.91133 | 7.70502 | 6.19719 | WT |
| | RNA5SP196 | | | | | | | |
| XLOC_021142 | CCL2 | 271.652 | 422.61 | 418.085 | 20.6963 | 19.7372 | 19.511 | WT |
| XLOC_018051 | CTXND1 | 0.289552 | 0.322075 | 0.439449 | 0.034945 | 0.025124 | 0 | WT |
| XLOC_014741 | SIX6 | 11.7499 | 22.4652 | 21.0377 | 1.31962 | 0.754259 | 1.16286 | WT |
| XLOC_034205 | HRH1 | 0.728556 | 2.35439 | 2.62183 | 0.083087 | 0.147918 | 0.10518 | WT |
| XLOC_043011 | AL589740.1 | 2.83443 | 1.99495 | 3.03891 | 0.26232 | 0.141783 | 0.070279 | WT |
| XLOC_026261 | AC011447.2, | 1.23477 | 2.09424 | 1.04945 | 0.128454 | 0.047166 | 0.098661 | WT |
| | AC011447.3 | | | | | | | |
| XLOC_005448 | DKK1 | 13.6083 | 16.1791 | 24.6678 | 0.691665 | 2.20212 | 0.519121 | WT |
| XLOC_020429 | ADAMTS18 | 20.9749 | 23.2548 | 27.0867 | 0.354768 | 3.82669 | 0.386582 | WT |
| XLOC_009925 | CRYAB | 366.654 | 1347.99 | 1734.04 | 50.3919 | 129.232 | 41.5954 | WT |
| XLOC_034818 | ZPLD1 | 0.454756 | 0.348382 | 0.944384 | 0.019678 | 0.072604 | 0.021541 | WT |
| XLOC_009328 | MS4A6A | 4.13338 | 14.4128 | 33.2609 | 2.25501 | 0.751567 | 0.495463 | WT |
| XLOC_008373 | GRIA4 | 4.46608 | 10.8139 | 10.2688 | 0.697804 | 0.619828 | 0.431725 | WT |
| XLOC_008341 | CNTN5 | 2.87317 | 6.12486 | 6.60276 | 0.560839 | 0.381828 | 0.155141 | WT |
| XLOC_002706 | TNFRSF9 | 0.955634 | 5.22558 | 3.9647 | 0.210562 | 0.179023 | 0.331729 | WT |
| XLOC_024733 | ZNF440 | 2.88092 | 2.96653 | 2.67963 | 0.586055 | 0.03355 | 0.007167 | WT |
| XLOC_027565 | VAX2 | 1.61982 | 2.62167 | 1.2574 | 0.166645 | 0.077023 | 0.18321 | WT |
| XLOC_049024 | ZMAT4 | 5.32469 | 8.89905 | 5.09229 | 0.778776 | 0.299055 | 0.508701 | WT |
| XLOC_002386 | TRIM67 | 0.166249 | 0.425185 | 1.14544 | 0.02324 | 0.061207 | 0.05832 | WT |
| XLOC_008662 | GLB1L3 | 0.164977 | 0.385587 | 0.696593 | 0.039122 | 0.022422 | 0.043744 | WT |
| XLOC_039183 | SFRP2 | 476.589 | 775.446 | 776.525 | 41.9701 | 59.0123 | 71.7994 | WT |
| XLOC_045037 | AC004947.2 | 0.595058 | 0.301741 | 0.228407 | 0.042196 | 0.019503 | 0.034793 | WT |
| XLOC_030694 | SPHKAP | 0.108141 | 0.243771 | 0.44131 | 0.023598 | 0.036358 | 0.008647 | WT |
| XLOC_024166 | KLHL14 | 5.4997 | 7.63757 | 10.9 | 0.689436 | 0.970581 | 0.514585 | WT |
| XLOC_007296 | CCKBR | 18.9483 | 29.6155 | 16.3185 | 1.23678 | 2.56527 | 2.18641 | WT |
| XLOC_050235 | AL353608.3, | 14.8368 | 9.57984 | 9.86534 | 0.437383 | 2.43281 | 0.394676 | WT |
| | PGM5 | | | | | | | |
| XLOC_048391 | SLC30A8 | 0.795451 | 0.053458 | 1.66155 | 0.121858 | 0.096619 | 0.024714 | WT |
| XLOC_053146 | ANOS1 | 11.8847 | 12.3872 | 21.3149 | 1.53808 | 1.55263 | 1.35711 | WT |
| XLOC_030190 | CYTIP | 0.890241 | 0.76244 | 0.753724 | 0.079225 | 0.069089 | 0.088407 | WT |
| XLOC_012216 | PMEL | 380.885 | 280.488 | 177.492 | 27.7829 | 20.9788 | 34.3185 | WT |
| XLOC_053685 | HDX | 3.01945 | 3.40599 | 2.8989 | 0.231165 | 0.289983 | 0.403669 | WT |
| XLOC_049217 | CPA6 | 0.274907 | 0.721336 | 0.287373 | 0.047606 | 0.029426 | 0.052338 | WT |
| XLOC_010972 | LGR5 | 19.3417 | 19.2728 | 20.0357 | 1.79896 | 2.47391 | 1.65587 | WT |
| XLOC_029247 | CYP1B1, | 12.7944 | 15.2601 | 26.2721 | 1.77355 | 2.09612 | 1.72755 | WT |
| | RMDN2-AS1 | | | | | | | |
| XLOC_019871 | EIF3CL | 2.88923 | 0.208908 | 0.197669 | 0.127808 | 0.126584 | 0.090328 | WT |
| XLOC_048112 | KCNB2 | 1.51573 | 1.68034 | 1.73448 | 0.177006 | 0.291676 | 0.050766 | WT |
| XLOC_012370 | CPM, | 2.64187 | 6.95502 | 4.36488 | 0.20834 | 1.17347 | 0.089223 | WT |
| | PRELID2P1 | | | | | | | |
| XLOC_040762 | AC138035.1 | 0.275702 | 4.87749 | 0.33735 | 0.331399 | 0.112627 | 0.146464 | WT |
| XLOC_030742 | AC064852.1, | 12.6033 | 9.78836 | 10.0916 | 1.69547 | 1.00666 | 0.820057 | WT |
| | KCNJ13 | | | | | | | |
| XLOC_030425 | TMEFF2 | 40.0945 | 26.9503 | 23.8186 | 5.34252 | 3.27494 | 1.51246 | WT |
| XLOC_050331 | NTRK2 | 4.07667 | 7.93482 | 8.77045 | 0.791528 | 0.741549 | 0.800643 | WT |
| XLOC_017031 | CRABP1 | 1195.31 | 2227.35 | 1893.36 | 231.873 | 162.117 | 220.572 | WT |
| XLOC_020676 | ZFP3 | 0.799269 | 1.24264 | 0.965845 | 0.223275 | 0.086397 | 0.051377 | WT |
| XLOC_002160 | C1orf186 | 0.63685 | 6.86482 | 5.34159 | 0.746386 | 0.256596 | 0.552408 | WT |
| XLOC_030368 | PDE1A | 0.908066 | 2.7045 | 1.46131 | 0.280628 | 0.158035 | 0.176116 | WT |
| XLOC_037964 | MAB21L2 | 47.595 | 42.199 | 40.2181 | 3.3336 | 8.70142 | 3.77638 | WT |
| XLOC_005996 | AL133461.1, | 69.0599 | 37.8241 | 76.9869 | 7.42752 | 8.09973 | 6.86225 | WT |
| | INPP5F | | | | | | | |
| XLOC_041354 | ANKRD34B | 2.39566 | 1.69226 | 3.27724 | 0.257529 | 0.437512 | 0.221912 | WT |
| XLOC_010676 | GPD1 | 1.08232 | 1.40149 | 1.99675 | 0.1406 | 0.265826 | 0.156384 | WT |
| XLOC_035638 | WNT7A | 5.38763 | 3.83589 | 5.53929 | 0.284464 | 1.24591 | 0.372312 | WT |
| XLOC_005857 | ELOVL3 | 0.278393 | 1.08916 | 0.837677 | 0.156789 | 0.103525 | 0.024625 | WT |
| XLOC_016442 | GABRG3 | 5.30885 | 4.53087 | 3.7671 | 0.686877 | 0.400254 | 0.677391 | WT |
| XLOC_011052 | ALX1 | 3.42173 | 1.64595 | 6.05004 | 0.505257 | 0.661662 | 0.277741 | WT |
| XLOC_042581 | MIR5004, | 0.059656 | 0.074733 | 1.99489 | 0.069121 | 0.085339 | 0.122918 | WT |
| | SYNGAP1, | | | | | | | |
| | ZBTB9 | | | | | | | |
| XLOC_040023 | NR2F1 | 121.883 | 94.1868 | 150.864 | 14.0297 | 20.7967 | 13.777 | WT |
| XLOC_050764 | CFAP77 | 12.5485 | 8.31813 | 19.407 | 2.20781 | 1.96837 | 1.18565 | WT |
| XLOC_029134 | POMC | 5.43884 | 2.014 | 1.88963 | 0.193116 | 0.564571 | 0.497085 | WT |
| XLOC_010672 | AQP5 | 1.44334 | 1.67927 | 2.11868 | 0.14677 | 0.213201 | 0.345772 | WT |
| XLOC_037612 | SPP1 | 205.264 | 907.926 | 297.504 | 44.3401 | 110.052 | 36.3142 | WT |
| XLOC_027527 | MEIS1 | 85.4694 | 87.4353 | 66.8553 | 9.0631 | 10.7953 | 12.6073 | WT |
| XLOC_027038 | ZNF132 | 0.683199 | 0.526368 | 0.67917 | 0.13304 | 0.078323 | 0.055072 | WT |
| XLOC_028175 | THSD7B | 2.67226 | 1.08222 | 1.82101 | 0.435101 | 0.319927 | 0.035385 | WT |
| XLOC_037904 | IL15 | 0.085657 | 0.318399 | 0.490296 | 0.026534 | 0.077714 | 0.023108 | WT |
| XLOC_032052 | APCDD1L | 2.60783 | 2.44671 | 2.12832 | 0.112974 | 0.77579 | 0.151569 | WT |
| XLOC_002049 | CRB1 | 2.30468 | 4.54208 | 3.18978 | 0.47868 | 0.516588 | 0.469603 | WT |
| XLOC_041731 | SPOCK1 | 145.596 | 162.91 | 145.988 | 20.6471 | 29.0646 | 16.7045 | WT |
| XLOC_021787 | SPHK1 | 23.2995 | 23.0011 | 37.1734 | 2.73315 | 5.2386 | 4.32917 | WT |
| XLOC_039364 | LINC02365 | 0.869773 | 2.04544 | 1.84125 | 0.29988 | 0.182768 | 0.219795 | WT |
| XLOC_013274 | PCDH17 | 3.06463 | 3.95002 | 5.43705 | 0.404765 | 1.05807 | 0.381317 | WT |
| XLOC_012994 | FGF9 | 3.59168 | 3.87117 | 4.71782 | 1.09445 | 0.542489 | 0.198115 | WT |
| XLOC_046363 | DGKB | 2.01428 | 2.09315 | 4.62416 | 0.214465 | 0.618696 | 0.489471 | WT |
| XLOC_024736 | ZNF69 | 4.05827 | 3.56241 | 2.67414 | 0.928156 | 0.208263 | 0.47991 | WT |
| XLOC_045760 | FOXP2 | 5.88623 | 4.84709 | 5.1874 | 0.929759 | 0.946861 | 0.631826 | WT |
| XLOC_051664 | TNC | 213.252 | 360.119 | 156.583 | 27.0482 | 82.8707 | 7.52532 | WT |
| XLOC_043359 | PPP1R14C | 2.46775 | 7.44666 | 9.84518 | 1.11001 | 1.47113 | 0.617525 | WT |
| XLOC_009884 | SLN | 37.5379 | 17.253 | 7.92598 | 2.14181 | 7.40106 | 0.728843 | WT |
| XLOC_002245 | PROX1 | 2.4965 | 5.75226 | 8.62622 | 0.853275 | 0.655323 | 1.27477 | WT |
| XLOC_013601 | GJA3 | 0.317404 | 0.736926 | 0.633042 | 0.098558 | 0.095901 | 0.085543 | WT |
| XLOC_037491 | AMBN | 13.1172 | 21.3811 | 6.3971 | 2.37706 | 1.81575 | 2.70325 | WT |
| XLOC_043013 | POU3F2 | 3.49554 | 4.31769 | 5.55051 | 0.672322 | 1.03239 | 0.555893 | WT |
| XLOC_026854 | HAS1 | 0.300599 | 0.742958 | 0.50416 | 0.01383 | 0.126367 | 0.121737 | WT |
| XLOC_049110 | OPRK1 | 0.32442 | 0.271044 | 0.25725 | 0.070695 | 0.048264 | 0.025511 | WT |
| XLOC_005951 | ADRB1 | 0.676098 | 0.543138 | 0.695966 | 0.049108 | 0.190659 | 0.086383 | WT |
| XLOC_041015 | SLC45A2 | 1.66043 | 0.787502 | 0.837097 | 0.270953 | 0.169311 | 0.119558 | WT |
| XLOC_007768 | P2RX3 | 11.4484 | 5.26939 | 10.4009 | 1.91682 | 2.05662 | 0.67398 | WT |
| XLOC_052548 | POU3F4 | 0.385523 | 0.485639 | 0.341154 | 0.021223 | 0.163484 | 0.023332 | WT |
| XLOC_013946 | PCDH9 | 2.76325 | 2.87375 | 3.69818 | 0.498438 | 0.778085 | 0.332542 | WT |
| XLOC_008890 | SCUBE2 | 13.2784 | 5.16013 | 8.37681 | 1.11418 | 1.9466 | 1.56159 | WT |
| XLOC_039304 | HPGD | 3.95569 | 6.09741 | 1.37806 | 0.628612 | 1.17793 | 0.172886 | WT |
| XLOC_015380 | SLC7A8 | 121.946 | 126.791 | 162.96 | 27.4085 | 23.3139 | 21.0448 | WT |
| XLOC_006001 | PLPP4 | 7.78586 | 3.5804 | 4.4459 | 0.314801 | 1.86296 | 0.59 | WT |
| XLOC_045083 | GHRHR | 1.17316 | 0.501402 | 2.24275 | 0.175292 | 0.324078 | 0.192718 | WT |
| XLOC_034738 | EPHA3 | 6.93189 | 6.01915 | 5.04062 | 1.26215 | 1.38313 | 0.634421 | WT |
| XLOC_051434 | LINC01508 | 1.16349 | 1.82433 | 1.59586 | 0.681035 | 0.119913 | 0.035654 | WT |
| XLOC_002251 | KCNK2 | 2.34228 | 3.29868 | 2.04425 | 0.363569 | 0.661815 | 0.37967 | WT |
| XLOC_017507 | GJD2 | 1.86204 | 3.68614 | 4.5392 | 0.232546 | 1.48683 | 0.127831 | WT |
| XLOC_020114 | CBLN1 | 2.041 | 5.46001 | 6.42341 | 1.36519 | 1.08027 | 0.118286 | WT |
| XLOC_003943 | AC239809.3 | 0.428285 | 0.493307 | 0.192264 | 0.044778 | 0.097086 | 0.063295 | WT |
| XLOC_012012 | AMIGO2 | 3.45442 | 4.11641 | 4.20926 | 0.662173 | 0.973802 | 0.548318 | WT |
| XLOC_027037 | AC012313.5 | 0.258241 | 0.461412 | 0.454127 | 0.101289 | 0.03121 | 0.086611 | WT |
| XLOC_037223 | LINC02506 | 6.23974 | 51.9183 | 13.6307 | 7.62237 | 2.99343 | 2.90291 | WT |
| XLOC_025549 | ZNF578 | 0.415504 | 0.660463 | 0.38739 | 0.128756 | 0.078826 | 0.070702 | WT |
| XLOC_003160 | COL9A2 | 66.9667 | 40.0417 | 68.2401 | 9.52235 | 12.0478 | 12.0696 | WT |
| XLOC_032087 | GATA5 | 0.416305 | 0.300992 | 0.128562 | 0.054428 | 0.060376 | 0.047871 | WT |
| XLOC_018083 | HOMER2 | 58.9068 | 72.7643 | 58.6159 | 14.1783 | 11.8055 | 10.6426 | WT |
| XLOC_006181 | GATA3-AS1 | 0.309749 | 0.111203 | 0.606512 | 0.025918 | 0.108609 | 0.064331 | WT |
| XLOC_036866 | SST | 95.6846 | 43.2791 | 93.8272 | 20.2596 | 22.4615 | 2.39652 | WT |
| XLOC_001171 | VCAM1 | 8.95852 | 10.043 | 10.7981 | 1.87309 | 1.34404 | 2.55954 | WT |
| XLOC_041413 | LINC00461, | 25.0832 | 13.5245 | 14.908 | 2.10078 | 6.15445 | 2.12843 | WT |
| | MIR9-2 | | | | | | | |
| XLOC_036625 | AC108729.3, | 27.0835 | 230.605 | 21.8681 | 15.8527 | 23.2701 | 15.1243 | WT |
| | PLCH1 | | | | | | | |
| XLOC_027645 | AC011754.1, | 51.0326 | 63.5114 | 84.49 | 13.8058 | 10.4825 | 14.3959 | WT |
| | CTNNA2 | | | | | | | |
| XLOC_032532 | U2AF1L5 | 40.9568 | 0.70273 | 16.055 | 1.15162 | 0.449138 | 9.77284 | WT |
| XLOC_043065 | NR2E1 | 8.03942 | 8.30737 | 5.79257 | 1.04822 | 1.67724 | 1.67425 | WT |
| XLOC_038190 | ZFP42 | 4.22012 | 5.08016 | 0.303557 | 0.217735 | 1.37515 | 0.31579 | WT |
| XLOC_049746 | MAFA | 0.615728 | 1.59732 | 0.741452 | 0.206569 | 0.146021 | 0.240463 | WT |
| XLOC_001567 | SPRR3 | 1.80877 | 2.14418 | 0.038401 | 0.201684 | 0.528135 | 0.073914 | WT |
| XLOC_038812 | SNCA | 120.444 | 90.172 | 102.326 | 24.1095 | 22.0605 | 17.3679 | WT |
| XLOC_051436 | DIRAS2 | 1.58807 | 0.610146 | 0.494273 | 0.142896 | 0.336471 | 0.075135 | WT |
| XLOC_042949 | HTR1E | 3.93668 | 0.968023 | 0.721655 | 0.197415 | 0.899408 | 0.062011 | WT |
| XLOC_025172 | AC020928.1 | 2.74776 | 3.61071 | 2.65458 | 0.654476 | 0.736452 | 0.466231 | WT |
| XLOC_004746 | SUSD4 | 3.80688 | 5.08067 | 6.9578 | 0.657414 | 1.50561 | 1.13032 | WT |
| XLOC_046915 | SEMA3E | 16.2428 | 10.2963 | 8.49356 | 0.847081 | 5.80519 | 0.668985 | WT |
| XLOC_012705 | TBX3 | 3.01063 | 1.33223 | 3.04844 | 0.229944 | 1.23852 | 0.082164 | WT |
| XLOC_004935 | GREM2 | 0.745461 | 0.730919 | 0.535223 | 0.157251 | 0.230156 | 0.036017 | WT |
| XLOC_044557 | MOXD1 | 6.29193 | 9.72216 | 8.92068 | 2.24031 | 2.22334 | 0.881091 | WT |
| XLOC_021148 | TMEM132E | 1.40988 | 0.486328 | 0.913432 | 0.177407 | 0.362391 | 0.073125 | WT |
| XLOC_030096 | NXPH2 | 3.28156 | 2.97772 | 2.0694 | 0.40198 | 1.08618 | 0.339954 | WT |
| XLOC_035763 | HSPD1P6 | 3.67747 | 0.422261 | 0.017363 | 0.15878 | 0.594349 | 0.155168 | WT |
| XLOC_000763 | DMBX1 | 0.399692 | 0.713761 | 0.602804 | 0.136058 | 0.224676 | 0.021369 | WT |
| XLOC_008424 | NCAM1 | 33.6777 | 64.3546 | 60.4432 | 10.2619 | 15.6837 | 10.8451 | WT |
| XLOC_019146 | LRRC36 | 0.082067 | 1.9616 | 0.160277 | 0.044185 | 0.218009 | 0.251872 | WT |
| XLOC_049099 | PXDNL | 0.175017 | 0.236865 | 0.356232 | 0.069786 | 0.046767 | 0.063936 | WT |
| XLOC_030539 | FZD5 | 6.3426 | 16.7834 | 14.869 | 2.12749 | 1.91867 | 4.93247 | WT |
| XLOC_031027 | SYNDIG1 | 1.17031 | 1.60609 | 1.62468 | 0.258288 | 0.581328 | 0.208485 | WT |
| XLOC_003694 | VAV3 | 7.82057 | 7.47898 | 7.65401 | 1.78451 | 1.96246 | 1.73747 | WT |
| XLOC_051965 | STS | 2.61304 | 1.7908 | 2.56444 | 0.64114 | 0.824413 | 0.203329 | WT |
| XLOC_037731 | EGF | 6.09219 | 7.1877 | 11.2359 | 2.36871 | 1.34669 | 2.1663 | WT |
| XLOC_001154 | PALMD | 4.296 | 3.60518 | 3.41303 | 1.18303 | 1.43369 | 0.098937 | WT |
| XLOC_038913 | COL25A1 | 2.676 | 4.13148 | 6.52599 | 0.782579 | 1.57097 | 0.861524 | WT |
| XLOC_039460 | ADAMTS16 | 3.25299 | 8.01238 | 12.5486 | 1.5952 | 3.13699 | 1.03624 | WT |
| XLOC_047896 | C8orf4 | 40.6436 | 47.2069 | 50.5633 | 6.11582 | 21.9563 | 5.52496 | WT |
| XLOC_023770 | TNFRSF11A | 0.265996 | 0.364042 | 0.518576 | 0.084356 | 0.078018 | 0.118104 | WT |
| XLOC_049601 | HAS2 | 3.02918 | 1.83986 | 1.75799 | 0.573686 | 0.783415 | 0.272354 | WT |
| XLOC_014587 | LRFN5 | 0.98166 | 0.563432 | 5.00363 | 0.472748 | 0.853526 | 0.284234 | WT |
| XLOC_023424 | APCDD1 | 46.322 | 33.9943 | 47.0792 | 9.35286 | 18.6627 | 3.39273 | WT |
| XLOC_009539 | FGF19 | 0.90998 | 1.37229 | 1.03953 | 0.127677 | 0.525744 | 0.178652 | WT |
| XLOC_015770 | SLC8A3 | 0.091533 | 0.567282 | 0.479065 | 0.173045 | 0.058317 | 0.054261 | WT |
| XLOC_009129 | CHRM4 | 2.88468 | 0.323874 | 0.491631 | 0.29972 | 0.480236 | 0.175741 | WT |
| XLOC_043180 | HEY2 | 3.19684 | 2.59449 | 1.69281 | 0.530738 | 0.927716 | 0.485845 | WT |
| XLOC_050369 | C9orf47, | 10.2433 | 24.3117 | 16.6353 | 4.7981 | 4.99107 | 3.58054 | WT |
| | S1PR3 | | | | | | | |
| XLOC_046916 | SEMA3A | 52.7525 | 42.2479 | 39.8948 | 8.52092 | 16.726 | 10.0119 | WT |
| XLOC_048409 | COL14A1 | 2.59648 | 2.10468 | 3.61912 | 0.460902 | 1.45697 | 0.257723 | WT |
| XLOC_002599 | AL645608.1 | 2.45427 | 1.86025 | 2.54578 | 0.488773 | 1.13383 | 0.173673 | WT |
| XLOC_003586 | BARHL2 | 0.477336 | 0.364019 | 2.26894 | 0.205578 | 0.434366 | 0.177582 | WT |
| XLOC_048138 | PI15 | 0.068439 | 1.31193 | 1.01489 | 0.314169 | 0.099225 | 0.218654 | WT |
| XLOC_027354 | PKDCC | 58.0919 | 68.659 | 69.506 | 11.9625 | 20.0005 | 19.9681 | WT |
| XLOC_038957 | NDST4 | 3.22527 | 4.07553 | 4.92237 | 1.7951 | 1.11636 | 0.347472 | WT |
| XLOC_049191 | AC022639.1 | 8.38118 | 4.04769 | 7.61059 | 0.648089 | 2.11512 | 2.60208 | WT |
| XLOC_041939 | EBF1 | 2.97424 | 1.79056 | 3.44582 | 0.773864 | 0.942278 | 0.494704 | WT |
| XLOC_006501 | VSTM4 | 0.979693 | 1.27858 | 1.48134 | 0.443312 | 0.329921 | 0.236163 | WT |
| XLOC_008690 | MIR210HG | 6.51315 | 6.97106 | 7.4745 | 2.77394 | 2.69544 | 0.191988 | WT |
| XLOC_044977 | AC019117.2, | 19.6648 | 19.1669 | 25.2133 | 5.04766 | 7.82262 | 4.5123 | WT |
| | AC019117.3, | | | | | | | |
| | AHR | | | | | | | |
| XLOC_003656 | PLPPR5 | 1.28076 | 0.491917 | 0.886722 | 0.188502 | 0.288333 | 0.248451 | WT |
| XLOC_038031 | AC098679.1, | 3.11484 | 5.06845 | 3.68229 | 1.74153 | 0.921543 | 0.575133 | WT |
| | TMEM144 | | | | | | | |
| XLOC_003193 | AC099795.1, | 302.092 | 424.578 | 349.152 | 124.511 | 114.662 | 56.2061 | WT |
| | SLC2A1 | | | | | | | |
| XLOC_034650 | MITF | 15.5231 | 10.5048 | 6.80536 | 3.73951 | 1.97018 | 3.37833 | WT |
| XLOC_039653 | SLC1A3 | 27.2071 | 12.7288 | 10.1635 | 2.6237 | 9.54537 | 1.71148 | WT |
| XLOC_013909 | CNMD | 1.06774 | 1.95535 | 1.94698 | 0.433365 | 0.300449 | 0.643193 | WT |
| XLOC_045036 | AC004540.1 | 14.9465 | 10.2668 | 11.4451 | 2.86409 | 4.07977 | 3.27308 | WT |
| XLOC_038757 | AC067942.1, | 8.80269 | 4.40508 | 5.31176 | 1.83023 | 2.31706 | 1.03181 | WT |
| | TMEM150C | | | | | | | |
| XLOC_036882 | CLDN1 | 50.106 | 58.8008 | 66.9105 | 18.1049 | 21.5352 | 9.55423 | WT |
| XLOC_003759 | KCND3 | 0.403729 | 0.196148 | 0.198671 | 0.06339 | 0.103425 | 0.059519 | WT |
| XLOC_050248 | MAMDC2 | 13.1192 | 37.5715 | 35.6402 | 8.35042 | 7.5923 | 8.6863 | WT |
| XLOC_050264 | ANXA1 | 182.386 | 241.636 | 170.57 | 44.5624 | 84.8142 | 41.0541 | WT |
| XLOC_017955 | STRA6 | 14.5523 | 9.21717 | 8.73985 | 2.94136 | 4.71492 | 1.6847 | WT |
| XLOC_013753 | SMAD9 | 9.16316 | 9.65936 | 15.792 | 2.85978 | 3.42595 | 3.77105 | WT |
| XLOC_019804 | CRYM | 6.70859 | 2.20224 | 6.2092 | 2.11623 | 1.02934 | 1.24895 | WT |
| XLOC_038358 | NKX3-2 | 1.22611 | 0.343896 | 1.52837 | 0.430815 | 0.305628 | 0.165224 | WT |
| XLOC_035320 | NLGN1 | 10.4027 | 13.5132 | 20.5439 | 3.90659 | 3.78384 | 5.30042 | WT |
| XLOC_005819 | CNNM1 | 1.38068 | 1.36346 | 0.935558 | 0.300308 | 0.548339 | 0.227938 | WT |
| XLOC_051394 | C9orf64 | 0.322402 | 1.69626 | 1.4076 | 0.255813 | 0.322465 | 0.424607 | WT |
| XLOC_047676 | SLC7A2 | 2.4262 | 2.09633 | 1.88957 | 0.553976 | 0.924381 | 0.39956 | WT |
| XLOC_031614 | BFSP1 | 1.78674 | 1.53542 | 1.82098 | 0.410567 | 0.68674 | 0.413244 | WT |
| XLOC_028345 | DLX1 | 2.18658 | 2.13871 | 1.68289 | 0.511862 | 0.908125 | 0.34467 | WT |
| XLOC_004684 | AC011700.1, | 0.984624 | 0.548826 | 0.787837 | 0.262949 | 0.276082 | 0.143138 | WT |
| | PROX1-AS1 | | | | | | | |
| XLOC_044494 | MAN1A1 | 11.7561 | 8.84561 | 8.97651 | 2.55425 | 3.32955 | 2.83191 | WT |
| XLOC_036549 | PLSCR1 | 20.3379 | 23.2319 | 15.3891 | 6.33228 | 7.30574 | 3.81245 | WT |
| XLOC_052577 | PCDH11X | 2.09792 | 1.4106 | 1.91599 | 0.489946 | 0.58398 | 0.538911 | WT |
| XLOC_006438 | CXCL12 | 14.1143 | 5.72239 | 6.24446 | 2.08741 | 4.14918 | 1.52762 | WT |
| XLOC_034437 | ZNF501 | 4.15659 | 3.97023 | 3.78555 | 1.24516 | 1.39296 | 0.928463 | WT |
| XLOC_013669 | SHISA2 | 13.3832 | 15.9522 | 8.82117 | 3.79089 | 4.16529 | 3.55388 | WT |
| XLOC_011800 | LMO3 | 33.4883 | 20.0635 | 30.1137 | 5.48863 | 9.67963 | 10.0803 | WT |
| XLOC_044069 | RCAN2 | 8.89083 | 14.0988 | 16.9536 | 4.0152 | 4.82664 | 3.21937 | WT |
| XLOC_009023 | LGR4 | 79.6971 | 89.8947 | 100.005 | 21.699 | 36.8237 | 23.3668 | WT |
| XLOC_038688 | CXCL5 | 0.309223 | 0.935753 | 1.82205 | 0.267663 | 0.103094 | 0.564018 | WT |
| XLOC_040718 | ZNF354C | 3.56214 | 4.4131 | 3.65209 | 1.48631 | 1.10653 | 0.9596 | WT |
| XLOC_035875 | CSPG5 | 6.19783 | 7.13338 | 5.1209 | 2.08813 | 3.11037 | 0.440654 | WT |
| XLOC_003270 | FOXD2-AS1 | 0.669449 | 1.16293 | 1.43382 | 0.429154 | 0.323629 | 0.248324 | WT |
| XLOC_005547 | COL13A1 | 8.17538 | 6.57123 | 11.3643 | 2.21423 | 3.93888 | 1.86529 | WT |
| XLOC_020764 | USP43 | 2.19927 | 2.23353 | 2.45209 | 0.506672 | 0.926482 | 0.682065 | WT |
| XLOC_019328 | AC099524.1, | 3.7346 | 2.84576 | 1.96519 | 0.957443 | 1.18674 | 0.482154 | WT |
| | PLCG2, | | | | | | | |
| | RN7SKP176 | | | | | | | |
| XLOC_039480 | ADCY2 | 21.3065 | 25.7335 | 32.2693 | 7.23576 | 11.0972 | 6.3124 | WT |
| XLOC_004455 | FAM129A | 3.98387 | 3.30211 | 2.84975 | 1.57121 | 1.15706 | 0.441431 | WT |
| XLOC_027332 | CYP1B1-AS1, | 0.754595 | 0.634051 | 0.475692 | 0.099235 | 0.311183 | 0.17336 | WT |
| | RPL7P12 | | | | | | | |
| XLOC_002198 | MIR205, | 134.681 | 25.2045 | 1.12706 | 4.0217 | 39.4582 | 7.92563 | WT |
| | MIR205HG | | | | | | | |
| XLOC_010645 | WNT1 | 2.32214 | 3.56237 | 12.7121 | 2.68864 | 1.65239 | 1.60969 | WT |
| XLOC_034696 | ROBO2 | 36.7143 | 29.1239 | 32.3251 | 9.79255 | 8.79265 | 12.8319 | WT |
| XLOC_000241 | CLCN6, | 104.284 | 9.35444 | 11.6016 | 9.38111 | 15.0175 | 16.2436 | WT |
| | NPPA-AS1, | | | | | | | |
| | NPPA-AS1_1, | | | | | | | |
| | NPPA-AS1_2, | | | | | | | |
| | NPPA-AS1_3 | | | | | | | |
| XLOC_039386 | AC018709.1, | 0.737531 | 1.38922 | 1.7777 | 0.385704 | 0.404767 | 0.477051 | WT |
| | F11-AS1, | | | | | | | |
| | MTNR1A | | | | | | | |
| XLOC_053212 | RAI2 | 2.66474 | 2.35815 | 3.02092 | 0.555775 | 1.29099 | 0.765886 | WT |
| XLOC_052663 | PLP1 | 101.388 | 65.3794 | 58.1229 | 21.2014 | 33.8691 | 17.9833 | WT |
| XLOC_039466 | LINC01018 | 1.13458 | 1.0697 | 0.850288 | 0.206416 | 0.488893 | 0.297852 | WT |
| XLOC_017654 | GATM | 12.517 | 16.0119 | 17.134 | 5.22856 | 5.40735 | 4.24186 | WT |
| XLOC_047223 | GPR37 | 6.98319 | 5.81067 | 5.38903 | 1.12652 | 3.11552 | 1.68518 | WT |
| XLOC_003629 | ABCA4 | 3.30242 | 0.849506 | 0.930069 | 0.585848 | 0.76658 | 0.308843 | WT |
| XLOC_010433 | PDE3A | 4.82166 | 3.41033 | 3.47629 | 0.845054 | 2.31846 | 0.671227 | WT |
| XLOC_026186 | CPAMD8 | 18.8752 | 17.1836 | 30.3108 | 6.43773 | 8.12333 | 7.33791 | WT |
| XLOC_038828 | UNC5C | 4.8716 | 3.9903 | 5.9887 | 1.83955 | 2.05809 | 1.01298 | WT |
| XLOC_006249 | CUBN | 0.43488 | 0.614983 | 0.773097 | 0.159042 | 0.177646 | 0.268887 | WT |
| XLOC_034292 | LRRC3B | 5.47996 | 5.31703 | 7.38437 | 1.18094 | 2.98884 | 1.88691 | WT |
| XLOC_053223 | ADGRG2 | 0.329738 | 0.394517 | 0.52786 | 0.091997 | 0.207805 | 0.11795 | WT |
| XLOC_011232 | WSCD2 | 0.548137 | 1.06166 | 1.80662 | 0.3877 | 0.569676 | 0.18264 | WT |
| XLOC_037288 | LIMCH1 | 15.1102 | 26.4054 | 23.2633 | 4.80342 | 5.6498 | 11.3906 | WT |
| XLOC_024145 | DSC3 | 7.46024 | 5.82594 | 7.47786 | 0.914163 | 4.28711 | 1.81923 | WT |
| XLOC_037647 | BMPR1B | 5.4436 | 6.14906 | 5.56825 | 1.96289 | 1.80047 | 2.06573 | WT |
| XLOC_002435 | GPR137B | 14.2021 | 16.6504 | 17.7312 | 4.28355 | 7.38344 | 4.86428 | WT |
| XLOC_007098 | TCERG1L | 7.90809 | 7.59907 | 9.50597 | 2.31325 | 3.95575 | 2.28896 | WT |
| XLOC_039218 | FAM198B | 12.132 | 23.6829 | 21.6214 | 5.08216 | 10.7467 | 3.86605 | WT |
| XLOC_050847 | UAP1L1 | 2.26919 | 2.88792 | 1.83242 | 0.719446 | 0.574443 | 1.13204 | WT |
| XLOC_049164 | CA8 | 6.18118 | 9.56937 | 12.5402 | 3.07264 | 2.60219 | 4.1673 | WT |
| XLOC_011248 | FAM222A | 4.12358 | 5.19282 | 4.7307 | 1.46199 | 2.24513 | 1.23364 | WT |
| XLOC_028509 | PLCL1 | 0.336936 | 1.68793 | 1.59929 | 0.40633 | 0.383856 | 0.486403 | WT |
| XLOC_038748 | RASGEF1B | 3.3694 | 4.66104 | 3.63024 | 1.32815 | 1.49526 | 1.28724 | WT |
| XLOC_049806 | DMRT3 | 33.7659 | 21.6655 | 28.0668 | 12.7937 | 9.80345 | 6.84585 | WT |
| XLOC_044981 | HDAC9 | 32.5955 | 34.5943 | 28.1826 | 8.1358 | 10.736 | 14.9456 | WT |
| XLOC_034156 | BHLHE40 | 28.7412 | 23.1729 | 21.563 | 6.4183 | 14.7712 | 5.06271 | WT |
| XLOC_012486 | LUM | 55.1738 | 29.3758 | 34.8269 | 7.63191 | 28.7091 | 6.42993 | WT |
| XLOC_017508 | ACTC1 | 244.194 | 242.898 | 338.343 | 37.584 | 210.993 | 47.443 | WT |
| XLOC_023287 | NOTUM | 2.79202 | 1.27364 | 1.8554 | 0.582345 | 0.961031 | 0.602072 | WT |
| XLOC_047196 | TSPAN12 | 11.4086 | 19.9723 | 13.6718 | 4.51696 | 5.83577 | 5.98992 | WT |
| XLOC_028947 | AC079779.3 | 0.430747 | 0.227429 | 0.527458 | 0.342024 | 0.063659 | 0.028894 | WT |
| XLOC_015553 | LINC00648 | 3.59956 | 2.56034 | 3.75192 | 1.60875 | 0.960154 | 1.10362 | WT |
| XLOC_003678 | OLFM3 | 26.1028 | 18.3198 | 18.3622 | 7.96972 | 4.76296 | 10.5544 | WT |
| XLOC_031529 | RASSF2 | 6.40228 | 6.28884 | 5.08508 | 2.0242 | 2.82447 | 1.78624 | WT |
| XLOC_007421 | LDHA | 347.627 | 1058.17 | 681.095 | 344.764 | 216.675 | 218.737 | WT |
| XLOC_004771 | AL117348.1, | 5.19131 | 11.1679 | 5.11138 | 2.10458 | 4.92555 | 1.03457 | WT |
| | LEFTY2 | | | | | | | |
| XLOC_018419 | IL32, | 8.52227 | 24.9515 | 18.2384 | 3.80855 | 9.37305 | 6.42134 | WT |
| | RNU1-125P | | | | | | | |
| XLOC_044600 | MAP7 | 6.74022 | 5.61752 | 4.8171 | 2.22027 | 2.97163 | 1.36232 | WT |
| XLOC_011113 | SOCS2 | 27.709 | 22.6784 | 24.1992 | 9.6694 | 10.5514 | 8.41716 | WT |
| XLOC_029655 | FOXI3 | 3.17654 | 6.6982 | 4.35328 | 2.12156 | 1.2951 | 2.0464 | WT |
| XLOC_025709 | ZNF542P | 10.593 | 16.7723 | 11.3415 | 4.3728 | 5.83969 | 4.84302 | WT |
| XLOC_035892 | PFKFB4, | 31.7161 | 45.5772 | 39.6038 | 22.5264 | 15.1506 | 7.79895 | WT |
| | UCN2 | | | | | | | |
| XLOC_045084 | ADCYAP1R1 | 0.799808 | 0.711924 | 0.849815 | 0.246107 | 0.420302 | 0.253653 | WT |
| XLOC_054143 | GABRA3 | 11.2281 | 6.73985 | 7.95941 | 3.24652 | 4.51821 | 2.35408 | WT |
| XLOC_008272 | NAALAD2 | 12.4697 | 6.18758 | 8.54354 | 3.37622 | 3.82636 | 3.43264 | WT |
| XLOC_044991 | ITGB8 | 2.96968 | 3.55486 | 5.29928 | 1.22536 | 1.7833 | 1.6292 | WT |
| XLOC_054197 | MPP1 | 5.29993 | 5.48431 | 5.71707 | 2.4062 | 2.59479 | 1.49679 | WT |
| XLOC_042802 | FAM83B | 4.43091 | 1.89135 | 1.16619 | 0.368648 | 2.1339 | 0.453543 | WT |
| XLOC_001178 | S1PR1 | 64.1762 | 39.7235 | 53.4509 | 16.0314 | 27.8809 | 18.2603 | WT |
| XLOC_048061 | MIR124-2HG | 2.57438 | 2.21446 | 2.93261 | 0.876443 | 1.1928 | 0.982823 | WT |
| XLOC_049026 | SFRP1 | 151.711 | 117.435 | 97.2726 | 33.3748 | 62.8436 | 48.9459 | WT |
| XLOC_049157 | TOX | 16.1585 | 21.7295 | 21.9477 | 6.26663 | 9.02873 | 8.47557 | WT |
| XLOC_016660 | WDR76 | 11.088 | 15.245 | 10.4123 | 7.49297 | 5.27396 | 1.83107 | WT |
| XLOC_034147 | CNTN4 | 15.5597 | 9.57895 | 10.8966 | 4.68208 | 6.66433 | 3.00201 | WT |
| XLOC_030666 | SCG2 | 16.4828 | 3.08644 | 2.8295 | 3.75284 | 4.169 | 1.02544 | WT |
| XLOC_006553 | RHOBTB1 | 21.4776 | 19.1662 | 28.446 | 7.49639 | 13.3863 | 6.86625 | WT |
| XLOC_028877 | ACKR3 | 135.064 | 81.1788 | 72.4411 | 25.9663 | 63.9494 | 26.5325 | WT |
| XLOC_043536 | TUBB2A | 58.1457 | 108.694 | 84.0267 | 31.8392 | 42.4562 | 27.0697 | WT |
| XLOC_036181 | DCBLD2 | 109.374 | 184.897 | 157.895 | 55.3599 | 73.8355 | 54.1726 | WT |
| XLOC_001874 | SUCO | 30.9669 | 40.0632 | 47.0039 | 15.7845 | 18.9654 | 13.3222 | WT |
| XLOC_048183 | AC015522.1 | 23.893 | 16.9284 | 17.7133 | 7.23568 | 14.5014 | 2.13915 | WT |
| XLOC_048884 | STC1 | 20.3656 | 24.8686 | 37.6981 | 17.2469 | 9.99402 | 6.77039 | WT |
| XLOC_027617 | HK2 | 18.6123 | 27.2675 | 32.8217 | 17.2275 | 8.62266 | 6.46639 | WT |
| XLOC_007792 | FAM111B | 3.37129 | 4.88065 | 2.38837 | 2.55485 | 1.44491 | 0.383996 | WT |
| XLOC_006019 | HTRA1 | 82.0626 | 110.945 | 168.516 | 43.3549 | 66.0043 | 40.1356 | WT |
| XLOC_024146 | DSC2 | 33.3603 | 31.4661 | 27.7113 | 10.736 | 15.8787 | 11.7608 | WT |
| XLOC_011021 | SYT1 | 21.0069 | 24.2448 | 21.1858 | 6.77428 | 7.73422 | 13.2987 | WT |
| XLOC_048829 | MTUS1 | 10.4964 | 5.65363 | 4.1125 | 2.9256 | 4.05888 | 1.50429 | WT |
| XLOC_014133 | EFNB2 | 120.975 | 80.3742 | 100.888 | 34.8005 | 55.8196 | 36.7636 | WT |
| XLOC_000084 | AL645608.3, | 3.69182 | 3.41905 | 3.57084 | 1.64182 | 1.66391 | 1.21103 | WT |
| | AL645608.5 | | | | | | | |
| XLOC_005777 | PLCE1 | 1.7972 | 3.12194 | 4.2477 | 1.23436 | 1.12803 | 1.53664 | WT |
| XLOC_023843 | TSHZ1 | 4.84742 | 3.26612 | 4.05506 | 2.05507 | 2.22777 | 0.905298 | WT |
| XLOC_040879 | CTNND2 | 120.806 | 99.3195 | 145.827 | 42.6494 | 50.8711 | 62.7772 | WT |
| XLOC_031878 | MAFB | 5.94784 | 5.82267 | 9.32437 | 5.04695 | 1.84655 | 2.23201 | WT |
| XLOC_046371 | SOSTDC1 | 6.21325 | 5.41999 | 7.58281 | 3.49217 | 2.62015 | 2.22095 | WT |
| XLOC_013777 | LHFPL6 | 42.1751 | 40.9514 | 39.9569 | 13.7108 | 24.8934 | 15.0136 | WT |
| XLOC_025056 | AC011504.1, | 4.70552 | 4.95074 | 4.79504 | 1.35452 | 2.38753 | 2.55534 | WT |
| | ZNF536 | | | | | | | |
| XLOC_026645 | AC245748.1, | 8.96518 | 10.3917 | 7.79196 | 3.94586 | 3.89789 | 4.05486 | WT |
| | AC245748.2, | | | | | | | |
| | ZNF112, | | | | | | | |
| | ZNF229, | | | | | | | |
| | ZNF285 | | | | | | | |
| XLOC_038883 | CXXC4 | 13.0536 | 14.411 | 17.3101 | 5.97946 | 8.61889 | 5.04901 | WT |
| XLOC_040599 | GABRP | 112.341 | 8.14752 | 0.311498 | 0.818228 | 51.5757 | 0.690326 | WT |
| XLOC_036879 | P3H2 | 92.3843 | 144.675 | 162.79 | 60.5479 | 57.382 | 58.1662 | WT |
| XLOC_053052 | DUSP9 | 4.17997 | 3.20362 | 3.76154 | 1.46141 | 2.09033 | 1.38697 | WT |
| XLOC_004061 | S100A6 | 42.6249 | 137.51 | 92.1337 | 31.4031 | 50.2319 | 39.2016 | WT |
| XLOC_010923 | HMGA2 | 152.861 | 173.045 | 160.431 | 71.7923 | 79.1206 | 65.2216 | WT |
| XLOC_051104 | CNTFR | 96.1047 | 80.3847 | 90.7169 | 25.9832 | 44.0011 | 48.7749 | WT |
| XLOC_000284 | EFHD2 | 36.94 | 33.3531 | 33.7508 | 13.5763 | 19.0367 | 13.7297 | WT |
| XLOC_006336 | JCAD | 1.88659 | 0.991218 | 1.77873 | 0.380901 | 1.37374 | 0.321158 | WT |
| XLOC_004451 | COLGALT2 | 18.8059 | 26.6688 | 34.1965 | 12.3101 | 10.3728 | 12.8618 | WT |
| XLOC_023416 | RAB31 | 39.4445 | 54.2819 | 58.6251 | 23.1114 | 30.7347 | 14.4563 | WT |
| XLOC_039312 | GPM6A | 142.331 | 112.945 | 123.321 | 43.4944 | 59.2376 | 67.203 | WT |
| XLOC_004870 | SIPA1L2 | 18.0438 | 20.8893 | 22.3521 | 7.37544 | 10.4743 | 9.66297 | WT |
| XLOC_028224 | LYPD6B | 13.0726 | 7.89584 | 6.28749 | 4.33828 | 4.85202 | 3.10465 | WT |
| XLOC_005776 | SLC35G1 | 12.0153 | 14.9362 | 16.9652 | 7.15247 | 5.82505 | 6.86676 | WT |
| XLOC_042028 | STC2 | 39.4103 | 108.815 | 94.6436 | 38.0803 | 34.8066 | 37.3934 | WT |
| XLOC_003817 | VTCN1 | 27.6071 | 6.08625 | 0.933748 | 0.635095 | 14.6027 | 0.54317 | WT |
| XLOC_044280 | ME1 | 1.86822 | 2.88857 | 2.22209 | 1.06733 | 1.38128 | 0.749188 | WT |
| XLOC_054067 | SOX3 | 38.6166 | 35.9208 | 45.9488 | 18.2425 | 14.315 | 22.6855 | WT |
| XLOC_002921 | ID3 | 364.53 | 373.319 | 384.034 | 205.614 | 166.384 | 142.731 | WT |
| XLOC_003395 | KANK4 | 14.5551 | 7.01567 | 9.79076 | 4.66697 | 7.34944 | 2.43661 | WT |
| XLOC_022130 | GAS7 | 20.1311 | 18.4577 | 28.9587 | 7.17824 | 14.8447 | 9.18089 | WT |
| XLOC_051379 | TLE1 | 71.3807 | 56.8456 | 47.3123 | 25.3284 | 35.7639 | 20.0236 | WT |
| XLOC_009139 | LRP4 | 28.067 | 23.2697 | 24.7974 | 12.7122 | 12.4672 | 10.0552 | WT |
| XLOC_042929 | PRSS35 | 9.9522 | 9.68062 | 11.5052 | 3.93552 | 4.98336 | 5.51723 | WT |
| XLOC_003035 | FABP3 | 39.2522 | 53.8655 | 39.3438 | 15.6095 | 20.9996 | 24.8469 | WT |
| XLOC_009886 | SLC35F2 | 10.758 | 2.90873 | 1.26999 | 0.868084 | 4.87141 | 1.19852 | WT |
| XLOC_031273 | AL121776.1, | 20.1298 | 13.4078 | 10.5815 | 6.29469 | 8.33691 | 5.90384 | WT |
| | EYA2 | | | | | | | |
| XLOC_027092 | ID2 | 373.105 | 325.418 | 419.779 | 205.248 | 192.706 | 122.606 | WT |
| XLOC_007077 | ADAM12 | 1.32462 | 1.23995 | 2.57228 | 0.912305 | 1.02765 | 0.454177 | WT |
| XLOC_007101 | BNIP3 | 116.414 | 159.143 | 193.044 | 96.4058 | 67.7156 | 54.3568 | WT |
| XLOC_006989 | AFAP1L2 | 8.82838 | 5.41901 | 8.72423 | 2.93265 | 4.89531 | 2.90856 | WT |
| XLOC_011223 | BTBD11, | 2.48152 | 2.91672 | 4.6216 | 1.3548 | 2.35844 | 0.981648 | WT |
| | Y_RNA | | | | | | | |
| XLOC_036975 | ZNF595 | 6.35102 | 7.28331 | 6.95044 | 3.03907 | 3.38489 | 3.23104 | WT |
| XLOC_028204 | TEX41 | 0.040081 | 2.34675 | 0.378411 | 0.659828 | 0.257957 | 0.380668 | WT |
| XLOC_048252 | ESRP1 | 15.7248 | 8.69141 | 0.520227 | 1.83753 | 7.89879 | 2.02362 | WT |
| XLOC_001688 | IFI16 | 3.77043 | 7.85987 | 4.36094 | 2.47178 | 3.34622 | 1.72406 | WT |
| XLOC_044959 | ARL4A | 17.9735 | 17.0069 | 14.9066 | 6.98548 | 7.51579 | 9.06534 | WT |
| XLOC_006957 | SORCS1 | 8.62962 | 10.8838 | 14.1475 | 5.93184 | 5.45304 | 4.71167 | WT |
| XLOC_040308 | TGFBI | 200.093 | 29.0639 | 14.9265 | 7.35185 | 104.66 | 5.53227 | WT |
| XLOC_040646 | MSX2 | 31.0896 | 24.3893 | 33.8733 | 15.8631 | 17.3248 | 9.90947 | WT |
| XLOC_048007 | LYN | 7.32157 | 10.3433 | 8.41474 | 3.87391 | 5.15716 | 3.70487 | WT |
| XLOC_012876 | TMEM132D | 5.48048 | 5.53652 | 9.67775 | 3.3135 | 4.61815 | 2.24926 | WT |
| XLOC_037005 | FGFR3 | 44.4408 | 26.2556 | 44.8459 | 9.20196 | 18.6645 | 28.9767 | WT |
| XLOC_039416 | PLEKHG4B | 28.082 | 30.4153 | 29.5133 | 14.1691 | 13.2916 | 15.9762 | WT |
| XLOC_031885 | EMILIN3 | 13.65 | 11.6531 | 15.0838 | 8.61827 | 6.86967 | 4.52668 | WT |
| XLOC_025480 | MYH14 | 17.9398 | 12.3933 | 8.72256 | 3.7543 | 10.287 | 5.38826 | WT |
| XLOC_053141 | PUDP | 29.7026 | 20.9633 | 33.8383 | 15.2146 | 14.4205 | 12.463 | WT |
| XLOC_011663 | SLC2A3 | 464.036 | 227.674 | 184.273 | 72.7496 | 286.109 | 78.3052 | WT |
| XLOC_015234 | KIF26A | 3.85465 | 3.16346 | 4.29761 | 3.79642 | 10.6498 | 8.18542 | WT |
| XLOC_011044 | TMTC2 | 8.37129 | 7.04274 | 6.2335 | 9.05075 | 12.5765 | 21.8112 | WT |
| XLOC_052987 | AFF2 | 9.11706 | 7.77758 | 12.4398 | 16.8524 | 18.2056 | 23.9891 | WT |
| XLOC_052880 | PHF6 | 54.5569 | 51.5495 | 52.6784 | 103.632 | 96.5019 | 119.661 | WT |
| XLOC_038761 | SCD5 | 54.105 | 32.4238 | 43.8686 | 86.6284 | 80.5695 | 95.8552 | WT |
| XLOC_026220 | JUND | 76.9367 | 58.9676 | 95.3929 | 83.3279 | 122.113 | 262.524 | WT |
| XLOC_023877 | NFATC1 | 4.83723 | 3.43985 | 4.79764 | 6.71892 | 6.71465 | 13.0217 | WT |
| XLOC_024463 | CDC34 | 72.5812 | 73.4232 | 90.1597 | 133.151 | 131.689 | 213.41 | WT |
| XLOC_054058 | FGF13 | 23.3113 | 23.436 | 25.4074 | 38.3834 | 48.0407 | 60.0019 | WT |
| XLOC_054039 | MMGT1 | 10.6187 | 11.1085 | 10.8511 | 22.3967 | 21.0978 | 22.6986 | WT |
| XLOC_020149 | AC007906.2 | 3.04474 | 4.98234 | 10.8918 | 19.1593 | 11.206 | 8.08555 | WT |
| XLOC_000135 | PRDM16 | 14.4241 | 11.5181 | 14.8482 | 24.2037 | 28.4051 | 30.7423 | WT |
| XLOC_034496 | KLHDC8B | 89.2608 | 66.6087 | 95.6019 | 128.112 | 173.384 | 213.239 | WT |
| XLOC_030700 | DNER | 19.8836 | 12.5189 | 11.532 | 22.448 | 36.9702 | 30.6572 | WT |
| XLOC_021959 | RTN4RL1 | 6.41516 | 4.7094 | 7.14963 | 12.6978 | 12.1662 | 12.9241 | WT |
| XLOC_053962 | ELF4 | 7.2023 | 4.58935 | 6.3294 | 9.36416 | 17.4292 | 10.7778 | WT |
| XLOC_036851 | DGKG, | 19.0948 | 15.8323 | 26.6101 | 20.7874 | 41.7254 | 65.1184 | WT |
| | ETV5 | | | | | | | |
| XLOC_007351 | ADM | 5.22835 | 9.08988 | 10.8682 | 18.6967 | 14.872 | 18.9515 | WT |
| XLOC_030393 | TFPI | 28.0628 | 49.8402 | 72.3747 | 114.022 | 69.8086 | 130.292 | WT |
| XLOC_045032 | NFE2L3 | 21.9362 | 16.2081 | 18.861 | 44.3013 | 42.6996 | 32.1673 | WT |
| XLOC_008588 | PKNOX2 | 36.5336 | 25.577 | 38.1836 | 63.1011 | 87.0611 | 59.6187 | WT |
| XLOC_048193 | CA2 | 46.3375 | 65.5845 | 84.7922 | 154.768 | 110.287 | 147.189 | WT |
| XLOC_053815 | MORC4 | 9.62064 | 9.91422 | 10.367 | 17.9793 | 22.3333 | 22.4979 | WT |
| XLOC_040597 | KCNIP1 | 8.63696 | 4.44387 | 11.0611 | 16.3806 | 24.6756 | 9.69929 | WT |
| XLOC_031446 | PPDPF | 171.891 | 130.648 | 197.12 | 307.787 | 249.93 | 500.784 | WT |
| XLOC_001360 | NBPF26 | 3.82194 | 3.14481 | 5.75055 | 7.10454 | 9.49809 | 10.3706 | WT |
| XLOC_053006 | MAMLD1 | 3.23 | 2.36486 | 3.42337 | 5.65034 | 8.46834 | 5.01126 | WT |
| XLOC_035138 | C3orf58 | 11.0446 | 13.1223 | 13.3748 | 19.4027 | 25.8112 | 34.5008 | WT |
| XLOC_035718 | EOMES | 1.89088 | 4.26007 | 1.36317 | 5.18376 | 8.41972 | 2.41592 | WT |
| XLOC_041572 | CDO1 | 54.537 | 58.1313 | 81.2553 | 117.738 | 123.911 | 172.199 | WT |
| XLOC_053840 | CAPN6 | 53.7524 | 50.2521 | 35.7015 | 80.8738 | 122.236 | 95.2684 | WT |
| XLOC_052847 | OCRL | 17.3435 | 19.6148 | 23.3017 | 41.2117 | 38.7067 | 49.3023 | WT |
| XLOC_024713 | LDLR, | 26.8963 | 32.2207 | 30.3653 | 35.251 | 49.3324 | 107.334 | WT |
| | MIR6886 | | | | | | | |
| XLOC_050476 | GALNT12 | 22.4471 | 13.5945 | 26.3889 | 52.9317 | 48.0713 | 34.5528 | WT |
| XLOC_051615 | LPAR1 | 8.88699 | 4.56556 | 6.04646 | 18.0084 | 12.243 | 12.2431 | WT |
| XLOC_038678 | ADAMTS3 | 1.41731 | 2.18983 | 2.41307 | 5.50186 | 4.4331 | 3.30322 | WT |
| XLOC_043700 | HIST1H1C | 7.05921 | 6.86671 | 12.4486 | 10.1674 | 12.6994 | 35.3308 | WT |
| XLOC_008078 | FOLR1 | 6.36364 | 7.46499 | 8.31793 | 18.0765 | 11.7625 | 19.1518 | WT |
| XLOC_046906 | SEMA3C | 55.9545 | 36.8157 | 81.8943 | 155.247 | 164.452 | 66.6996 | WT |
| XLOC_002145 | CNTN2 | 3.65929 | 3.05096 | 2.60199 | 10.3732 | 6.17839 | 4.12765 | WT |
| XLOC_005982 | AL354863.1 | 39.2131 | 25.4606 | 20.3032 | 88.0046 | 52.2735 | 48.8231 | WT |
| XLOC_002143 | NFASC | 2.18191 | 1.86701 | 2.09763 | 5.3183 | 5.62301 | 2.77272 | WT |
| XLOC_004604 | BLACAT1, | 31.3434 | 36.2722 | 22.3452 | 62.4107 | 88.9478 | 49.5512 | WT |
| | LEMD1 | | | | | | | |
| XLOC_023130 | BTBD17 | 4.75019 | 2.78016 | 3.04609 | 9.30145 | 8.5229 | 5.83836 | WT |
| XLOC_053821 | TSC22D3 | 22.2958 | 24.1012 | 24.4761 | 45.6652 | 50.2699 | 63.2403 | WT |
| XLOC_000957 | GADD45A | 21.0363 | 31.0184 | 26.6929 | 42.5988 | 33.7499 | 102.592 | WT |
| XLOC_045574 | GNG11 | 1.86665 | 2.82708 | 3.10673 | 6.40457 | 5.52396 | 5.86769 | WT |
| XLOC_052690 | FRMPD3 | 1.1497 | 0.932159 | 1.3669 | 1.80633 | 2.93777 | 3.14082 | WT |
| XLOC_052629 | TCEAL2 | 19.9932 | 6.70028 | 21.5307 | 41.2382 | 32.9835 | 36.1429 | WT |
| XLOC_046843 | CLDN3 | 9.13919 | 5.96728 | 9.35964 | 13.4217 | 15.7968 | 27.0659 | WT |
| XLOC_052878 | CCDC160 | 13.1492 | 9.10177 | 12.0863 | 21.0438 | 23.8397 | 34.1269 | WT |
| XLOC_025629 | PRKCG | 3.95921 | 2.16936 | 3.70145 | 6.22587 | 6.35995 | 10.1207 | WT |
| XLOC_042340 | HIST1H2BD | 9.99424 | 9.24429 | 18.8462 | 10.5886 | 29.9465 | 47.5109 | WT |
| XLOC_028258 | GALNT5 | 2.93333 | 0.900819 | 1.33843 | 3.68405 | 6.51704 | 1.88076 | WT |
| XLOC_036012 | IL17RD | 23.3246 | 20.3603 | 38.1309 | 69.8297 | 46.7799 | 75.4779 | WT |
| XLOC_002280 | C1orf115 | 2.89723 | 2.57253 | 3.35716 | 8.55616 | 6.14166 | 6.19987 | WT |
| XLOC_054123 | AC244197.2, | 64.5571 | 43.8138 | 70.3316 | 128.204 | 143.547 | 152.639 | WT |
| | AC244197.3, | | | | | | | |
| | IDS, | | | | | | | |
| | LINC00893 | | | | | | | |
| XLOC_045653 | NYAP1 | 5.51019 | 4.72303 | 6.63899 | 8.98153 | 10.5585 | 20.7961 | WT |
| XLOC_047007 | TMEM130 | 15.3733 | 9.12391 | 22.0796 | 21.1394 | 35.76 | 54.809 | WT |
| XLOC_042338 | HIST1H2AC | 3.77259 | 5.18168 | 5.23739 | 5.53757 | 9.43995 | 19.1058 | WT |
| XLOC_054062 | ATP11C | 9.00299 | 7.13128 | 8.40206 | 20.4838 | 21.2517 | 17.3818 | WT |
| XLOC_031168 | NNAT | 142.051 | 244.295 | 98.5143 | 460.313 | 487.265 | 222.813 | WT |
| XLOC_049732 | LYNX1, | 4.41757 | 4.93709 | 5.28431 | 11.8904 | 10.2397 | 13.2654 | WT |
| | SLURP2 | | | | | | | |
| XLOC_046218 | INSIG1 | 22.6961 | 31.755 | 31.0139 | 32.6581 | 42.0509 | 133.821 | WT |
| XLOC_040610 | FGF18 | 0.978869 | 1.15747 | 1.82814 | 3.20313 | 3.92089 | 2.5889 | WT |
| XLOC_044277 | UBE3D | 13.258 | 12.0643 | 17.6985 | 14.563 | 34.8525 | 56.1947 | WT |
| XLOC_005833 | WNT8B | 133.599 | 49.4232 | 130.886 | 307.123 | 281.806 | 181.85 | WT |
| XLOC_033519 | FBLN1 | 116.331 | 62.4759 | 55.5671 | 208.747 | 209.518 | 158.837 | WT |
| XLOC_036029 | FAM107A | 13.7191 | 9.12034 | 5.94778 | 16.0175 | 40.5482 | 14.337 | WT |
| XLOC_032423 | PCP4 | 9.13445 | 19.093 | 16.0801 | 54.1004 | 21.3556 | 33.681 | WT |
| XLOC_052071 | SAT1 | 56.3048 | 77.4788 | 73.6709 | 160.181 | 170.499 | 182.884 | WT |
| XLOC_048551 | ADGRB1 | 2.22192 | 1.66561 | 2.13119 | 3.87596 | 6.34666 | 4.70261 | WT |
| XLOC_007003 | VAX1 | 3.77645 | 7.98793 | 7.35952 | 21.586 | 13.9139 | 12.3501 | WT |
| XLOC_030738 | ECEL1 | 9.3731 | 3.88375 | 9.07631 | 13.783 | 29.7727 | 12.7174 | WT |
| XLOC_026026 | COL5A3 | 0.796907 | 1.31037 | 1.24016 | 3.61755 | 1.82847 | 2.9932 | WT |
| XLOC_017540 | AC023908.2, | 2.62604 | 4.25401 | 5.21893 | 11.7612 | 9.04747 | 9.71639 | WT |
| | FSIP1, | | | | | | | |
| | GPR176 | | | | | | | |
| XLOC_033561 | PIM3 | 13.3732 | 14.059 | 17.7079 | 19.3155 | 21.0904 | 73.5991 | WT |
| XLOC_030077 | TMEM163 | 1.18136 | 1.45792 | 1.12487 | 2.57937 | 3.44166 | 3.51647 | WT |
| XLOC_010023 | C1QTNF5, | 6.28492 | 4.45959 | 6.78377 | 12.2525 | 17.6149 | 14.5641 | WT |
| | MFRP | | | | | | | |
| XLOC_048928 | DUSP4 | 6.90588 | 10.7838 | 10.1329 | 16.7994 | 17.1467 | 36.6906 | WT |
| XLOC_048992 | RAB11FIP1 | 18.2559 | 17.6462 | 23.3217 | 35.1087 | 29.3611 | 86.7308 | WT |
| XLOC_005542 | TSPAN15 | 43.5694 | 28.2963 | 42.6198 | 116.925 | 86.9998 | 91.7988 | WT |
| XLOC_054006 | FAM122B | 11.0814 | 14.8125 | 13.16 | 35.6611 | 25.4969 | 39.926 | WT |
| XLOC_038088 | PALLD | 91.1424 | 95.3422 | 111.917 | 188.823 | 173.813 | 410.679 | WT |
| XLOC_026199 | BST2 | 18.6328 | 41.7776 | 25.707 | 60.773 | 45.88 | 116.923 | WT |
| XLOC_009683 | FAM181B | 2.06403 | 1.75194 | 4.16686 | 8.95673 | 8.8737 | 3.01097 | WT |
| XLOC_054131 | CXorf40B | 13.2865 | 12.8844 | 13.002 | 28.8197 | 30.2012 | 43.2512 | WT |
| XLOC_032441 | ABCG1 | 3.54189 | 2.29292 | 2.82606 | 8.16075 | 9.58416 | 4.93166 | WT |
| XLOC_003976 | HIST2H2BE | 6.59545 | 6.66469 | 10.7701 | 7.48744 | 13.3224 | 42.238 | WT |
| XLOC_053841 | DCX | 15.4712 | 8.76769 | 15.4486 | 22.941 | 41.4257 | 41.2549 | WT |
| XLOC_005843 | KAZALD1 | 5.29693 | 3.61388 | 4.57153 | 13.1899 | 13.7831 | 8.97227 | WT |
| XLOC_012477 | DUSP6 | 32.578 | 35.9242 | 57.5001 | 72.4462 | 125.635 | 140.899 | WT |
| XLOC_014816 | SMOC1 | 20.1662 | 23.7982 | 10.5509 | 46.2095 | 64.3087 | 36.9327 | WT |
| XLOC_040656 | HRH2 | 0.837311 | 0.634602 | 1.32313 | 1.99127 | 1.62424 | 3.94527 | WT |
| XLOC_006803 | FGFBP3 | 33.6678 | 77.1798 | 49.4306 | 109.801 | 167.569 | 158.38 | WT |
| XLOC_052691 | PRPS1 | 183.224 | 189.988 | 179.066 | 453.895 | 545.065 | 502.77 | WT |
| XLOC_005775 | AL157396.1, | 69.2823 | 68.8436 | 93.8269 | 203.796 | 192.153 | 238.139 | WT |
| | LGI1 | | | | | | | |
| XLOC_052372 | MAGEH1 | 24.0826 | 19.4032 | 28.612 | 57.957 | 68.7396 | 71.5261 | WT |
| XLOC_000088 | ISG15 | 6.21447 | 6.95642 | 6.98725 | 11.8814 | 10.3321 | 33.5355 | WT |
| XLOC_024612 | TNFSF9 | 0.717224 | 3.03131 | 1.10745 | 3.94731 | 1.3869 | 8.28667 | WT |
| XLOC_026219 | KIAA1683 | 0.87993 | 0.708561 | 1.21511 | 1.36168 | 2.4304 | 4.0732 | WT |
| XLOC_053827 | COL4A6 | 115.036 | 99.1192 | 130.266 | 346.703 | 285.01 | 335.723 | WT |
| XLOC_053972 | IGSF1 | 6.52065 | 3.54768 | 6.49959 | 15.8002 | 17.7319 | 13.0285 | WT |
| XLOC_013498 | MYO16 | 0.221357 | 0.355848 | 0.495329 | 1.484 | 0.945527 | 0.623607 | WT |
| XLOC_031173 | VSTM2L | 9.62772 | 8.65123 | 17.2476 | 34.2098 | 27.5505 | 39.8025 | WT |
| XLOC_052853 | RAB33A | 2.68172 | 3.10035 | 2.85824 | 6.57785 | 9.1093 | 9.52405 | WT |
| XLOC_043236 | EYA4 | 10.4845 | 5.94823 | 8.2757 | 23.4297 | 25.1694 | 23.9862 | WT |
| XLOC_046163 | LRRC61 | 0.86421 | 1.6791 | 1.53381 | 1.73302 | 3.93292 | 6.32881 | WT |
| XLOC_053995 | GPC3 | 356.448 | 305.126 | 420.88 | 937.281 | 973.583 | 1280.35 | WT |
| XLOC_016656 | CKMT1A | 0.195764 | 0.252631 | 0.065192 | 0.61273 | 0.535787 | 0.367446 | WT |
| XLOC_035032 | TRH | 41.0091 | 27.1132 | 39.3515 | 81.1771 | 106.609 | 131.166 | WT |
| XLOC_007911 | AP003774.1 | 0.396296 | 0.267346 | 0.587915 | 1.13377 | 1.32979 | 1.28669 | WT |
| XLOC_009044 | DCDC1 | 1.4494 | 1.47018 | 2.47011 | 5.69078 | 6.21219 | 4.51809 | WT |
| XLOC_025875 | SMIM24 | 3.74159 | 5.42114 | 11.6057 | 12.6931 | 23.5603 | 27.0304 | WT |
| XLOC_051389 | FRMD3 | 1.83018 | 2.1865 | 4.43258 | 8.30475 | 5.60456 | 11.875 | WT |
| XLOC_028631 | UNC80 | 0.217954 | 0.44451 | 0.425888 | 1.89622 | 0.598572 | 0.881806 | WT |
| XLOC_035814 | CCK | 4.32176 | 1.89858 | 1.00456 | 1.6504 | 2.01784 | 18.8127 | WT |
| XLOC_028335 | GAD1 | 3.76327 | 2.3603 | 2.71097 | 8.06297 | 9.04451 | 11.0829 | WT |
| XLOC_042640 | PIM1 | 22.8367 | 25.5602 | 29.2301 | 33.1164 | 54.0663 | 161.469 | WT |
| XLOC_037275 | RHOH | 0.617003 | 1.02472 | 1.41109 | 4.20766 | 3.06835 | 2.5333 | WT |
| XLOC_022915 | CA10 | 1.19926 | 1.0558 | 2.24358 | 9.02446 | 4.47401 | 1.0871 | WT |
| XLOC_014449 | DHRS2 | 0.207272 | 0.738887 | 0.203989 | 2.33486 | 0.275137 | 1.13645 | WT |
| XLOC_006704 | SFTPA2 | 0.501864 | 0.437923 | 0.852414 | 0.787377 | 2.13541 | 2.93168 | WT |
| XLOC_004924 | LINC01139 | 3.24003 | 2.89495 | 3.70155 | 15.9453 | 7.33362 | 9.19588 | WT |
| XLOC_032387 | SIM2 | 1.88357 | 2.09546 | 7.89405 | 19.4132 | 12.1409 | 7.74329 | WT |
| XLOC_017398 | ATP10A | 0.36411 | 0.211242 | 0.194995 | 0.847112 | 1.28466 | 0.423302 | WT |
| XLOC_052195 | MAOA | 3.36078 | 2.21257 | 3.23231 | 9.66039 | 7.3758 | 12.227 | WT |
| XLOC_037118 | AC097493.4 | 21.2395 | 19.5519 | 12.6137 | 49.5568 | 58.4472 | 70.4522 | WT |
| XLOC_053014 | GPR50 | 2.08277 | 3.44278 | 4.40543 | 10.3401 | 8.1101 | 14.8621 | WT |
| XLOC_015083 | AL117190.1, | 0.407776 | 1.35024 | 0.252559 | 2.45727 | 1.99503 | 2.35652 | WT |
| | MEG3, MEG3_2, | | | | | | | |
| | MEG8, MEG81, | | | | | | | |
| | MEG8_2, | | | | | | | |
| | SNORD112, | | | | | | | |
| | SNORD113, | | | | | | | |
| | SNORD113-4, | | | | | | | |
| | SNORD114-10, | | | | | | | |
| | SNORD114-26 | | | | | | | |
| XLOC_022019 | ALOX15 | 0.436382 | 0.489093 | 0.472337 | 2.86337 | 1.17703 | 0.71001 | WT |
| XLOC_051724 | NR5A1 | 1.70203 | 0.836837 | 1.68223 | 2.71166 | 8.61071 | 3.14169 | WT |
| XLOC_000121 | GABRD | 0.080031 | 0.380221 | 0.258136 | 0.779533 | 0.894608 | 0.821325 | WT |
| XLOC_000327 | PADI3 | 0.191586 | 0.357198 | 0.171492 | 0.984504 | 0.4349 | 1.10153 | WT |
| XLOC_039801 | RGS7BP | 0.488525 | 0.345355 | 0.943862 | 1.95493 | 3.47323 | 0.808829 | WT |
| XLOC_045580 | PEG10 | 180.739 | 195.875 | 173.401 | 548.084 | 687.495 | 700.931 | WT |
| XLOC_014868 | VRTN | 0.388638 | 1.2995 | 0.156878 | 2.46285 | 2.00403 | 2.08552 | WT |
| XLOC_014085 | SLC15A1 | 0.24021 | 0.076908 | 0.217821 | 0.546709 | 0.621361 | 0.78827 | WT |
| XLOC_023568 | TTR | 7.64744 | 5.28695 | 9.74179 | 4.19376 | 76.4427 | 3.12691 | WT |
| XLOC_037820 | SPRY1 | 9.82548 | 9.72238 | 17.1053 | 45.8217 | 47.4373 | 42.2053 | WT |
| XLOC_025948 | CD70 | 0.393748 | 4.95025 | 1.24036 | 7.43782 | 0.82236 | 16.5319 | WT |
| XLOC_016521 | GREM1 | 0.234164 | 0.121837 | 0.408728 | 1.12876 | 0.984358 | 0.772676 | WT |
| XLOC_037153 | CD38 | 0.097742 | 0.579579 | 0.551301 | 2.28771 | 1.65186 | 0.730318 | WT |
| XLOC_034645 | PSMC1P1 | 0.44804 | 0.848938 | 0.461207 | 1.2887 | 2.64245 | 2.80786 | WT |
| XLOC_037405 | LINC02380 | 0.481051 | 0.329814 | 1.9498 | 4.40254 | 5.40685 | 0.864318 | WT |
| XLOC_045797 | FEZF1-AS1 | 66.8407 | 70.4759 | 66.7804 | 241.268 | 249.816 | 298.805 | WT |
| XLOC_004325 | F5 | 0.759453 | 0.634342 | 0.089888 | 4.71328 | 0.607308 | 0.434241 | WT |
| XLOC_051882 | AL355987.1, | 0.110907 | 0.044809 | 0.219375 | 0.165498 | 1.1634 | 0.16398 | WT |
| | LCN10, | | | | | | | |
| | LCN6 | | | | | | | |
| XLOC_017869 | MEGF11 | 0.422346 | 0.071042 | 0.22406 | 1.2853 | 1.17064 | 0.430566 | WT |
| XLOC_030369 | FRZB | 18.0479 | 17.487 | 17.9293 | 35.2304 | 89.8978 | 91.2878 | WT |
| XLOC_047511 | VIPR2 | 0.192779 | 0.079507 | 0.089813 | 0.434345 | 0.670121 | 0.415995 | WT |
| XLOC_046984 | PDK4 | 0.375952 | 0.154405 | 0.948847 | 2.17342 | 2.13958 | 1.93709 | WT |
| XLOC_007385 | SPON1 | 9.90189 | 10.394 | 7.00568 | 28.4848 | 43.7573 | 43.3548 | WT |
| XLOC_025926 | FUT3 | 0.790168 | 0.206398 | 1.19875 | 0.669821 | 2.65624 | 6.11659 | WT |
| XLOC_040637 | C5orf47 | 0.150548 | 0.230716 | 0.224914 | 0.747187 | 0.650913 | 1.23161 | WT |
| XLOC_012724 | TESC | 0.606087 | 0.714566 | 0.419522 | 2.66894 | 2.93782 | 2.12762 | WT |
| XLOC_006913 | FGF8 | 8.06329 | 8.99878 | 18.6454 | 50.3332 | 64.3412 | 47.0516 | WT |
| XLOC_049314 | PAG1 | 2.12542 | 2.18011 | 2.94257 | 14.4441 | 7.45301 | 11.0131 | WT |
| XLOC_016027 | RTL1 | 0.168161 | 0.888182 | 0.294915 | 2.2409 | 2.72784 | 1.16737 | WT |
| XLOC_015082 | DLK1 | 108.58 | 445.412 | 155.099 | 1060.44 | 1117.53 | 1048.09 | WT |
| XLOC_015979 | GSC | 0.896664 | 1.92959 | 3.30342 | 9.3785 | 7.02679 | 11.5861 | WT |
| XLOC_024337 | RNF152 | 6.17501 | 2.53135 | 6.97134 | 18.5501 | 28.2161 | 26.9696 | WT |
| XLOC_035303 | CLDN11 | 0.31711 | 0.409501 | 0.916247 | 2.76374 | 2.30665 | 2.72639 | WT |
| XLOC_003362 | FYB2 | 1.2895 | 1.05917 | 1.41076 | 6.16659 | 5.3411 | 6.53754 | WT |
| XLOC_032392 | RIPPLY3 | 3.3058 | 2.73499 | 3.88712 | 20.8166 | 16.3022 | 13.4339 | WT |
| XLOC_052942 | LINC00632 | 2.86458 | 2.34239 | 6.29908 | 12.5445 | 13.613 | 32.7025 | WT |
| XLOC_036567 | TM4SF18 | 0.324446 | 0.551776 | 1.09341 | 4.26705 | 3.42361 | 2.49461 | WT |
| XLOC_045798 | AC004594.1 | 0.265679 | 0.267242 | 0 | 0.966884 | 1.09454 | 0.701641 | WT |
| XLOC_047230 | GRM8 | 3.54174 | 2.7208 | 3.39742 | 17.0717 | 23.1995 | 12.4775 | WT |
| XLOC_052560 | DACH2 | 1.33421 | 1.73229 | 1.11884 | 6.14873 | 7.13163 | 9.59335 | WT |
| XLOC_003458 | RPE65 | 0.08274 | 0.113455 | 0.170342 | 0.930307 | 0.599978 | 0.475714 | WT |
| XLOC_034204 | SLC6A1 | 0.060979 | 0.156361 | 0.214192 | 0.730038 | 1.09308 | 0.544853 | WT |
| XLOC_049383 | CALB1 | 4.01979 | 4.37203 | 4.07709 | 21.985 | 44.1714 | 2.57769 | WT |
| XLOC_005163 | SLC39A12 | 0.28621 | 0.163049 | 0.445538 | 1.63096 | 2.30924 | 1.03106 | WT |
| XLOC_054060 | MCF2 | 0.132052 | 0.237883 | 0.098161 | 1.39861 | 0.465809 | 0.758701 | WT |
| XLOC_049559 | KCNV1 | 0.26381 | 0.0548 | 0.176721 | 1.23458 | 1.30239 | 0.29791 | WT |
| XLOC_022729 | MEOX1 | 3.91054 | 1.09259 | 2.46675 | 20.9349 | 19.2186 | 3.91746 | WT |
| XLOC_023335 | ADCYAP1 | 0.465905 | 0.567205 | 0.929481 | 4.02343 | 2.71566 | 4.87488 | WT |
| XLOC_043927 | CUTA | 0.293006 | 0.233478 | 0.509997 | 5.54604 | 0.452025 | 0.247716 | WT |
| XLOC_028901 | TWIST2 | 0.208211 | 0.186875 | 0.21433 | 1.41554 | 0.583798 | 1.70933 | WT |
| XLOC_021584 | CA4 | 1.61206 | 1.2634 | 1.32575 | 6.48404 | 5.48449 | 13.6196 | WT |
| XLOC_031893 | PTPRT | 0.28729 | 0.205393 | 0.316373 | 1.54923 | 1.668 | 1.71211 | WT |
| XLOC_047531 | MYOM2 | 0.149096 | 0.298459 | 0.211717 | 0.156465 | 3.86903 | 0.162238 | WT |
| XLOC_030352 | ZNF385B | 9.47793 | 14.9801 | 18.3758 | 70.8253 | 56.3005 | 146.728 | WT |
| XLOC_047205 | FEZF1 | 41.7884 | 48.1114 | 40.6682 | 268.798 | 232.235 | 337.644 | WT |
| XLOC_037406 | AC013724.1 | 1.26243 | 0.607775 | 2.7783 | 12.1569 | 16.1201 | 1.9022 | WT |
| XLOC_036569 | TM4SF1 | 5.40712 | 3.62359 | 7.60791 | 31.4867 | 28.7339 | 51.5129 | WT |
| XLOC_024906 | GDF15 | 4.30613 | 12.7602 | 12.9116 | 44.6215 | 16.8273 | 147.763 | WT |
| XLOC_027347 | SLC8A1-AS1 | 0.103871 | 0.072015 | 0 | 0.606054 | 0.349679 | 0.281893 | WT |
| XLOC_000114 | MMP23B | 0.103909 | 0 | 0.161306 | 0.352486 | 0.419838 | 1.10201 | WT |
| XLOC_013839 | SIAH3 | 0.123135 | 0.19503 | 0.111841 | 1.38261 | 1.37262 | 0.487249 | WT |
| XLOC_001202 | NTNG1 | 1.08523 | 0.722204 | 1.30259 | 9.20546 | 9.03608 | 5.6575 | WT |
| XLOC_049751 | EEF1D | 1.81141 | 1.32743 | 1.88277 | 1.65909 | 1.42378 | 37.2556 | WT |
| XLOC_050257 | GDA | 0.014489 | 0.033737 | 0.097653 | 0.63289 | 0.190831 | 0.394516 | WT |
| XLOC_026965 | AC008735.6 | 0.045967 | 0.458412 | 0.023659 | 0.961576 | 0.999984 | 2.56362 | WT |
| XLOC_051526 | GABBR2 | 0.457344 | 0.648445 | 0.400772 | 0.548775 | 6.17456 | 7.34725 | WT |
| XLOC_025815 | SBNO2 | 0.11578 | 0.079897 | 0.108348 | 2.2357 | 0.217395 | 0.490183 | WT |
| XLOC_000992 | LHX8 | 0.079931 | 0.510155 | 0.032912 | 2.20661 | 3.15652 | 0.683668 | WT |
| XLOC_017767 | ALDH1A2 | 0.994996 | 0.891748 | 1.23284 | 11.8578 | 6.66132 | 12.7266 | WT |
| XLOC_017689 | HDC | 0.89838 | 0.989162 | 0.752232 | 10.9699 | 11.0578 | 5.70934 | WT |
| XLOC_014541 | INSM2 | 0.017707 | 0.256047 | 0.054682 | 1.12048 | 1.94313 | 0.529398 | WT |
| XLOC_035850 | SLC6A20 | 0.014771 | 0.035495 | 0.111345 | 0.596965 | 1.15708 | 0.24817 | WT |
| XLOC_022342 | AC005697.1, | 20.6119 | 30.5279 | 25.7636 | 334.055 | 213.318 | 425.604 | WT |
| | LYRM9, | | | | | | | |
| | NOS2 | | | | | | | |
| XLOC_015003 | AL110118.2, | 0.009336 | 0.982384 | 0.169714 | 0.074207 | 14.1377 | 0.487161 | WT |
| | TMEM251, | | | | | | | |
| | UBR7 | | | | | | | |
| XLOC_036088 | PROK2 | 0.122643 | 0.307396 | 0.133767 | 1.93546 | 3.6817 | 2.57632 | WT |
| XLOC_054232 | AKAP17A | 0.037678 | 0.033052 | 0.038786 | 0.065554 | 1.56317 | 0.136579 | WT |
| XLOC_047719 | FGF17 | 0.181709 | 0.709779 | 1.50313 | 19.5062 | 14.7706 | 4.55528 | WT |
| XLOC_053187 | PIGA | 9.98582 | 10.5065 | 9.63516 | 17.4441 | 644.333 | 18.5628 | WT |
| XLOC_052734 | HTR2C | 0.700825 | 1.81764 | 1.30142 | 30.2338 | 34.0218 | 44.1372 | WT |
| XLOC_030000 | RAB6C-AS1 | 0.089992 | 0.033102 | 0.031389 | 0.096716 | 20.4848 | 0.035065 | WT |
| XLOC_025431 | AC026803.1 | 0 | 0 | 0 | 0.758489 | 0.350571 | 1.66778 | WT |
| XLOC_036777 | AC109779.1 | 0 | 0 | 0 | 0.61836 | 0.131894 | 0.23794 | WT |
| XLOC_027001 | ZNF835 | 0.053623 | 0.658148 | 0.340527 | 0 | 0 | 0 | WT |
| XLOC_025714 | ZNF471 | 0.682554 | 1.15445 | 0.470161 | 0 | 0 | 0 | WT |
| XLOC_045348 | ZNF736 | 0.624218 | 1.92792 | 0.552082 | 0 | 0 | 0 | WT |
| XLOC_006399 | AL132657.1, | 0.863026 | 4.17081 | 1.05968 | 0 | 0 | 0 | WT |
| | ZNF248 | | | | | | | |
| XLOC_050265 | AL451127.1 | 0.422805 | 0.421643 | 0.373054 | 0 | 0 | 0 | WT |
| XLOC_032449 | CRYAA | 0.53568 | 0.400657 | 1.15799 | 0 | 0 | 0 | WT |
| XLOC_051343 | LINC01474 | 0.179453 | 0.190138 | 0.39052 | 0 | 0 | 0 | WT |
| XLOC_031712 | AL035252.2 | 0.597144 | 0.151676 | 0.438592 | 0 | 0 | 0 | WT |
| XLOC_033655 | PRODH | 0 | 0 | 3.55524 | 0 | 0 | 0 | WT |
| XLOC_010032 | AP001360.2 | 0.213455 | 0.496694 | 0.146485 | 0 | 0 | 0 | WT |
| XLOC_050737 | AL136141.1 | 0.636613 | 0.634865 | 0.491491 | 0 | 0 | 0 | WT |
| XLOC_049474 | RGS22 | 0.417502 | 0.237326 | 0.261315 | 0 | 0 | 0 | WT |
| XLOC_053524 | ZXDA | 0.413137 | 0.088739 | 0.510333 | 0 | 0 | 0 | WT |
| XLOC_027708 | THNSL2 | 0.347969 | 0.618049 | 0.70932 | 0 | 0 | 0 | WT |
| XLOC_040716 | AC104117.3 | 0.5384 | 0.483229 | 0.332533 | 0 | 0 | 0 | WT |
| XLOC_031042 | AL390198.1 | 0.456256 | 0.910005 | 0.234832 | 0 | 0 | 0 | WT |
| XLOC_050453 | AL589843.1 | 0.351823 | 0.818665 | 0.241441 | 0 | 0 | 0 | WT |
| XLOC_024731 | ZNF441 | 2.82232 | 2.53353 | 2.82785 | 0 | 0 | 0 | WT |

| | | | | | | | | signif- |
|---|---|---|---|---|---|---|---|---|
| tracking_id | sample_2 | value_1 | value_2 | log2(fold_change) | test_stat | p_value | q_value | icant |
| XLOC_002465 | KO | 12.1058 | 0.002618 | −12.1752 | −0.17692 | 5.00E−05 | 0.002735 | yes |
| XLOC_014867 | KO | 37.3025 | 0.028226 | −10.368 | −9.698 | 0.00025 | 0.010784 | yes |
| XLOC_032376 | KO | 4.28914 | 0.007477 | −9.16397 | −0.54615 | 0.0002 | 0.008981 | yes |
| XLOC_051344 | KO | 319.839 | 0.721764 | −8.7916 | −8.20366 | 5.00E−05 | 0.002735 | yes |
| XLOC_019330 | KO | 88.2719 | 0.252207 | −8.4512 | −5.25973 | 0.0001 | 0.005033 | yes |
| XLOC_053625 | KO | 18.1989 | 0.055653 | −8.35318 | −0.70916 | 5.00E−05 | 0.002735 | yes |
| XLOC_027099 | KO | 13.8546 | 0.042941 | −8.3338 | −0.59415 | 5.00E−05 | 0.002735 | yes |
| XLOC_000776 | KO | 32.2388 | 0.111802 | −8.17171 | −9.87898 | 5.00E−05 | 0.002735 | yes |
| XLOC_023004 | KO | 14.8868 | 0.066548 | −7.80542 | −0.94361 | 0.0012 | 0.037051 | yes |
| XLOC_049879 | KO | 110.799 | 0.505128 | −7.77708 | −4.1145 | 5.00E−05 | 0.002735 | yes |
| XLOC_021600 | KO | 10.0484 | 0.050286 | −7.64259 | −1.6683 | 0.0009 | 0.029595 | yes |
| XLOC_017703 | KO | 0.322438 | 0.002003 | −7.33062 | −0.04206 | 5.00E−05 | 0.002735 | yes |
| XLOC_039936 | KO | 90.8139 | 0.830727 | −6.77239 | −5.80423 | 5.00E−05 | 0.002735 | yes |
| XLOC_014052 | KO | 51.0324 | 0.563355 | −6.50123 | −7.32949 | 5.00E−05 | 0.002735 | yes |
| XLOC_048042 | KO | 25.6828 | 0.293896 | −6.44935 | −1.00648 | 5.00E−05 | 0.002735 | yes |
| XLOC_025713 | KO | 6.35971 | 0.07438 | −6.41791 | −2.20203 | 5.00E−05 | 0.002735 | yes |
| XLOC_036590 | KO | 3.22484 | 0.042806 | −6.23527 | −1.28538 | 5.00E−05 | 0.002735 | yes |
| XLOC_023764 | KO | 36.99 | 0.528318 | −6.12959 | −8.72795 | 5.00E−05 | 0.002735 | yes |
| XLOC_026995 | KO | 1.97436 | 0.029986 | −6.04093 | −1.37314 | 5.00E−05 | 0.002735 | yes |
| XLOC_002958 | KO | 13.7831 | 0.215454 | −5.99938 | −1.97937 | 0.00025 | 0.010784 | yes |
| XLOC_006922 | KO | 4.25081 | 0.070798 | −5.90789 | −4.46714 | 0.0001 | 0.005033 | yes |
| XLOC_034614 | KO | 12.7807 | 0.215727 | −5.88862 | −3.90651 | 5.00E−05 | 0.002735 | yes |
| XLOC_036565 | KO | 33.6845 | 0.601204 | −5.80809 | −1.83522 | 5.00E−05 | 0.002735 | yes |
| XLOC_018597 | KO | 18.1238 | 0.378467 | −5.58158 | −5.5681 | 5.00E−05 | 0.002735 | yes |
| XLOC_030767 | KO | 2.87445 | 0.067814 | −5.40557 | −2.46073 | 5.00E−05 | 0.002735 | yes |
| XLOC_008243 | KO | 3.12687 | 0.074384 | −5.39358 | −3.86149 | 0.0001 | 0.005033 | yes |
| XLOC_024400 | KO | 10.1296 | 0.243486 | −5.37859 | −6.01904 | 5.00E−05 | 0.002735 | yes |
| XLOC_048281 | KO | 35.1109 | 0.902144 | −5.28242 | −6.55776 | 5.00E−05 | 0.002735 | yes |
| XLOC_033225 | KO | 4.36783 | 0.1225 | −5.15606 | −4.02057 | 0.00025 | 0.010784 | yes |
| XLOC_050766 | KO | 21.0381 | 0.638988 | −5.04107 | −7.07495 | 5.00E−05 | 0.002735 | yes |
| XLOC_027566 | KO | 7.32415 | 0.230465 | −4.99004 | −1.02362 | 0.0001 | 0.005033 | yes |
| XLOC_001908 | KO | 17.7272 | 0.595294 | −4.89622 | −3.51223 | 5.00E−05 | 0.002735 | yes |
| XLOC_040715 | KO | 1.44082 | 0.049585 | −4.86083 | −2.19002 | 5.00E−05 | 0.002735 | yes |
| XLOC_005323 | KO | 5.62995 | 0.206268 | −4.77053 | −2.63121 | 5.00E−05 | 0.002735 | yes |
| XLOC_025547 | KO | 14.1547 | 0.537814 | −4.71803 | −3.33463 | 5.00E−05 | 0.002735 | yes |
| XLOC_041138 | KO | 3.51763 | 0.144547 | −4.605 | −1.64935 | 5.00E−05 | 0.002735 | yes |
| XLOC_030921 | KO | 7.64404 | 0.315819 | −4.59717 | −6.26788 | 5.00E−05 | 0.002735 | yes |
| XLOC_002728 | KO | 41.8801 | 1.75235 | −4.57891 | −8.46904 | 5.00E−05 | 0.002735 | yes |
| XLOC_025503 | KO | 0.331699 | 0.014069 | −4.55928 | −0.51132 | 0.0007 | 0.02448 | yes |
| XLOC_017273 | KO | 65.9526 | 2.86618 | −4.52423 | −5.23236 | 5.00E−05 | 0.002735 | yes |
| XLOC_048386 | KO | 1.57989 | 0.071283 | −4.47012 | −1.73879 | 0.0001 | 0.005033 | yes |
| XLOC_048495 | KO | 3.05449 | 0.147096 | −4.3761 | −3.00176 | 5.00E−05 | 0.002735 | yes |
| XLOC_005835 | KO | 1.85907 | 0.0966 | −4.26641 | −1.87816 | 5.00E−05 | 0.002735 | yes |
| XLOC_027327 | KO | 3.12486 | 0.165476 | −4.2391 | −3.03169 | 5.00E−05 | 0.002735 | yes |
| XLOC_041832 | KO | 132.265 | 7.00698 | −4.2385 | −11.3056 | 5.00E−05 | 0.002735 | yes |
| XLOC_021142 | KO | 374.477 | 20.1806 | −4.21383 | −10.2 | 5.00E−05 | 0.002735 | yes |
| XLOC_018051 | KO | 0.353852 | 0.020222 | −4.12912 | −3.88113 | 5.00E−05 | 0.002735 | yes |
| XLOC_014741 | KO | 18.6011 | 1.08966 | −4.09343 | −6.72095 | 5.00E−05 | 0.002735 | yes |
| XLOC_034205 | KO | 1.92053 | 0.113179 | −4.08483 | −4.04539 | 5.00E−05 | 0.002735 | yes |
| XLOC_043011 | KO | 2.64893 | 0.159703 | −4.05195 | −2.64303 | 5.00E−05 | 0.002735 | yes |
| XLOC_026261 | KO | 1.47403 | 0.092338 | −3.9967 | −0.61431 | 0.0006 | 0.021697 | yes |
| XLOC_005448 | KO | 18.3327 | 1.14897 | −3.996 | −3.06836 | 5.00E−05 | 0.002735 | yes |
| XLOC_020429 | KO | 24.0091 | 1.53786 | −3.96459 | −2.15827 | 0.0004 | 0.015799 | yes |
| XLOC_009925 | KO | 1161.01 | 74.4747 | −3.96248 | −8.12613 | 5.00E−05 | 0.002735 | yes |
| XLOC_034818 | KO | 0.588316 | 0.038319 | −3.94045 | −2.45388 | 0.00015 | 0.007086 | yes |
| XLOC_009328 | KO | 17.4411 | 1.17897 | −3.88689 | −3.67756 | 5.00E−05 | 0.002735 | yes |
| XLOC_008373 | KO | 8.60107 | 0.588929 | −3.86835 | −3.3272 | 5.00E−05 | 0.002735 | yes |
| XLOC_008341 | KO | 5.25207 | 0.369581 | −3.82892 | −2.63127 | 5.00E−05 | 0.002735 | yes |
| XLOC_002706 | KO | 3.41564 | 0.242834 | −3.81411 | −2.45042 | 5.00E−05 | 0.002735 | yes |
| XLOC_024733 | KO | 2.8707 | 0.211004 | −3.76606 | −2.60337 | 5.00E−05 | 0.002735 | yes |
| XLOC_027565 | KO | 1.85122 | 0.14371 | −3.68724 | −3.58858 | 5.00E−05 | 0.002735 | yes |
| XLOC_049024 | KO | 6.50282 | 0.534113 | −3.60585 | −3.36237 | 5.00E−05 | 0.002735 | yes |
| XLOC_002386 | KO | 0.584725 | 0.048063 | −3.60475 | −2.73747 | 5.00E−05 | 0.002735 | yes |
| XLOC_008662 | KO | 0.419861 | 0.035446 | −3.56623 | −1.21575 | 5.00E−05 | 0.002735 | yes |
| XLOC_039183 | KO | 682.925 | 58.1679 | −3.55343 | −10.4794 | 5.00E−05 | 0.002735 | yes |
| XLOC_045037 | KO | 0.378811 | 0.032484 | −3.54366 | −2.94724 | 0.0015 | 0.04365 | yes |
| XLOC_030694 | KO | 0.267042 | 0.023095 | −3.53139 | −1.12835 | 0.0008 | 0.027077 | yes |
| XLOC_024166 | KO | 8.09229 | 0.732091 | −3.46645 | −5.96158 | 5.00E−05 | 0.002735 | yes |
| XLOC_007296 | KO | 21.8429 | 2.01605 | −3.43756 | −6.09094 | 5.00E−05 | 0.002735 | yes |
| XLOC_050235 | KO | 11.5413 | 1.09914 | −3.39236 | −3.4741 | 5.00E−05 | 0.002735 | yes |
| XLOC_048391 | KO | 0.845173 | 0.081871 | −3.36782 | −1.30179 | 5.00E−05 | 0.002735 | yes |
| XLOC_053146 | KO | 15.3471 | 1.49738 | −3.35745 | −4.94741 | 5.00E−05 | 0.002735 | yes |
| XLOC_030190 | KO | 0.810134 | 0.079693 | −3.34563 | −1.13004 | 5.00E−05 | 0.002735 | yes |
| XLOC_012216 | KO | 282.411 | 27.9694 | −3.33588 | −4.85247 | 5.00E−05 | 0.002735 | yes |
| XLOC_053685 | KO | 3.1391 | 0.311345 | −3.33376 | −2.21131 | 0.0002 | 0.008981 | yes |
| XLOC_049217 | KO | 0.432132 | 0.043553 | −3.31064 | −1.28478 | 0.00025 | 0.010784 | yes |
| XLOC_010972 | KO | 19.745 | 1.99594 | −3.30634 | −6.19126 | 5.00E−05 | 0.002735 | yes |
| XLOC_029247 | KO | 18.2894 | 1.88433 | −3.27888 | −4.79568 | 5.00E−05 | 0.002735 | yes |
| XLOC_019871 | KO | 1.10959 | 0.116052 | −3.25718 | −3.55328 | 5.00E−05 | 0.002735 | yes |
| XLOC_048112 | KO | 1.6599 | 0.174874 | −3.24671 | −4.02046 | 5.00E−05 | 0.002735 | yes |
| XLOC_012370 | KO | 4.70027 | 0.495232 | −3.24657 | −0.77627 | 0.00015 | 0.007086 | yes |
| XLOC_040762 | KO | 1.84837 | 0.198791 | −3.21693 | −2.7891 | 5.00E−05 | 0.002735 | yes |
| XLOC_030742 | KO | 10.9357 | 1.18576 | −3.20517 | −1.40992 | 0.00025 | 0.010784 | yes |
| XLOC_030425 | KO | 30.5899 | 3.41028 | −3.16509 | −2.38833 | 5.00E−05 | 0.002735 | yes |
| XLOC_050331 | KO | 6.99633 | 0.785658 | −3.15462 | −6.34637 | 5.00E−05 | 0.002735 | yes |
| XLOC_017031 | KO | 1789.66 | 206.895 | −3.11271 | −8.17714 | 5.00E−05 | 0.002735 | yes |
| XLOC_020676 | KO | 1.01258 | 0.121548 | −3.05843 | −3.73207 | 5.00E−05 | 0.002735 | yes |
| XLOC_002160 | KO | 4.32369 | 0.523629 | −3.04565 | −2.71452 | 0.00015 | 0.007086 | yes |
| XLOC_030368 | KO | 1.70813 | 0.206968 | −3.04494 | −1.73896 | 5.00E−05 | 0.002735 | yes |
| XLOC_037964 | KO | 43.7695 | 5.323 | −3.03961 | −3.04785 | 5.00E−05 | 0.002735 | yes |
| XLOC_005996 | KO | 61.9017 | 7.53754 | −3.03781 | −3.54682 | 5.00E−05 | 0.002735 | yes |
| XLOC_041354 | KO | 2.47954 | 0.308697 | −3.00581 | −3.72353 | 5.00E−05 | 0.002735 | yes |
| XLOC_010676 | KO | 1.50841 | 0.189473 | −2.99296 | −1.95847 | 5.00E−05 | 0.002735 | yes |
| XLOC_035638 | KO | 4.97002 | 0.640552 | −2.95586 | −4.39647 | 5.00E−05 | 0.002735 | yes |
| XLOC_005857 | KO | 0.742394 | 0.095926 | −2.95219 | −2.54529 | 0.0006 | 0.021697 | yes |
| XLOC_016442 | KO | 4.58084 | 0.594035 | −2.94699 | −2.77007 | 5.00E−05 | 0.002735 | yes |
| XLOC_011052 | KO | 3.74288 | 0.486352 | −2.94408 | −3.69824 | 5.00E−05 | 0.002735 | yes |
| XLOC_042581 | KO | 0.716836 | 0.093381 | −2.94045 | −0.19716 | 5.00E−05 | 0.002735 | yes |
| XLOC_040023 | KO | 123.531 | 16.3626 | −2.9164 | −6.98086 | 5.00E−05 | 0.002735 | yes |
| XLOC_050764 | KO | 13.5584 | 1.80508 | −2.90905 | −4.76345 | 5.00E−05 | 0.002735 | yes |
| XLOC_029134 | KO | 3.14525 | 0.422426 | −2.8964 | −2.9707 | 5.00E−05 | 0.002735 | yes |
| XLOC_010672 | KO | 1.76451 | 0.237593 | −2.89271 | −3.03246 | 5.00E−05 | 0.002735 | yes |
| XLOC_037612 | KO | 474.91 | 64.2023 | −2.88696 | −3.3835 | 5.00E−05 | 0.002735 | yes |
| XLOC_027527 | KO | 80.7168 | 10.9298 | −2.88461 | −3.75531 | 5.00E−05 | 0.002735 | yes |
| XLOC_027038 | KO | 0.635858 | 0.089697 | −2.82558 | −2.69339 | 5.00E−05 | 0.002735 | yes |
| XLOC_028175 | KO | 1.87705 | 0.266095 | −2.81845 | −2.0512 | 5.00E−05 | 0.002735 | yes |
| XLOC_037904 | KO | 0.301087 | 0.042875 | −2.81198 | −0.70888 | 0.00015 | 0.007086 | yes |
| XLOC_032052 | KO | 2.41816 | 0.350235 | −2.78752 | −2.84646 | 5.00E−05 | 0.002735 | yes |
| XLOC_002049 | KO | 3.37884 | 0.493156 | −2.77641 | −2.75377 | 5.00E−05 | 0.002735 | yes |
| XLOC_041731 | KO | 153.008 | 22.3593 | −2.77466 | −3.96717 | 5.00E−05 | 0.002735 | yes |
| XLOC_021787 | KO | 28.1021 | 4.14117 | −2.76256 | −2.97761 | 0.00015 | 0.007086 | yes |
| XLOC_039364 | KO | 1.60128 | 0.236481 | −2.75943 | −2.01375 | 0.00175 | 0.049136 | yes |
| XLOC_013274 | KO | 4.19194 | 0.620843 | −2.75531 | −2.31599 | 5.00E−05 | 0.002735 | yes |
| XLOC_012994 | KO | 4.1007 | 0.617778 | −2.73071 | −2.30382 | 0.0011 | 0.034628 | yes |
| XLOC_046363 | KO | 2.93954 | 0.445272 | −2.72283 | −1.41 | 0.0015 | 0.04365 | yes |
| XLOC_024736 | KO | 3.46582 | 0.544143 | −2.67114 | −2.63657 | 0.00045 | 0.017298 | yes |
| XLOC_045760 | KO | 5.35983 | 0.84448 | −2.66605 | −2.6947 | 5.00E−05 | 0.002735 | yes |
| XLOC_051664 | KO | 245.742 | 39.5382 | −2.63582 | −2.59316 | 5.00E−05 | 0.002735 | yes |
| XLOC_043359 | KO | 6.65213 | 1.07684 | −2.62701 | −4.13675 | 5.00E−05 | 0.002735 | yes |
| XLOC_009884 | KO | 21.1143 | 3.45803 | −2.6102 | −3.6504 | 5.00E−05 | 0.002735 | yes |
| XLOC_002245 | KO | 5.68103 | 0.937036 | −2.59998 | −2.27507 | 5.00E−05 | 0.002735 | yes |
| XLOC_013601 | KO | 0.56806 | 0.094264 | −2.59127 | −3.12484 | 5.00E−05 | 0.002735 | yes |
| XLOC_037491 | KO | 13.7676 | 2.32159 | −2.56809 | −4.67039 | 5.00E−05 | 0.002735 | yes |
| XLOC_043013 | KO | 4.49898 | 0.761045 | −2.56354 | −4.64282 | 5.00E−05 | 0.002735 | yes |
| XLOC_026854 | KO | 0.521044 | 0.088182 | −2.56285 | −1.6689 | 0.0006 | 0.021697 | yes |
| XLOC_049110 | KO | 0.287072 | 0.048636 | −2.5613 | −1.83311 | 5.00E−05 | 0.002735 | yes |
| XLOC_005951 | KO | 0.644767 | 0.1098 | −2.5539 | −2.68709 | 0.00015 | 0.007086 | yes |
| XLOC_041015 | KO | 1.10594 | 0.188466 | −2.55289 | −1.84408 | 0.0004 | 0.015799 | yes |
| XLOC_007768 | KO | 9.12975 | 1.56458 | −2.5448 | −2.42375 | 0.00085 | 0.028328 | yes |
| XLOC_052548 | KO | 0.408133 | 0.070038 | −2.54283 | −2.3707 | 0.00165 | 0.0471 | yes |
| XLOC_013946 | KO | 3.14275 | 0.5417 | −2.53646 | −2.33541 | 5.00E−05 | 0.002735 | yes |
| XLOC_008890 | KO | 9.02766 | 1.55615 | −2.53638 | −2.35921 | 5.00E−05 | 0.002735 | yes |
| XLOC_039304 | KO | 3.84835 | 0.666384 | −2.52981 | −2.4045 | 5.00E−05 | 0.002735 | yes |
| XLOC_015380 | KO | 138.6 | 24.1608 | −2.52019 | −5.78785 | 5.00E−05 | 0.002735 | yes |
| XLOC_006001 | KO | 5.32332 | 0.931784 | −2.51426 | −2.89723 | 0.0001 | 0.005033 | yes |
| XLOC_045083 | KO | 1.3188 | 0.232995 | −2.50085 | −1.31698 | 0.0002 | 0.008981 | yes |
| XLOC_034738 | KO | 6.05702 | 1.10413 | −2.4557 | −4.4028 | 5.00E−05 | 0.002735 | yes |
| XLOC_051434 | KO | 1.54312 | 0.281644 | −2.4539 | −2.18811 | 0.0008 | 0.027077 | yes |
| XLOC_002251 | KO | 2.58726 | 0.473019 | −2.45146 | −1.79353 | 0.00065 | 0.023143 | yes |
| XLOC_017507 | KO | 3.39596 | 0.621874 | −2.44913 | −3.29809 | 5.00E−05 | 0.002735 | yes |
| XLOC_020114 | KO | 4.68771 | 0.863093 | −2.44129 | −2.18362 | 0.00035 | 0.014242 | yes |
| XLOC_003943 | KO | 0.374986 | 0.069068 | −2.44075 | −0.63106 | 0.00075 | 0.025855 | yes |
| XLOC_012012 | KO | 3.96584 | 0.735353 | −2.43112 | −3.54944 | 5.00E−05 | 0.002735 | yes |
| XLOC_027037 | KO | 0.395158 | 0.073764 | −2.42143 | −2.31832 | 0.00145 | 0.042686 | yes |
| XLOC_037223 | KO | 24.1676 | 4.55112 | −2.40878 | −3.72524 | 5.00E−05 | 0.002735 | yes |
| XLOC_025549 | KO | 0.492645 | 0.093686 | −2.39465 | −1.26382 | 0.0009 | 0.029595 | yes |
| XLOC_003160 | KO | 58.9989 | 11.325 | −2.38118 | −5.06659 | 5.00E−05 | 0.002735 | yes |
| XLOC_032087 | KO | 0.284765 | 0.054765 | −2.37844 | −2.16421 | 0.0013 | 0.039328 | yes |
| XLOC_018083 | KO | 64.0612 | 12.3305 | −2.37722 | −1.17307 | 0.00015 | 0.007086 | yes |
| XLOC_006181 | KO | 0.345905 | 0.066947 | −2.36929 | −1.0104 | 0.00145 | 0.042686 | yes |
| XLOC_036866 | KO | 78.3712 | 15.189 | −2.36729 | −4.63835 | 5.00E−05 | 0.002735 | yes |
| XLOC_001171 | KO | 10.0322 | 1.94475 | −2.36699 | −4.51053 | 5.00E−05 | 0.002735 | yes |
| XLOC_041413 | KO | 18.0166 | 3.49572 | −2.36566 | −3.08834 | 5.00E−05 | 0.002735 | yes |
| XLOC_036625 | KO | 94.1119 | 18.2626 | −2.36549 | −5.07047 | 5.00E−05 | 0.002735 | yes |
| XLOC_027645 | KO | 67.006 | 13.0232 | −2.3632 | −5.39545 | 5.00E−05 | 0.002735 | yes |
| XLOC_032532 | KO | 19.4305 | 3.82899 | −2.34328 | −3.13535 | 5.00E−05 | 0.002735 | yes |
| XLOC_043065 | KO | 7.45336 | 1.48119 | −2.33114 | −4.10649 | 5.00E−05 | 0.002735 | yes |
| XLOC_038190 | KO | 3.23318 | 0.642567 | −2.33104 | −3.0731 | 5.00E−05 | 0.002735 | yes |
| XLOC_049746 | KO | 0.994641 | 0.199654 | −2.31667 | −2.23615 | 0.0002 | 0.008981 | yes |
| XLOC_001567 | KO | 1.34371 | 0.270581 | −2.31209 | −1.91751 | 0.0006 | 0.021697 | yes |
| XLOC_038812 | KO | 105.354 | 21.3903 | −2.30022 | −2.53955 | 0.0001 | 0.005033 | yes |
| XLOC_051436 | KO | 0.906457 | 0.186676 | −2.2797 | −1.5455 | 0.00145 | 0.042686 | yes |
| XLOC_042949 | KO | 1.89419 | 0.390128 | −2.27956 | −2.47285 | 0.00015 | 0.007086 | yes |
| XLOC_025172 | KO | 3.03429 | 0.625222 | −2.27892 | −2.43247 | 0.00025 | 0.010784 | yes |
| XLOC_004746 | KO | 5.33443 | 1.10872 | −2.26644 | −2.09819 | 5.00E−05 | 0.002735 | yes |
| XLOC_046915 | KO | 11.7941 | 2.46475 | −2.25855 | −2.59082 | 5.00E−05 | 0.002735 | yes |
| XLOC_012705 | KO | 2.48835 | 0.522027 | −2.25299 | −2.65756 | 5.00E−05 | 0.002735 | yes |
| XLOC_004935 | KO | 0.677219 | 0.142548 | −2.24818 | −2.6221 | 5.00E−05 | 0.002735 | yes |
| XLOC_044557 | KO | 8.39441 | 1.79933 | −2.22197 | −4.11767 | 5.00E−05 | 0.002735 | yes |
| XLOC_021148 | KO | 0.945894 | 0.206344 | −2.19663 | −1.71145 | 0.00075 | 0.025855 | yes |
| XLOC_030096 | KO | 2.80391 | 0.615444 | −2.18774 | −2.51092 | 0.00025 | 0.010784 | yes |
| XLOC_035763 | KO | 1.38609 | 0.305784 | −2.18044 | −1.87038 | 0.0011 | 0.034628 | yes |
| XLOC_000763 | KO | 0.577785 | 0.128637 | −2.16723 | −2.2213 | 0.00125 | 0.038196 | yes |
| XLOC_008424 | KO | 53.3514 | 12.3858 | −2.10684 | −3.62016 | 5.00E−05 | 0.002735 | yes |
| XLOC_019146 | KO | 0.741948 | 0.173064 | −2.10002 | −1.0304 | 0.0005 | 0.018851 | yes |
| XLOC_049099 | KO | 0.25859 | 0.060763 | −2.08941 | −1.37344 | 0.00115 | 0.035883 | yes |
| XLOC_030539 | KO | 12.7911 | 3.0227 | −2.08123 | −5.23097 | 5.00E−05 | 0.002735 | yes |
| XLOC_031027 | KO | 1.48165 | 0.352849 | −2.07008 | −2.39076 | 0.00035 | 0.014242 | yes |
| XLOC_003694 | KO | 7.72747 | 1.84636 | −2.06531 | −2.57245 | 5.00E−05 | 0.002735 | yes |
| XLOC_051965 | KO | 2.34593 | 0.561837 | −2.06193 | −3.55348 | 5.00E−05 | 0.002735 | yes |
| XLOC_037731 | KO | 8.25338 | 1.9801 | −2.05941 | −2.31193 | 5.00E−05 | 0.002735 | yes |
| XLOC_001154 | KO | 3.80901 | 0.914235 | −2.05878 | −2.71511 | 0.0001 | 0.005033 | yes |
| XLOC_038913 | KO | 4.48878 | 1.08237 | −2.05213 | −2.1674 | 5.00E−05 | 0.002735 | yes |
| XLOC_039460 | KO | 8.01707 | 1.94197 | −2.04555 | −1.38561 | 5.00E−05 | 0.002735 | yes |
| XLOC_047896 | KO | 46.5979 | 11.3107 | −2.04258 | −5.04385 | 5.00E−05 | 0.002735 | yes |
| XLOC_023770 | KO | 0.386687 | 0.094425 | −2.03393 | −1.29814 | 0.00025 | 0.010784 | yes |
| XLOC_049601 | KO | 2.23105 | 0.548564 | −2.02399 | −3.08791 | 5.00E−05 | 0.002735 | yes |
| XLOC_014587 | KO | 2.20468 | 0.542186 | −2.02371 | −1.75118 | 0.0014 | 0.041595 | yes |
| XLOC_023424 | KO | 42.8887 | 10.5738 | −2.02011 | −3.23999 | 5.00E−05 | 0.002735 | yes |
| XLOC_009539 | KO | 1.1183 | 0.280123 | −1.99718 | −2.28459 | 0.00025 | 0.010784 | yes |
| XLOC_015770 | KO | 0.383069 | 0.096156 | −1.99416 | −1.48569 | 0.0012 | 0.037051 | yes |
| XLOC_009129 | KO | 1.24572 | 0.32174 | −1.95302 | −1.99375 | 0.0015 | 0.04365 | yes |
| XLOC_043180 | KO | 2.51959 | 0.654558 | −1.9446 | −2.75987 | 5.00E−05 | 0.002735 | yes |
| XLOC_050369 | KO | 17.2334 | 4.50098 | −1.93689 | −4.90384 | 5.00E−05 | 0.002735 | yes |
| XLOC_046916 | KO | 45.4135 | 11.8701 | −1.93579 | −3.21121 | 5.00E−05 | 0.002735 | yes |
| XLOC_048409 | KO | 2.80108 | 0.732425 | −1.93523 | −2.15143 | 0.00035 | 0.014242 | yes |
| XLOC_002599 | KO | 2.30957 | 0.604726 | −1.93327 | −2.33816 | 5.00E−05 | 0.002735 | yes |
| XLOC_003586 | KO | 1.0471 | 0.275225 | −1.92772 | −2.06343 | 0.00075 | 0.025855 | yes |
| XLOC_048138 | KO | 0.806364 | 0.212782 | −1.92206 | −2.05689 | 0.00095 | 0.030847 | yes |
| XLOC_027354 | KO | 66.0711 | 17.4829 | −1.91807 | −4.01842 | 5.00E−05 | 0.002735 | yes |
| XLOC_038957 | KO | 4.115 | 1.09713 | −1.90716 | −2.90982 | 5.00E−05 | 0.002735 | yes |
| XLOC_049191 | KO | 6.74647 | 1.80626 | −1.90113 | −2.21047 | 0.0008 | 0.027077 | yes |
| XLOC_041939 | KO | 2.76418 | 0.744292 | −1.89291 | −1.73006 | 0.00045 | 0.017298 | yes |
| XLOC_006501 | KO | 1.25896 | 0.339818 | −1.8894 | −2.56666 | 5.00E−05 | 0.002735 | yes |
| XLOC_008690 | KO | 7.05588 | 1.90592 | −1.88834 | −2.13602 | 0.0008 | 0.027077 | yes |
| XLOC_044977 | KO | 21.5612 | 5.85194 | −1.88145 | −2.50564 | 5.00E−05 | 0.002735 | yes |
| XLOC_003656 | KO | 0.895315 | 0.244172 | −1.8745 | −1.83209 | 0.00055 | 0.020318 | yes |
| XLOC_038031 | KO | 3.9946 | 1.09015 | −1.87352 | −0.8676 | 0.0012 | 0.037051 | yes |
| XLOC_003193 | KO | 362.181 | 99.4405 | −1.8648 | −5.45065 | 5.00E−05 | 0.002735 | yes |
| XLOC_034650 | KO | 11.0536 | 3.05952 | −1.85314 | −2.77236 | 5.00E−05 | 0.002735 | yes |
| XLOC_039653 | KO | 16.8665 | 4.67297 | −1.85175 | −2.13999 | 5.00E−05 | 0.002735 | yes |
| XLOC_013909 | KO | 1.6732 | 0.463576 | −1.85173 | −2.13849 | 0.00055 | 0.020318 | yes |
| XLOC_045036 | KO | 12.3413 | 3.43959 | −1.84319 | −2.53212 | 0.00025 | 0.010784 | yes |
| XLOC_038757 | KO | 6.23477 | 1.74357 | −1.83829 | −3.22673 | 5.00E-05 | 0.002735 | yes |
| XLOC_036882 | KO | 59.19 | 16.5615 | −1.83752 | −5.15489 | 5.00E−05 | 0.002735 | yes |
| XLOC_003759 | KO | 0.268839 | 0.076196 | −1.81895 | −2.00925 | 0.00025 | 0.010784 | yes |
| XLOC_050248 | KO | 29.0636 | 8.29148 | −1.80951 | −4.88885 | 5.00E−05 | 0.002735 | yes |
| XLOC_050264 | KO | 200.172 | 57.3764 | −1.80271 | −5.1028 | 5.00E−05 | 0.002735 | yes |
| XLOC_017955 | KO | 10.9445 | 3.14469 | −1.79922 | −2.14123 | 0.00015 | 0.007086 | yes |
| XLOC_013753 | KO | 11.6532 | 3.38567 | −1.78321 | −2.51568 | 5.00E−05 | 0.002735 | yes |
| XLOC_019804 | KO | 5.09031 | 1.47943 | −1.78271 | −1.97354 | 0.0007 | 0.02448 | yes |
| XLOC_038358 | KO | 1.0431 | 0.30355 | −1.78087 | −2.18074 | 0.00035 | 0.014242 | yes |
| XLOC_035320 | KO | 14.9677 | 4.37344 | −1.77501 | −3.07021 | 5.00E−05 | 0.002735 | yes |
| XLOC_005819 | KO | 1.23879 | 0.362438 | −1.77313 | −2.27118 | 0.00015 | 0.007086 | yes |
| XLOC_051394 | KO | 1.15346 | 0.337627 | −1.77247 | −1.7974 | 0.0015 | 0.04365 | yes |
| XLOC_047676 | KO | 2.15868 | 0.63221 | −1.77167 | −3.28716 | 5.00E−05 | 0.002735 | yes |
| XLOC_031614 | KO | 1.73147 | 0.508535 | −1.76758 | −2.12415 | 0.0003 | 0.012524 | yes |
| XLOC_028345 | KO | 2.02269 | 0.594081 | −1.76754 | −1.73029 | 0.00075 | 0.025855 | yes |
| XLOC_004684 | KO | 0.781482 | 0.229655 | −1.76674 | −0.43377 | 5.00E−05 | 0.002735 | yes |
| XLOC_044494 | KO | 9.95773 | 2.93419 | −1.76285 | −4.29488 | 5.00E−05 | 0.002735 | yes |
| XLOC_036549 | KO | 19.8488 | 5.87479 | −1.75645 | −2.93158 | 5.00E−05 | 0.002735 | yes |
| XLOC_052577 | KO | 1.82621 | 0.54297 | −1.74991 | −2.59037 | 0.00015 | 0.007086 | yes |
| XLOC_006438 | KO | 8.7805 | 2.61387 | −1.74812 | −2.6046 | 5.00E−05 | 0.002735 | yes |
| XLOC_034437 | KO | 4.01038 | 1.20071 | −1.73985 | −2.00304 | 5.00E−05 | 0.002735 | yes |
| XLOC_013669 | KO | 12.8456 | 3.87492 | −1.72904 | −3.91062 | 5.00E−05 | 0.002735 | yes |
| XLOC_011800 | KO | 28.1667 | 8.50007 | −1.72844 | −2.00598 | 0.0004 | 0.015799 | yes |
| XLOC_044069 | KO | 13.4471 | 4.06047 | −1.72758 | −3.82443 | 5.00E−05 | 0.002735 | yes |
| XLOC_009023 | KO | 90.7614 | 27.5685 | −1.71906 | −1.77635 | 0.0011 | 0.034628 | yes |
| XLOC_038688 | KO | 1.03253 | 0.314697 | −1.71414 | −1.96261 | 0.00135 | 0.040437 | yes |
| XLOC_040718 | KO | 3.9144 | 1.19594 | −1.71064 | −3.37438 | 5.00E−05 | 0.002735 | yes |
| XLOC_035875 | KO | 6.21201 | 1.89845 | −1.71024 | −2.85077 | 5.00E−05 | 0.002735 | yes |
| XLOC_003270 | KO | 1.09958 | 0.337027 | −1.70602 | −2.10497 | 0.00055 | 0.020318 | yes |
| XLOC_005547 | KO | 8.79043 | 2.69944 | −1.70328 | −2.21258 | 0.00045 | 0.017298 | yes |
| XLOC_020764 | KO | 2.31784 | 0.7121 | −1.70263 | −2.14435 | 0.0009 | 0.029595 | yes |
| XLOC_019328 | KO | 2.87693 | 0.884168 | −1.70214 | −0.95366 | 0.0006 | 0.021697 | yes |
| XLOC_039480 | KO | 26.7 | 8.297 | −1.68618 | −1.86175 | 0.00135 | 0.040437 | yes |
| XLOC_004455 | KO | 3.41227 | 1.0671 | −1.67704 | −3.10681 | 5.00E−05 | 0.002735 | yes |
| XLOC_027332 | KO | 0.627643 | 0.196532 | −1.67518 | −0.34345 | 0.0003 | 0.012524 | yes |
| XLOC_002198 | KO | 54.2074 | 17.306 | −1.64722 | −2.72479 | 0.00015 | 0.007086 | yes |
| XLOC_010645 | KO | 6.26065 | 2.00333 | −1.64391 | −2.32636 | 0.00035 | 0.014242 | yes |
| XLOC_034696 | KO | 33.0474 | 10.5767 | −1.64364 | −2.52373 | 5.00E−05 | 0.002735 | yes |
| XLOC_000241 | KO | 42.1639 | 13.6825 | −1.62368 | −4.36818 | 5.00E−05 | 0.002735 | yes |
| XLOC_039386 | KO | 1.31445 | 0.426718 | −1.62311 | −1.48548 | 0.00125 | 0.038196 | yes |
| XLOC_053212 | KO | 2.708 | 0.879563 | −1.62237 | −2.24753 | 0.0004 | 0.015799 | yes |
| XLOC_052663 | KO | 75.7113 | 24.5939 | −1.62221 | −3.04523 | 5.00E−05 | 0.002735 | yes |
| XLOC_039466 | KO | 1.02834 | 0.334353 | −1.62087 | −1.83078 | 0.0008 | 0.027077 | yes |
| XLOC_017654 | KO | 15.3727 | 5.00867 | −1.61787 | −2.12866 | 0.0008 | 0.027077 | yes |
| XLOC_047223 | KO | 6.1214 | 1.99544 | −1.61716 | −3.08689 | 5.00E−05 | 0.002735 | yes |
| XLOC_003629 | KO | 1.71092 | 0.559275 | −1.61314 | −1.05619 | 0.0004 | 0.015799 | yes |
| XLOC_010433 | KO | 3.94169 | 1.29099 | −1.61034 | −2.61798 | 5.00E−05 | 0.002735 | yes |
| XLOC_026186 | KO | 22.3438 | 7.3724 | −1.59967 | −2.32535 | 5.00E−05 | 0.002735 | yes |
| XLOC_038828 | KO | 4.99956 | 1.65319 | −1.59655 | −2.2868 | 0.0002 | 0.008981 | yes |
| XLOC_006249 | KO | 0.613709 | 0.20387 | −1.58991 | −1.19609 | 0.0015 | 0.04365 | yes |
| XLOC_034292 | KO | 6.12088 | 2.03902 | −1.58586 | −2.43569 | 5.00E−05 | 0.002735 | yes |
| XLOC_053223 | KO | 0.421532 | 0.140638 | −1.58365 | −1.75284 | 0.0003 | 0.012524 | yes |
| XLOC_011232 | KO | 1.15015 | 0.383792 | −1.58343 | −1.11315 | 0.00075 | 0.025855 | yes |
| XLOC_037288 | KO | 21.8081 | 7.35384 | −1.56829 | −2.21847 | 5.00E−05 | 0.002735 | yes |
| XLOC_024145 | KO | 6.99037 | 2.3635 | −1.56445 | −2.28907 | 5.00E−05 | 0.002735 | yes |
| XLOC_037647 | KO | 5.77732 | 1.96239 | −1.55779 | −2.1471 | 0.00035 | 0.014242 | yes |
| XLOC_002435 | KO | 16.356 | 5.56534 | −1.55528 | −2.33608 | 0.00055 | 0.020318 | yes |
| XLOC_007098 | KO | 8.42084 | 2.88108 | −1.54735 | −3.12138 | 5.00E−05 | 0.002735 | yes |
| XLOC_039218 | KO | 19.3362 | 6.63039 | −1.54413 | −3.40354 | 5.00E−05 | 0.002735 | yes |
| XLOC_050847 | KO | 2.35306 | 0.816701 | −1.52666 | −2.35945 | 5.00E−05 | 0.002735 | yes |
| XLOC_049164 | KO | 9.52422 | 3.3134 | −1.52329 | −2.79672 | 5.00E−05 | 0.002735 | yes |
| XLOC_011248 | KO | 4.72903 | 1.66333 | −1.50747 | −2.67397 | 5.00E−05 | 0.002735 | yes |
| XLOC_028509 | KO | 1.22008 | 0.42977 | −1.50534 | −1.74354 | 0.00105 | 0.03336 | yes |
| XLOC_038748 | KO | 3.92563 | 1.38387 | −1.50421 | −2.18318 | 5.00E−05 | 0.002735 | yes |
| XLOC_049806 | KO | 28.1104 | 9.91212 | −1.50384 | −3.7685 | 5.00E−05 | 0.002735 | yes |
| XLOC_044981 | KO | 32.1077 | 11.3848 | −1.49581 | −2.92334 | 5.00E−05 | 0.002735 | yes |
| XLOC_034156 | KO | 24.7366 | 8.83794 | −1.48487 | −3.8475 | 5.00E−05 | 0.002735 | yes |
| XLOC_012486 | KO | 40.1892 | 14.3991 | −1.48083 | −2.86755 | 5.00E−05 | 0.002735 | yes |
| XLOC_017508 | KO | 277.888 | 99.6569 | −1.47946 | −2.58013 | 5.00E−05 | 0.002735 | yes |
| XLOC_023287 | KO | 1.99338 | 0.722277 | −1.4646 | −1.88344 | 0.00055 | 0.020318 | yes |
| XLOC_047196 | KO | 15.1672 | 5.50184 | −1.46297 | −2.86786 | 5.00E−05 | 0.002735 | yes |
| XLOC_028947 | KO | 0.399154 | 0.146302 | −1.448 | −0.555 | 5.00E−05 | 0.002735 | yes |
| XLOC_015553 | KO | 3.33689 | 1.23637 | −1.43239 | −1.09507 | 0.00115 | 0.035883 | yes |
| XLOC_003678 | KO | 21.137 | 7.8397 | −1.4309 | −3.52603 | 5.00E−05 | 0.002735 | yes |
| XLOC_031529 | KO | 5.98448 | 2.23368 | −1.4218 | −3.25278 | 5.00E−05 | 0.002735 | yes |
| XLOC_007421 | KO | 702.559 | 262.65 | −1.41948 | −3.20587 | 5.00E−05 | 0.002735 | yes |
| XLOC_004771 | KO | 7.22815 | 2.71502 | −1.41266 | −2.24459 | 5.00E−05 | 0.002735 | yes |
| XLOC_018419 | KO | 17.409 | 6.59944 | −1.39942 | −1.99618 | 0.0009 | 0.029595 | yes |
| XLOC_044600 | KO | 5.78204 | 2.20651 | −1.38981 | −2.54026 | 0.0001 | 0.005033 | yes |
| XLOC_011113 | KO | 25.1101 | 9.64113 | −1.381 | −2.41814 | 5.00E−05 | 0.002735 | yes |
| XLOC_029655 | KO | 4.78991 | 1.83917 | −1.38095 | −2.49374 | 0.0001 | 0.005033 | yes |
| XLOC_025709 | KO | 13.0308 | 5.06852 | −1.36229 | −1.76943 | 0.00135 | 0.040437 | yes |
| XLOC_035892 | KO | 39.354 | 15.3097 | −1.36207 | −2.18675 | 0.0002 | 0.008981 | yes |
| XLOC_045084 | KO | 0.795032 | 0.309744 | −1.35994 | −1.42356 | 0.00115 | 0.035883 | yes |
| XLOC_054143 | KO | 8.72867 | 3.40655 | −1.35745 | −1.72119 | 0.0006 | 0.021697 | yes |
| XLOC_008272 | KO | 9.15741 | 3.5804 | −1.35482 | −1.78976 | 0.00125 | 0.038196 | yes |
| XLOC_044991 | KO | 3.98056 | 1.56136 | −1.35017 | −2.71689 | 5.00E−05 | 0.002735 | yes |
| XLOC_054197 | KO | 5.55527 | 2.18751 | −1.34457 | −1.67642 | 0.00135 | 0.040437 | yes |
| XLOC_042802 | KO | 2.52107 | 0.995187 | −1.341 | −1.98022 | 0.0016 | 0.045919 | yes |
| XLOC_001178 | KO | 52.9734 | 20.9307 | −1.33964 | −2.76855 | 5.00E−05 | 0.002735 | yes |
| XLOC_048061 | KO | 2.59948 | 1.0275 | −1.33909 | −1.87764 | 0.00105 | 0.03336 | yes |
| XLOC_049026 | KO | 123.358 | 48.8704 | −1.33582 | −4.20545 | 5.00E−05 | 0.002735 | yes |
| XLOC_049157 | KO | 20.144 | 8.00261 | −1.33181 | −3.63713 | 5.00E−05 | 0.002735 | yes |
| XLOC_016660 | KO | 12.3705 | 4.91447 | −1.33179 | −2.08266 | 0.0016 | 0.045919 | yes |
| XLOC_034147 | KO | 12.1316 | 4.83047 | −1.32853 | −1.81676 | 5.00E−05 | 0.002735 | yes |
| XLOC_030666 | KO | 7.54085 | 3.01214 | −1.32394 | −2.44532 | 5.00E−05 | 0.002735 | yes |
| XLOC_006553 | KO | 23.2596 | 9.34184 | −1.31605 | −1.6284 | 5.00E−05 | 0.002735 | yes |
| XLOC_028877 | KO | 97.1879 | 39.2029 | −1.30982 | −3.05022 | 5.00E−05 | 0.002735 | yes |
| XLOC_043536 | KO | 84.4552 | 34.1251 | −1.30735 | −3.84165 | 5.00E−05 | 0.002735 | yes |
| XLOC_036181 | KO | 152.224 | 61.7318 | −1.30211 | −1.98636 | 0.00085 | 0.028328 | yes |
| XLOC_001874 | KO | 39.7368 | 16.1837 | −1.29594 | −3.42486 | 5.00E−05 | 0.002735 | yes |
| XLOC_048183 | KO | 19.7062 | 8.03804 | −1.29373 | −2.03513 | 0.0012 | 0.037051 | yes |
| XLOC_048884 | KO | 27.9196 | 11.45 | −1.28593 | −3.24215 | 5.00E−05 | 0.002735 | yes |
| XLOC_027617 | KO | 26.4953 | 10.8795 | −1.28412 | −3.31733 | 5.00E−05 | 0.002735 | yes |
| XLOC_007792 | KO | 3.58211 | 1.47581 | −1.27931 | −2.14108 | 0.00035 | 0.014242 | yes |
| XLOC_006019 | KO | 121.709 | 50.3282 | −1.27399 | −3.67188 | 5.00E−05 | 0.002735 | yes |
| XLOC_024146 | KO | 31.1534 | 12.9193 | −1.26986 | −3.78622 | 5.00E−05 | 0.002735 | yes |
| XLOC_011021 | KO | 22.3665 | 9.36143 | −1.25654 | −2.03252 | 0.0007 | 0.02448 | yes |
| XLOC_048829 | KO | 6.82158 | 2.85779 | −1.25521 | −1.90499 | 5.00E−05 | 0.002735 | yes |
| XLOC_014133 | KO | 101.751 | 42.8844 | −1.24651 | −3.93452 | 5.00E−05 | 0.002735 | yes |
| XLOC_000084 | KO | 3.59607 | 1.52059 | −1.24179 | −2.07865 | 0.00045 | 0.017298 | yes |
| XLOC_005777 | KO | 3.08606 | 1.31263 | −1.23331 | −2.03421 | 5.00E−05 | 0.002735 | yes |
| XLOC_023843 | KO | 4.09666 | 1.74661 | −1.22989 | −2.10578 | 0.0002 | 0.008981 | yes |
| XLOC_040879 | KO | 123.201 | 52.6185 | −1.22737 | −1.9734 | 0.00145 | 0.042686 | yes |
| XLOC_031878 | KO | 7.10173 | 3.07214 | −1.20893 | −2.55301 | 5.00E−05 | 0.002735 | yes |
| XLOC_046371 | KO | 6.46922 | 2.80543 | −1.20537 | −2.12413 | 0.00095 | 0.030847 | yes |
| XLOC_013777 | KO | 41.4369 | 18.0507 | −1.19886 | −3.37099 | 5.00E−05 | 0.002735 | yes |
| XLOC_025056 | KO | 4.86512 | 2.12005 | −1.19838 | −1.9063 | 0.00025 | 0.010784 | yes |
| XLOC_026645 | KO | 9.13983 | 4.00573 | −1.1901 | −1.83082 | 0.00015 | 0.007086 | yes |
| XLOC_038883 | KO | 15.0737 | 6.61438 | −1.18835 | −1.48691 | 0.0004 | 0.015799 | yes |
| XLOC_040599 | KO | 40.6696 | 17.8712 | −1.18631 | −1.4713 | 0.0007 | 0.02448 | yes |
| XLOC_036879 | KO | 134.611 | 59.2836 | −1.18309 | −2.1043 | 5.00E−05 | 0.002735 | yes |
| XLOC_053052 | KO | 3.75209 | 1.66264 | −1.17422 | −1.94954 | 0.0012 | 0.037051 | yes |
| XLOC_004061 | KO | 91.66 | 40.6803 | −1.17196 | −2.84108 | 5.00E−05 | 0.002735 | yes |
| XLOC_010923 | KO | 163.728 | 72.7628 | −1.17003 | −3.60833 | 5.00E−05 | 0.002735 | yes |
| XLOC_051104 | KO | 89.957 | 39.981 | −1.16992 | −3.55585 | 5.00E−05 | 0.002735 | yes |
| XLOC_000284 | KO | 35.0271 | 15.6015 | −1.16678 | −3.30325 | 5.00E−05 | 0.002735 | yes |
| XLOC_006336 | KO | 1.56767 | 0.698831 | −1.1656 | −1.99677 | 0.00055 | 0.020318 | yes |
| XLOC_004451 | KO | 26.8217 | 11.9663 | −1.16443 | −3.43778 | 5.00E−05 | 0.002735 | yes |
| XLOC_023416 | KO | 51.2899 | 22.9943 | −1.1574 | −1.50185 | 0.00015 | 0.007086 | yes |
| XLOC_039312 | KO | 127.457 | 57.2095 | −1.15569 | −2.23865 | 5.00E−05 | 0.002735 | yes |
| XLOC_004870 | KO | 20.632 | 9.26229 | −1.15544 | −2.53388 | 5.00E−05 | 0.002735 | yes |
| XLOC_028224 | KO | 9.17595 | 4.13915 | −1.14852 | −1.87923 | 0.00105 | 0.03336 | yes |
| XLOC_005776 | KO | 14.7848 | 6.68067 | −1.14605 | −2.62459 | 5.00E−05 | 0.002735 | yes |
| XLOC_042028 | KO | 81.7624 | 37.1264 | −1.13899 | −1.92487 | 0.0008 | 0.027077 | yes |
| XLOC_003817 | KO | 11.6577 | 5.31277 | −1.13375 | −2.00654 | 0.001 | 0.032108 | yes |
| XLOC_044280 | KO | 2.34947 | 1.07655 | −1.12592 | −1.85318 | 0.0013 | 0.039328 | yes |
| XLOC_054067 | KO | 40.5625 | 18.5979 | −1.12501 | −3.19935 | 5.00E−05 | 0.002735 | yes |
| XLOC_002921 | KO | 377.689 | 173.286 | −1.12405 | −3.56475 | 5.00E−05 | 0.002735 | yes |
| XLOC_003395 | KO | 10.5582 | 4.86568 | −1.11764 | −2.49284 | 5.00E−05 | 0.002735 | yes |
| XLOC_022130 | KO | 22.7403 | 10.5049 | −1.11419 | −1.42844 | 0.00095 | 0.030847 | yes |
| XLOC_051379 | KO | 59.0964 | 27.3081 | −1.11374 | −1.98619 | 0.00065 | 0.023143 | yes |
| XLOC_009139 | KO | 25.6311 | 11.8619 | −1.11156 | −2.66798 | 5.00E−05 | 0.002735 | yes |
| XLOC_042929 | KO | 10.4828 | 4.85999 | −1.109 | −2.4495 | 5.00E−05 | 0.002735 | yes |
| XLOC_003035 | KO | 44.5938 | 20.6895 | −1.10795 | −2.27019 | 0.00045 | 0.017298 | yes |
| XLOC_009886 | KO | 5.02865 | 2.33572 | −1.1063 | −1.79174 | 0.0008 | 0.027077 | yes |
| XLOC_031273 | KO | 14.8531 | 6.91336 | −1.1033 | −1.8769 | 0.00105 | 0.03336 | yes |
| XLOC_027092 | KO | 376.484 | 175.249 | −1.10318 | −3.02031 | 5.00E−05 | 0.002735 | yes |
| XLOC_007077 | KO | 1.72936 | 0.805994 | −1.10139 | −1.28393 | 0.00175 | 0.049136 | yes |
| XLOC_007101 | KO | 157.757 | 73.5517 | −1.10088 | −2.99542 | 5.00E−05 | 0.002735 | yes |
| XLOC_006989 | KO | 7.73359 | 3.61451 | −1.09734 | −1.93991 | 0.00025 | 0.010784 | yes |
| XLOC_011223 | KO | 3.37324 | 1.58056 | −1.0937 | −1.74645 | 0.00085 | 0.028328 | yes |
| XLOC_036975 | KO | 6.92999 | 3.2504 | −1.09223 | −2.08058 | 0.0005 | 0.018851 | yes |
| XLOC_028204 | KO | 0.930909 | 0.437129 | −1.09058 | −0.45143 | 5.00E−05 | 0.002735 | yes |
| XLOC_048252 | KO | 8.3951 | 3.95905 | −1.08439 | −1.84487 | 0.00105 | 0.03336 | yes |
| XLOC_001688 | KO | 5.3835 | 2.53907 | −1.08424 | −1.69198 | 0.00175 | 0.049136 | yes |
| XLOC_044959 | KO | 16.7948 | 7.93382 | −1.08193 | −2.00541 | 0.0013 | 0.039328 | yes |
| XLOC_006957 | KO | 11.3322 | 5.41898 | −1.06433 | −1.91703 | 0.00015 | 0.007086 | yes |
| XLOC_040308 | KO | 82.1743 | 39.5718 | −1.05421 | −2.18327 | 5.00E−05 | 0.002735 | yes |
| XLOC_040646 | KO | 30.0811 | 14.5089 | −1.05192 | −2.8137 | 5.00E−05 | 0.002735 | yes |
| XLOC_048007 | KO | 8.77985 | 4.28762 | −1.03402 | −2.01858 | 0.00045 | 0.017298 | yes |
| XLOC_012876 | KO | 6.96702 | 3.42746 | −1.0234 | −2.53986 | 5.00E−05 | 0.002735 | yes |
| XLOC_037005 | KO | 38.8983 | 19.1366 | −1.02338 | −2.09803 | 0.0002 | 0.008981 | yes |
| XLOC_039416 | KO | 29.6293 | 14.6233 | −1.01876 | −1.8837 | 0.00105 | 0.03336 | yes |
| XLOC_031885 | KO | 13.5965 | 6.73801 | −1.01284 | −2.48442 | 5.00E−05 | 0.002735 | yes |
| XLOC_025480 | KO | 13.1484 | 6.54108 | −1.00729 | −2.54596 | 5.00E−05 | 0.002735 | yes |
| XLOC_053141 | KO | 28.449 | 14.1725 | −1.00528 | −2.4841 | 5.00E−05 | 0.002735 | yes |
| XLOC_011663 | KO | 294.908 | 147.174 | −1.00274 | −2.00927 | 5.00E−05 | 0.002735 | yes |
| XLOC_015234 | KO | 3.80952 | 7.61908 | 1.00001 | 2.61342 | 5.00E−05 | 0.002735 | yes |
| XLOC_011044 | KO | 7.2878 | 14.6238 | 1.00476 | 2.36644 | 5.00E−05 | 0.002735 | yes |
| XLOC_052987 | KO | 9.87566 | 19.8785 | 1.00926 | 2.6223 | 5.00E−05 | 0.002735 | yes |
| XLOC_052880 | KO | 53.456 | 107.661 | 1.01007 | 2.43261 | 5.00E−05 | 0.002735 | yes |
| XLOC_038761 | KO | 43.8994 | 88.5581 | 1.01242 | 3.21263 | 5.00E−05 | 0.002735 | yes |
| XLOC_026220 | KO | 77.868 | 157.543 | 1.01664 | 3.04357 | 5.00E−05 | 0.002735 | yes |
| XLOC_023877 | KO | 4.40171 | 8.90632 | 1.01676 | 2.05122 | 0.0002 | 0.008981 | yes |
| XLOC_024463 | KO | 79.5062 | 161.005 | 1.01797 | 2.06394 | 0.00025 | 0.010784 | yes |
| XLOC_054058 | KO | 24.2914 | 49.2951 | 1.021 | 2.68099 | 5.00E−05 | 0.002735 | yes |
| XLOC_054039 | KO | 10.9677 | 22.2842 | 1.02276 | 2.39519 | 5.00E−05 | 0.002735 | yes |
| XLOC_020149 | KO | 6.36915 | 12.9446 | 1.02318 | 2.08145 | 0.0003 | 0.012524 | yes |
| XLOC_000135 | KO | 13.7324 | 28.0606 | 1.03096 | 2.04272 | 5.00E−05 | 0.002735 | yes |
| XLOC_034496 | KO | 84.6598 | 173.288 | 1.03342 | 2.8 | 5.00E−05 | 0.002735 | yes |
| XLOC_030700 | KO | 14.7909 | 30.3244 | 1.03576 | 3.00311 | 5.00E−05 | 0.002735 | yes |
| XLOC_021959 | KO | 6.15215 | 12.7215 | 1.04811 | 2.70824 | 5.00E−05 | 0.002735 | yes |
| XLOC_053962 | KO | 6.1006 | 12.6485 | 1.05195 | 2.71416 | 5.00E−05 | 0.002735 | yes |
| XLOC_036851 | KO | 20.7169 | 42.9678 | 1.05244 | 1.73382 | 0.0014 | 0.041595 | yes |
| XLOC_007351 | KO | 8.47914 | 17.6812 | 1.06022 | 1.99481 | 0.0007 | 0.02448 | yes |
| XLOC_030393 | KO | 50.5917 | 105.751 | 1.06369 | 2.05265 | 0.0007 | 0.02448 | yes |
| XLOC_045032 | KO | 19.1913 | 40.1185 | 1.06382 | 1.74262 | 0.0002 | 0.008981 | yes |
| XLOC_008588 | KO | 33.7648 | 70.6238 | 1.06463 | 1.93305 | 0.00165 | 0.0471 | yes |
| XLOC_048193 | KO | 66.2249 | 138.784 | 1.0674 | 2.68073 | 5.00E−05 | 0.002735 | yes |
| XLOC_053815 | KO | 10.0667 | 21.1455 | 1.07076 | 2.49548 | 5.00E−05 | 0.002735 | yes |
| XLOC_040597 | KO | 8.1276 | 17.0871 | 1.07201 | 2.16635 | 0.0004 | 0.015799 | yes |
| XLOC_031446 | KO | 168.214 | 356.35 | 1.083 | 3.26649 | 5.00E−05 | 0.002735 | yes |
| XLOC_001360 | KO | 4.28137 | 9.08067 | 1.08473 | 1.89544 | 0.00155 | 0.04486 | yes |
| XLOC_053006 | KO | 3.03606 | 6.44019 | 1.0849 | 2.27783 | 0.00025 | 0.010784 | yes |
| XLOC_035138 | KO | 12.6386 | 26.8364 | 1.08635 | 2.73585 | 5.00E−05 | 0.002735 | yes |
| XLOC_035718 | KO | 2.52965 | 5.39302 | 1.09216 | 1.9143 | 0.00165 | 0.0471 | yes |
| XLOC_041572 | KO | 65.2856 | 139.324 | 1.09361 | 2.67672 | 5.00E−05 | 0.002735 | yes |
| XLOC_053840 | KO | 47.033 | 100.451 | 1.09474 | 3.47853 | 5.00E−05 | 0.002735 | yes |
| XLOC_052847 | KO | 20.2869 | 43.5028 | 1.10056 | 3.12745 | 5.00E−05 | 0.002735 | yes |
| XLOC_024713 | KO | 30.1247 | 64.6101 | 1.10081 | 2.33619 | 5.00E−05 | 0.002735 | yes |
| XLOC_050476 | KO | 21.0177 | 45.6355 | 1.11855 | 2.10784 | 0.0008 | 0.027077 | yes |
| XLOC_051615 | KO | 6.56452 | 14.306 | 1.12385 | 2.71612 | 5.00E−05 | 0.002735 | yes |
| XLOC_038678 | KO | 2.02673 | 4.45669 | 1.13682 | 2.13386 | 0.0002 | 0.008981 | yes |
| XLOC_043700 | KO | 8.87916 | 19.5925 | 1.14181 | 1.89003 | 0.0014 | 0.041595 | yes |
| XLOC_008078 | KO | 7.45577 | 16.493 | 1.14542 | 2.19163 | 0.00025 | 0.010784 | yes |
| XLOC_046906 | KO | 58.8022 | 130.083 | 1.14549 | 2.04206 | 0.0006 | 0.021697 | yes |
| XLOC_002145 | KO | 3.13504 | 6.96175 | 1.15097 | 1.63792 | 0.0002 | 0.008981 | yes |
| XLOC_005982 | KO | 28.6082 | 63.6617 | 1.154 | 2.09932 | 0.0004 | 0.015799 | yes |
| XLOC_002143 | KO | 2.06928 | 4.6169 | 1.15779 | 1.48046 | 0.0017 | 0.048164 | yes |
| XLOC_004604 | KO | 30.2858 | 67.6373 | 1.15918 | 1.96696 | 0.0005 | 0.018851 | yes |
| XLOC_023130 | KO | 3.56065 | 7.96616 | 1.16174 | 2.18367 | 0.0003 | 0.012524 | yes |
| XLOC_053821 | KO | 23.8599 | 53.5873 | 1.1673 | 2.89986 | 5.00E−05 | 0.002735 | yes |
| XLOC_000957 | KO | 26.5108 | 60.2415 | 1.18418 | 3.04613 | 5.00E−05 | 0.002735 | yes |
| XLOC_045574 | KO | 2.62606 | 5.99118 | 1.18994 | 2.41286 | 0.00015 | 0.007086 | yes |
| XLOC_052690 | KO | 1.16105 | 2.6545 | 1.19301 | 2.30912 | 5.00E−05 | 0.002735 | yes |
| XLOC_052629 | KO | 16.2352 | 37.1548 | 1.19443 | 2.78829 | 5.00E−05 | 0.002735 | yes |
| XLOC_046843 | KO | 8.23671 | 18.9484 | 1.20194 | 2.51911 | 5.00E−05 | 0.002735 | yes |
| XLOC_052878 | KO | 11.5599 | 26.5993 | 1.20225 | 2.78574 | 5.00E−05 | 0.002735 | yes |
| XLOC_025629 | KO | 3.30936 | 7.64425 | 1.20782 | 2.5271 | 5.00E−05 | 0.002735 | yes |
| XLOC_042340 | KO | 12.8215 | 29.6412 | 1.20904 | 2.2655 | 0.0002 | 0.008981 | yes |
| XLOC_028258 | KO | 1.74141 | 4.06742 | 1.22386 | 2.15101 | 0.00025 | 0.010784 | yes |
| XLOC_036012 | KO | 27.5439 | 64.6672 | 1.2313 | 2.76425 | 5.00E−05 | 0.002735 | yes |
| XLOC_002280 | KO | 2.97165 | 7.03531 | 1.24335 | 2.68067 | 5.00E−05 | 0.002735 | yes |
| XLOC_054123 | KO | 60.1616 | 142.873 | 1.24782 | 1.29666 | 0.00045 | 0.017298 | yes |
| XLOC_045653 | KO | 5.68015 | 13.5794 | 1.25742 | 3.00513 | 5.00E−05 | 0.002735 | yes |
| XLOC_047007 | KO | 15.6805 | 37.6073 | 1.26204 | 2.4299 | 0.0001 | 0.005033 | yes |
| XLOC_042338 | KO | 4.7777 | 11.4744 | 1.26403 | 2.42558 | 0.0001 | 0.005033 | yes |
| XLOC_054062 | KO | 8.26034 | 19.9021 | 1.26865 | 3.23466 | 5.00E−05 | 0.002735 | yes |
| XLOC_031168 | KO | 163.23 | 394.018 | 1.27135 | 3.37261 | 5.00E−05 | 0.002735 | yes |
| XLOC_049732 | KO | 4.9283 | 11.9161 | 1.27375 | 1.83503 | 0.001 | 0.032108 | yes |
| XLOC_046218 | KO | 28.7723 | 70.2028 | 1.28685 | 3.11403 | 5.00E−05 | 0.002735 | yes |
| XLOC_040610 | KO | 1.33467 | 3.2699 | 1.29277 | 1.96742 | 0.0015 | 0.04365 | yes |
| XLOC_044277 | KO | 14.4832 | 35.5543 | 1.29564 | 2.82827 | 0.00025 | 0.010784 | yes |
| XLOC_005833 | KO | 105.68 | 259.486 | 1.29595 | 3.98504 | 5.00E−05 | 0.002735 | yes |
| XLOC_033519 | KO | 78.9043 | 194.284 | 1.29999 | 3.88223 | 5.00E−05 | 0.002735 | yes |
| XLOC_036029 | KO | 9.69148 | 23.8698 | 1.3004 | 3.28336 | 5.00E−05 | 0.002735 | yes |
| XLOC_032423 | KO | 14.9163 | 36.7415 | 1.30052 | 2.5059 | 5.00E−05 | 0.002735 | yes |
| XLOC_052071 | KO | 69.8407 | 172.894 | 1.30775 | 3.9905 | 5.00E−05 | 0.002735 | yes |
| XLOC_048551 | KO | 2.02625 | 5.02466 | 1.31022 | 1.76829 | 0.00075 | 0.025855 | yes |
| XLOC_007003 | KO | 6.43813 | 16.1089 | 1.32315 | 2.7716 | 5.00E−05 | 0.002735 | yes |
| XLOC_030738 | KO | 7.51868 | 18.9446 | 1.33324 | 2.26785 | 0.0002 | 0.008981 | yes |
| XLOC_026026 | KO | 1.12693 | 2.8411 | 1.33405 | 2.47726 | 5.00E−05 | 0.002735 | yes |
| XLOC_017540 | KO | 4.07318 | 10.2764 | 1.33511 | 1.99525 | 5.00E−05 | 0.002735 | yes |
| XLOC_033561 | KO | 15.1967 | 38.3805 | 1.33661 | 3.51652 | 5.00E−05 | 0.002735 | yes |
| XLOC_030077 | KO | 1.26723 | 3.21085 | 1.34128 | 1.98788 | 0.00075 | 0.025855 | yes |
| XLOC_010023 | KO | 5.90104 | 14.9581 | 1.34189 | 1.96949 | 0.0008 | 0.027077 | yes |
| XLOC_048928 | KO | 9.36661 | 23.7803 | 1.34417 | 3.90375 | 5.00E−05 | 0.002735 | yes |
| XLOC_048992 | KO | 19.9381 | 50.9025 | 1.35221 | 3.17818 | 5.00E−05 | 0.002735 | yes |
| XLOC_005542 | KO | 38.5425 | 99.5566 | 1.36907 | 3.29681 | 5.00E−05 | 0.002735 | yes |
| XLOC_054006 | KO | 13.1477 | 34.0304 | 1.37201 | 2.67697 | 5.00E−05 | 0.002735 | yes |
| XLOC_038088 | KO | 100.459 | 260.341 | 1.3738 | 2.8337 | 5.00E−05 | 0.002735 | yes |
| XLOC_026199 | KO | 28.9917 | 75.2681 | 1.3764 | 3.22416 | 5.00E−05 | 0.002735 | yes |
| XLOC_009683 | KO | 2.68748 | 7.01635 | 1.38447 | 2.97624 | 5.00E−05 | 0.002735 | yes |
| XLOC_054131 | KO | 13.1878 | 34.4304 | 1.38448 | 2.66963 | 5.00E−05 | 0.002735 | yes |
| XLOC_032441 | KO | 2.91575 | 7.63418 | 1.3886 | 2.64816 | 5.00E−05 | 0.002735 | yes |
| XLOC_003976 | KO | 8.08992 | 21.2254 | 1.3916 | 3.21685 | 5.00E−05 | 0.002735 | yes |
| XLOC_053841 | KO | 13.3611 | 35.5581 | 1.41214 | 3.06779 | 5.00E−05 | 0.002735 | yes |
| XLOC_005843 | KO | 4.53894 | 12.1011 | 1.41472 | 1.9081 | 0.0005 | 0.018851 | yes |
| XLOC_012477 | KO | 42.4195 | 114.119 | 1.42775 | 3.06511 | 5.00E−05 | 0.002735 | yes |
| XLOC_014816 | KO | 18.3529 | 49.6401 | 1.4355 | 2.11039 | 0.00055 | 0.020318 | yes |
| XLOC_040656 | KO | 0.940972 | 2.54538 | 1.43566 | 2.42751 | 5.00E−05 | 0.002735 | yes |
| XLOC_006803 | KO | 53.9582 | 146.698 | 1.44293 | 4.46487 | 5.00E−05 | 0.002735 | yes |
| XLOC_052691 | KO | 185.928 | 505.565 | 1.44316 | 3.8742 | 5.00E−05 | 0.002735 | yes |
| XLOC_005775 | KO | 78.0884 | 213.469 | 1.45085 | 3.08272 | 5.00E−05 | 0.002735 | yes |
| XLOC_052372 | KO | 24.2723 | 66.7327 | 1.45908 | 4.07862 | 5.00E−05 | 0.002735 | yes |
| XLOC_000088 | KO | 6.78636 | 18.7682 | 1.46758 | 2.41851 | 0.0001 | 0.005033 | yes |
| XLOC_024612 | KO | 1.63477 | 4.58554 | 1.488 | 2.15573 | 0.00055 | 0.020318 | yes |
| XLOC_026219 | KO | 0.943851 | 2.64789 | 1.48821 | 2.06279 | 5.00E−05 | 0.002735 | yes |
| XLOC_053827 | KO | 115.952 | 325.692 | 1.48998 | 3.69007 | 5.00E−05 | 0.002735 | yes |
| XLOC_053972 | KO | 5.57773 | 15.6749 | 1.4907 | 2.85032 | 5.00E−05 | 0.002735 | yes |
| XLOC_013498 | KO | 0.361074 | 1.02785 | 1.50926 | 1.3166 | 0.0014 | 0.041595 | yes |
| XLOC_031173 | KO | 11.9603 | 34.1916 | 1.5154 | 3.90307 | 5.00E−05 | 0.002735 | yes |
| XLOC_052853 | KO | 2.90881 | 8.48748 | 1.54491 | 2.54431 | 0.0002 | 0.008981 | yes |
| XLOC_043236 | KO | 8.31831 | 24.4362 | 1.55466 | 2.93167 | 5.00E−05 | 0.002735 | yes |
| XLOC_046163 | KO | 1.37258 | 4.0381 | 1.55679 | 2.26618 | 0.0001 | 0.005033 | yes |
| XLOC_053995 | KO | 364.416 | 1074.34 | 1.55979 | 3.77603 | 5.00E−05 | 0.002735 | yes |
| XLOC_016656 | KO | 0.172902 | 0.510356 | 1.56155 | 1.16971 | 0.00165 | 0.0471 | yes |
| XLOC_035032 | KO | 36.1819 | 107.377 | 1.56935 | 4.68112 | 5.00E−05 | 0.002735 | yes |
| XLOC_007911 | KO | 0.421347 | 1.26254 | 1.58325 | 2.00489 | 0.00145 | 0.042686 | yes |
| XLOC_009044 | KO | 1.81447 | 5.52823 | 1.60727 | 1.99538 | 0.00045 | 0.017298 | yes |
| XLOC_025875 | KO | 6.9918 | 21.3048 | 1.60744 | 2.63403 | 5.00E−05 | 0.002735 | yes |
| XLOC_051389 | KO | 2.8445 | 8.68041 | 1.60959 | 2.37565 | 5.00E−05 | 0.002735 | yes |
| XLOC_028631 | KO | 0.366398 | 1.13674 | 1.63342 | 1.7807 | 5.00E−05 | 0.002735 | yes |
| XLOC_035814 | KO | 2.43234 | 7.56835 | 1.63763 | 2.06284 | 0.0007 | 0.02448 | yes |
| XLOC_028335 | KO | 2.97422 | 9.49043 | 1.67396 | 2.44973 | 5.00E−05 | 0.002735 | yes |
| XLOC_042640 | KO | 26.1336 | 83.71 | 1.67949 | 3.34677 | 5.00E−05 | 0.002735 | yes |
| XLOC_037275 | KO | 1.02775 | 3.30235 | 1.68401 | 1.63491 | 5.00E−05 | 0.002735 | yes |
| XLOC_022915 | KO | 1.5145 | 4.91028 | 1.69696 | 2.60539 | 0.0001 | 0.005033 | yes |
| XLOC_014449 | KO | 0.387198 | 1.26125 | 1.70372 | 1.50627 | 0.00095 | 0.030847 | yes |
| XLOC_006704 | KO | 0.603357 | 1.97094 | 1.7078 | 1.64241 | 0.0002 | 0.008981 | yes |
| XLOC_004924 | KO | 3.31154 | 10.9328 | 1.72309 | 3.11148 | 5.00E−05 | 0.002735 | yes |
| XLOC_032387 | KO | 3.99715 | 13.2296 | 1.72673 | 2.84469 | 5.00E−05 | 0.002735 | yes |
| XLOC_017398 | KO | 0.259344 | 0.860178 | 1.72977 | 1.15779 | 0.0002 | 0.008981 | yes |
| XLOC_052195 | KO | 2.9645 | 9.85161 | 1.73257 | 2.35485 | 0.0001 | 0.005033 | yes |
| XLOC_037118 | KO | 17.9792 | 60.0782 | 1.74051 | 2.03774 | 0.00125 | 0.038196 | yes |
| XLOC_053014 | KO | 3.34332 | 11.2148 | 1.74605 | 3.38868 | 5.00E−05 | 0.002735 | yes |
| XLOC_015083 | KO | 0.676862 | 2.29222 | 1.75981 | 0.614236 | 5.00E−05 | 0.002735 | yes |
| XLOC_022019 | KO | 0.470582 | 1.59924 | 1.76487 | 1.58847 | 0.00025 | 0.010784 | yes |
| XLOC_051724 | KO | 1.42107 | 4.86941 | 1.77677 | 2.87987 | 5.00E−05 | 0.002735 | yes |
| XLOC_000121 | KO | 0.241846 | 0.840111 | 1.79649 | 1.25909 | 0.0017 | 0.048164 | yes |
| XLOC_000327 | KO | 0.242484 | 0.848684 | 1.80734 | 2.1924 | 0.0005 | 0.018851 | yes |
| XLOC_039801 | KO | 0.598491 | 2.09971 | 1.81079 | 2.51873 | 5.00E−05 | 0.002735 | yes |
| XLOC_045580 | KO | 185.166 | 651.936 | 1.81591 | 3.07949 | 5.00E−05 | 0.002735 | yes |
| XLOC_014868 | KO | 0.621126 | 2.2059 | 1.82841 | 2.50044 | 0.0001 | 0.005033 | yes |
| XLOC_014085 | KO | 0.180093 | 0.658612 | 1.87069 | 2.0777 | 0.00075 | 0.025855 | yes |
| XLOC_023568 | KO | 7.63411 | 28.1995 | 1.88514 | 2.84838 | 5.00E−05 | 0.002735 | yes |
| XLOC_037820 | KO | 12.3395 | 45.6047 | 1.8859 | 4.08253 | 5.00E−05 | 0.002735 | yes |
| XLOC_025948 | KO | 2.21661 | 8.34637 | 1.91279 | 2.4035 | 0.00065 | 0.023143 | yes |
| XLOC_016521 | KO | 0.257452 | 0.971515 | 1.91593 | 2.09525 | 0.0005 | 0.018851 | yes |
| XLOC_037153 | KO | 0.413618 | 1.57214 | 1.92636 | 1.28723 | 0.00065 | 0.023143 | yes |
| XLOC_034645 | KO | 0.5919 | 2.26873 | 1.93846 | 2.36179 | 0.00025 | 0.010784 | yes |
| XLOC_037405 | KO | 0.929399 | 3.59335 | 1.95096 | 2.16965 | 0.0013 | 0.039328 | yes |
| XLOC_045797 | KO | 68.7105 | 265.92 | 1.95239 | 3.58335 | 5.00E−05 | 0.002735 | yes |
| XLOC_004325 | KO | 0.499493 | 1.93738 | 1.95557 | 1.51517 | 0.0011 | 0.034628 | yes |
| XLOC_051882 | KO | 0.126278 | 0.502587 | 1.99277 | 0.782285 | 5.00E−05 | 0.002735 | yes |
| XLOC_017869 | KO | 0.241538 | 0.971756 | 2.00834 | 1.17849 | 0.0009 | 0.029595 | yes |
| XLOC_030369 | KO | 17.9991 | 72.8577 | 2.01716 | 5.9886 | 5.00E−05 | 0.002735 | yes |
| XLOC_047511 | KO | 0.121904 | 0.511871 | 2.07003 | 1.8145 | 0.0002 | 0.008981 | yes |
| XLOC_046984 | KO | 0.497986 | 2.10412 | 2.07904 | 2.40101 | 5.00E−05 | 0.002735 | yes |
| XLOC_007385 | KO | 9.19124 | 38.9163 | 2.08204 | 5.8176 | 5.00E−05 | 0.002735 | yes |
| XLOC_025926 | KO | 0.739075 | 3.17893 | 2.10475 | 1.96094 | 0.0005 | 0.018851 | yes |
| XLOC_040637 | KO | 0.204073 | 0.885306 | 2.11709 | 2.3131 | 0.00015 | 0.007086 | yes |
| XLOC_012724 | KO | 0.58584 | 2.60382 | 2.15205 | 1.90326 | 0.0002 | 0.008981 | yes |
| XLOC_006913 | KO | 12.0212 | 54.4459 | 2.17925 | 4.21676 | 5.00E−05 | 0.002735 | yes |
| XLOC_049314 | KO | 2.44012 | 11.0794 | 2.18285 | 1.90716 | 5.00E−05 | 0.002735 | yes |
| XLOC_016027 | KO | 0.454901 | 2.06575 | 2.18304 | 2.84692 | 5.00E−05 | 0.002735 | yes |
| XLOC_015082 | KO | 238.716 | 1086.07 | 2.18575 | 5.44884 | 5.00E−05 | 0.002735 | yes |
| XLOC_015979 | KO | 2.06358 | 9.42343 | 2.1911 | 3.30441 | 5.00E−05 | 0.002735 | yes |
| XLOC_024337 | KO | 5.27805 | 24.8236 | 2.23363 | 2.72284 | 5.00E−05 | 0.002735 | yes |
| XLOC_035303 | KO | 0.553078 | 2.62482 | 2.24667 | 2.66454 | 5.00E−05 | 0.002735 | yes |
| XLOC_003362 | KO | 1.26564 | 6.07502 | 2.26302 | 3.30599 | 5.00E−05 | 0.002735 | yes |
| XLOC_032392 | KO | 3.34231 | 17.0188 | 2.34822 | 4.7306 | 5.00E−05 | 0.002735 | yes |
| XLOC_052942 | KO | 3.87359 | 19.8155 | 2.35489 | 3.39603 | 5.00E−05 | 0.002735 | yes |
| XLOC_036567 | KO | 0.663086 | 3.42892 | 2.37049 | 2.09127 | 0.00015 | 0.007086 | yes |
| XLOC_045798 | KO | 0.179411 | 0.9302 | 2.37427 | 0.396793 | 0.00085 | 0.028328 | yes |
| XLOC_047230 | KO | 3.2521 | 17.7581 | 2.44903 | 3.67184 | 5.00E−05 | 0.002735 | yes |
| XLOC_052560 | KO | 1.40902 | 7.70056 | 2.45027 | 3.91713 | 5.00E−05 | 0.002735 | yes |
| XLOC_003458 | KO | 0.123397 | 0.675328 | 2.45229 | 2.68551 | 5.00E−05 | 0.002735 | yes |
| XLOC_034204 | KO | 0.145277 | 0.797188 | 2.45612 | 1.49544 | 0.0001 | 0.005033 | yes |
| XLOC_049383 | KO | 4.19774 | 23.1397 | 2.46269 | 3.89288 | 5.00E−05 | 0.002735 | yes |
| XLOC_005163 | KO | 0.301241 | 1.6736 | 2.47396 | 3.15373 | 5.00E−05 | 0.002735 | yes |
| XLOC_054060 | KO | 0.157586 | 0.883085 | 2.48641 | 2.27535 | 5.00E−05 | 0.002735 | yes |
| XLOC_049559 | KO | 0.166759 | 0.954374 | 2.51679 | 2.98061 | 0.0001 | 0.005033 | yes |
| XLOC_022729 | KO | 2.51482 | 14.8367 | 2.56064 | 4.00418 | 5.00E−05 | 0.002735 | yes |
| XLOC_023335 | KO | 0.660718 | 3.9099 | 2.56502 | 2.62295 | 5.00E−05 | 0.002735 | yes |
| XLOC_043927 | KO | 0.348939 | 2.10265 | 2.59117 | 1.61414 | 5.00E−05 | 0.002735 | yes |
| XLOC_028901 | KO | 0.205164 | 1.24854 | 2.60539 | 2.73024 | 0.00015 | 0.007086 | yes |
| XLOC_021584 | KO | 1.41437 | 8.61438 | 2.60659 | 3.66797 | 5.00E−05 | 0.002735 | yes |
| XLOC_031893 | KO | 0.272375 | 1.65949 | 2.60707 | 2.44215 | 5.00E−05 | 0.002735 | yes |
| XLOC_047531 | KO | 0.221946 | 1.40983 | 2.66724 | 1.58133 | 5.00E−05 | 0.002735 | yes |
| XLOC_030352 | KO | 14.4202 | 92.1945 | 2.67659 | 4.82284 | 5.00E−05 | 0.002735 | yes |
| XLOC_047205 | KO | 43.9565 | 282.345 | 2.68331 | 5.56708 | 5.00E−05 | 0.002735 | yes |
| XLOC_037406 | KO | 1.56496 | 10.16 | 2.6987 | 3.4056 | 5.00E−05 | 0.002735 | yes |
| XLOC_036569 | KO | 5.60152 | 37.6157 | 2.74744 | 5.87658 | 5.00E−05 | 0.002735 | yes |
| XLOC_024906 | KO | 10.0921 | 70.4324 | 2.80301 | 3.39614 | 5.00E−05 | 0.002735 | yes |
| XLOC_027347 | KO | 0.059214 | 0.416652 | 2.81484 | 0.191488 | 5.00E−05 | 0.002735 | yes |
| XLOC_000114 | KO | 0.089288 | 0.631005 | 2.82112 | 0.337765 | 0.0017 | 0.048164 | yes |
| XLOC_013839 | KO | 0.144764 | 1.09159 | 2.91466 | 4.01475 | 5.00E−05 | 0.002735 | yes |
| XLOC_001202 | KO | 1.04701 | 8.04572 | 2.94194 | 4.55678 | 5.00E−05 | 0.002735 | yes |
| XLOC_049751 | KO | 1.69057 | 13.5802 | 3.00593 | 1.59705 | 0.00065 | 0.023143 | yes |
| XLOC_050257 | KO | 0.049111 | 0.410124 | 3.06195 | 1.31082 | 5.00E−05 | 0.002735 | yes |
| XLOC_026965 | KO | 0.177762 | 1.52343 | 3.0993 | 2.70468 | 0.0003 | 0.012524 | yes |
| XLOC_051526 | KO | 0.507191 | 4.73696 | 3.22336 | 2.5458 | 5.00E−05 | 0.002735 | yes |
| XLOC_025815 | KO | 0.102352 | 0.990864 | 3.27514 | 1.26515 | 0.0002 | 0.008981 | yes |
| XLOC_000992 | KO | 0.209731 | 2.03568 | 3.2789 | 3.16275 | 5.00E−05 | 0.002735 | yes |
| XLOC_017767 | KO | 1.05023 | 10.519 | 3.32422 | 2.84592 | 5.00E−05 | 0.002735 | yes |
| XLOC_017689 | KO | 0.888696 | 9.33779 | 3.39332 | 3.92041 | 5.00E−05 | 0.002735 | yes |
| XLOC_014541 | KO | 0.110567 | 1.20961 | 3.45154 | 3.44299 | 5.00E−05 | 0.002735 | yes |
| XLOC_035850 | KO | 0.054407 | 0.674055 | 3.631 | 1.00843 | 0.00135 | 0.040437 | yes |
| XLOC_022342 | KO | 25.8899 | 327.557 | 3.66129 | 9.12956 | 5.00E−05 | 0.002735 | yes |
| XLOC_015003 | KO | 0.390992 | 4.94855 | 3.66179 | 2.8336 | 5.00E−05 | 0.002735 | yes |
| XLOC_036088 | KO | 0.189807 | 2.75838 | 3.86122 | 3.81947 | 5.00E−05 | 0.002735 | yes |
| XLOC_054232 | KO | 0.036869 | 0.594302 | 4.01071 | 2.84046 | 5.00E−05 | 0.002735 | yes |
| XLOC_047719 | KO | 0.806157 | 13.073 | 4.01938 | 5.27775 | 5.00E−05 | 0.002735 | yes |
| XLOC_053187 | KO | 10.1426 | 229.041 | 4.4971 | 8.80028 | 5.00E−05 | 0.002735 | yes |
| XLOC_052734 | KO | 1.28598 | 36.491 | 4.8266 | 9.40326 | 5.00E−05 | 0.002735 | yes |
| XLOC_030000 | KO | 0.052009 | 6.94072 | 7.06019 | 1.201 | 0.00025 | 0.010784 | yes |
| XLOC_025431 | KO | 0 | 0.934836 | inf | #NAME? | 0.00075 | 0.025855 | yes |
| XLOC_036777 | KO | 0 | 0.332679 | inf | #NAME? | 0.0008 | 0.027077 | yes |
| XLOC_027001 | KO | 0.354255 | 0 | #NAME? | #NAME? | 5.00E−05 | 0.002735 | yes |
| XLOC_025714 | KO | 0.776716 | 0 | #NAME? | #NAME? | 5.00E−05 | 0.002735 | yes |
| XLOC_045348 | KO | 1.04504 | 0 | #NAME? | #NAME? | 5.00E−05 | 0.002735 | yes |
| XLOC_006399 | KO | 2.05138 | 0 | #NAME? | #NAME? | 5.00E−05 | 0.002735 | yes |
| XLOC_050265 | KO | 0.40988 | 0 | #NAME? | #NAME? | 5.00E−05 | 0.002735 | yes |
| XLOC_032449 | KO | 0.705069 | 0 | #NAME? | #NAME? | 5.00E−05 | 0.002735 | yes |
| XLOC_051343 | KO | 0.255896 | 0 | #NAME? | #NAME? | 5.00E−05 | 0.002735 | yes |
| XLOC_031712 | KO | 0.399756 | 0 | #NAME? | #NAME? | 5.00E−05 | 0.002735 | yes |
| XLOC_033655 | KO | 1.1969 | 0 | #NAME? | #NAME? | 5.00E−05 | 0.002735 | yes |
| XLOC_010032 | KO | 0.288388 | 0 | #NAME? | #NAME? | 0.0001 | 0.005033 | yes |
| XLOC_050737 | KO | 0.593515 | 0 | #NAME? | #NAME? | 5.00E−05 | 0.002735 | yes |
| XLOC_049474 | KO | 0.308428 | 0 | #NAME? | #NAME? | 5.00E−05 | 0.002735 | yes |
| XLOC_053524 | KO | 0.340771 | 0 | #NAME? | #NAME? | 5.00E−05 | 0.002735 | yes |
| XLOC_027708 | KO | 0.56401 | 0 | #NAME? | #NAME? | 5.00E−05 | 0.002735 | yes |
| XLOC_040716 | KO | 0.455888 | 0 | #NAME? | #NAME? | 5.00E−05 | 0.002735 | yes |
| XLOC_031042 | KO | 0.539013 | 0 | #NAME? | #NAME? | 0.00075 | 0.025855 | yes |
| XLOC_050453 | KO | 0.475329 | 0 | #NAME? | #NAME? | 0.0001 | 0.005033 | yes |
| XLOC_024731 | KO | 2.7551 | 0 | #NAME? | #NAME? | 5.00E−05 | 0.002735 | yes |

SUPPLEMENTARY TABLE 1.B
PAX6DKO VS WT

| tracking_id | gene | WT_1 | WT_0 | WT_2 | DKO_0 | DKO_1 | DKO_2 | gene |
|---|---|---|---|---|---|---|---|---|
| XLOC_027099 | IAH1 | 6.35612 | 26.3106 | 8.48768 | 0.018812 | 0.113338 | 0 | IAH1 |
| XLOC_044162 | AL021368.4, | 2.2691 | 3.09331 | 8.54334 | 0 | 0.065594 | 0 | AL021368.4, |
| | GUSBP4, | | | | | | | GUSBP4, |
| | LINC00680 | | | | | | | LINC00680 |
| XLOC_033655 | PRODH | 0 | 0 | 3.55524 | 0 | 0 | 0.017005 | PRODH |
| XLOC_014867 | VSX2 | 24.0414 | 42.2013 | 44.5609 | 0.436724 | 0.009159 | 0.15957 | VSX2 |
| XLOC_049453 | NIPAL2, | 0.540427 | 1.67815 | 169.136 | 0.418319 | 1.11934 | 0.338185 | NIPAL2, |
| | RNU6-914P | | | | | | | RNU6-914P |
| XLOC_028271 | DAPL1, | 77.5916 | 125.672 | 120.717 | 3.67888 | 0.989218 | 1.47695 | DAPL1, |
| | OR7E28P, | | | | | | | OR7E28P, |
| | OR7E89P | | | | | | | OR7E89P |
| XLOC_039936 | BHMT | 37.3323 | 111.92 | 120.503 | 1.69085 | 1.44365 | 2.45093 | BHMT |
| XLOC_000776 | FOXE3 | 14.8087 | 39.8849 | 41.069 | 0.631188 | 0.619772 | 1.46708 | FOXE3 |
| XLOC_044161 | AL021368.2 | 0.709032 | 0.298226 | 0.769589 | 0.009649 | 0.01983 | 0.02273 | AL021368.2 |
| XLOC_012702 | TBX5 | 1.94744 | 0.731017 | 5.94325 | 0.014702 | 0.015108 | 0.253743 | TBX5 |
| XLOC_002160 | C1orf186 | 0.63685 | 6.86482 | 5.34159 | 0.10146 | 0.342507 | 0.0258 | C1orf186 |
| XLOC_012591 | PAH | 0.066411 | 1.6998 | 3.42992 | 0.110606 | 0.006534 | 0.105655 | PAH |
| XLOC_009482 | CTSF | 2.91724 | 14.9852 | 7.31819 | 0.015297 | 0.584739 | 0.574229 | CTSF |
| XLOC_002728 | MIR34A, | 1.4038 | 120.464 | 2.53687 | 1.97198 | 1.36587 | 2.79904 | MIR34A, |
| | MIR34AHG | | | | | | | MIR34AHG |
| XLOC_027566 | ATP6V1B1 | 4.78079 | 11.3255 | 5.64947 | 0.65953 | 0.139568 | 0.343879 | ATP6V1B1 |
| XLOC_025026 | ZNF726, | 1.61892 | 1.56815 | 0.946504 | 0.077959 | 0.068264 | 0.073718 | ZNF726, |
| | ZNF92P3 | | | | | | | ZNF92P3 |
| XLOC_018597 | CLEC19A | 23.9987 | 10.9556 | 18.88 | 0.86652 | 0.875949 | 1.17152 | CLEC19A |
| XLOC_036565 | CP | 6.26707 | 50.9784 | 42.8121 | 1.08762 | 0.47466 | 4.23101 | CP |
| XLOC_040762 | AC138035.1 | 0.275702 | 4.87749 | 0.33735 | 0.148363 | 0.065456 | 0.10979 | AC138035.1 |
| XLOC_006922 | PITX3 | 1.52713 | 5.05783 | 6.0417 | 0.203747 | 0.173092 | 0.377088 | PITX3 |
| XLOC_033225 | CRYBB3 | 1.85882 | 7.03654 | 4.079 | 0.227666 | 0.233945 | 0.32182 | CRYBB3 |
| XLOC_002465 | KMO | 5.58462 | 16.448 | 13.9268 | 1.0157 | 0.515373 | 0.796776 | KMO |
| XLOC_048453 | ZNF572 | 0.412887 | 0.750842 | 0.300014 | 0.024293 | 0.041605 | 0.053412 | ZNF572 |
| XLOC_005226 | GAD2 | 0.329415 | 1.20134 | 1.25571 | 0.113463 | 0.013539 | 0.108748 | GAD2 |
| XLOC_019871 | EIF3CL | 2.88923 | 0.208908 | 0.197669 | 0.087299 | 0.080736 | 0.115168 | EIF3CL |
| XLOC_030539 | FZD5 | 6.3426 | 16.7834 | 14.869 | 1.75585 | 0.902139 | 0.784099 | FZD5 |
| XLOC_041832 | PPP2R2B, | 370.411 | 12.3097 | 10.1457 | 12.964 | 12.9812 | 10.2564 | PPP2R2B, |
| | RNA5SP196 | | | | | | | RNA5SP196 |
| XLOC_024322 | RAX | 9.60122 | 8.87129 | 16.2795 | 0.827653 | 1.63647 | 0.855323 | RAX |
| XLOC_030921 | LAMP5 | 3.28604 | 11.1172 | 8.30284 | 0.678143 | 0.739653 | 0.765154 | LAMP5 |
| XLOC_008341 | CNTN5 | 2.87317 | 6.12486 | 6.60276 | 0.887016 | 0.287429 | 0.337658 | CNTN5 |
| XLOC_038319 | HMX1 | 2.73849 | 4.31346 | 5.84262 | 0.235902 | 0.27271 | 0.75775 | HMX1 |
| XLOC_041731 | SPOCK1 | 145.596 | 162.91 | 145.988 | 15.7719 | 13.4086 | 15.6346 | SPOCK1 |
| XLOC_009925 | CRYAB | 366.654 | 1347.99 | 1734.04 | 124.208 | 103.316 | 125.801 | CRYAB |
| XLOC_048642 | CSMD1 | 17.263 | 0.301447 | 0.798707 | 0.47554 | 1.03686 | 0.37402 | CSMD1 |
| XLOC_001908 | PAPPA2 | 11.3668 | 17.3899 | 23.9001 | 1.75662 | 2.06159 | 1.907 | PAPPA2 |
| XLOC_034205 | HRH1 | 0.728556 | 2.35439 | 2.62183 | 0.134182 | 0.204387 | 0.299929 | HRH1 |
| XLOC_010676 | GPD1 | 1.08232 | 1.40149 | 1.99675 | 0.172709 | 0.11047 | 0.227419 | GPD1 |
| XLOC_031377 | APCDD1L-AS1 | 1.25226 | 2.13926 | 1.38036 | 0.139581 | 0.434297 | 0.022103 | APCDD1L-AS1 |
| XLOC_038358 | NKX3-2 | 1.22611 | 0.343896 | 1.52837 | 0.057486 | 0.206751 | 0.14445 | NKX3-2 |
| XLOC_027565 | VAX2 | 1.61982 | 2.62167 | 1.2574 | 0.318721 | 0.109171 | 0.350383 | VAX2 |
| XLOC_051344 | ALDH1A1 | 268.278 | 337.186 | 344.585 | 45.5472 | 35.4898 | 55.3302 | ALDH1A1 |
| XLOC_036590 | AC020636.2, | 1.64049 | 4.57607 | 3.36258 | 0.771193 | 0.15853 | 0.463221 | AC020636.2, |
| | CLRN1, | | | | | | | CLRN1, |
| | MINDY4B | | | | | | | MINDY4B |
| XLOC_036625 | AC108729.3, | 27.0835 | 230.605 | 21.8681 | 12.2886 | 14.8895 | 13.5729 | AC108729.3, |
| | PLCH1 | | | | | | | PLCH1 |
| XLOC_017273 | AC015712.1, | 61.8026 | 83.4586 | 50.6442 | 10.5314 | 6.87879 | 11.3318 | AC015712.1, |
| | AC015712.4, | | | | | | | AC015712.4, |
| | AC015712.5, | | | | | | | AC015712.5, |
| | AC015712.6, | | | | | | | AC015712.6, |
| | ALDH1A3 | | | | | | | ALDH1A3 |
| XLOC_016033 | DIO3OS, | 3.35376 | 3.63927 | 4.21183 | 0.199066 | 0.480621 | 0.97296 | DIO3OS, |
| | MIR1247 | | | | | | | MIR1247 |
| XLOC_029247 | CYP1B1, | 12.7944 | 15.2601 | 26.2721 | 3.0588 | 2.10553 | 2.94246 | CYP1B1, |
| | RMDN2-AS1 | | | | | | | RMDN2-AS1 |
| XLOC_018711 | EIF3C | 2.87618 | 34.5653 | 31.8651 | 4.72684 | 3.15636 | 2.47979 | EIF3C |
| XLOC_032052 | APCDD1L | 2.60783 | 2.44671 | 2.12832 | 0.293242 | 0.570942 | 0.246149 | APCDD1L |
| XLOC_019330 | HSD17B2 | 108.573 | 79.3757 | 74.2518 | 11.3214 | 9.48488 | 19.7587 | HSD17B2 |
| XLOC_005448 | DKK1 | 13.6083 | 16.1791 | 24.6678 | 2.34378 | 2.91815 | 3.17139 | DKK1 |
| XLOC_014052 | DCT | 116.653 | 24.9895 | 9.93978 | 10.1663 | 6.90676 | 8.14239 | DCT |
| XLOC_002706 | TNFRSF9 | 0.955634 | 5.22558 | 3.9647 | 0.95628 | 0.267551 | 0.486885 | TNFRSF9 |
| XLOC_044950 | AC004160.1 | 0.053175 | 0.764147 | 0.924094 | 0 | 0.096271 | 0.202499 | AC004160.1 |
| XLOC_042581 | MIR5004, | 0.059656 | 0.074733 | 1.99489 | 0.232952 | 0.098403 | 0.049351 | MIR5004, |
| | SYNGAP1, | | | | | | | SYNGAP1, |
| | ZBTB9 | | | | | | | ZBTB9 |
| XLOC_012370 | CPM, | 2.64187 | 6.95502 | 4.36488 | 0.870542 | 0.80487 | 0.84699 | CPM, |
| | PRELID2P1 | | | | | | | PRELID2P1 |
| XLOC_015185 | DIO3 | 3.26808 | 6.67527 | 3.70188 | 0.853295 | 0.553079 | 1.15041 | DIO3 |
| XLOC_008373 | GRIA4 | 4.46608 | 10.8139 | 10.2688 | 1.75957 | 1.17946 | 1.92112 | GRIA4 |
| XLOC_021142 | CCL2 | 271.652 | 422.61 | 418.085 | 80.2066 | 49.6472 | 84.926 | CCL2 |
| XLOC_005951 | ADRB1 | 0.676098 | 0.543138 | 0.695966 | 0.08453 | 0.250934 | 0.035401 | ADRB1 |
| XLOC_020429 | ADAMTS18 | 20.9749 | 23.2548 | 27.0867 | 4.17636 | 5.37847 | 4.26227 | ADAMTS18 |
| XLOC_002055 | LHX9 | 3.70858 | 1.54463 | 3.77319 | 0.872661 | 0.252278 | 0.660088 | LHX9 |
| XLOC_007296 | CCKBR | 18.9483 | 29.6155 | 16.3185 | 4.54304 | 4.3604 | 4.49649 | CCKBR |
| XLOC_050766 | BARHL1 | 7.75494 | 3.28797 | 51.4482 | 0.45201 | 2.47151 | 10.3857 | BARHL1 |
| XLOC_030190 | CYTIP | 0.890241 | 0.76244 | 0.753724 | 0.150365 | 0.293362 | 0.07193 | CYTIP |
| XLOC_024400 | FAM69C | 11.6468 | 9.87495 | 8.56703 | 2.08273 | 2.59406 | 1.91915 | FAM69C |
| XLOC_039183 | SFRP2 | 476.589 | 775.446 | 776.525 | 112.159 | 165.839 | 168.877 | SFRP2 |
| XLOC_034614 | SYNPR | 15.8505 | 11.9411 | 10.1719 | 3.28918 | 1.98699 | 3.27429 | SYNPR |
| XLOC_017031 | CRABP1 | 1195.31 | 2227.35 | 1893.36 | 477.443 | 378.147 | 379.316 | CRABP1 |
| XLOC_043011 | AL589740.1 | 2.83443 | 1.99495 | 3.03891 | 0.423911 | 0.413746 | 0.999699 | AL589740.1 |
| XLOC_035457 | CLDN16 | 0.459136 | 0.442408 | 0.865385 | 0.070995 | 0.155026 | 0.186995 | CLDN16 |
| XLOC_003488 | CRYZ | 18.4011 | 18.1655 | 19.5315 | 2.13505 | 4.87919 | 6.20487 | CRYZ |
| XLOC_049746 | MAFA | 0.615728 | 1.59732 | 0.741452 | 0.26726 | 0.191048 | 0.262786 | MAFA |
| XLOC_013669 | SHISA2 | 13.3832 | 15.9522 | 8.82117 | 2.92263 | 2.65672 | 3.73375 | SHISA2 |
| XLOC_015082 | DLK1 | 108.58 | 445.412 | 155.099 | 116.197 | 31.0653 | 30.0931 | DLK1 |
| XLOC_047097 | RELN | 8.19846 | 15.0825 | 15.7761 | 2.35415 | 1.98371 | 5.5128 | RELN |
| XLOC_029306 | SIX3-AS1 | 5.17672 | 6.65778 | 12.9058 | 2.80865 | 1.72649 | 1.76511 | SIX3-AS1 |
| XLOC_037223 | LINC02506 | 6.23974 | 51.9183 | 13.6307 | 10.4812 | 2.98631 | 4.98306 | LINC02506 |
| XLOC_019146 | LRRC36 | 0.082067 | 1.9616 | 0.160277 | 0.261932 | 0.143079 | 0.162177 | LRRC36 |
| XLOC_005835 | PAX2 | 1.3601 | 1.26266 | 2.89939 | 0.372508 | 0.486074 | 0.576677 | PAX2 |
| XLOC_002251 | KCNK2 | 2.34228 | 3.29868 | 2.04425 | 0.750383 | 0.787665 | 0.463977 | KCNK2 |
| XLOC_029304 | LINC01833 | 18.4764 | 41.3797 | 38.9724 | 11.1224 | 6.64704 | 8.07079 | LINC01833 |
| XLOC_018083 | HOMER2 | 58.9068 | 72.7643 | 58.6159 | 20.1414 | 15.1953 | 15.6611 | HOMER2 |
| XLOC_027327 | QPCT | 2.4727 | 4.20444 | 2.60496 | 0.392047 | 0.790209 | 1.32658 | QPCT |
| XLOC_002049 | CRB1 | 2.30468 | 4.54208 | 3.18978 | 1.5315 | 0.60078 | 0.689177 | CRB1 |
| XLOC_023764 | CDH20 | 37.685 | 38.8728 | 33.3169 | 9.58835 | 13.0342 | 8.27094 | CDH20 |
| XLOC_048281 | ERICH5 | 16.2999 | 41.7988 | 46.1951 | 11.1174 | 4.84185 | 13.7767 | ERICH5 |
| XLOC_027527 | MEIS1 | 85.4694 | 87.4353 | 66.8553 | 28.7246 | 17.1714 | 22.4992 | MEIS1 |
| XLOC_013601 | GJA3 | 0.317404 | 0.736926 | 0.633042 | 0.10417 | 0.142725 | 0.238386 | GJA3 |
| XLOC_008880 | LMO1 | 6.80765 | 8.85465 | 4.16178 | 2.45708 | 1.88068 | 1.41516 | LMO1 |
| XLOC_018419 | IL32, | 8.52227 | 24.9515 | 18.2384 | 5.3936 | 4.73932 | 4.90601 | IL32, |
| | RNU1-125P | | | | | | | RNU1-125P |
| XLOC_036882 | CLDN1 | 50.106 | 58.8008 | 66.9105 | 13.5883 | 15.0468 | 23.5422 | CLDN1 |
| XLOC_051664 | TNC | 213.252 | 360.119 | 156.583 | 73.7033 | 108.252 | 34.7334 | TNC |
| XLOC_024388 | CBLN2 | 0.291008 | 2.26125 | 0.966416 | 0.430244 | 0.154044 | 0.480505 | CBLN2 |
| XLOC_041354 | ANKRD34B | 2.39566 | 1.69226 | 3.27724 | 0.314782 | 1.49788 | 0.420214 | ANKRD34B |
| XLOC_002245 | PROX1 | 2.4965 | 5.75226 | 8.62622 | 1.71467 | 1.61718 | 1.81632 | PROX1 |
| XLOC_043359 | PPP1R14C | 2.46775 | 7.44666 | 9.84518 | 2.26365 | 1.3536 | 2.51865 | PPP1R14C |
| XLOC_053790 | TCEAL5 | 11.4788 | 35.8439 | 10.9339 | 2.77939 | 6.19258 | 9.30113 | TCEAL5 |
| XLOC_039957 | RASGRF2 | 5.12876 | 3.6573 | 5.43401 | 0.997472 | 1.78609 | 1.89001 | RASGRF2 |
| XLOC_015824 | LTBP2 | 12.0873 | 10.9274 | 10.0768 | 2.89807 | 5.10566 | 3.04485 | LTBP2 |
| XLOC_041138 | AC034238.1, | 1.17155 | 1.49775 | 7.77941 | 0.650085 | 0.550149 | 2.40026 | AC034238.1, |
| | ESM1 | | | | | | | ESM1 |
| XLOC_039386 | AC018709.1, | 0.737531 | 1.38922 | 1.7777 | 0.609106 | 0.284264 | 0.453301 | AC018709.1, |
| | F11-AS1, | | | | | | | F11-AS1, |
| | MTNR1A | | | | | | | MTNR1A |
| XLOC_027382 | SIX3 | 26.4761 | 43.6453 | 49.5259 | 18.0016 | 12.0784 | 13.6248 | SIX3 |
| XLOC_034342 | ARPP21 | 0.498718 | 1.92141 | 1.34695 | 0.521731 | 0.179582 | 0.6858 | ARPP21 |
| XLOC_022663 | KRT17 | 95.5863 | 29.2402 | 2.37791 | 11.1458 | 33.3079 | 2.61583 | KRT17 |
| XLOC_044494 | MAN1A1 | 11.7561 | 8.84561 | 8.97651 | 3.99183 | 3.59782 | 3.41598 | MAN1A1 |
| XLOC_050369 | C9orf47, | 10.2433 | 24.3117 | 16.6353 | 6.53462 | 6.42303 | 6.12603 | C9orf47, |
| | S1PR3 | | | | | | | S1PR3 |
| XLOC_004771 | AL117348.1, | 5.19131 | 11.1679 | 5.11138 | 3.81669 | 2.39961 | 1.80643 | AL117348.1, |
| | LEFTY2 | | | | | | | LEFTY2 |
| XLOC_012012 | AMIGO2 | 3.45442 | 4.11641 | 4.20926 | 1.19783 | 1.44253 | 1.78408 | AMIGO2 |
| XLOC_053069 | TKTL1 | 2.95503 | 5.38513 | 3.74899 | 1.99216 | 1.65393 | 1.0042 | TKTL1 |
| XLOC_011248 | FAM222A | 4.12358 | 5.19282 | 4.7307 | 1.51599 | 1.40564 | 2.48574 | FAM222A |
| XLOC_013946 | PCDH9 | 2.76325 | 2.87375 | 3.69818 | 0.904305 | 1.35518 | 1.34963 | PCDH9 |
| XLOC_030425 | TMEFF2 | 40.0945 | 26.9503 | 23.8186 | 10.8991 | 10.3532 | 14.1006 | TMEFF2 |
| XLOC_001972 | LAMC2 | 18.2713 | 9.66125 | 4.30548 | 3.20981 | 7.36387 | 1.98432 | LAMC2 |
| XLOC_038812 | SNCA | 120.444 | 90.172 | 102.326 | 33.7833 | 32.2981 | 56.3382 | SNCA |
| XLOC_031027 | SYNDIG1 | 1.17031 | 1.60609 | 1.62468 | 0.579908 | 0.60694 | 0.546443 | SYNDIG1 |
| XLOC_050268 | RORB | 1.59684 | 2.26923 | 1.99551 | 1.11116 | 0.7857 | 0.426796 | RORB |
| XLOC_050235 | AL353608.3, | 14.8368 | 9.57984 | 9.86534 | 3.35358 | 5.24648 | 5.16355 | AL353608.3, |
| | PGM5 | | | | | | | PGM5 |
| XLOC_049879 | TYRP1 | 235.705 | 54.4611 | 38.9429 | 59.2856 | 26.1865 | 48.6274 | TYRP1 |
| XLOC_035875 | CSPG5 | 6.19783 | 7.13338 | 5.1209 | 3.51239 | 2.12591 | 2.0641 | CSPG5 |
| XLOC_047896 | C8orf4 | 40.6436 | 47.2069 | 50.5633 | 13.5105 | 31.6952 | 12.6026 | C8orf4 |
| XLOC_048409 | COL14A1 | 2.59648 | 2.10468 | 3.61912 | 1.07687 | 1.56109 | 0.85661 | COL14A1 |
| XLOC_010972 | LGR5 | 19.3417 | 19.2728 | 20.0357 | 7.97384 | 8.60957 | 8.1073 | LGR5 |
| XLOC_050248 | MAMDC2 | 13.1192 | 37.5715 | 35.6402 | 12.7504 | 9.82286 | 14.0674 | MAMDC2 |
| XLOC_004451 | COLGALT2 | 18.8059 | 26.6688 | 34.1965 | 11.2487 | 9.74781 | 12.9374 | COLGALT2 |
| XLOC_026186 | CPAMD8 | 18.8752 | 17.1836 | 30.3108 | 7.77265 | 10.4191 | 10.2379 | CPAMD8 |
| XLOC_004455 | FAM129A | 3.98387 | 3.30211 | 2.84975 | 1.89095 | 1.25215 | 1.20355 | FAM129A |
| XLOC_023416 | RAB31 | 39.4445 | 54.2819 | 58.6251 | 18.3149 | 23.4672 | 23.8651 | RAB31 |
| XLOC_027383 | AC012354.1 | 2.27649 | 4.02042 | 4.51128 | 1.78547 | 1.59765 | 1.28546 | AC012354.1 |
| XLOC_051679 | BRINP1 | 2.92763 | 3.40757 | 2.76609 | 1.55953 | 1.22748 | 1.28975 | BRINP1 |
| XLOC_002264 | TGFB2, | 45.5307 | 68.4717 | 84.3122 | 30.2041 | 32.3378 | 28.2233 | TGFB2, |
| | TGFB2-OT1 | | | | | | | TGFB2-OT1 |
| XLOC_006249 | CUBN | 0.43488 | 0.614983 | 0.773097 | 0.219685 | 0.238769 | 0.378187 | CUBN |
| XLOC_034650 | MITF | 15.5231 | 10.5048 | 6.80536 | 4.89256 | 3.1139 | 7.07399 | MITF |
| XLOC_030257 | LRP2 | 27.3764 | 34.6716 | 38.5213 | 13.4993 | 17.2224 | 15.633 | LRP2 |
| XLOC_017507 | GJD2 | 1.86204 | 3.68614 | 4.5392 | 1.48254 | 1.51391 | 1.70258 | GJD2 |
| XLOC_017508 | ACTC1 | 244.194 | 242.898 | 338.343 | 121.789 | 166.507 | 101.795 | ACTC1 |
| XLOC_000284 | EFHD2 | 36.94 | 33.3531 | 33.7508 | 15.2872 | 16.4366 | 17.5493 | EFHD2 |
| XLOC_027354 | PKDCC | 58.0919 | 68.659 | 69.506 | 27.3853 | 31.4243 | 34.6239 | PKDCC |
| XLOC_003193 | AC099795.1, | 302.092 | 424.578 | 349.152 | 176.956 | 184.007 | 151.787 | AC099795.1, |
| | SLC2A1 | | | | | | | SLC2A1 |
| XLOC_027319 | CRIM1 | 28.1111 | 39.825 | 54.7239 | 19.0044 | 18.1419 | 21.6203 | CRIM1 |
| XLOC_048183 | AC015522.1 | 23.893 | 16.9284 | 17.7133 | 6.62867 | 11.233 | 10.8486 | AC015522.1 |
| XLOC_012216 | PMEL | 380.885 | 280.488 | 177.492 | 123.798 | 90.577 | 199.36 | PMEL |
| XLOC_034156 | BHLHE40 | 28.7412 | 23.1729 | 21.563 | 12.1994 | 14.1307 | 10.3034 | BHLHE40 |
| XLOC_047196 | TSPAN12 | 11.4086 | 19.9723 | 13.6718 | 7.21286 | 9.09143 | 6.16548 | TSPAN12 |
| XLOC_030171 | RPRM | 5.40098 | 4.65882 | 5.19447 | 8.53696 | 10.2919 | 11.89 | RPRM |
| XLOC_038414 | LGI2 | 3.34797 | 2.21911 | 3.12553 | 3.90375 | 5.02253 | 8.64864 | LGI2 |
| XLOC_044149 | BMP5 | 3.02977 | 1.71237 | 2.09512 | 3.95214 | 4.59264 | 5.41114 | BMP5 |
| XLOC_015662 | RTN1 | 9.13956 | 9.31148 | 8.33665 | 14.1267 | 16.4955 | 24.1101 | RTN1 |
| XLOC_004016 | CELF3 | 3.94652 | 6.17983 | 4.15166 | 7.71013 | 7.3096 | 14.2807 | CELF3 |
| XLOC_035933 | CAMKV | 3.09309 | 3.67909 | 2.58475 | 4.5568 | 5.55017 | 9.23923 | CAMKV |
| XLOC_041385 | HAPLN1 | 57.6882 | 39.4065 | 34.4343 | 82.0823 | 102.661 | 87.9429 | HAPLN1 |
| XLOC_004282 | LMX1A | 33.4135 | 21.9197 | 49.0861 | 70.9296 | 71.3898 | 75.9027 | LMX1A |
| XLOC_028427 | ITGA4 | 2.05187 | 1.25071 | 2.62518 | 2.11168 | 3.59207 | 6.87503 | ITGA4 |
| XLOC_053428 | SYP | 2.5879 | 3.11914 | 2.16079 | 5.13716 | 4.68135 | 7.29417 | SYP |
| XLOC_010398 | PTPRO | 4.68262 | 3.8616 | 4.0351 | 15.7455 | 7.02845 | 4.65822 | PTPRO |
| XLOC_033238 | SEZ6L | 1.77989 | 2.12385 | 1.38471 | 2.7291 | 3.26834 | 5.54343 | SEZ6L |
| XLOC_051389 | FRMD3 | 1.83018 | 2.1865 | 4.43258 | 5.52025 | 5.27143 | 7.91202 | FRMD3 |
| XLOC_031307 | SNAI1 | 4.02313 | 3.05522 | 3.24031 | 4.44491 | 6.83397 | 11.7402 | SNAI1 |
| XLOC_030989 | INSM1 | 1.6489 | 2.00348 | 1.96073 | 2.57814 | 3.52584 | 6.53783 | INSM1 |
| XLOC_050669 | LMX1B | 3.02009 | 1.22639 | 2.83911 | 2.88407 | 3.57748 | 9.73892 | LMX1B |
| XLOC_052629 | TCEAL2 | 19.9932 | 6.70028 | 21.5307 | 45.2251 | 34.9479 | 31.9153 | TCEAL2 |
| XLOC_006747 | GRID1, | 1.16152 | 0.89719 | 1.76359 | 1.27619 | 1.29953 | 6.46301 | GRID1, |
| | uc_338 | | | | | | | uc_338 |
| XLOC_052734 | HTR2C | 0.700825 | 1.81764 | 1.30142 | 5.75477 | 1.86245 | 1.56679 | HTR2C |
| XLOC_047751 | NEFM | 17.7394 | 20.2093 | 27.1897 | 27.2862 | 42.1514 | 90.5211 | NEFM |
| XLOC_042079 | COL23A1 | 3.01085 | 2.66369 | 4.69091 | 4.21772 | 5.63487 | 15.6218 | COL23A1 |
| XLOC_048890 | NEFL | 10.1258 | 15.8403 | 16.4345 | 24.3631 | 25.7532 | 54.1677 | NEFL |
| XLOC_018031 | CHRNB4 | 1.2344 | 0.980568 | 0.904825 | 1.21408 | 2.39138 | 4.14531 | CHRNB4 |
| XLOC_004935 | GREM2 | 0.745461 | 0.730919 | 0.535223 | 1.14036 | 1.4873 | 2.46793 | GREM2 |
| XLOC_042770 | TFAP2B | 30.3385 | 5.0023 | 8.23905 | 15.8348 | 47.749 | 48.956 | TFAP2B |
| XLOC_030899 | CHGB | 1.56999 | 1.76668 | 1.1029 | 2.7693 | 3.48896 | 5.28896 | CHGB |
| XLOC_052372 | MAGEH1 | 24.0826 | 19.4032 | 28.612 | 63.5455 | 64.074 | 60.3462 | MAGEH1 |
| XLOC_043938 | GRM4 | 0.475366 | 0.356607 | 0.357952 | 0.610677 | 1.08307 | 1.4304 | GRM4 |
| XLOC_026219 | KIAA1683 | 0.87993 | 0.708561 | 1.21511 | 0.87429 | 2.28624 | 4.25022 | KIAA1683 |
| XLOC_009683 | FAM181B | 2.06403 | 1.75194 | 4.16686 | 5.45181 | 5.16281 | 10.627 | FAM181B |
| XLOC_022915 | CA10 | 1.19926 | 1.0558 | 2.24358 | 2.89383 | 2.37375 | 6.802 | CA10 |
| XLOC_041742 | GFRA3 | 0.651745 | 0.624342 | 0.936448 | 1.26301 | 1.29785 | 3.37587 | GFRA3 |
| XLOC_032857 | ERVH48-1 | 2.30878 | 2.91941 | 0.336689 | 6.00408 | 4.66685 | 4.27947 | ERVH48-1 |
| XLOC_032392 | RIPPLY3 | 3.3058 | 2.73499 | 3.88712 | 9.4948 | 6.18763 | 10.994 | RIPPLY3 |
| XLOC_045787 | CPED1, | 3.8268 | 0.805165 | 4.70118 | 3.95265 | 5.94346 | 15.7302 | CPED1, |
| | HMGN1P18 | | | | | | | HMGN1P18 |
| XLOC_048897 | EBF2 | 1.08758 | 1.07544 | 1.13791 | 1.58855 | 2.89245 | 4.63734 | EBF2 |
| XLOC_004252 | MPZ | 7.07102 | 4.30449 | 8.68341 | 7.3039 | 15.9612 | 32.4706 | MPZ |
| XLOC_037044 | MSX1 | 17.6647 | 15.2965 | 18.4083 | 41.1999 | 31.0716 | 70.4766 | MSX1 |
| XLOC_028714 | CDK5R2 | 0.615729 | 0.570948 | 0.555986 | 1.40459 | 1.42113 | 2.04639 | CDK5R2 |
| XLOC_000794 | ELAVL4 | 5.55111 | 9.7948 | 6.60765 | 12.4444 | 20.7901 | 28.7352 | ELAVL4 |
| XLOC_029809 | RNF149, | 10.09 | 7.77506 | 11.1104 | 8.96474 | 61.4119 | 11.7368 | RNF149, |
| | SNORD89 | | | | | | | SNORD89 |
| XLOC_003798 | NHLH2 | 0.487362 | 0.637494 | 0.669737 | 1.15678 | 1.74957 | 2.21569 | NHLH2 |
| XLOC_047230 | GRM8 | 3.54174 | 2.7208 | 3.39742 | 11.7384 | 7.85823 | 8.20732 | GRM8 |
| XLOC_021373 | ADAM11 | 5.84158 | 3.45392 | 3.89677 | 7.41243 | 9.9017 | 20.6655 | ADAM11 |
| XLOC_017731 | WDR72 | 0.650121 | 0.732428 | 1.04974 | 2.27034 | 1.79787 | 2.96327 | WDR72 |
| XLOC_031394 | PHACTR3 | 1.68606 | 0.475005 | 1.2944 | 1.70091 | 3.66888 | 4.66095 | PHACTR3 |
| XLOC_004924 | LINC01139 | 3.24003 | 2.89495 | 3.70155 | 12.1761 | 7.4302 | 9.3553 | LINC01139 |
| XLOC_038929 | PITX2 | 4.66547 | 1.82763 | 4.11564 | 9.09252 | 9.39538 | 12.8157 | PITX2 |
| XLOC_024906 | GDF15 | 4.30613 | 12.7602 | 12.9116 | 37.6506 | 18.5757 | 32.9894 | GDF15 |
| XLOC_048154 | STMN2 | 69.4526 | 63.3122 | 60.4211 | 126.499 | 196.35 | 255.076 | STMN2 |
| XLOC_041939 | EBF1 | 2.97424 | 1.79056 | 3.44582 | 4.15686 | 7.83243 | 12.7996 | EBF1 |
| XLOC_004283 | RXRG | 1.54887 | 1.02803 | 2.97637 | 2.8608 | 4.18203 | 9.86821 | RXRG |
| XLOC_007091 | EBF3 | 2.91282 | 2.9235 | 5.21086 | 6.21312 | 9.81381 | 17.7005 | EBF3 |
| XLOC_005825 | ABCC2 | 1.04029 | 0.159979 | 0.460372 | 0.450145 | 2.01261 | 2.65277 | ABCC2 |
| XLOC_025095 | CHST8 | 0.69729 | 1.04306 | 0.824119 | 1.48245 | 2.77272 | 3.77663 | CHST8 |
| XLOC_015674 | SIX1 | 3.84063 | 2.32059 | 6.29803 | 7.51272 | 10.717 | 21.5869 | SIX1 |
| XLOC_034818 | ZPLD1 | 0.454756 | 0.348382 | 0.944384 | 0.59692 | 0.94142 | 4.06766 | ZPLD1 |
| XLOC_026209 | UNC13A | 0.310463 | 0.385926 | 0.358126 | 0.661015 | 0.941611 | 1.78312 | UNC13A |
| XLOC_047719 | FGF17 | 0.181709 | 0.709779 | 1.50313 | 3.01003 | 1.14096 | 3.5756 | FGF17 |
| XLOC_008137 | TPBGL | 0.221 | 0.24914 | 0.18793 | 0.326765 | 0.612302 | 1.18635 | TPBGL |
| XLOC_045774 | MIR6132, | 21.369 | 19.6749 | 37.513 | 195.309 | 26.2393 | 32.2836 | MIR6132, |
| | ST7, | | | | | | | ST7, |
| | ST7-AS1_2, | | | | | | | ST7-AS1_2, |
| | ST7-OT3_1, | | | | | | | ST7-OT3_1, |
| | ST7-OT3_2, | | | | | | | ST7-OT3_2, |
| | ST7-OT3_3, | | | | | | | ST7-OT3_3, |
| | ST7-OT4, | | | | | | | ST7-OT4, |
| | ST7-OT4_1, | | | | | | | ST7-OT4_1, |
| | ST7-OT4_2, | | | | | | | ST7-OT4_2, |
| | ST7-OT4_3, | | | | | | | ST7-OT4_3, |
| | ST7-OT4_4 | | | | | | | ST7-OT4_4 |
| XLOC_010779 | NEUROD4 | 0.855001 | 0.535386 | 0.545815 | 0.841925 | 2.15946 | 3.29204 | NEUROD4 |
| XLOC_030738 | ECEL1 | 9.3731 | 3.88375 | 9.07631 | 21.1135 | 21.0309 | 30.5115 | ECEL1 |
| XLOC_028834 | CHRND | 0.133893 | 0.162252 | 0.169808 | 0.210794 | 0.282629 | 1.04517 | CHRND |
| XLOC_038132 | AC078881.1 | 0.483632 | 0.609226 | 0.537148 | 1.26026 | 1.68745 | 2.51901 | AC078881.1 |
| XLOC_003734 | KCNA2 | 0.119848 | 0.114483 | 0.121454 | 0.196014 | 0.357263 | 0.641855 | KCNA2 |
| XLOC_016656 | CKMT1A | 0.195764 | 0.252631 | 0.065192 | 0.744312 | 0.751534 | 0.271323 | CKMT1A |
| XLOC_023042 | CSHL1, | 0.147814 | 0.083878 | 0.086755 | 0.105186 | 0.444118 | 0.546804 | CSHL1, |
| | GH1 | | | | | | | GH1 |
| XLOC_021365 | RUNDC3A | 3.05171 | 3.43395 | 1.7738 | 7.60929 | 6.82062 | 14.3788 | RUNDC3A |
| XLOC_037406 | AC013724.1 | 1.26243 | 0.607775 | 2.7783 | 2.30769 | 5.23486 | 8.90323 | AC013724.1 |
| XLOC_031942 | TNNC2 | 1.19843 | 0.597568 | 0.616824 | 0.948961 | 3.5413 | 4.04744 | TNNC2 |
| XLOC_007768 | P2RX3 | 11.4484 | 5.26939 | 10.4009 | 17.4595 | 29.3766 | 49.982 | P2RX3 |
| XLOC_033962 | SOX10 | 12.2156 | 5.89624 | 15.1764 | 13.7585 | 33.604 | 72.0709 | SOX10 |
| XLOC_023568 | TTR | 7.64744 | 5.28695 | 9.74179 | 44.2219 | 26.8182 | 11.0765 | TTR |
| XLOC_001718 | NHLH1 | 6.836 | 6.19652 | 5.38626 | 13.3689 | 16.7953 | 36.7185 | NHLH1 |
| XLOC_049809 | DMRT2 | 0.060428 | 0.144392 | 0.05972 | 0.425798 | 0.402112 | 0.132896 | DMRT2 |
| XLOC_018647 | HS3ST2 | 0.464535 | 0.258888 | 0.307657 | 0.687753 | 1.01603 | 2.07308 | HS3ST2 |
| XLOC_030682 | COL4A4 | 0.304637 | 0.135306 | 0.181829 | 0.652932 | 0.405197 | 1.22085 | COL4A4 |
| XLOC_001956 | CACNA1E | 0.17295 | 0.115689 | 0.13369 | 0.440042 | 0.452217 | 0.658248 | CACNA1E |
| XLOC_004290 | FAM78B | 0.345477 | 0.279269 | 0.333669 | 0.646977 | 0.982876 | 1.90918 | FAM78B |
| XLOC_028175 | THSD7B | 2.67226 | 1.08222 | 1.82101 | 4.0935 | 6.58361 | 10.0276 | THSD7B |
| XLOC_031081 | XKR7 | 0.94844 | 0.508548 | 0.484812 | 1.11108 | 2.39362 | 3.70723 | XKR7 |
| XLOC_021148 | TMEM132E | 1.40988 | 0.486328 | 0.913432 | 1.32051 | 2.89112 | 6.25745 | TMEM132E |
| XLOC_006505 | DRGX | 0.451418 | 0.137073 | 0.333515 | 0.540105 | 1.09737 | 1.95555 | DRGX |
| XLOC_016464 | APBA2 | 0.147763 | 0.102978 | 0.116272 | 0.621117 | 0.30874 | 0.50119 | APBA2 |
| XLOC_023046 | ICAM2 | 0.049211 | 0 | 0.154296 | 0.289586 | 0.187764 | 0.322514 | ICAM2 |
| XLOC_005844 | TLX1 | 0.807038 | 0.347483 | 0.883956 | 1.03245 | 3.05458 | 3.94125 | TLX1 |
| XLOC_005701 | SNCG | 13.499 | 10.2857 | 11.1359 | 24.9947 | 39.0555 | 73.5977 | SNCG |
| XLOC_039723 | ISL1 | 3.80905 | 1.01111 | 1.95895 | 3.02885 | 9.21573 | 14.5762 | ISL1 |
| XLOC_024513 | ONECUT3 | 0.073395 | 0.085392 | 0.116475 | 0.113174 | 0.295455 | 0.685975 | ONECUT3 |
| XLOC_007448 | SLC17A6 | 0.54407 | 0.608742 | 0.758128 | 1.27419 | 2.06446 | 4.30237 | SLC17A6 |
| XLOC_053775 | BEX5 | 1.27132 | 0.998891 | 1.58627 | 6.6662 | 4.7515 | 4.21188 | BEX5 |
| XLOC_019423 | CDH15 | 0.360616 | 0.084472 | 0.334736 | 0.218565 | 0.870598 | 2.08424 | CDH15 |
| XLOC_000925 | FOXD3 | 1.2178 | 0.699633 | 1.34897 | 1.61526 | 3.91824 | 7.85973 | FOXD3 |
| XLOC_001202 | NTNG1 | 1.08523 | 0.722204 | 1.30259 | 4.82741 | 2.79911 | 5.15245 | NTNG1 |
| XLOC_036925 | APOD | 0.174434 | 0.268206 | 0.139212 | 0.378946 | 0.715705 | 1.3068 | APOD |
| XLOC_041716 | NEUROG1 | 2.45398 | 1.73346 | 1.21042 | 2.01133 | 7.79432 | 12.5119 | NEUROG1 |
| XLOC_036866 | SST | 95.6846 | 43.2791 | 93.8272 | 173.63 | 389.789 | 428.784 | SST |
| XLOC_030364 | CERKL, | 3.11322 | 2.10865 | 2.06817 | 4.77815 | 9.36537 | 17.3522 | CERKL, |
| | NEUROD1 | | | | | | | NEUROD1 |
| XLOC_041120 | AC010478.1 | 0.670449 | 0.335633 | 0.558476 | 1.1048 | 2.14114 | 3.58328 | AC010478.1 |
| XLOC_042144 | FOXQ1 | 0.13011 | 0.176936 | 0.133934 | 0.556044 | 0.340398 | 1.03676 | FOXQ1 |
| XLOC_033193 | PCAT14 | 0.347555 | 3.34943 | 0.356329 | 9.4583 | 3.31814 | 5.48905 | PCAT14 |
| XLOC_002873 | PLA2G2A | 0.33933 | 11.6496 | 0.232865 | 34.2602 | 7.99949 | 13.8025 | PLA2G2A |
| XLOC_051150 | PAX5 | 0.041797 | 0.070755 | 0.110252 | 0.054935 | 0.794357 | 0.183891 | PAX5 |
| XLOC_046221 | EN2 | 0.077904 | 0.038845 | 0.216523 | 0.405189 | 0.280246 | 0.888435 | EN2 |
| XLOC_045087 | PPP1R17 | 7.65666 | 2.80618 | 6.70317 | 9.32798 | 28.1052 | 44.2009 | PPP1R17 |
| XLOC_050747 | PRDM12 | 0.602529 | 0.15295 | 0.304479 | 0.273935 | 1.8128 | 2.99427 | PRDM12 |
| XLOC_007587 | AC087521.3, | 0.470656 | 0.343695 | 0.236177 | 0.746018 | 1.47777 | 3.00638 | AC087521.3, |
| | C11orf96 | | | | | | | C11orf96 |
| XLOC_042003 | AC091980.2 | 0.125791 | 0.048248 | 0.229093 | 0.377453 | 0.447536 | 1.18557 | AC091980.2 |
| XLOC_003769 | LINC01356 | 1.61564 | 14.0389 | 2.64513 | 61.8663 | 13.3663 | 16.6268 | LINC01356 |
| XLOC_044934 | NXPH1 | 0.387357 | 0.155816 | 0.277031 | 0.776078 | 0.911533 | 2.50384 | NXPH1 |
| XLOC_027613 | TLX2 | 0.535173 | 0.465132 | 0.599038 | 0.839251 | 2.06084 | 5.2989 | TLX2 |
| XLOC_022838 | HOXB2 | 1.07836 | 0.403277 | 0.378429 | 2.09591 | 2.64729 | 4.9233 | HOXB2 |
| XLOC_028330 | SP5 | 2.12602 | 0.923064 | 1.94033 | 3.68214 | 5.66955 | 16.8732 | SP5 |
| XLOC_031003 | PAX1 | 0.088564 | 0.112961 | 0.078424 | 0.236148 | 0.449801 | 0.803336 | PAX1 |
| XLOC_001608 | CHRNB2 | 0.554063 | 0.657415 | 0.285777 | 0.782043 | 3.97458 | 3.69563 | CHRNB2 |
| XLOC_003403 | FOXD3-AS1 | 2.73328 | 2.74194 | 3.39322 | 7.61602 | 13.3845 | 30.4011 | FOXD3-AS1 |
| XLOC_015958 | ITPK1 | 0.193583 | 0.201487 | 0.219044 | 0.281163 | 3.02305 | 0.256659 | ITPK1 |
| XLOC_009251 | APLNR | 1.01519 | 0.570663 | 0.285534 | 2.03327 | 4.7726 | 4.06639 | APLNR |
| XLOC_038240 | CPLX1 | 0.337301 | 0.41944 | 0.337179 | 0.959054 | 1.89178 | 3.5133 | CPLX1 |
| XLOC_037482 | ODAM | 0.189997 | 0.456696 | 0.130386 | 1.23694 | 0.990273 | 2.52954 | ODAM |
| XLOC_045014 | GPNMB | 4.09897 | 2.67489 | 9.39444 | 20.0764 | 15.4035 | 64.2422 | GPNMB |
| XLOC_047629 | GATA4 | 0.154121 | 0.397169 | 0.267523 | 2.25432 | 1.15064 | 1.80146 | GATA4 |
| XLOC_037508 | AFP | 0.377022 | 0.440999 | 0.232247 | 1.07117 | 3.97382 | 1.70775 | AFP |
| XLOC_022839 | AC103702.1, | 0.352998 | 0.148958 | 0.209501 | 0.520492 | 1.21586 | 2.93308 | AC103702.1, |
| | HOXB3, | | | | | | | HOXB3, |
| | HOXB4, | | | | | | | HOXB4, |
| | HOXB6 | | | | | | | HOXB6 |
| XLOC_021627 | KCNH6 | 0.19152 | 0.116356 | 0.088714 | 0.504774 | 0.817851 | 1.40296 | KCNH6 |
| XLOC_028833 | PRSS56 | 0.765201 | 0.190726 | 0.485575 | 1.51774 | 1.82147 | 6.57563 | PRSS56 |
| XLOC_013913 | LINC00458 | 0.145233 | 0.796138 | 0.082385 | 4.77692 | 1.01213 | 1.83404 | LINC00458 |
| XLOC_040604 | TLX3 | 0.774906 | 0.115917 | 0.797678 | 1.51139 | 3.60394 | 7.50428 | TLX3 |
| XLOC_038604 | TECRL | 0.179064 | 0.047349 | 0.048874 | 1.02139 | 0.545223 | 0.655368 | TECRL |
| XLOC_034414 | KLHL40 | 0.215201 | 0.064383 | 0.099686 | 0.247534 | 1.0838 | 1.79501 | KLHL40 |
| XLOC_024084 | GATA6-AS1 | 0.032246 | 0.052996 | 0.524759 | 3.21282 | 0.866333 | 1.05875 | GATA6-AS1 |
| XLOC_052291 | PAGE4 | 0.093019 | 0.271256 | 0.280021 | 1.179 | 1.99725 | 2.29819 | PAGE4 |
| XLOC_050257 | GDA | 0.014489 | 0.033737 | 0.097653 | 0.602566 | 0.318894 | 0.400425 | GDA |
| XLOC_032924 | SPATC1L | 0.051189 | 0.118893 | 0.105387 | 1.7402 | 0.815051 | 0.02236 | SPATC1L |
| XLOC_004239 | ITLN2 | 0.079323 | 0.184579 | 0.221167 | 1.27032 | 0.733759 | 2.56027 | ITLN2 |
| XLOC_019449 | IL9RP3, | 0.136845 | 0.287302 | 0.3649 | 3.12169 | 1.35714 | 3.95977 | IL9RP3, |
| | WASH4P, | | | | | | | WASH4P, |
| | Z84723.1 | | | | | | | Z84723.1 |
| XLOC_018543 | NOMO1 | 0.027914 | 0.983765 | 0.259636 | 0.307088 | 0.11189 | 18.7616 | NOMO1 |
| XLOC_053103 | AJ271736.1, | 0.055779 | 0.043502 | 0.420968 | 0.125549 | 8.17491 | 0.229129 | AJ271736.1, |
| | IL9R, | | | | | | | IL9R, |
| | WASH6P | | | | | | | WASH6P |
| XLOC_015003 | AL110118.2, | 0.009336 | 0.982384 | 0.169714 | 18.721 | 0.249948 | 0.075754 | AL110118.2, |
| | TMEM251, | | | | | | | TMEM251, |
| | UBR7 | | | | | | | UBR7 |
| XLOC_021436 | HOXB-AS1, | 0.742093 | 0.109409 | 0.167643 | 2.17336 | 5.49078 | 9.27938 | HOXB-AS1, |
| | HOXB-AS3 | | | | | | | HOXB-AS3 |
| XLOC_047531 | MYOM2 | 0.149096 | 0.298459 | 0.211717 | 0.149216 | 0.192236 | 10.8701 | MYOM2 |
| XLOC_022539 | SYNRG | 0.076614 | 0.015469 | 0.18385 | 0.304336 | 0.088185 | 4.70465 | SYNRG |
| XLOC_001584 | INTS3, | 0.145904 | 0.037429 | 0.24386 | 0.250705 | 8.01942 | 0.053786 | INTS3, |
| | SLC27A3 | | | | | | | SLC27A3 |
| XLOC_030625 | IHH | 0.027396 | 0.054641 | 0.028201 | 0.671339 | 0.464598 | 1.07154 | IHH |
| XLOC_025815 | SBNO2 | 0.11578 | 0.079897 | 0.108348 | 0.140676 | 6.62508 | 0.297605 | SBNO2 |
| XLOC_031698 | CST1 | 1.8543 | 12.9097 | 0.923864 | 166.671 | 79.0722 | 125.455 | CST1 |
| XLOC_018539 | BFAR | 0.203068 | 0.200661 | 0.115413 | 0.392574 | 13.2101 | 0.357776 | BFAR |
| XLOC_042579 | KIFC1 | 0.019888 | 0.049667 | 0.061418 | 4.95185 | 0.048665 | 0.009372 | KIFC1 |
| XLOC_013689 | CDX2 | 0.100395 | 0.080095 | 0.041338 | 1.45376 | 3.82786 | 7.68326 | CDX2 |
| XLOC_031669 | AL121722.1, | 0.011317 | 0.089115 | 0.011004 | 2.03396 | 1.72079 | 3.13084 | AL121722.1, |
| | FOXA2, | | | | | | | FOXA2, |
| | LINC00261 | | | | | | | LINCO0261 |
| XLOC_022563 | PIP4K2B | 0.165703 | 0.09556 | 0.02213 | 0.240704 | 0.039356 | 20.7652 | PIP4K2B |
| XLOC_030000 | RAB6C-AS1 | 0.089992 | 0.033102 | 0.031389 | 0.011065 | 22.0506 | 0.043111 | RAB6C-AS1 |
| XLOC_019729 | NTAN1 | 0 | 0 | 0.05628 | 8.25715 | 0 | 0.051527 | NTAN1 |
| XLOC_022262 | AC004448.4 | 0 | 0 | 0 | 7.24031 | 7.20388 | 0 | AC004448.4 |
| XLOC_008457 | APOC3 | 0 | 0 | 0 | 0.385105 | 0.902871 | 0.213859 | APOC3 |
| XLOC_030866 | GNRH2 | 0 | 0 | 0 | 0.353917 | 0.363679 | 0.249198 | GNRH2 |
| XLOC_048664 | DEFA6 | 0 | 0 | 0 | 1.45633 | 0.598601 | 1.46378 | DEFA6 |
| XLOC_026995 | ZNF667 | 1.0593 | 3.4673 | 1.3381 | 0 | 0 | 0 | ZNF667 |
| XLOC_050265 | AL451127.1 | 0.422805 | 0.421643 | 0.373054 | 0 | 0 | 0 | AL451127.1 |
| XLOC_053024 | MAGEA4 | 0.35109 | 0.384778 | 0.181792 | 0 | 0 | 0 | MAGEA4 |
| XLOC_025713 | ZNF667-AS1 | 4.73676 | 9.32947 | 4.82475 | 0 | 0 | 0 | ZNF667-AS1 |
| XLOC_027001 | ZNF835 | 0.053623 | 0.658148 | 0.340527 | 0 | 0 | 0 | ZNF835 |
| XLOC_025714 | ZNF471 | 0.682554 | 1.15445 | 0.470161 | 0 | 0 | 0 | ZNF471 |
| XLOC_045348 | ZNF736 | 0.624218 | 1.92792 | 0.552082 | 0 | 0 | 0 | ZNF736 |
| XLOC_006399 | AL132657.1, | 0.863026 | 4.17081 | 1.05968 | 0 | 0 | 0 | AL132657.1, |
| | ZNF248 | | | | | | | ZNF248 |

| | | | | | | | | | | signif- |
|---|---|---|---|---|---|---|---|---|---|---|
| tracking_id | sample_1 | sample_2 | status | value_1 | value_2 | log2(fold_change) | test_stat | p_value | q_value | icant |
| XLOC_027099 | WT | PAX6DKO | OK | 13.8546 | 0.044489 | −8.2827 | −0.62237 | 0.00005 | 0.002735 | yes |
| XLOC_044162 | WT | PAX6DKO | OK | 4.68145 | 0.022083 | −7.72791 | −0.68483 | 0.00005 | 0.002735 | yes |
| XLOC_033655 | WT | PAX6DKO | OK | 1.1969 | 0.005725 | −7.70782 | −0.24225 | 0.00005 | 0.002735 | yes |
| XLOC_014867 | WT | PAX6DKO | OK | 37.3025 | 0.203829 | −7.51577 | −9.25545 | 0.00005 | 0.002735 | yes |
| XLOC_049453 | WT | PAX6DKO | OK | 57.6877 | 0.631516 | −6.5133 | −4.41408 | 0.00005 | 0.002735 | yes |
| XLOC_028271 | WT | PAX6DKO | OK | 109.069 | 2.06876 | −5.72033 | −7.62692 | 0.00005 | 0.002735 | yes |
| XLOC_039936 | WT | PAX6DKO | OK | 90.8139 | 1.88038 | −5.59382 | −5.77908 | 0.00005 | 0.002735 | yes |
| XLOC_000776 | WT | PAX6DKO | OK | 32.2388 | 0.915053 | −5.1388 | −8.24877 | 0.00005 | 0.002735 | yes |
| XLOC_044161 | WT | PAX6DKO | OK | 0.598192 | 0.017576 | −5.0889 | −4.59663 | 0.00015 | 0.007086 | yes |
| XLOC_012702 | WT | PAX6DKO | OK | 2.90257 | 0.095462 | −4.92627 | −3.09453 | 0.00005 | 0.002735 | yes |
| XLOC_002160 | WT | PAX6DKO | OK | 4.32369 | 0.15815 | −4.7729 | −2.4677 | 0.00005 | 0.002735 | yes |
| XLOC_012591 | WT | PAX6DKO | OK | 1.74929 | 0.075006 | −4.54363 | −0.74023 | 0.00005 | 0.002735 | yes |
| XLOC_009482 | WT | PAX6DKO | OK | 8.49054 | 0.395327 | −4.42474 | −4.22442 | 0.00005 | 0.002735 | yes |
| XLOC_002728 | WT | PAX6DKO | OK | 41.8801 | 2.06603 | −4.34133 | −8.6155 | 0.00005 | 0.002735 | yes |
| XLOC_027566 | WT | PAX6DKO | OK | 7.32415 | 0.384791 | −4.25052 | −0.98028 | 0.0002 | 0.008981 | yes |
| XLOC_025026 | WT | PAX6DKO | OK | 1.3916 | 0.074045 | −4.23221 | −2.49309 | 0.00005 | 0.002735 | yes |
| XLOC_018597 | WT | PAX6DKO | OK | 18.1238 | 0.981018 | −4.20747 | −4.07109 | 0.00005 | 0.002735 | yes |
| XLOC_036565 | WT | PAX6DKO | OK | 33.6845 | 1.95037 | −4.11027 | −2.15162 | 0.00005 | 0.002735 | yes |
| XLOC_040762 | WT | PAX6DKO | OK | 1.84837 | 0.108945 | −4.08458 | −2.94393 | 0.00005 | 0.002735 | yes |
| XLOC_006922 | WT | PAX6DKO | OK | 4.25081 | 0.253816 | −4.06588 | −4.43349 | 0.00005 | 0.002735 | yes |
| XLOC_033225 | WT | PAX6DKO | OK | 4.36783 | 0.263748 | −4.04968 | −3.96378 | 0.00005 | 0.002735 | yes |
| XLOC_002465 | WT | PAX6DKO | OK | 12.1058 | 0.783686 | −3.94928 | −1.30593 | 0.00005 | 0.002735 | yes |
| XLOC_048453 | WT | PAX6DKO | OK | 0.492774 | 0.040166 | −3.61687 | −3.4056 | 0.0001 | 0.005033 | yes |
| XLOC_005226 | WT | PAX6DKO | OK | 0.938073 | 0.079367 | −3.56309 | −1.53774 | 0.0006 | 0.021697 | yes |
| XLOC_019871 | WT | PAX6DKO | OK | 1.10959 | 0.095342 | −3.54077 | −3.77585 | 0.00005 | 0.002735 | yes |
| XLOC_030539 | WT | PAX6DKO | OK | 12.7911 | 1.1588 | −3.46444 | −7.53615 | 0.00005 | 0.002735 | yes |
| XLOC_041832 | WT | PAX6DKO | OK | 132.265 | 12.1875 | −3.43996 | −10.1016 | 0.00005 | 0.002735 | yes |
| XLOC_024322 | WT | PAX6DKO | OK | 11.6995 | 1.11752 | −3.38808 | −3.55778 | 0.00005 | 0.002735 | yes |
| XLOC_030921 | WT | PAX6DKO | OK | 7.64404 | 0.734907 | −3.3787 | −4.87076 | 0.00005 | 0.002735 | yes |
| XLOC_008341 | WT | PAX6DKO | OK | 5.25207 | 0.509058 | −3.36698 | −2.30725 | 0.00005 | 0.002735 | yes |
| XLOC_038319 | WT | PAX6DKO | OK | 4.34103 | 0.426332 | −3.34799 | −3.86927 | 0.00005 | 0.002735 | yes |
| XLOC_041731 | WT | PAX6DKO | OK | 153.008 | 15.0873 | −3.3422 | −4.69609 | 0.00005 | 0.002735 | yes |
| XLOC_009925 | WT | PAX6DKO | OK | 1161.01 | 118.95 | −3.28696 | −7.33262 | 0.00005 | 0.002735 | yes |
| XLOC_048642 | WT | PAX6DKO | OK | 6.18229 | 0.635076 | −3.28314 | −2.33246 | 0.00005 | 0.002735 | yes |
| XLOC_001908 | WT | PAX6DKO | OK | 17.7272 | 1.92744 | −3.20121 | −2.68441 | 0.00005 | 0.002735 | yes |
| XLOC_034205 | WT | PAX6DKO | OK | 1.92053 | 0.214956 | −3.15939 | −3.47113 | 0.00005 | 0.002735 | yes |
| XLOC_010676 | WT | PAX6DKO | OK | 1.50841 | 0.171897 | −3.13341 | −2.10811 | 0.00005 | 0.002735 | yes |
| XLOC_031377 | WT | PAX6DKO | OK | 1.60647 | 0.200641 | −3.00121 | −1.85466 | 0.00025 | 0.010784 | yes |
| XLOC_038358 | WT | PAX6DKO | OK | 1.0431 | 0.137588 | −2.92245 | −3.24058 | 0.00005 | 0.002735 | yes |
| XLOC_027565 | WT | PAX6DKO | OK | 1.85122 | 0.262012 | −2.82077 | −2.93627 | 0.0001 | 0.005033 | yes |
| XLOC_051344 | WT | PAX6DKO | OK | 319.839 | 45.909 | −2.8005 | −7.23473 | 0.00005 | 0.002735 | yes |
| XLOC_036590 | WT | PAX6DKO | OK | 3.22484 | 0.468944 | −2.78174 | −2.30445 | 0.00005 | 0.002735 | yes |
| XLOC_036625 | WT | PAX6DKO | OK | 94.1119 | 13.7191 | −2.77819 | −6.1448 | 0.00005 | 0.002735 | yes |
| XLOC_017273 | WT | PAX6DKO | OK | 65.9526 | 9.67621 | −2.76891 | −4.34404 | 0.00005 | 0.002735 | yes |
| XLOC_016033 | WT | PAX6DKO | OK | 3.77219 | 0.556379 | −2.76126 | −2.49978 | 0.00005 | 0.002735 | yes |
| XLOC_029247 | WT | PAX6DKO | OK | 18.2894 | 2.72921 | −2.74445 | −4.43616 | 0.00005 | 0.002735 | yes |
| XLOC_018711 | WT | PAX6DKO | OK | 23.3321 | 3.48877 | −2.74153 | −3.66123 | 0.00005 | 0.002735 | yes |
| XLOC_032052 | WT | PAX6DKO | OK | 2.41816 | 0.373801 | −2.69357 | −3.46766 | 0.00005 | 0.002735 | yes |
| XLOC_019330 | WT | PAX6DKO | OK | 88.2719 | 13.6565 | −2.69236 | −5.75902 | 0.00005 | 0.002735 | yes |
| XLOC_005448 | WT | PAX6DKO | OK | 18.3327 | 2.83914 | −2.69089 | −3.58048 | 0.00005 | 0.002735 | yes |
| XLOC_014052 | WT | PAX6DKO | OK | 51.0324 | 8.48895 | −2.58776 | −5.30577 | 0.00005 | 0.002735 | yes |
| XLOC_002706 | WT | PAX6DKO | OK | 3.41564 | 0.575923 | −2.56821 | −2.78288 | 0.00005 | 0.002735 | yes |
| XLOC_044950 | WT | PAX6DKO | OK | 0.58625 | 0.100584 | −2.54311 | −0.40351 | 0.00025 | 0.010784 | yes |
| XLOC_042581 | WT | PAX6DKO | OK | 0.716836 | 0.128166 | −2.48362 | −0.22491 | 0.00005 | 0.002735 | yes |
| XLOC_012370 | WT | PAX6DKO | OK | 4.70027 | 0.849185 | −2.46859 | −0.65345 | 0.0002 | 0.008981 | yes |
| XLOC_015185 | WT | PAX6DKO | OK | 4.59371 | 0.860763 | −2.41597 | −3.59669 | 0.00005 | 0.002735 | yes |
| XLOC_008373 | WT | PAX6DKO | OK | 8.60107 | 1.63621 | −2.39416 | −2.38418 | 0.00005 | 0.002735 | yes |
| XLOC_021142 | WT | PAX6DKO | OK | 374.477 | 72.3072 | −2.37267 | −6.92448 | 0.00005 | 0.002735 | yes |
| XLOC_005951 | WT | PAX6DKO | OK | 0.644767 | 0.124854 | −2.36853 | −2.43987 | 0.0004 | 0.015799 | yes |
| XLOC_020429 | WT | PAX6DKO | OK | 24.0091 | 4.65163 | −2.36778 | −3.85529 | 0.00005 | 0.002735 | yes |
| XLOC_002055 | WT | PAX6DKO | OK | 3.03883 | 0.600941 | −2.33822 | −1.98223 | 0.00005 | 0.002735 | yes |
| XLOC_007296 | WT | PAX6DKO | OK | 21.8429 | 4.51119 | −2.27559 | −4.5159 | 0.00005 | 0.002735 | yes |
| XLOC_050766 | WT | PAX6DKO | OK | 21.0381 | 4.48071 | −2.23121 | −3.75951 | 0.00005 | 0.002735 | yes |
| XLOC_030190 | WT | PAX6DKO | OK | 0.810134 | 0.173599 | −2.2224 | −1.07744 | 0.00175 | 0.049136 | yes |
| XLOC_024400 | WT | PAX6DKO | OK | 10.1296 | 2.22057 | −2.18958 | −3.49985 | 0.00005 | 0.002735 | yes |
| XLOC_039183 | WT | PAX6DKO | OK | 682.925 | 150.444 | −2.1825 | −6.55481 | 0.00005 | 0.002735 | yes |
| XLOC_034614 | WT | PAX6DKO | OK | 12.7807 | 2.87857 | −2.15054 | −3.40514 | 0.00005 | 0.002735 | yes |
| XLOC_017031 | WT | PAX6DKO | OK | 1789.66 | 415.74 | −2.10593 | −5.7299 | 0.00005 | 0.002735 | yes |
| XLOC_043011 | WT | PAX6DKO | OK | 2.64893 | 0.618562 | −2.09842 | −1.99805 | 0.0015 | 0.04365 | yes |
| XLOC_035457 | WT | PAX6DKO | OK | 0.594849 | 0.139045 | −2.09697 | −1.54422 | 0.00075 | 0.025855 | yes |
| XLOC_003488 | WT | PAX6DKO | OK | 18.8858 | 4.45033 | −2.08532 | −2.39096 | 0.00005 | 0.002735 | yes |
| XLOC_049746 | WT | PAX6DKO | OK | 0.994641 | 0.242762 | −2.03464 | −1.9756 | 0.0003 | 0.012524 | yes |
| XLOC_013669 | WT | PAX6DKO | OK | 12.8456 | 3.13533 | −2.03459 | −4.43719 | 0.00005 | 0.002735 | yes |
| XLOC_015082 | WT | PAX6DKO | OK | 238.716 | 59.7075 | −1.99931 | −4.78287 | 0.00005 | 0.002735 | yes |
| XLOC_047097 | WT | PAX6DKO | OK | 13.1487 | 3.31631 | −1.98727 | −2.24776 | 0.00135 | 0.040437 | yes |
| XLOC_029306 | WT | PAX6DKO | OK | 8.32896 | 2.12102 | −1.97338 | −2.42373 | 0.0001 | 0.005033 | yes |
| XLOC_037223 | WT | PAX6DKO | OK | 24.1676 | 6.21149 | −1.96006 | −3.17172 | 0.00005 | 0.002735 | yes |
| XLOC_019146 | WT | PAX6DKO | OK | 0.741948 | 0.190948 | −1.95814 | −1.09518 | 0.00105 | 0.03336 | yes |
| XLOC_005835 | WT | PAX6DKO | OK | 1.85907 | 0.483192 | −1.94391 | −2.02822 | 0.0001 | 0.005033 | yes |
| XLOC_002251 | WT | PAX6DKO | OK | 2.58726 | 0.673995 | −1.94062 | −2.36786 | 0.00005 | 0.002735 | yes |
| XLOC_029304 | WT | PAX6DKO | OK | 33.271 | 8.6993 | −1.93529 | −2.49815 | 0.00005 | 0.002735 | yes |
| XLOC_018083 | WT | PAX6DKO | OK | 64.0612 | 17.1688 | −1.89966 | −1.27121 | 0.0012 | 0.037051 | yes |
| XLOC_027327 | WT | PAX6DKO | OK | 3.12486 | 0.844622 | −1.88741 | −1.90915 | 0.00175 | 0.049136 | yes |
| XLOC_002049 | WT | PAX6DKO | OK | 3.37884 | 0.949859 | −1.83074 | −2.20655 | 0.0008 | 0.027077 | yes |
| XLOC_023764 | WT | PAX6DKO | OK | 36.99 | 10.4005 | −1.83048 | −4.96708 | 0.00005 | 0.002735 | yes |
| XLOC_048281 | WT | PAX6DKO | OK | 35.1109 | 10.0108 | −1.81036 | −3.73645 | 0.00005 | 0.002735 | yes |
| XLOC_027527 | WT | PAX6DKO | OK | 80.7168 | 23.0257 | −1.80962 | −2.72362 | 0.00005 | 0.002735 | yes |
| XLOC_013601 | WT | PAX6DKO | OK | 0.56806 | 0.163374 | −1.79786 | −2.24658 | 0.0005 | 0.018851 | yes |
| XLOC_008880 | WT | PAX6DKO | OK | 6.67388 | 1.93676 | −1.78488 | −2.32845 | 0.00045 | 0.017298 | yes |
| XLOC_018419 | WT | PAX6DKO | OK | 17.409 | 5.06296 | −1.78178 | −2.77842 | 0.00005 | 0.002735 | yes |
| XLOC_036882 | WT | PAX6DKO | OK | 59.19 | 17.5659 | −1.75257 | −5.16907 | 0.00005 | 0.002735 | yes |
| XLOC_051664 | WT | PAX6DKO | OK | 245.742 | 72.9495 | −1.75218 | −1.8673 | 0.0004 | 0.015799 | yes |
| XLOC_024388 | WT | PAX6DKO | OK | 1.18456 | 0.358471 | −1.72442 | −1.24752 | 0.0014 | 0.041595 | yes |
| XLOC_041354 | WT | PAX6DKO | OK | 2.47954 | 0.751713 | −1.72182 | −2.696 | 0.00005 | 0.002735 | yes |
| XLOC_002245 | WT | PAX6DKO | OK | 5.68103 | 1.73317 | −1.71274 | −2.04898 | 0.00005 | 0.002735 | yes |
| XLOC_043359 | WT | PAX6DKO | OK | 6.65213 | 2.0657 | −1.68719 | −2.96494 | 0.00005 | 0.002735 | yes |
| XLOC_053790 | WT | PAX6DKO | OK | 19.6122 | 6.1518 | −1.67267 | −3.15806 | 0.00005 | 0.002735 | yes |
| XLOC_039957 | WT | PAX6DKO | OK | 4.7873 | 1.5734 | −1.60533 | −2.23791 | 0.0005 | 0.018851 | yes |
| XLOC_015824 | WT | PAX6DKO | OK | 11.1405 | 3.71958 | −1.5826 | −3.56596 | 0.00005 | 0.002735 | yes |
| XLOC_041138 | WT | PAX6DKO | OK | 3.51763 | 1.21214 | −1.53704 | −1.60527 | 0.0004 | 0.015799 | yes |
| XLOC_039386 | WT | PAX6DKO | OK | 1.31445 | 0.453366 | −1.53571 | −1.43606 | 0.00175 | 0.049136 | yes |
| XLOC_027382 | WT | PAX6DKO | OK | 40.2798 | 14.7135 | −1.45292 | −4.10509 | 0.00005 | 0.002735 | yes |
| XLOC_034342 | WT | PAX6DKO | OK | 1.26819 | 0.466983 | −1.44133 | −0.88485 | 0.00025 | 0.010784 | yes |
| XLOC_022663 | WT | PAX6DKO | OK | 42.825 | 15.8462 | −1.43431 | −2.72198 | 0.00005 | 0.002735 | yes |
| XLOC_044494 | WT | PAX6DKO | OK | 9.95773 | 3.70512 | −1.4263 | −3.61353 | 0.00005 | 0.002735 | yes |
| XLOC_050369 | WT | PAX6DKO | OK | 17.2334 | 6.42465 | −1.42351 | −3.8051 | 0.00005 | 0.002735 | yes |
| XLOC_004771 | WT | PAX6DKO | OK | 7.22815 | 2.7009 | −1.42019 | −1.72971 | 0.00005 | 0.002735 | yes |
| XLOC_012012 | WT | PAX6DKO | OK | 3.96584 | 1.48953 | −1.41277 | −2.31021 | 0.00015 | 0.007086 | yes |
| XLOC_053069 | WT | PAX6DKO | OK | 4.06986 | 1.56555 | −1.37831 | −1.46478 | 0.00055 | 0.020318 | yes |
| XLOC_011248 | WT | PAX6DKO | OK | 4.72903 | 1.82043 | −1.37726 | −2.48377 | 0.0001 | 0.005033 | yes |
| XLOC_013946 | WT | PAX6DKO | OK | 3.14275 | 1.21504 | −1.37103 | −2.05781 | 0.00005 | 0.002735 | yes |
| XLOC_030425 | WT | PAX6DKO | OK | 30.5899 | 11.9018 | −1.36187 | −1.63676 | 0.00045 | 0.017298 | yes |
| XLOC_001972 | WT | PAX6DKO | OK | 10.8533 | 4.22773 | −1.36017 | −3.15697 | 0.00005 | 0.002735 | yes |
| XLOC_038812 | WT | PAX6DKO | OK | 105.354 | 41.2136 | −1.35406 | −1.9301 | 0.0013 | 0.039328 | yes |
| XLOC_031027 | WT | PAX6DKO | OK | 1.48165 | 0.583525 | −1.34434 | −1.7836 | 0.0016 | 0.045919 | yes |
| XLOC_050268 | WT | PAX6DKO | OK | 1.97333 | 0.782273 | −1.33489 | −1.94623 | 0.0012 | 0.037051 | yes |
| XLOC_050235 | WT | PAX6DKO | OK | 11.5413 | 4.63363 | −1.31659 | −2.06184 | 0.0002 | 0.008981 | yes |
| XLOC_049879 | WT | PAX6DKO | OK | 110.799 | 45.1455 | −1.29529 | −1.60085 | 0.0012 | 0.037051 | yes |
| XLOC_035875 | WT | PAX6DKO | OK | 6.21201 | 2.59306 | −1.2604 | −2.26997 | 0.00015 | 0.007086 | yes |
| XLOC_047896 | WT | PAX6DKO | OK | 46.5979 | 19.4616 | −1.25964 | −3.43478 | 0.00005 | 0.002735 | yes |
| XLOC_048409 | WT | PAX6DKO | OK | 2.80108 | 1.17647 | −1.25152 | −1.87145 | 0.001 | 0.032108 | yes |
| XLOC_010972 | WT | PAX6DKO | OK | 19.745 | 8.31231 | −1.24816 | −2.88655 | 0.00005 | 0.002735 | yes |
| XLOC_050248 | WT | PAX6DKO | OK | 29.0636 | 12.3354 | −1.23641 | −3.46933 | 0.00005 | 0.002735 | yes |
| XLOC_004451 | WT | PAX6DKO | OK | 26.8217 | 11.4241 | −1.23132 | −3.69478 | 0.00005 | 0.002735 | yes |
| XLOC_026186 | WT | PAX6DKO | OK | 22.3438 | 9.57108 | −1.22312 | −1.78212 | 0.0001 | 0.005033 | yes |
| XLOC_004455 | WT | PAX6DKO | OK | 3.41227 | 1.46333 | −1.22148 | −2.40389 | 0.00005 | 0.002735 | yes |
| XLOC_023416 | WT | PAX6DKO | OK | 51.2899 | 22.1006 | −1.21459 | −1.69767 | 0.00005 | 0.002735 | yes |
| XLOC_027383 | WT | PAX6DKO | OK | 3.63863 | 1.57171 | −1.21106 | −2.03937 | 0.00085 | 0.028328 | yes |
| XLOC_051679 | WT | PAX6DKO | OK | 3.064 | 1.37247 | −1.15865 | −1.98142 | 0.00035 | 0.014242 | yes |
| XLOC_002264 | WT | PAX6DKO | OK | 66.7636 | 30.5567 | −1.12757 | −2.58715 | 0.00005 | 0.002735 | yes |
| XLOC_006249 | WT | PAX6DKO | OK | 0.613709 | 0.281662 | −1.12359 | −1.32247 | 0.0014 | 0.041595 | yes |
| XLOC_034650 | WT | PAX6DKO | OK | 11.0536 | 5.07696 | −1.12248 | −1.93694 | 0.00065 | 0.023143 | yes |
| XLOC_030257 | WT | PAX6DKO | OK | 33.8572 | 15.6057 | −1.11739 | −1.88201 | 0.00005 | 0.002735 | yes |
| XLOC_017507 | WT | PAX6DKO | OK | 3.39596 | 1.58196 | −1.1021 | −1.90966 | 0.00135 | 0.040437 | yes |
| XLOC_017508 | WT | PAX6DKO | OK | 277.888 | 131.327 | −1.08134 | −2.14047 | 0.0001 | 0.005033 | yes |
| XLOC_000284 | WT | PAX6DKO | OK | 35.0271 | 16.5882 | −1.07832 | −3.0893 | 0.00005 | 0.002735 | yes |
| XLOC_027354 | WT | PAX6DKO | OK | 66.0711 | 31.4551 | −1.07072 | −2.4031 | 0.00005 | 0.002735 | yes |
| XLOC_003193 | WT | PAX6DKO | OK | 362.181 | 172.621 | −1.0691 | −3.17841 | 0.00005 | 0.002735 | yes |
| XLOC_027319 | WT | PAX6DKO | OK | 41.2941 | 19.7842 | −1.06159 | −1.94693 | 0.00005 | 0.002735 | yes |
| XLOC_048183 | WT | PAX6DKO | OK | 19.7062 | 9.66554 | −1.02773 | −1.76888 | 0.0017 | 0.048164 | yes |
| XLOC_012216 | WT | PAX6DKO | OK | 282.411 | 139.287 | −1.01973 | −2.01013 | 0.00105 | 0.03336 | yes |
| XLOC_034156 | WT | PAX6DKO | OK | 24.7366 | 12.3329 | −1.00413 | −2.52425 | 0.00005 | 0.002735 | yes |
| XLOC_047196 | WT | PAX6DKO | OK | 15.1672 | 7.5646 | −1.00362 | −2.02233 | 0.0004 | 0.015799 | yes |
| XLOC_030171 | WT | PAX6DKO | OK | 5.13546 | 10.3417 | 1.00991 | 1.94489 | 0.0005 | 0.018851 | yes |
| XLOC_038414 | WT | PAX6DKO | OK | 2.92644 | 5.91675 | 1.01566 | 1.4877 | 0.0014 | 0.041595 | yes |
| XLOC_044149 | WT | PAX6DKO | OK | 2.30183 | 4.69837 | 1.02938 | 2.13182 | 0.0004 | 0.015799 | yes |
| XLOC_015662 | WT | PAX6DKO | OK | 9.01825 | 18.4261 | 1.03083 | 1.8448 | 0.0013 | 0.039328 | yes |
| XLOC_004016 | WT | PAX6DKO | OK | 4.80676 | 9.86422 | 1.03714 | 2.3304 | 0.00015 | 0.007086 | yes |
| XLOC_035933 | WT | PAX6DKO | OK | 3.15007 | 6.51306 | 1.04795 | 1.74582 | 0.00135 | 0.040437 | yes |
| XLOC_041385 | WT | PAX6DKO | OK | 44.2804 | 91.8018 | 1.05186 | 1.99707 | 0.00075 | 0.025855 | yes |
| XLOC_004282 | WT | PAX6DKO | OK | 35.1536 | 73.4661 | 1.06341 | 2.81571 | 0.00005 | 0.002735 | yes |
| XLOC_028427 | WT | PAX6DKO | OK | 1.99563 | 4.23476 | 1.08544 | 1.63442 | 0.0015 | 0.04365 | yes |
| XLOC_053428 | WT | PAX6DKO | OK | 2.64875 | 5.76112 | 1.12104 | 2.03743 | 0.00035 | 0.014242 | yes |
| XLOC_010398 | WT | PAX6DKO | OK | 4.23492 | 9.2352 | 1.12481 | 1.77959 | 0.0013 | 0.039328 | yes |
| XLOC_033238 | WT | PAX6DKO | OK | 1.78039 | 3.88533 | 1.12585 | 1.66333 | 0.0007 | 0.02448 | yes |
| XLOC_051389 | WT | PAX6DKO | OK | 2.8445 | 6.29675 | 1.14643 | 1.71262 | 0.001 | 0.032108 | yes |
| XLOC_031307 | WT | PAX6DKO | OK | 3.47386 | 7.74958 | 1.15758 | 2.11251 | 0.0006 | 0.021697 | yes |
| XLOC_030989 | WT | PAX6DKO | OK | 1.88969 | 4.25598 | 1.17134 | 2.12288 | 0.00055 | 0.020318 | yes |
| XLOC_050669 | WT | PAX6DKO | OK | 2.38543 | 5.45404 | 1.19307 | 2.65481 | 0.00005 | 0.002735 | yes |
| XLOC_052629 | WT | PAX6DKO | OK | 16.2352 | 37.7353 | 1.21679 | 2.84303 | 0.00005 | 0.002735 | yes |
| XLOC_006747 | WT | PAX6DKO | OK | 1.28681 | 3.04298 | 1.24169 | 1.86443 | 0.00155 | 0.04486 | yes |
| XLOC_052734 | WT | PAX6DKO | OK | 1.28598 | 3.09185 | 1.2656 | 2.16144 | 0.0005 | 0.018851 | yes |
| XLOC_047751 | WT | PAX6DKO | OK | 21.9292 | 53.8516 | 1.29613 | 2.82541 | 0.00005 | 0.002735 | yes |
| XLOC_042079 | WT | PAX6DKO | OK | 3.4896 | 8.57621 | 1.29728 | 2.57594 | 0.00005 | 0.002735 | yes |
| XLOC_048890 | WT | PAX6DKO | OK | 14.2743 | 35.1081 | 1.29838 | 3.37409 | 0.00005 | 0.002735 | yes |
| XLOC_018031 | WT | PAX6DKO | OK | 1.0503 | 2.60937 | 1.3129 | 1.95059 | 0.00045 | 0.017298 | yes |
| XLOC_004935 | WT | PAX6DKO | OK | 0.677219 | 1.71548 | 1.34091 | 2.11038 | 0.00055 | 0.020318 | yes |
| XLOC_042770 | WT | PAX6DKO | OK | 14.6718 | 37.8875 | 1.36868 | 2.06195 | 0.00085 | 0.028328 | yes |
| XLOC_030899 | WT | PAX6DKO | OK | 1.49461 | 3.88747 | 1.37906 | 2.32225 | 0.0001 | 0.005033 | yes |
| XLOC_052372 | WT | PAX6DKO | OK | 24.2723 | 63.28 | 1.38244 | 3.86078 | 0.00005 | 0.002735 | yes |
| XLOC_043938 | WT | PAX6DKO | OK | 0.400598 | 1.05177 | 1.3926 | 1.62064 | 0.0008 | 0.027077 | yes |
| XLOC_026219 | WT | PAX6DKO | OK | 0.943851 | 2.4949 | 1.40235 | 2.0374 | 0.00005 | 0.002735 | yes |
| XLOC_009683 | WT | PAX6DKO | OK | 2.68748 | 7.15116 | 1.41192 | 3.05202 | 0.00005 | 0.002735 | yes |
| XLOC_022915 | WT | PAX6DKO | OK | 1.5145 | 4.06333 | 1.42382 | 2.14183 | 0.0002 | 0.008981 | yes |
| XLOC_041742 | WT | PAX6DKO | OK | 0.744865 | 1.99865 | 1.42398 | 1.90038 | 0.00145 | 0.042686 | yes |
| XLOC_032857 | WT | PAX6DKO | OK | 1.87345 | 5.03315 | 1.42577 | 2.49258 | 0.0001 | 0.005033 | yes |
| XLOC_032392 | WT | PAX6DKO | OK | 3.34231 | 8.98084 | 1.42601 | 2.74364 | 0.00005 | 0.002735 | yes |
| XLOC_045787 | WT | PAX6DKO | OK | 3.1421 | 8.62736 | 1.45719 | 1.10044 | 0.00175 | 0.049136 | yes |
| XLOC_048897 | WT | PAX6DKO | OK | 1.11128 | 3.06977 | 1.46591 | 2.23328 | 0.0002 | 0.008981 | yes |
| XLOC_004252 | WT | PAX6DKO | OK | 6.753 | 18.764 | 1.47436 | 3.08096 | 0.00005 | 0.002735 | yes |
| XLOC_037044 | WT | PAX6DKO | OK | 17.2939 | 48.0574 | 1.47449 | 3.84479 | 0.00005 | 0.002735 | yes |
| XLOC_028714 | WT | PAX6DKO | OK | 0.586679 | 1.64024 | 1.48326 | 2.0213 | 0.0008 | 0.027077 | yes |
| XLOC_000794 | WT | PAX6DKO | OK | 7.39076 | 20.8626 | 1.49713 | 2.19203 | 0.00045 | 0.017298 | yes |
| XLOC_029809 | WT | PAX6DKO | OK | 9.75481 | 27.6441 | 1.50278 | 3.01748 | 0.00005 | 0.002735 | yes |
| XLOC_003798 | WT | PAX6DKO | OK | 0.604159 | 1.72438 | 1.51308 | 2.02515 | 0.00105 | 0.03336 | yes |
| XLOC_047230 | WT | PAX6DKO | OK | 3.2521 | 9.3604 | 1.5252 | 2.16529 | 0.0005 | 0.018851 | yes |
| XLOC_021373 | WT | PAX6DKO | OK | 4.44129 | 12.7862 | 1.52553 | 3.58304 | 0.00005 | 0.002735 | yes |
| XLOC_017731 | WT | PAX6DKO | OK | 0.818844 | 2.36721 | 1.53152 | 2.25864 | 0.00005 | 0.002735 | yes |
| XLOC_031394 | WT | PAX6DKO | OK | 1.16332 | 3.37693 | 1.53746 | 2.16234 | 0.0006 | 0.021697 | yes |
| XLOC_004924 | WT | PAX6DKO | OK | 3.31154 | 9.75013 | 1.55792 | 2.79598 | 0.00005 | 0.002735 | yes |
| XLOC_038929 | WT | PAX6DKO | OK | 3.57154 | 10.5386 | 1.56107 | 1.96682 | 0.0017 | 0.048164 | yes |
| XLOC_024906 | WT | PAX6DKO | OK | 10.0921 | 30.0351 | 1.57342 | 1.752 | 0.00095 | 0.030847 | yes |
| XLOC_048154 | WT | PAX6DKO | OK | 65.0374 | 194.563 | 1.5809 | 3.55432 | 0.00005 | 0.002735 | yes |
| XLOC_041939 | WT | PAX6DKO | OK | 2.76418 | 8.34542 | 1.59413 | 1.75806 | 0.0007 | 0.02448 | yes |
| XLOC_004283 | WT | PAX6DKO | OK | 1.86955 | 5.69326 | 1.60656 | 2.55355 | 0.00005 | 0.002735 | yes |
| XLOC_007091 | WT | PAX6DKO | OK | 3.7191 | 11.3546 | 1.61025 | 2.08594 | 0.0008 | 0.027077 | yes |
| XLOC_005825 | WT | PAX6DKO | OK | 0.559078 | 1.72219 | 1.62312 | 1.9006 | 0.0003 | 0.012524 | yes |
| XLOC_025095 | WT | PAX6DKO | OK | 0.863342 | 2.70398 | 1.64708 | 2.28107 | 0.00015 | 0.007086 | yes |
| XLOC_015674 | WT | PAX6DKO | OK | 4.19451 | 13.4046 | 1.67616 | 3.22236 | 0.00005 | 0.002735 | yes |
| XLOC_034818 | WT | PAX6DKO | OK | 0.588316 | 1.88732 | 1.68167 | 2.35027 | 0.00015 | 0.007086 | yes |
| XLOC_026209 | WT | PAX6DKO | OK | 0.355009 | 1.13984 | 1.68291 | 1.63427 | 0.00005 | 0.002735 | yes |
| XLOC_047719 | WT | PAX6DKO | OK | 0.806157 | 2.60122 | 1.69005 | 2.0028 | 0.0013 | 0.039328 | yes |
| XLOC_008137 | WT | PAX6DKO | OK | 0.221543 | 0.715542 | 1.69145 | 1.9544 | 0.00135 | 0.040437 | yes |
| XLOC_045774 | WT | PAX6DKO | OK | 26.4467 | 85.4535 | 1.69205 | 2.07728 | 0.00005 | 0.002735 | yes |
| XLOC_010779 | WT | PAX6DKO | OK | 0.651839 | 2.11874 | 1.70062 | 2.60841 | 0.00005 | 0.002735 | yes |
| XLOC_030738 | WT | PAX6DKO | OK | 7.51868 | 24.4602 | 1.70188 | 3.01336 | 0.00005 | 0.002735 | yes |
| XLOC_028834 | WT | PAX6DKO | OK | 0.156866 | 0.517984 | 1.72337 | 1.34856 | 0.00165 | 0.0471 | yes |
| XLOC_038132 | WT | PAX6DKO | OK | 0.54875 | 1.84042 | 1.74581 | 2.32617 | 0.0001 | 0.005033 | yes |
| XLOC_003734 | WT | PAX6DKO | OK | 0.119777 | 0.402352 | 1.7481 | 0.962856 | 0.00115 | 0.035883 | yes |
| XLOC_016656 | WT | PAX6DKO | OK | 0.172902 | 0.594928 | 1.78276 | 1.33988 | 0.0004 | 0.015799 | yes |
| XLOC_023042 | WT | PAX6DKO | OK | 0.107208 | 0.369015 | 1.78326 | 0.676355 | 0.0012 | 0.037051 | yes |
| XLOC_021365 | WT | PAX6DKO | OK | 2.7806 | 9.69871 | 1.8024 | 2.08758 | 0.00135 | 0.040437 | yes |
| XLOC_037406 | WT | PAX6DKO | OK | 1.56496 | 5.53663 | 1.82288 | 2.20517 | 0.0009 | 0.029595 | yes |
| XLOC_031942 | WT | PAX6DKO | OK | 0.812301 | 2.87429 | 1.82312 | 1.97116 | 0.00095 | 0.030847 | yes |
| XLOC_007768 | WT | PAX6DKO | OK | 9.12975 | 32.5947 | 1.83599 | 2.6328 | 0.00005 | 0.002735 | yes |
| XLOC_033962 | WT | PAX6DKO | OK | 11.2068 | 40.2084 | 1.84312 | 2.40779 | 0.00055 | 0.020318 | yes |
| XLOC_023568 | WT | PAX6DKO | OK | 7.63411 | 27.645 | 1.85649 | 2.99161 | 0.00005 | 0.002735 | yes |
| XLOC_001718 | WT | PAX6DKO | OK | 6.20081 | 22.5167 | 1.86047 | 4.47011 | 0.00005 | 0.002735 | yes |
| XLOC_049809 | WT | PAX6DKO | OK | 0.089058 | 0.323461 | 1.86078 | 1.25404 | 0.00045 | 0.017298 | yes |
| XLOC_018647 | WT | PAX6DKO | OK | 0.347122 | 1.27152 | 1.87303 | 2.25569 | 0.0002 | 0.008981 | yes |
| XLOC_030682 | WT | PAX6DKO | OK | 0.209325 | 0.767239 | 1.87393 | 2.72322 | 0.00005 | 0.002735 | yes |
| XLOC_001956 | WT | PAX6DKO | OK | 0.142181 | 0.521991 | 1.8763 | 2.0694 | 0.00005 | 0.002735 | yes |
| XLOC_004290 | WT | PAX6DKO | OK | 0.322658 | 1.19145 | 1.88464 | 2.2972 | 0.00005 | 0.002735 | yes |
| XLOC_028175 | WT | PAX6DKO | OK | 1.87705 | 6.97041 | 1.89278 | 1.70293 | 0.0003 | 0.012524 | yes |
| XLOC_031081 | WT | PAX6DKO | OK | 0.653725 | 2.42796 | 1.89299 | 3.52866 | 0.00005 | 0.002735 | yes |
| XLOC_021148 | WT | PAX6DKO | OK | 0.945894 | 3.52452 | 1.89768 | 2.00343 | 0.0002 | 0.008981 | yes |
| XLOC_006505 | WT | PAX6DKO | OK | 0.310403 | 1.20963 | 1.96235 | 2.38315 | 0.00005 | 0.002735 | yes |
| XLOC_016464 | WT | PAX6DKO | OK | 0.123558 | 0.481772 | 1.96316 | 1.25874 | 0.00005 | 0.002735 | yes |
| XLOC_023046 | WT | PAX6DKO | OK | 0.068513 | 0.26928 | 1.97466 | 0.368828 | 0.00095 | 0.030847 | yes |
| XLOC_005844 | WT | PAX6DKO | OK | 0.686272 | 2.70279 | 1.9776 | 1.85024 | 0.0003 | 0.012524 | yes |
| XLOC_005701 | WT | PAX6DKO | OK | 11.7563 | 46.3404 | 1.97884 | 3.27062 | 0.00005 | 0.002735 | yes |
| XLOC_039723 | WT | PAX6DKO | OK | 2.28227 | 9.02946 | 1.98417 | 3.60708 | 0.00005 | 0.002735 | yes |
| XLOC_024513 | WT | PAX6DKO | OK | 0.092669 | 0.368509 | 1.99155 | 2.46564 | 0.0002 | 0.008981 | yes |
| XLOC_007448 | WT | PAX6DKO | OK | 0.64333 | 2.57242 | 1.9995 | 2.96906 | 0.00005 | 0.002735 | yes |
| XLOC_053775 | WT | PAX6DKO | OK | 1.29831 | 5.2618 | 2.01892 | 2.51909 | 0.00005 | 0.002735 | yes |
| XLOC_019423 | WT | PAX6DKO | OK | 0.262537 | 1.06836 | 2.02481 | 2.24821 | 0.0001 | 0.005033 | yes |
| XLOC_000925 | WT | PAX6DKO | OK | 1.09966 | 4.50896 | 2.03573 | 2.55158 | 0.00015 | 0.007086 | yes |
| XLOC_001202 | WT | PAX6DKO | OK | 1.04701 | 4.30213 | 2.03877 | 3.2984 | 0.00005 | 0.002735 | yes |
| XLOC_036925 | WT | PAX6DKO | OK | 0.195883 | 0.808472 | 2.04521 | 1.55 | 0.0015 | 0.04365 | yes |
| XLOC_041716 | WT | PAX6DKO | OK | 1.81723 | 7.51343 | 2.04773 | 3.19824 | 0.00005 | 0.002735 | yes |
| XLOC_036866 | WT | PAX6DKO | OK | 78.3712 | 334.033 | 2.0916 | 5.81836 | 0.00005 | 0.002735 | yes |
| XLOC_030364 | WT | PAX6DKO | OK | 2.45425 | 10.6033 | 2.11116 | 3.89638 | 0.00005 | 0.002735 | yes |
| XLOC_041120 | WT | PAX6DKO | OK | 0.526723 | 2.29912 | 2.12596 | 2.66651 | 0.00005 | 0.002735 | yes |
| XLOC_042144 | WT | PAX6DKO | OK | 0.148458 | 0.65083 | 2.13223 | 2.3216 | 0.0004 | 0.015799 | yes |
| XLOC_033193 | WT | PAX6DKO | OK | 1.36454 | 6.14919 | 2.17199 | 2.48166 | 0.00035 | 0.014242 | yes |
| XLOC_002873 | WT | PAX6DKO | OK | 4.1144 | 18.8736 | 2.19762 | 2.85284 | 0.00005 | 0.002735 | yes |
| XLOC_051150 | WT | PAX6DKO | OK | 0.075008 | 0.347829 | 2.21326 | 0.638108 | 0.00005 | 0.002735 | yes |
| XLOC_046221 | WT | PAX6DKO | OK | 0.112199 | 0.529858 | 2.23955 | 2.31334 | 0.00115 | 0.035883 | yes |
| XLOC_045087 | WT | PAX6DKO | OK | 5.77911 | 27.4829 | 2.24962 | 3.02589 | 0.00005 | 0.002735 | yes |
| XLOC_050747 | WT | PAX6DKO | OK | 0.356847 | 1.71057 | 2.2611 | 2.71887 | 0.00005 | 0.002735 | yes |
| XLOC_007587 | WT | PAX6DKO | OK | 0.353669 | 1.76078 | 2.31575 | 2.48798 | 0.00005 | 0.002735 | yes |
| XLOC_042003 | WT | PAX6DKO | OK | 0.135718 | 0.676873 | 2.31827 | 2.46951 | 0.00005 | 0.002735 | yes |
| XLOC_003769 | WT | PAX6DKO | OK | 6.16053 | 30.9249 | 2.32764 | 3.62326 | 0.00005 | 0.002735 | yes |
| XLOC_044934 | WT | PAX6DKO | OK | 0.27613 | 1.41109 | 2.35339 | 1.88149 | 0.0002 | 0.008981 | yes |
| XLOC_027613 | WT | PAX6DKO | OK | 0.53843 | 2.76027 | 2.35798 | 1.8376 | 0.0002 | 0.008981 | yes |
| XLOC_022838 | WT | PAX6DKO | OK | 0.62621 | 3.25431 | 2.37763 | 2.23104 | 0.00055 | 0.020318 | yes |
| XLOC_028330 | WT | PAX6DKO | OK | 1.67974 | 8.82888 | 2.394 | 1.3508 | 0.00045 | 0.017298 | yes |
| XLOC_031003 | WT | PAX6DKO | OK | 0.094246 | 0.501382 | 2.4114 | 1.50172 | 0.0013 | 0.039328 | yes |
| XLOC_001608 | WT | PAX6DKO | OK | 0.504059 | 2.84553 | 2.49703 | 2.56798 | 0.00005 | 0.002735 | yes |
| XLOC_003403 | WT | PAX6DKO | OK | 2.98562 | 17.3049 | 2.53508 | 2.49491 | 0.0003 | 0.012524 | yes |
| XLOC_015958 | WT | PAX6DKO | OK | 0.206745 | 1.19879 | 2.53565 | 1.70275 | 0.0005 | 0.018851 | yes |
| XLOC_009251 | WT | PAX6DKO | OK | 0.630021 | 3.66024 | 2.53847 | 3.45081 | 0.00005 | 0.002735 | yes |
| XLOC_038240 | WT | PAX6DKO | OK | 0.368274 | 2.14254 | 2.54047 | 2.03383 | 0.00005 | 0.002735 | yes |
| XLOC_037482 | WT | PAX6DKO | OK | 0.261605 | 1.60141 | 2.61387 | 1.97763 | 0.00015 | 0.007086 | yes |
| XLOC_045014 | WT | PAX6DKO | OK | 5.44318 | 33.5724 | 2.62475 | 5.6374 | 0.00005 | 0.002735 | yes |
| XLOC_047629 | WT | PAX6DKO | OK | 0.275656 | 1.75278 | 2.6687 | 2.59232 | 0.00005 | 0.002735 | yes |
| XLOC_037508 | WT | PAX6DKO | OK | 0.353579 | 2.27336 | 2.68472 | 1.88461 | 0.00015 | 0.007086 | yes |
| XLOC_022839 | WT | PAX6DKO | OK | 0.239519 | 1.57201 | 2.7144 | 1.6182 | 0.00005 | 0.002735 | yes |
| XLOC_021627 | WT | PAX6DKO | OK | 0.133515 | 0.917594 | 2.78085 | 3.00406 | 0.00005 | 0.002735 | yes |
| XLOC_028833 | WT | PAX6DKO | OK | 0.485298 | 3.33793 | 2.78201 | 3.45453 | 0.00005 | 0.002735 | yes |
| XLOC_013913 | WT | PAX6DKO | OK | 0.344646 | 2.56635 | 2.89653 | 2.28732 | 0.00025 | 0.010784 | yes |
| XLOC_040604 | WT | PAX6DKO | OK | 0.568453 | 4.24851 | 2.90185 | 3.58871 | 0.00005 | 0.002735 | yes |
| XLOC_038604 | WT | PAX6DKO | OK | 0.092679 | 0.748044 | 3.01281 | 1.10653 | 0.00175 | 0.049136 | yes |
| XLOC_034414 | WT | PAX6DKO | OK | 0.127686 | 1.05251 | 3.04317 | 3.24623 | 0.00005 | 0.002735 | yes |
| XLOC_024084 | WT | PAX6DKO | OK | 0.205361 | 1.7297 | 3.07429 | 1.86385 | 0.0007 | 0.02448 | yes |
| XLOC_052291 | WT | PAX6DKO | OK | 0.216904 | 1.84302 | 3.08694 | 1.83488 | 0.00035 | 0.014242 | yes |
| XLOC_050257 | WT | PAX6DKO | OK | 0.049111 | 0.445021 | 3.17976 | 1.34396 | 0.00005 | 0.002735 | yes |
| XLOC_032924 | WT | PAX6DKO | OK | 0.092738 | 0.867763 | 3.22607 | 2.67967 | 0.00065 | 0.023143 | yes |
| XLOC_004239 | WT | PAX6DKO | OK | 0.1633 | 1.53663 | 3.23417 | 2.83008 | 0.00005 | 0.002735 | yes |
| XLOC_019449 | WT | PAX6DKO | OK | 0.265636 | 2.84092 | 3.41884 | 2.93252 | 0.00045 | 0.017298 | yes |
| XLOC_018543 | WT | PAX6DKO | OK | 0.427984 | 6.45736 | 3.91531 | 1.20815 | 0.00015 | 0.007086 | yes |
| XLOC_053103 | WT | PAX6DKO | OK | 0.175146 | 2.87155 | 4.0352 | 2.15261 | 0.00005 | 0.002735 | yes |
| XLOC_015003 | WT | PAX6DKO | OK | 0.390992 | 6.41213 | 4.03559 | 3.09823 | 0.00005 | 0.002735 | yes |
| XLOC_021436 | WT | PAX6DKO | OK | 0.34311 | 5.7042 | 4.05528 | 1.45322 | 0.00005 | 0.002735 | yes |
| XLOC_047531 | WT | PAX6DKO | OK | 0.221946 | 3.77451 | 4.08801 | 2.49886 | 0.00005 | 0.002735 | yes |
| XLOC_022539 | WT | PAX6DKO | OK | 0.092896 | 1.71602 | 4.20732 | 1.67582 | 0.00005 | 0.002735 | yes |
| XLOC_001584 | WT | PAX6DKO | OK | 0.143819 | 2.8023 | 4.28429 | 2.11615 | 0.00005 | 0.002735 | yes |
| XLOC_030625 | WT | PAX6DKO | OK | 0.037112 | 0.743167 | 4.32373 | 3.49567 | 0.00115 | 0.035883 | yes |
| XLOC_025815 | WT | PAX6DKO | OK | 0.102352 | 2.37793 | 4.53809 | 1.95084 | 0.00005 | 0.002735 | yes |
| XLOC_031698 | WT | PAX6DKO | OK | 5.28127 | 124.966 | 4.56451 | 6.94681 | 0.00005 | 0.002735 | yes |
| XLOC_018539 | WT | PAX6DKO | OK | 0.174772 | 4.69986 | 4.74907 | 3.08367 | 0.00005 | 0.002735 | yes |
| XLOC_042579 | WT | PAX6DKO | OK | 0.044093 | 1.68659 | 5.25744 | 2.10568 | 0.00005 | 0.002735 | yes |
| XLOC_013689 | WT | PAX6DKO | OK | 0.07468 | 4.36475 | 5.86904 | 3.75178 | 0.00005 | 0.002735 | yes |
| XLOC_031669 | WT | PAX6DKO | OK | 0.037515 | 2.31809 | 5.94935 | 2.0074 | 0.00005 | 0.002735 | yes |
| XLOC_022563 | WT | PAX6DKO | OK | 0.095407 | 7.08516 | 6.21456 | 3.36088 | 0.00005 | 0.002735 | yes |
| XLOC_030000 | WT | PAX6DKO | OK | 0.052009 | 7.44173 | 7.16075 | 1.21812 | 0.00055 | 0.020318 | yes |
| XLOC_019729 | WT | PAX6DKO | OK | 0.018947 | 2.79714 | 7.20584 | 0.529428 | 0.00005 | 0.002735 | yes |
| XLOC_022262 | WT | PAX6DKO | OK | 0 | 4.8627 | inf | #NAME? | 0.00005 | 0.002735 | yes |
| XLOC_008457 | WT | PAX6DKO | OK | 0 | 0.505603 | inf | #NAME? | 0.00005 | 0.002735 | yes |
| XLOC_030866 | WT | PAX6DKO | OK | 0 | 0.325478 | inf | #NAME? | 0.00005 | 0.002735 | yes |
| XLOC_048664 | WT | PAX6DKO | OK | 0 | 1.1846 | inf | #NAME? | 0.00005 | 0.002735 | yes |
| XLOC_026995 | WT | PAX6DKO | OK | 1.97436 | 0 | #NAME? | #NAME? | 0.00005 | 0.002735 | yes |
| XLOC_050265 | WT | PAX6DKO | OK | 0.40988 | 0 | #NAME? | #NAME? | 0.00005 | 0.002735 | yes |
| XLOC_053024 | WT | PAX6DKO | OK | 0.308936 | 0 | #NAME? | #NAME? | 0.00005 | 0.002735 | yes |
| XLOC_025713 | WT | PAX6DKO | OK | 6.35971 | 0 | #NAME? | #NAME? | 0.00005 | 0.002735 | yes |
| XLOC_027001 | WT | PAX6DKO | OK | 0.354255 | 0 | #NAME? | #NAME? | 0.00005 | 0.002735 | yes |
| XLOC_025714 | WT | PAX6DKO | OK | 0.776716 | 0 | #NAME? | #NAME? | 0.00005 | 0.002735 | yes |
| XLOC_045348 | WT | PAX6DKO | OK | 1.04504 | 0 | #NAME? | #NAME? | 0.00005 | 0.002735 | yes |
| XLOC_006399 | WT | PAX6DKO | OK | 2.05138 | 0 | #NAME? | #NAME? | 0.00005 | 0.002735 | yes |

TABLE 3

| 290 genes (PAX6 KO only) | 109 genes (shared) | 42 genes (PAX6D KO only) |
|---|---|---|
| CBR1 | KMO | AL021368.4, GUSBP4, LINC00680 |
| XIST, XIST_intron, Xist_exon4 | VSX2 | NIPAL2, RNU6-914P |
| TBX2-AS1 | ALDH1A1 | DAPL1, OR7E28P, OR7E89P |
| TBX2 | HSD17B2 | AL021368.2 |
| CYP19A1 | IAH1 | TBX5 |
| CLVS1 | FOXE3 | PAH |
| AL391650.1 | TYRP1 | CTSF |
| IQCA1 | BHMT | ZNF726, ZNF92P3 |
| TYR | DCT | ZNF572 |
| ZNF454 | ZNF667-AS1 | GAD2 |
| AL117339.1, AL117339.2, AL117339.5, AL133217.1, | AC020636.2, CLRN1, MINDY4B | RAX |
| HSD17B7P2, ZNF37A | | |
| ZNF528 | CDH20 | HMX1 |
| SIGLEC7 | ZNF667 | CSMD1 |
| AC064802.1 | PITX3 | APCDD1L-AS1 |
| AC104257.1 | SYNPR | DIO3OS, MIR1247 |
| CTXND1 | CP | EIF3C |
| SIX6 | CLEC19A | AC004160.1 |
| AC011447.2, AC011447.3 | FAM69C | DIO3 |
| ZPLD1 | ERICH5 | LHX9 |
| MS4A6A | CRYBB3 | CLDN16 |
| ZNF440 | BARHL1 | CRYZ |
| ZMAT4 | ATP6V1B1 | DLK1 |
| TRIM67 | PAPPA2 | RELN |
| GLB1L3 | AC034238.1, ESM1 | SIX3-AS1 |
| AC004947.2 | LAMP5 | LINC01833 |
| SPHKAP | MIR34A, MIR34AHG | LMO1 |
| KLHL14 | AC015712.1, AC015712.4, AC015712.5, | CBLN2 |
| | ACO15712.6, ALDH1A3 | |
| SLC30A8 | PAX2 | TCEAL5 |
| ANOS1 | QPCT | RASGRF2 |
| HDX | PPP2R2B, RNA5SP196 | LTBP2 |
| CPA6 | CCL2 | SIX3 |
| KCNB2 | HRH1 | ARPP21 |
| AC064852.1, KCNJ13 | AL589740.1 | KRT17 |
| NTRK2 | DKK1 | TKTL1 |
| ZFP3 | ADAMTS18 | LAMC2 |
| PDE1A | CRYAB | RORB |
| MAB21L2 | GRIA4 | AC012354.1 |
| AL133461.1, INPP5F | CNTN5 | BRINP1 |
| WNT7A | TNFRSF9 | TGFB2, TGFB2-OT1 |
| ELOVL3 | VAX2 | LRP2 |
| GABRG3 | SFRP2 | CRIM1 |
| ALX1 | CCKBR | MAGEA4 |
| NR2F1 | AL353608.3, PGM5 | |
| CFAP77 | CYTIP | |
| POMC | PMEL | |
| AQP5 | LGR5 | |
| SPP1 | CYP1B1, RMDN2-AS1 | |
| ZNF132 | EIF3CL | |
| THSD7B | CPM, PRELID2P1 | |
| IL15 | AC138035.1 | |
| SPHK1 | TMEFF2 | |
| LINC02365 | CRABP1 | |
| PCDH17 | C1orf186 | |
| FGF9 | ANKRD34B | |
| DGKB | GPD1 | |
| ZNF69 | MIR5004, SYNGAP1, ZBTB9 | |
| FOXP2 | MEIS1 | |
| SLN | APCDD1L | |
| AMBN | CRB1 | |
| POU3F2 | SPOCK1 | |
| HAS1 | TNC | |
| OPRK1 | PPP1R14C | |
| SLC45A2 | PROX1 | |
| P2RX3 | GJA3 | |
| POU3F4 | ADRB1 | |
| SCUBE2 | PCDH9 | |
| HPGD | KCNK2 | |
| SLC7A8 | GJD2 | |
| PLPP4 | AMIGO2 | |
| GHRHR | LINC02506 | |
| EPHA3 | HOMER2 | |
| LINC01508 | AC108729.3, PLCH1 | |
| CBLN1 | MAFA | |
| AC239809.3 | SNCA | |
| AC012313.5 | LRRC36 | |
| ZNF578 | FZD5 | |
| COL9A2 | SYNDIG1 | |
| GATA5 | C8orf4 | |
| GATA3-AS1 | C9orf47, S1PR3 | |
| SST | COL14A1 | |
| VCAM1 | PKDCC | |
| LINC00461, MIR9-2 | AC099795.1, SLC2A1 | |
| AC011754.1, CTNNA2 | MITF | |
| U2AF1L5 | CLDN1 | |
| NR2E1 | MAMDC2 | |
| ZFP42 | NKX3-2 | |
| SPRR3 | MAN1A1 | |
| DIRAS2 | SHISA2 | |
| HTR1E | CSPG5 | |
| AC020928.1 | FAM129A | |
| SUSD4 | AC018709.1, F11-AS1, MTNR1A | |
| SEMA3E | CPAMD8 | |
| TBX3 | CUBN | |
| GREM2 | FAM222A | |
| MOXD1 | BHLHE40 | |
| TMEM132E | ACTC1 | |
| NXPH2 | TSPAN12 | |
| HSPD1P6 | AL117348.1, LEFTY2 | |
| DMBX1 | IL32, RNU1-125P | |
| NCAM1 | AC015522.1 | |
| PXDNL | EFHD2 | |
| VAV3 | COLGALT2 | |
| STS | RAB31 | |
| EGF | ZNF835 | |
| PALMD | ZNF471 | |
| COL25A1 | ZNF736 | |
| ADAMTS16 | AL132657.1, ZNF248 | |
| TNFRSF11A | AL451127.1 | |
| HAS2 | PRODH | |
| LRFN5 | 109 genes (shared) | |
| APCDD1 | KMO | |
| FGF19 | VSX2 | |
| SLC8A3 | ALDH1A1 | |
| CHRM4 | HSD17B2 | |
| HEY2 | IAH1 | |
| SEMA3A | FOXE3 | |
| AL645608.1 | TYRP1 | |
| BARHL2 | BHMT | |
| PI15 | DCT | |
| NDST4 | ZNF667-AS1 | |
| AC022639.1 | AC020636.2, CLRN1, MINDY4B | |
| EBF1 | CDH20 | |
| VSTM4 | ZNF667 | |
| MIR210HG | PITX3 | |
| AC019117.2, AC019117.3, AHR | SYNPR | |
| PLPPR5 | CP | |
| AC098679.1, TMEM144 | CLEC19A | |
| SLC1A3 | FAM69C | |
| CNMD | ERICH5 | |
| AC004540.1 | CRYBB3 | |
| AC067942.1, TMEM150C | BARHL1 | |
| KCND3 | ATP6V1B1 | |
| ANXA1 | PAPPA2 | |
| STRA6 | AC034238.1, ESM1 | |
| SMAD9 | LAMP5 | |
| CRYM | MIR34A, MIR34AHG | |
| NLGN1 | AC015712.1, AC015712.4, AC015712.5, | |
| | AC015712.6, ALDH1A3 | |
| CNNM1 | PAX2 | |
| C9orf64 | QPCT | |
| SLC7A2 | PPP2R2B, RNA5SP196 | |
| BFSP1 | CCL2 | |
| DLX1 | HRH1 | |
| AC011700.1, PROX1-AS1 | AL589740.1 | |
| PLSCR1 | DKK1 | |
| PCDH11X | ADAMTS18 | |
| CXCL12 | CRYAB | |
| ZNF501 | GRIA4 | |
| LMO3 | CNTN5 | |
| RCAN2 | TNFRSF9 | |
| LGR4 | VAX2 | |
| CXCL5 | SFRP2 | |
| ZNF354C | CCKBR | |
| FOXD2-AS1 | AL353608.3, PGM5 | |
| COL13A1 | CYTIP | |
| USP43 | PMEL | |
| AC099524.1, PLCG2, RN7SKP176 | LGR5 | |
| ADCY2 | CYP1B1, RMDN2-AS1 | |
| CYP1B1-AS1, RPL7P12 | EIF3CL | |
| MIR205, MIR205HG | CPM, PRELID2P1 | |
| WNT1 | AC138035.1 | |
| ROBO2 | TMEFF2 | |
| CLCN6, NPPA-AS1, NPPA-AS1_1, NPPA-AS1_2, | CRABP1 | |
| NPPA-AS1_3 | | |
| RAI2 | C1orf186 | |
| PLP1 | ANKRD34B | |
| LINC01018 | GPD1 | |
| GATM | MIR5004, SYNGAP1, ZBTB9 | |
| GPR37 | MEIS1 | |
| ABCA4 | APCDD1L | |
| PDE3A | CRB1 | |
| UNC5C | SPOCK1 | |
| LRRC3B | TNC | |
| ADGRG2 | PPP1R14C | |
| WSCD2 | PROX1 | |
| LIMCH1 | GJA3 | |
| DSC3 | ADRB1 | |
| BMPR1B | PCDH9 | |
| GPR137B | KCNK2 | |
| TCERG1L | GJD2 | |
| FAM198B | AMIGO2 | |
| UAP1L1 | LINC02506 | |
| CA8 | HOMER2 | |
| PLCL1 | AC108729.3, PLCH1 | |
| RASGEF1B | MAFA | |
| DMRT3 | SNCA | |
| HDAC9 | LRRC36 | |
| LUM | FZD5 | |
| NOTUM | SYNDIG1 | |
| AC079779.3 | C8orf4 | |
| LINC00648 | C9orf47, S1PR3 | |
| OLFM3 | COL14A1 | |
| RASSF2 | PKDCC | |
| LDHA | AC099795.1, SLC2A1 | |
| MAP7 | MITF | |
| SOCS2 | CLDN1 | |
| FOXI3 | MAMDC2 | |
| ZNF542P | NKX3-2 | |
| PFKFB4, UCN2 | MAN1A1 | |
| ADCYAP1R1 | SHISA2 | |
| GABRA3 | CSPG5 | |
| NAALAD2 | FAM129A | |
| ITGB8 | AC018709.1, F11-AS1, MTNR1A | |
| MPP1 | CPAMD8 | |
| FAM83B | CUBN | |
| S1PR1 | FAM222A | |
| MIR124-2HG | BHLHE40 | |
| SFRP1 | ACTC1 | |
| TOX | TSPAN12 | |
| WDR76 | AL117348.1, LEFTY2 | |
| CNTN4 | IL32, RNU1-125P | |
| SCG2 | AC015522.1 | |
| RHOBTB1 | EFHD2 | |
| ACKR3 | COLGALT2 | |
| TUBB2A | RAB31 | |
| DCBLD2 | ZNF835 | |
| SUCO | ZNF471 | |
| STC1 | ZNF736 | |
| HK2 | AL132657.1, ZNF248 | |
| FAM111B | AL451127.1 | |
| HTRA1 | PRODH | |
| DSC2 | 109 genes (shared) | |
| SYT1 | KMO | |
| MTUS1 | VSX2 | |
| EFNB2 | ALDH1A1 | |
| AL645608.3, AL645608.5 | HSD17B2 | |
| PLCE1 | IAH1 | |
| TSHZ1 | FOXE3 | |
| CTNND2 | TYRP1 | |
| MAFB | BHMT | |
| SOSTDC1 | DCT | |
| LHFPL6 | ZNF667-AS1 | |
| AC011504.1, ZNF536 | AC020636.2, CLRN1, MINDY4B | |
| AC245748.1, AC245748.2, ZNF112, ZNF229, | CDH20 | |
| ZNF285 | | |
| CXXC4 | ZNF667 | |
| GABRP | PITX3 | |
| P3H2 | SYNPR | |
| DUSP9 | CP | |
| S100A6 | CLEC19A | |
| HMGA2 | FAM69C | |
| CNTFR | ERICH5 | |
| JCAD | CRYBB3 | |
| GPM6A | BARHL1 | |
| SIPA1L2 | ATP6V1B1 | |
| LYPD6B | PAPPA2 | |
| SLC35G1 | AC034238.1, ESM1 | |
| STC2 | LAMP5 | |
| VTCN1 | MIR34A, MIR34AHG | |
| ME1 | AC015712.1, AC015712.4, AC015712.5, | |
| | AC015712.6, ALDH1A3 | |
| SOX3 | PAX2 | |
| ID3 | QPCT | |
| KANK4 | PPP2R2B, RNA5SP196 | |
| GAS7 | CCL2 | |
| TLE1 | HRH1 | |
| LRP4 | AL589740.1 | |
| PRSS35 | DKK1 | |
| FABP3 | ADAMTS18 | |
| SLC35F2 | CRYAB | |
| AL121776.1, EYA2 | GRIA4 | |
| ID2 | CNTN5 | |
| ADAM12 | TNFRSF9 | |
| BNIP3 | VAX2 | |
| AFAP1L2 | SFRP2 | |
| BTBD11, Y_RNA | CCKBR | |
| ZNF595 | AL353608.3, PGM5 | |
| TEX41 | CYTIP | |
| ESRP1 | PMEL | |
| IFI16 | LGR5 | |
| ARL4A | CYP1B1, RMDN2-AS1 | |
| SORCS1 | EIF3CL | |
| TGFBI | CPM, PRELID2P1 | |
| MSX2 | AC138035.1 | |
| LYN | TMEFF2 | |
| TMEM132D | CRABP1 | |
| FGFR3 | C1orf186 | |
| PLEKHG4B | ANKRD34B | |
| EMILIN3 | GPD1 | |
| MYH14 | MIR5004, SYNGAP1, ZBTB9 | |
| PUDP | MEIS1 | |
| SLC2A3 | APCDD1L | |
| CRYAA | CRB1 | |
| LINC01474 | SPOCK1 | |
| AL035252.2 | TNC | |
| AP001360.2 | PPP1R14C | |
| AL136141.1 | PROX1 | |
| RGS22 | GJA3 | |
| ZXDA | ADRB1 | |
| THNSL2 | PCDH9 | |
| AC104117.3 | KCNK2 | |
| AL390198.1 | GJD2 | |
| AL589843.1 | AMIGO2 | |
| ZNF441 | LINC02506 | |

### REFERENCES

1. Antinucci, P., Nikolaou, N., Meyer, M. P., and Hindges, R. (2013). Teneurin-3 specifies morphological and functional connectivity of retinal ganglion cells in the vertebrate visual system. Cell Rep 5, 582-592.
Ashery-Padan, R., Marquardt, T., Zhou, X., and Gruss, P. (2000). Pax6 activity in the lens primordium is required for lens formation and for correct placement of a single retina in the eye. Genes Dev 14, 2701-2711.
Bernard, M., and Voisin, P. (2008). Photoreceptor-specific expression, light-dependent localization, and transcriptional targets of the zinc-finger protein Yin Yang 1 in the chicken retina. J Neurochem 105, 595-604.
Burmeister, M., Novak, J., Liang, M. Y., Basu, S., Ploder, L., Hawes, N. L., Vidgen, D., Hoover, F., Goldman, D., Kalnins, V. I., et al. (1996). Ocular retardation mouse caused by Chx10 homeobox null allele: impaired retinal progenitor proliferation and bipolar cell differentiation. Nat Genet 12, 376-384.
Capes-Davis, A., Tolhurst, O., Dunn, J. M., and Jeffrey, P. L. (2005). Expression of doublecortin (DCX) and doublecortin-like kinase (DCLK) within the developing chick brain. Dev Dyn 232, 457-467.
Capowski, E. E., Wright, L. S., Liang, K., Phillips, M. J., Wallace, K., Petelinsek, A., Hagstrom, A., Pinilla, I., Borys, K., Lien, J., et al. (2016). Regulation of WNT Signaling by VSX2 During Optic Vesicle Patterning in Human Induced Pluripotent Stem Cells. Stem Cells 34, 2625-2634.
Cavodeassi, F., Carreira-Barbosa, F., Young, R. M., Concha, M. L., Allende, M. L., Houart, C., Tada, M., and Wilson, S. W. (2005). Early stages of zebrafish eye formation require the coordinated activity of Wnt11, Fz5, and the Wnt/beta-catenin pathway. Neuron 47, 43-56.
Chambers, S. M., Fasano, C. A., Papapetrou, E. P., Tomishima, M., Sadelain, M., and Studer, L. (2009). Highly efficient neural conversion of human ES and iPS cells by dual inhibition of SMAD signaling. Nat Biotechnol 27, 275-280.
Chen, J., Ma, L., Wang, S., Wang, X., Sun, Y., Gao, L., Li, J., and Zhou, G. (2017). Analysis of expression of transcription factors in early human retina. Int J Dev Neurosci 60, 94-102.
Chen, Y., Cao, J., Xiong, M., Petersen, A. J., Dong, Y., Tao, Y., Huang, C. T., Du, Z., and Zhang, S. C. (2015). Engineering Human Stem Cell Lines with Inducible Gene Knockout using CRISPR/Cas9. Cell Stem Cell 17, 233-244.
Collinson, J. M., Hill, R. E., and West, J. D. (2000). Different roles for Pax6 in the optic vesicle and facial epithelium mediate early morphogenesis of the murine eye. Development 127, 945-956.
Couillard-Despres, S., Winner, B., Schaubeck, S., Aigner, R., Vroemen, M., Weidner, N., Bogdahn, U., Winkler, J., Kuhn, H. G., and Aigner, L. (2005). Doublecortin expression levels in adult brain reflect neurogenesis. Eur J Neurosci 21, 1-14.
Dharmaratne, N., Glendining, K. A., Young, T. R., Tran, H., Sawatari, A., and Leamey, C. A. (2012). Ten-m3 is required for the development of topography in the ipsilateral retinocollicular pathway. PLOS One 7, e43083.
Du, Z. W., Chen, H., Liu, H., Lu, J., Qian, K., Huang, C. L., Zhong, X., Fan, F., and Zhang, S. C. (2015). Generation and expansion of highly pure motor neuron progenitors from human pluripotent stem cells. Nat Commun 6, 6626.
Epstein, J. A., Glaser, T., Cai, J., Jepeal, L., Walton, D. S., and Maas, R. L. (1994). Two independent and interactive DNA-binding subdomains of the Pax6 paired domain are regulated by alternative splicing. Genes Dev 8, 2022-2034.
Georgala, P. A., Carr, C. B., and Price, D. J. (2011). The role of Pax6 in forebrain development. Dev Neurobiol 71, 690-709.
Glaser, T., Walton, D. S., and Maas, R. L. (1992). Genomic structure, evolutionary conservation and aniridia mutations in the human PAX6 gene. Nat Genet 2, 232-239.
Glendining, K. A., Liu, S. C., Nguyen, M., Dharmaratne, N., Nagarajah, R., Iglesias, M. A., Sawatari, A., and Leamey, C. A. (2017). Downstream mediators of Ten-m3 signalling in the developing visual pathway. BMC Neurosci 18, 78.
Green, E. S., Rendahl, K. G., Zhou, S., Ladner, M., Coyne, M., Srivastava, R., Manning, W. C., and Flannery, J. G. (2001). Two animal models of retinal degeneration are rescued by recombinant adeno-associated virus-mediated production of FGF-5 and FGF-18. Mol Ther 3, 507-515.
Grindley, J. C., Davidson, D. R., and Hill, R. E. (1995). The role of Pax-6 in eye and nasal development. Development 121, 1433-1442.
Heavner, W., and Pevny, L. (2012). Eye development and retinogenesis. Cold Spring Harb Perspect Biol 4.
Hill, R. E., Favor, J., Hogan, B. L., Ton, C. C., Saunders, G. F., Hanson, I. M., Prosser, J., Jordan, T., Hastie, N. D., and van Heyningen, V. (1991). Mouse small eye results from mutations in a paired-like homeobox-containing gene. Nature 354, 522-525.
Hogan, B. L., Hirst, E. M., Horsburgh, G., and Hetherington, C. M. (1988). Small eye (Sey): a mouse model for the genetic analysis of craniofacial abnormalities. Development 103 Suppl, 115-119.
Hogan, B. L., Horsburgh, G., Cohen, J., Hetherington, C. M., Fisher, G., and Lyon, M. F. (1986). Small eyes (Sey): a homozygous lethal mutation on chromosome 2 which affects the differentiation of both lens and nasal placodes in the mouse. J Embryol Exp Morphol 97, 95-110.
Horsford, D. J., Nguyen, M. T., Sellar, G. C., Kothary, R., Arnheiter, H., and McInnes, R. R. (2005). Chx10 repression of Mitf is required for the maintenance of mammalian neuroretinal identity. Development 132, 177-187.
Huang, C. T., Tao, Y., Lu, J., Jones, J. R., Fowler, L., Weick, J. P., and Zhang, S. C. (2016). Time-Course Gene Expression Profiling Reveals a Novel Role of Non-Canonical WNT Signaling During Neural Induction. Sci Rep 6, 32600.
Huang, H., Wahlin, K. J., McNally, M., Irving, N. D., and Adler, R. (2008). Developmental regulation of muscleblind-like (MBNL) gene expression in the chicken embryo retina. Dev Dyn 237, 286-296.
Jami, A., Gadi, J., Lee, M. J., Kim, E. J., Lee, M. J., Jung, H. S., Kim, H. H., and Lim, S. K. (2013). Pax6 expressed in osteocytes inhibits canonical Wnt signaling. Mol Cells 35, 305-312.
Jones, L., Lopez-Bendito, G., Gruss, P., Stoykova, A., and Molnar, Z. (2002). Pax6 is required for the normal development of the forebrain axonal connections. Development 129, 5041-5052.
Kamachi, Y., Uchikawa, M., Collignon, J., Lovell-Badge, R., and Kondoh, H. (1998). Involvement of Sox1, 2 and 3 in the early and subsequent molecular events of lens induction. Development 125, 2521-2532.
Kammandel, B., Chowdhury, K., Stoykova, A., Aparicio, S., Brenner, S., and Gruss, P. (1999). Distinct cis-essential modules direct the time-space pattern of the Pax6 gene activity. Dev Biol 205, 79-97.
Kim, J., and Lauderdale, J. D. (2006). Analysis of Pax6 expression using a BAC transgene reveals the presence of a paired-less isoform of Pax6 in the eye and olfactory bulb. Dev Biol 292, 486-505.
Kim, J., and Lauderdale, J. D. (2008). Overexpression of pairedless Pax6 in the retina disrupts corneal development and affects lens cell survival. Dev Biol 313, 434-454.
Kitaoka, T., Aotaki-Keen, A. E., and Hjelmeland, L. M. (1994). Distribution of FGF-5 in the rhesus macaque retina. Invest Ophthalmol Vis Sci 35, 3189-3198.
Kleinjan, D. A., Seawright, A., Childs, A. J., and van Heyningen, V. (2004). Conserved elements in Pax6 intron 7 involved in (auto) regulation and alternative transcription. Dev Biol 265, 462-477.
Klimova, L., and Kozmik, Z. (2014). Stage-dependent requirement of neuroretinal Pax6 for lens and retina development. Development 141, 1292-1302.
Kwon, H. J., and Chung, H. M. (2003). Yin Yang 1, a vertebrate polycomb group gene, regulates antero-posterior neural patterning. Biochem Biophys Res Commun 306, 1008-1013.
Lad, E. M., Cheshier, S. H., and Kalani, M. Y. (2009). Wnt-signaling in retinal development and disease. Stem Cells Dev 18, 7-16.
Lako, M., Lindsay, S., Bullen, P., Wilson, D. I., Robson, S. C., and Strachan, T. (1998). A novel mammalian wnt gene, WNT8B, shows brain-restricted expression in early development, with sharply delimited expression boundaries in the developing forebrain. Hum Mol Genet 7, 813-822.
Lakowski, J., Majumder, A., and Lauderdale, J. D. (2007). Mechanisms controlling Pax6 isoform expression in the retina have been conserved between teleosts and mammals. Dev Biol 307, 498-520.
Li, S., Goldowitz, D., and Swanson, D. J. (2007). The requirement of pax6 for postnatal eye development: evidence from experimental mouse chimeras. Invest Ophthalmol Vis Sci 48, 3292-3300.
Liu, H., Mohamed, O., Dufort, D., and Wallace, V. A. (2003). Characterization of Wnt signaling components and activation of the Wnt canonical pathway in the murine retina. Dev Dyn 227, 323-334.
Liu, H., Thurig, S., Mohamed, O., Dufort, D., and Wallace, V. A. (2006). Mapping canonical Wnt signaling in the developing and adult retina. Invest Ophthalmol Vis Sci 47, 5088-5097.
Liu, W. (2012). Focus on molecules: Wnt8b: a suppressor of early eye and retinal progenitor formation. Exp Eye Res 101, 113-114.
Liu, W., Lagutin, O., Swindell, E., Jamrich, M., and Oliver, G. (2010). Neuroretina specification in mouse embryos requires Six3-mediated suppression of Wnt8b in the anterior neural plate. J Clin Invest 120, 3568-3577.
Lorente, M., Perez, C., Sanchez, C., Donohoe, M., Shi, Y., and Vidal, M. (2006). Homeotic transformations of the axial skeleton of YY1 mutant mice and genetic interaction with the Polycomb group gene Ring1/Ring1A. Mech Dev 123, 312-320.
Mall, M., Kareta, M. S., Chanda, S., Ahlenius, H., Perotti, N., Zhou, B., Grieder, S. D., Ge, X., Drake, S., Euong Ang, C., et al. (2017). Mytll safeguards neuronal identity by actively repressing many non-neuronal fates. Nature 544, 245-249.
Marquardt, T., Ashery-Padan, R., Andrejewski, N., Scardigli, R., Guillemot, F., and Gruss, P. (2001). Pax6 is required for the multipotent state of retinal progenitor cells. Cell 105, 43-55.
Meyer, J. S., Howden, S. E., Wallace, K. A., Verhoeven, A. D., Wright, L. S., Capowski, E. E., Pinilla, I., Martin, J. M., Tian, S., Stewart, R., et al. (2011). Optic vesicle-like structures derived from human pluripotent stem cells facilitate a customized approach to retinal disease treatment. Stem Cells 29, 1206-1218.
Meyer, J. S., Shearer, R. L., Capowski, E. E., Wright, L. S., Wallace, K. A., McMillan, E. L., Zhang, S. C., and Gamm, D. M. (2009). Modeling early retinal development with human embryonic and induced pluripotent stem cells. Proc Natl Acad Sci USA 106, 16698-16703.
Miles, A., and Tropepe, V. (2016). Coordinating progenitor cell cycle exit and differentiation in the developing vertebrate retina. Neurogenesis (Austin) 3, e1161697.
Phillips, M. J., Perez, E. T., Martin, J. M., Reshel, S. T., Wallace, K. A., Capowski, E. E., Singh, R., Wright, L. S., Clark, E. M., Barney, P. M., et al. (2014). Modeling human retinal development with patient-specific induced pluripotent stem cells reveals multiple roles for visual system homeobox 2. Stem Cells 32, 1480-1492.
Quinn, J. C., West, J. D., and Hill, R. E. (1996). Multiple functions for Pax6 in mouse eye and nasal development. Genes Dev 10, 435-446.
Raviv, S., Bharti, K., Rencus-Lazar, S., Cohen-Tayar, Y., Schyr, R., Evantal, N., Meshorer, E., Zilberberg, A., Idelson, M., Reubinoff, B., et al. (2014). PAX6 regulates melanogenesis in the retinal pigmented epithelium through feed-forward regulatory interactions with MITF. PLOS Genet 10, e1004360.
Romm, E., Nielsen, J. A., Kim, J. G., and Hudson, L. D. (2005). Myt1 family recruits histone deacetylase to regulate neural transcription. J Neurochem 93, 1444-1453.
Rousso, D. L., Qiao, M., Kagan, R. D., Yamagata, M., Palmiter, R. D., and Sanes, J. R. (2016). Two Pairs of ON and OFF Retinal Ganglion Cells Are Defined by Intersectional Patterns of Transcription Factor Expression. Cell Rep 15, 1930-1944.
Sato, C., Iwai-Takekoshi, L., Ichikawa, Y., and Kawasaki, H. (2017). Cell type-specific expression of FoxP2 in the ferret and mouse retina. Neurosci Res 117, 1-13.
Shaham, O., Menuchin, Y., Farhy, C., and Ashery-Padan, R. (2012). Pax6: a multi-level regulator of ocular development. Prog Retin Eye Res 31, 351-376.
Tao, Y., and Zhang, S. C. (2016). Neural Subtype Specification from Human Pluripotent Stem Cells. Cell Stem Cell 19, 573-586.
Vasconcelos, F. F., Sessa, A., Laranjeira, C., Raposo, A., Teixeira, V., Hagey, D. W., Tomaz, D. M., Muhr, J., Broccoli, V., and Castro, D. S. (2016). MyT1 Counteracts the Neural Progenitor Program to Promote Vertebrate Neurogenesis. Cell Rep 17, 469-483.
Vuong, L., Brobst, D. E., Saadi, A., Ivanovic, I., and Al-Ubaidi, M. R. (2012). Pattern of expression of p53, its family members, and regulators during early ocular development and in the post-mitotic retina. Invest Ophthalmol Vis Sci 53, 4821-4831.
Wang, X., Xiong, K., Lin, C., Lv, L., Chen, J., Xu, C., Wang, S., Gu, D., Zheng, H., Yu, H., et al. (2015). New medium used in the differentiation of human pluripotent stem cells to retinal cells is comparable to fetal human eye tissue. Biomaterials 53, 40-49.
Wittenmayer, N., Korber, C., Liu, H., Kremer, T., Varoqueaux, F., Chapman, E. R., Brose, N., Kuner, T., and Dresbach, T. (2009). Postsynaptic Neuroligin1 regulates presynaptic maturation. Proc Natl Acad Sci USA 106, 13564-13569.
Wong, L., Power, N., Miles, A., and Tropepe, V. (2015). Mutual antagonism of the paired-type homeobox genes, vsx2 and dmbx1, regulates retinal progenitor cell cycle exit upstream of cond1 expression. Dev Biol 402, 216-228.
Wong, L., Weadick, C. J., Kuo, C., Chang, B. S., and Tropepe, V. (2010). Duplicate dmbx1 genes regulate progenitor cell cycle and differentiation during zebrafish midbrain and retinal development. BMC Dev Biol 10, 100.
Yuan, F., Fang, K. H., Cao, S. Y., Qu, Z. Y., Li, Q., Krencik, R., Xu, M., Bhattacharyya, A., Su, Y. W., Zhu, D. Y., et al. (2015). Efficient generation of region-specific forebrain neurons from human pluripotent stem cells under highly defined condition. Sci Rep 5, 18550.
Zhang, X., Huang, C. T., Chen, J., Pankratz, M. T., Xi, J., Li, J., Yang, Y., Lavaute, T. M., Li, X. J., Ayala, M., et al. (2010). Pax6 is a human neuroectoderm cell fate determinant. Cell Stem Cell 7, 90-100.
Zhong, X., Gutierrez, C., Xue, T., Hampton, C., Vergara, M. N., Cao, L. H., Peters, A., Park, T. S., Zambidis, E. T., Meyer, J. S., et al. (2014). Generation of three-dimensional retinal tissue with functional photoreceptors from human iPSCs. Nat Commun 5, 4047.

1: A method of producing a substantially pure population of human retinal progenitor cells, the method comprising:
(a) culturing human pluripotent stem cells (hPSCs) in suspension culture for about 6 days in a neural induction medium whereby embryoid bodies are formed, wherein the neural induction medium is supplemented with N2 supplement and Non-Essential Amino Acid (NEAA) cell culture supplement beginning on culturing day 3;
(b) dissociating the embryoid bodies formed in step (a) into a single cell suspension;
(c) culturing the single cell suspension as an adherent monolayer for about 15 to about 22 days in a retinal differentiation medium, whereby a substantially pure population comprising human retinal progenitor cells is obtained.

2: The method of claim 1, further comprising sorting the cell population of (c) to isolate Pax6D-expressing human retinal progenitor cells from non-Pax6D-expressing cells.

3: The method of claim 2, wherein Pax6D-expressing human retinal progenitor cells are selected and sorted based on expression of a Pax6D-reporter construct.

4: The method of claim 1, wherein the neural induction medium is a chemically defined medium comprising DMEM/F-12.

5: The method of claim 1, wherein the neural induction medium is E8 medium.

6: The method of claim 1, wherein the retinal differentiation medium is a chemically defined medium comprising DMEM/F-12, B27 supplement, and NEAA cell culture supplement.

7: The method of claim 1, further comprising introducing into the hPSCs an agent that reduces expression of WNT8B and increases expression of retinal progenitor-specific genes.

8: The method of claim 7, wherein the agent is a WNT8B short hairpin interfering RNA (shRNA).

9-16. (canceled)
